SEQUENCE LISTING (255 sequences)

SEQ ID NO: 1 - protein, 503 aa, Homo sapiens
MAGLSDLELRRELQALGFQPGPITDTTRDVYRNKLRRLRGEARLRDEERLREEARPRGEERLREEARLREDAPLRARPAAASPRAEPWLSQPASGSAYATPGAYGDIRPSAASWVGSRGLAYPARPAQLRRRASVRGSSEEDEDARTPDRATQGPGLAARRWWAASPAPARLPSSLLGPDPRPGLRATRAGPAGAARARPEVGRRLERWLSRLLLWASLGLLLVFLGILWVKMGKPSAPQEAEDNMKLLPVDCERKTDEFCQAKQKAALLELLHELYNFLAIQAGNFECGNPENLKSKCIPVMEAQEYIANVTSSSSAKFEAALTWILSSNKDVGIWLKGEDQSELVTTVDKVVCLESAHPRMGVGCRLSRALLTAVTNVLIFFWCLAFLWGLLILLKYRWRKLEEEEQAMYEMVKKIIDVVQDHYVDWEQDMERYPYVGILHVRDSLIPPQSRRRMKRVWDRAVEFLASNESRIQTESHRVAGEDMLVWRWTKPSSFSDSER

SEQ ID NO: 2 - DNA, 1512 bp, Homo sapiens
  source 1..1512: mol_type = other DNA
atggccggcctgtcggacctggaactgcggcgggagctgcaggccctgggcttccagccaggacccatcaccgacaccacccgggatgtctaccgcaacaagctgcgccgcctgcggggcgaggcccggctgcgcgacgaggagcggctgcgggaggaggcccggccgcggggcgaggagcggttacgggaagaggcccggttacgcgaggatgcgccgctgcgcgcccggcccgccgcggcctctccgcgggcggagccctggctctcccagccggcctcgggctcggcctacgcgacccctggggcctacggtgatatccggccctccgcggcttcctgggtagggagccgcggcctcgcctatcctgcccgcccggcgcaactcaggcgccgcgcctcggtccggggcagctccgaggaggacgaggacgcccggacgcccgacagggccacgcagggcccgggtctcgcggcccgccgctggtgggcagcgtctcccgccccggcgcggctgccttcctccctcctcggtcccgacccgcgcccgggcctgcgggcgactcgagcgggccctgctggcgcggcgagggcccggcctgaggtggggcgccggctggagcgctggctctctcggcttctgctctgggccagcctagggctactgctcgtcttcctgggcatcctttgggtgaagatgggcaagccctcagcgccgcaggaggcggaggacaacatgaagttattgccagtggactgtgagagaaaaacagatgagttctgtcaggccaagcagaaggcagccttgctggagctgctgcatgaactctacaatttcctggccatccaagctggtaattttgagtgtggaaatccagagaatctaaaaagcaaatgcattcctgttatggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataa

SEQ ID NO: 3 - DNA, 1512 bp, synthetic construct
  misc_feature 1..1512: note = Synthetic
  source 1..1512: mol_type = other DNA
atggctggactgtctgatctggaactgcggagagaactgcaggccctgggatttcagcctggacctatcaccgacaccaccagggacgtgtaccggaacaagctgagaaggctgagaggcgaggcccggctgagagatgaggaaagactgagagaggaagcccggcctagaggcgaagagagactgcgggaagaagccaggctgagggaagatgctcccctgagagctagacctgccgccgcttctcctagagctgaaccttggctgtctcagcctgccagcggaagcgcttatgctacacctggcgcctacggcgacattagaccatctgctgccagctgggtcggaagcagaggactggcttatcctgccagacctgctcagcttcggagaagggcctctgtgcggggaagctctgaagaggacgaggacgccagaacacccgacagagctacacaaggacctggactggctgctagacgttggtgggctgcttctcctgctcctgctagactgccttcttctctgctgggccccgatcctagaccaggactgagagcaacaagagccggacctgctggtgccgctagagccagaccagaagttggtagacggctggaacggtggctgagcagactgctgctttgggcttctctgggcctgctgctggtgtttctgggcatcctgtgggtcaagatgggcaagcctagcgctcctcaagaggccgaggataacatgaagctgctgcccgtggactgcgagagaaagaccgatgagttctgccaggccaagcagaaggccgctctgctggaactcctgcacgagctgtacaactttctggccatccaggccggcaacttcgagtgcggcaatcccgagaacctgaagtccaagtgcatccccgtgatggaagcccaagagtatatcgccaacgtgaccagcagctccagcgccaagtttgaggctgccctgacatggatcctgtccagcaacaaggacgtcggcatctggctgaagggcgaagatcagagcgagctggtcaccaccgtggacaaggtcgtgtgtctggaaagcgctcaccccagaatgggcgtgggctgtagactgtctagagcactgctgaccgccgtgaccaacgtgctgatcttcttctggtgcctggccttcctgtggggactgctgatcctgctgaagtacagatggcggaagctggaagaggaagaacaggctatgtacgagatggtcaagaaaatcatcgacgtggtgcaggaccactacgtggactgggagcaagacatggaaagatacccctacgtggggatcctgcatgtgcgggacagtctgatccctccacagagcaggcggcggatgaagagagtgtgggatagagccgtggaattcctggcctccaacgagagcagaatccagaccgagtctcacagagtggccggcgaggatatgctcgtttggagatggaccaagccaagcagcttctccgacagcgagagatga

SEQ ID NO: 4 - DNA, 2941 bp, Homo sapiens
  source 1..2941: mol_type = other DNA
gcggcgtcctggccatggccggcctgtcggacctggaactgcggcgggagctgcaggccctgggcttccagccaggacccatcaccgacaccacccgggatgtctaccgcaacaagctgcgccgcctgcggggcgaggcccggctgcgcgacgaggagcggctgcgggaggaggcccggccgcggggcgaggagcggttacgggaagaggcccggttacgcgaggatgcgccgctgcgcgcccggcccgccgcggcctctccgcgggcggagccctggctctcccagccggcctcgggctcggcctacgcgacccctggggcctacggtgatatccggccctccgcggcttcctgggtagggagccgcggcctcgcctatcctgcccgcccggcgcaactcaggcgccgcgcctcggtccggggcagctccgaggaggacgaggacgcccggacgcccgacagggccacgcagggcccgggtctcgcggcccgccgctggtgggcagcgtctcccgccccggcgcggctgccttcctccctcctcggtcccgacccgcgcccgggcctgcgggcgactcgagcgggccctgctggcgcggcgagggcccggcctgaggtggggcgccggctggagcgctggctctctcggcttctgctctgggccagcctagggctactgctcgtcttcctgggcatcctttgggtgaagatgggcaagccctcagcgccgcaggaggcggaggacaacatgaagttattgccagtggactgtgagagaaaaacagatgagttctgtcaggccaagcagaaggcagccttgctggagctgctgcatgaactctacaatttcctggccatccaagctggtaattttgagtgtggaaatccagagaatctaaaaagcaaatgcattcctgttatggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataagccccgggcggggacttgttcccggtcagcctgtcccaggacagcccactccagaggcacaggagggtcaccaggcctgcggtgctgaattcacacttgccttgactttcagtctcgtcctgacacgattccaaaggtctgccatactcttcagaggagttcggctcgcaggaaaatgtgggcttcctttaaagggagggggagaagccagggtgatagcccagactctgccctgttgtgctggcagaaacggggagaagggttgagttttcatgaatagaaaacaagagcagcagctgtcaggcccagctgacaagcgtggggctggtgggctggggcatggccacgccagggagtctgtcacctgggaggtggggcagggatggtgtttcgggtggcgggatggggagcagcttggtccatgtgccttcagggggttgttacttgagagctccagggtgaagggagaaagcctgtgctctggctggctgggcctctatcctgaagagggaaggtgctagaaattagaacaagtgactgtaggggggaccgtctagaaacaccaggcccagacacacggtccctcaggtcaggaggagtgagcagccggcacagcctcctgggaggatcccgaagtagggactggggctgagagcagagaggagggtcgggcagccccagggcgggcgggcccgcctcagtgttgcttctcacagaagctgcgactctagaccaggcctgcagccagaacgccgattccgggagcttgggagccctgcgtcagggcccagagcctcgcacttgctgtgtgcgaagatcgccttgctttactgtgaccacagccccgacgcggggctgtttaacaagaacttcagcgcagccggcgtttctgttaccccggccgcggctcttggcggcgggagaggcgcagtggctgcaggctgccccctgccggccacaagggagcagcgtccgcgccgccgcccaccggccctcccgccagctcctcagcctgctccctgggaggcaggctccagaagcaccccgacggggggccttattaagtgcgagaagcctctccagcgagtcttccccgtgtgtcctcatctgcggcaggcaggagagggggagcaaacaggaaattccacagctgcctccagaaacattaaccacccctctctagtcttactggtattttttgtttccttaataaagctccagtctccctcatttgagcaatactctcatttcctctgatgtctggatgatcggcagggcaggaatctgagcgctggccccgtggtgaggccatgttctcataatcaggctccagccagaaaaataagaaacaccgcaacaggcttcagtatcagggctgaaactgctggatgaataaactatttattaaaaacgga

SEQ ID NO: 5 - protein, 201 aa, Homo sapiens
MEAQEYIANVTSSSSAKFEAALTWILSSNKDVGIWLKGEDQSELVTTVDKVVCLESAHPRMGVGCRLSRALLTAVTNVLIFFWCLAFLWGLLILLKYRWRKLEEEEQAMYEMVKKIIDVVQDHYVDWEQDMERYPYVGILHVRDSLIPPQSRRRMKRVWDRAVEFLASNESRIQTESHRVAGEDMLVWRWTKPSSFSDSER

SEQ ID NO: 6 - DNA, 606 bp, Homo sapiens
  source 1..606: mol_type = other DNA
atggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataa

SEQ ID NO: 7 - DNA, 606 bp, synthetic construct
  misc_feature 1..606: note = Synthetic
  source 1..606: mol_type = other DNA
atggaagcccaagagtatatcgccaacgtgaccagcagcagctccgccaagtttgaggctgccctgacatggatcctgagcagcaacaaggacgtcggcatctggctgaaaggcgaggaccagtctgagctggtcaccaccgtggataaggtcgtgtgcctggaaagcgctcaccccagaatgggcgtgggctgtagactgtctagagcactgctgaccgccgtgaccaacgtgctgatcttcttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggcggatgaagagagtgtgggatagagccgtggaattcctggccagcaacgagagcagaatccagaccgagtctcacagagtggccggcgaggatatgctcgtttggagatggaccaagcctagcagcttcagcgacagcgagagatga

SEQ ID NO: 8 - DNA, 2376 bp, Homo sapiens
  source 1..2376: mol_type = other DNA
cattttgctgtctgcattttaaaagtgccttccttagatgacatctttctaatccccacccttggcttggtaatgctttactccctccaaatgttgtgagtagtctctttttgtattcctttctcaacccacacttaatttcctctgtgtttcttttcttggtctcagtgaagttattgccagtggactgtgagagaaaaacagatgagttctgtcaggccaagcagaaggcagccttgctggagctgctgcatgaactctacaatttcctggccatccaagctggtaattttgagtgtggaaatccagagaatctaaaaagcaaatgcattcctgttatggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataagccccgggcggggacttgttcccggtcagcctgtcccaggacagcccactccagaggcacaggagggtcaccaggcctgcggtgctgaattcacacttgccttgactttcagtctcgtcctgacacgattccaaaggtctgccatactcttcagaggagttcggctcgcaggaaaatgtgggcttcctttaaagggagggggagaagccagggtgatagcccagactctgccctgttgtgctggcagaaacggggagaagggttgagttttcatgaatagaaaacaagagcagcagctgtcaggcccagctgacaagcgtggggctggtgggctggggcatggccacgccagggagtctgtcacctgggaggtggggcagggatggtgtttcgggtggcgggatggggagcagcttggtccatgtgccttcagggggttgttacttgagagctccagggtgaagggagaaagcctgtgctctggctggctgggcctctatcctgaagagggaaggtgctagaaattagaacaagtgactgtaggggggaccgtctagaaacaccaggcccagacacacggtccctcaggtcaggaggagtgagcagccggcacagcctcctgggaggatcccgaagtagggactggggctgagagcagagaggagggtcgggcagccccagggcgggcgggcccgcctcagtgttgcttctcacagaagctgcgactctagaccaggcctgcagccagaacgccgattccgggagcttgggagccctgcgtcagggcccagagcctcgcacttgctgtgtgcgaagatcgccttgctttactgtgaccacagccccgacgcggggctgtttaacaagaacttcagcgcagccggcgtttctgttaccccggccgcggctcttggcggcgggagaggcgcagtggctgcaggctgccccctgccggccacaagggagcagcgtccgcgccgccgcccaccggccctcccgccagctcctcagcctgctccctgggaggcaggctccagaagcaccccgacggggggccttattaagtgcgagaagcctctccagcgagtcttccccgtgtgtcctcatctgcggcaggcaggagagggggagcaaacaggaaattccacagctgcctccagaaacattaaccacccctctctagtcttactggtattttttgtttccttaataaagctccagtctccctcatttgagcaatactctcatttcctctgatgtctggatgatcggcagggcaggaatctgagcgctggccccgtggtgaggccatgttctcataatcaggctccagccagaaaaataagaaacaccgcaacaggcttcagtatcagggctgaaactgctggatgaataaactatttattaaaaacggaaaaaaaaaaaaaaaaaa

SEQ ID NO: 9 - DNA, 2547 bp, Homo sapiens
  source 1..2547: mol_type = other DNA
gcggcgtcctggccatggccggcctgtcggacctggaactgcggcgggagctgcaggccctgggcttccagccaggacccatcaccgacaccacccgggatgtctaccgcaacaagctgcgccgcctgcggggcgaggcccggctgcgcgacgaggagcggctgcgggaggaggcccggccgcggggcgaggagcggttacgggaagaggcccggttacgcgaggatgcgccgctgcgcgcccggcccgccgcggcctctccgcgggcggagccctggctctcccagccggcctcgggctcggcctacgcgacccctggggcctacggtgatatccggccctccgcggcttcctggtgaagttattgccagtggactgtgagagaaaaacagatgagttctgtcaggccaagcagaaggcagccttgctggagctgctgcatgaactctacaatttcctggccatccaagctggtaattttgagtgtggaaatccagagaatctaaaaagcaaatgcattcctgttatggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataagccccgggcggggacttgttcccggtcagcctgtcccaggacagcccactccagaggcacaggagggtcaccaggcctgcggtgctgaattcacacttgccttgactttcagtctcgtcctgacacgattccaaaggtctgccatactcttcagaggagttcggctcgcaggaaaatgtgggcttcctttaaagggagggggagaagccagggtgatagcccagactctgccctgttgtgctggcagaaacggggagaagggttgagttttcatgaatagaaaacaagagcagcagctgtcaggcccagctgacaagcgtggggctggtgggctggggcatggccacgccagggagtctgtcacctgggaggtggggcagggatggtgtttcgggtggcgggatggggagcagcttggtccatgtgccttcagggggttgttacttgagagctccagggtgaagggagaaagcctgtgctctggctggctgggcctctatcctgaagagggaaggtgctagaaattagaacaagtgactgtaggggggaccgtctagaaacaccaggcccagacacacggtccctcaggtcaggaggagtgagcagccggcacagcctcctgggaggatcccgaagtagggactggggctgagagcagagaggagggtcgggcagccccagggcgggcgggcccgcctcagtgttgcttctcacagaagctgcgactctagaccaggcctgcagccagaacgccgattccgggagcttgggagccctgcgtcagggcccagagcctcgcacttgctgtgtgcgaagatcgccttgctttactgtgaccacagccccgacgcggggctgtttaacaagaacttcagcgcagccggcgtttctgttaccccggccgcggctcttggcggcgggagaggcgcagtggctgcaggctgccccctgccggccacaagggagcagcgtccgcgccgccgcccaccggccctcccgccagctcctcagcctgctccctgggaggcaggctccagaagcaccccgacggggggccttattaagtgcgagaagcctctccagcgagtcttccccgtgtgtcctcatctgcggcaggcaggagagggggagcaaacaggaaattccacagctgcctccagaaacattaaccacccctctctagtcttactggtattttttgtttccttaataaagctccagtctccctcatttgagcaatactctcatttcctctgatgtctggatgatcggcagggcaggaatctgagcgctggccccgtggtgaggccatgttctcataatcaggctccagccagaaaaataagaaacaccgcaacaggcttcagtatcagggctgaaactgctggatgaataaactatttattaaaaacgga

SEQ ID NO: 10 - protein, 511 aa, Mus musculus
MAGLSDLELRRELQALGFQPGPITDTTRNVYRNKLRRLRGEARLRDDERLREDAGPREDAGPRGPERQREEARLREEAPLRARPAASVLRSEPWPLSPSPPAPSAASDASGPYGNFGASASPWAASRGLSYPPHAGPGPLRRRASVRGSSEDDEDTRTPDRHAPGRGRHWWAPPSASARPHSALLGADARPGLKGSRTGSAGAGRTRPEVGRWLERCLSRLLLWASLGLLLGFLAILWVKMGKPSAPQEAEDNMKLLPVDCERKTDEFCQAKQKAALLELLHELYNFLAIQAGNFECGNPEKLKSKCIPVLEAQEYIANVTSSPSSRFKAALTWILSSNKDVGIWLKGEDPSELATTVDKVVCLESARPRMGIGCRLSRALLTAVTHVLIFFWCLAFLWGLLILLKYRWRKLEEEEQAMYEMVKKIIDVVQDHYVDWEQDMERYPYVGILHVRDSLIPPQSRRRMKRVWDRAVEFLASNESRIQTESHRVAGEDMLVWRWTKPSSFSDSER

SEQ ID NO: 11 - DNA, 1536 bp, Mus musculus
  source 1..1536: mol_type = other DNA
atggccggcctgtcggacctggagttgcggcgagagctgcaggccctgggcttccagccaggccccatcaccgacaccacgcggaacgtctaccgcaacaagctgcgccgcctgcggggcgaggcccggctgcgcgacgacgagcggctgcgggaggacgccgggccgcgggaggacgccgggccgcggggccccgagcggcagcgggaggaggcccggctacgcgaggaagcgccgctgcgcgcgcggcccgccgccagcgtcctgcgctcggagccctggccgctgtcgccttccccgccggcgcctagcgcggcctccgacgcctcggggccgtacggcaacttcggggcctccgcctctccctgggccgcgagccgcggcctctcctacccgccccacgccgggcccgggccgctgcgacgccgcgcctcggtccggggcagctcggaggatgacgaggatacacgaacgccggacaggcacgccccgggccggggccgccactggtgggccccgccgtcggcctctgcgcggccgcactcggcgctcctcggcgccgacgctcgccccggcctgaagggctcgcgcaccggctcggcgggcgccgggcggacccgacccgaggtgggccggtggctggagcgctgcctgtctcggctcctgctctgggccagcctggggctgctgctcggcttcttggccatcctgtgggtgaagatgggcaagccctcggcgccgcaggaggcggaggacaacatgaaattgttgccggtcgactgtgagagaaaaacagatgagttctgccaggccaagcagaaggcggccctgttggagctgctgcacgaactgtacaacttcctggccatccaggcaggtaattttgaatgtggcaacccggagaagctgaagagcaagtgcatcccagtcctggaggcgcaggagtacatagctaatgtgaccagcagtccctcttcgaggtttaaggctgcgctgacctggatcctgagcagcaacaaggacgtgggcatctggctgaaaggggaagacccatctgagctggcgactacagtggacaaagtggtctgcctggagtccgcccggccccggatgggcataggctgccgcctcagccgcgccctgctcaccgctgtcacccacgtgctcatcttcttctggtgcctggccttcctgtgggggctgctgatcctcctgaagtaccgctggcggaagctggaggaggaggagcaggccatgtatgagatggtgaagaagatcatagatgtggttcaggaccactatgtggactgggaacaggacatggagcgctacccctacgtgggcatcctccacgtgcgggacagtctaatccccccacagagccggcggcgcatgaagcgagtatgggaccgtgctgtggaattcctggcttccaatgaatctcggatccagactgagtcccaccgagtggccggggaggacatgctggtgtggagatggactaagccttcctctttctctgattcagagcgatag

SEQ ID NO: 12 - DNA, 1536 bp, synthetic construct
  misc_feature 1..1536: note = Synthetic
  source 1..1536: mol_type = other DNA
atggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggattctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggaattcctggccagcaacgagtccagaatccagaccgagtctcacagagtggccggcgaggatatgctcgtttggagatggaccaagcctagcagcttctccgacagcgagagatga

SEQ ID NO: 13 - DNA, 1536 bp, synthetic construct
  misc_feature 1..1536: note = Synthetic
  source 1..1536: mol_type = other DNA
atggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggatcctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggagttcctggccagcaacgagtccagaatccagaccgagtctcacagagtggccggcgaggatatgctcgtttggagatggaccaagcctagcagcttctccgacagcgagagatga

SEQ ID NO: 14 - DNA, 2603 bp, Mus musculus
  source 1..2603: mol_type = other DNA
gagggggcggtgtcctggccatggccggcctgtcggacctggagttgcggcgagagctgcaggccctgggcttccagccaggccccatcaccgacaccacgcggaacgtctaccgcaacaagctgcgccgcctgcggggcgaggcccggctgcgcgacgacgagcggctgcgggaggacgccgggccgcgggaggacgccgggccgcggggccccgagcggcagcgggaggaggcccggctacgcgaggaagcgccgctgcgcgcgcggcccgccgccagcgtcctgcgctcggagccctggccgctgtcgccttccccgccggcgcctagcgcggcctccgacgcctcggggccgtacggcaacttcggggcctccgcctctccctgggccgcgagccgcggcctctcctacccgccccacgccgggcccgggccgctgcgacgccgcgcctcggtccggggcagctcggaggatgacgaggatacacgaacgccggacaggcacgccccgggccggggccgccactggtgggccccgccgtcggcctctgcgcggccgcactcggcgctcctcggcgccgacgctcgccccggcctgaagggctcgcgcaccggctcggcgggcgccgggcggacccgacccgaggtgggccggtggctggagcgctgcctgtctcggctcctgctctgggccagcctggggctgctgctcggcttcttggccatcctgtgggtgaagatgggcaagccctcggcgccgcaggaggcggaggacaacatgaaattgttgccggtcgactgtgagagaaaaacagatgagttctgccaggccaagcagaaggcggccctgttggagctgctgcacgaactgtacaacttcctggccatccaggcaggtaattttgaatgtggcaacccggagaagctgaagagcaagtgcatcccagtcctggaggcgcaggagtacatagctaatgtgaccagcagtccctcttcgaggtttaaggctgcgctgacctggatcctgagcagcaacaaggacgtgggcatctggctgaaaggggaagacccatctgagctggcgactacagtggacaaagtggtctgcctggagtccgcccggccccggatgggcataggctgccgcctcagccgcgccctgctcaccgctgtcacccacgtgctcatcttcttctggtgcctggccttcctgtgggggctgctgatcctcctgaagtaccgctggcggaagctggaggaggaggagcaggccatgtatgagatggtgaagaagatcatagatgtggttcaggaccactatgtggactgggaacaggacatggagcgctacccctacgtgggcatcctccacgtgcgggacagtctaatccccccacagagccggcggcgcatgaagcgagtatgggaccgtgctgtggaattcctggcttccaatgaatctcggatccagactgagtcccaccgagtggccggggaggacatgctggtgtggagatggactaagccttcctctttctctgattcagagcgatagggcagggcctgttaccgccagcctgacccagccacccagctggggcaaggacacagacctttggtgctcattcacacctgccttgattgccctccgggtctcaggtctggaacatgtgggcttacttagggtagccgggagaacaggggagcccttcctctatgtcagtattactgactgacaggggagagtggcttgttctccctggataaaaagatgggcagtggttggaaccctggaggtcctgcacacagctagacagcacggtgatcctgaagcccctcgcttggaaggtgggttgggtggtggggcaaagcttggtccatgtgccttttcgcttccagggagcttaaggggaggggaggaacccagcccactcctgcatgggcctctgtccagaaggggacgatgttatagaaggtaggagcagctggaggggaccaagcagaggtggctggggtctggaaggcgtggggagtccagccacagctgcctgtgcctcagagctgctcacaggtcagcagtgccacctgagccagggtggagctcagcctcctcacctacttactgtgcacaggcttcctggctgcttagccacacaggtagggtgcagcatcgtggctgcacactgccctctgcaggccacatggagtagtgtgggccattagccctcggctatggaggacctgctctaaatacacccagtggggggcctcattagctgagagggaggctttcgagcaactcttcaggcgggtctcacctacagcagcaggaggacagcccctggaggtgacgtgcagctgcctccagaagacacaagacacccccttttggacctattgctggtctctgtttcttaataaagtgtggtctccatgtagcagtatgctcatttcggtctccgtgtagcagtatgctcattgccgtgtgtctgggtggtcagcaggaccgggactgagcaggccccagccagaagacaatggcttttgtattgggggtggaactgttggaaaaataaaatatttattgagaacggaaaaaaaa

SEQ ID NO: 15 - protein, 453 aa, Homo sapiens
MWSPEREAEAPAGGDPAGLLPPEWEEDEERMSFLFSAFKRSREVNSTDWDSKMGFWAPLVLSHSRRQGVVRLRLRDLQEAFQRKGSVPLGLATVLQDLLRRGELQRESDFMASVDSSWISWGVGVFLLKPLKWTLSNMLGDNKVPAEEVLVAVELLKEKAEEVYRLYQNSPLSSHPVVALSELSTLCANSCPDERTFYLVLLQLQKEKRVTVLEQNGEKIVKFARGPRAKVSPVNDVDVGVYQLMQSEQLLSRKVESLSQEAERCKEEARRACRAGKKQLALRSLKAKQRTEKRIEALHAKLDTVQGILDRIYASQTDQMVFNAYQAGVGALKLSMKDVTVEKAESLVDQIQELCDTQDEVSQTLAGGVTNGLDFDSEELEKELDILLQDTTKEPLDLPDNPRNRHFTNSVPNPRISDAELEAELEKLSLSEGGLVPSSKSPKRQLEPTLKPL

SEQ ID NO: 16 - DNA, 1362 bp, Homo sapiens
  source 1..1362: mol_type = other DNA
atgtggtccccggagcgggaggccgaggccccagccgggggagacccggcgggccttctgccccccgagtgggaggaggacgaggagcgcatgtccttcctgttctccgctttcaagaggagtcgcgaggtgaacagcaccgactgggacagcaagatgggcttctgggcgccgttggtgctgagccacagccgccgccagggggtggtgcgcctgcgtctgcgggacttgcaggaggcctttcagcgcaaggggagcgtcccgctggggctggccacggtgctgcaggacctgctgcgtcgaggggagctgcagcgggagtcagacttcatggccagtgtagacagcagctggatctcctggggggttggggtcttcctgctgaagcctctcaagtggactctttctaacatgctgggagataataaggttccagctgaggaggtccttgtcgctgtggagctgttgaaggaaaaggctgaggaggtgtatcgtctgtatcagaactcgcccctctcctcccaccccgtggtggccctgtcagagctcagcaccctctgtgctaactcctgcccagatgagaggaccttctacttggtgttgctgcagctgcagaaggagaagagggtcacagtcctcgagcagaacggggagaagattgtgaagtttgcccgagggccacgtgccaaggtctctccagtcaatgacgtagatgttggggtgtaccagctgatgcagagtgaacagcttctctcacgcaaagtggagtccttatcccaggaagcagagaggtgtaaagaagaagcccgccgggcatgccgagcaggaaagaagcagctggcactgaggtctctcaaggccaagcaacggacagagaagcgcatcgaggccttgcatgccaagctggacactgttcaaggcatcctggaccggatctatgcctcccagacagatcagatggtttttaacgcctaccaggctggggtaggagcactcaaactctccatgaaggatgtcacagtggagaaggcagagagcctcgtggatcagatccaagagctctgtgacacccaggatgaagtttctcagactctggctggtggggtaacaaatggcttagattttgacagtgaagaactggagaaggaattggacatcctccttcaggataccaccaaagaacctttggatctgcctgacaacccccgcaataggcattttaccaacagcgtgcctaaccctaggatctcagatgctgaacttgaagctgaacttgagaaactgtccttatcagagggaggtttggtcccaagcagtaaatctccaaaaaggcaattggaaccgactctaaagccattgtag

SEQ ID NO: 17 - DNA, 1362 bp, synthetic construct
  misc_feature 1..1362: note = Synthetic
  source 1..1362: mol_type = other DNA
atgtggtcccccgagcgggaagctgaggcccctgccggaggagatcctgctggactgctgcctccagagtgggaggaagatgaggagagaatgtccttcctgttcagcgccttcaagagatccagagaggtgaactctacagactgggacagcaagatgggcttctgggcccctctggtgctgtctcacagccggcggcagggcgtggtgcggctcagactgcgggacctgcaagaggcttttcagcggaagggcagcgtgcccctcggcctggccaccgtgctgcaggacctgctgcggagaggagaactgcagagagaatctgacttcatggccagcgtggatagcagctggatcagctggggcgtgggcgtctttctgcttaagcctctgaagtggaccctatctaatatgctgggcgacaacaaggtgccagctgaagaggtgctggtggccgtggaactgctgaaggaaaaagccgaggaagtgtacagactgtaccaaaacagccctctgagcagtcatcctgtggtggccctgagcgagctgagcaccctgtgcgccaacagctgccccgacgagcggaccttctacctggtgctgctacaactgcagaaggaaaaacgcgtgaccgtgctggagcagaacggcgagaagatcgtgaagttcgccaggggccctagagccaaagtgtcccctgtaaacgacgtggacgtgggcgtttaccagctgatgcagagcgagcagctgctgagcagaaaggtggaaagcctgagccaggaggccgaacggtgcaaggaggaagctagaagagcctgcagagccggcaagaaacagctggctctgagatctctgaaagccaaacagcgcaccgagaagcggatcgaggccctgcacgcgaagctggatacagtgcagggcatcctggatagaatctacgcctctcaaacagatcagatggtgttcaacgcctatcaggccggcgtgggcgccctgaagctgagcatgaaggacgtgaccgtggagaaggccgagagcctggtcgaccagatccaggagctgtgtgacacccaggatgaggtgtcacagaccctggctggtggcgttacaaatggcctcgactttgacagcgaggaactggaaaaggaactggacatcctgctgcaagacaccaccaaggaacccctggacctgcctgataatcctagaaacagacacttcacaaactccgtgcctaaccccagaattagcgacgccgagctggaagccgagctggaaaaactgagcctgtctgagggcggcctggtccccagcagcaagtctcctaagagacagctggagccaacactgaagcccctgtga

SEQ ID NO: 18 - DNA, 3410 bp, Homo sapiens
  source 1..3410: mol_type = other DNA
gctgcctcctctgactgttatgcagcaggccggcaggtcaataaacttgcttgcctgactttgagtatactcgtcttttctcttggccaaccttacggtagctgtaccagtttgcatttccaccagcaatgaatgagagttcctgttgctctgcatcctcaccagcatttggtgtggtcagtattttggattttcaccattctaatagagattcacggatccatccgggtggtccgggcaccactggcgcccctctgcggctattccccagttccattttctgaagccacaggtcagaagccgctgtacaacgctttgccgaactccagaatcttgaaggagacgtaaggtgcagccacctgccgcgcaggcgcaagcctttctttcggcacaaagaccgtgggaggaggggtcggcgcaagcgctcggtgtctctctgaaaagaacttcatcatactgcctcctggctgacggagcgcagcgcaacgcatgcgccttgaagacttatggaacttatttcacgctcagggcggcggtgacgtgtgaacgagaaggaggtggtcaaggaacggaagccgggagggaacgagggcggaagcggaccagggccaggcttgtgttcgcagccttgccggggctggggttccgatgtggtccccggagcgggaggccgaggccccagccgggggagacccggcgggccttctgccccccgagtgggaggaggacgaggagcgcatgtccttcctgttctccgctttcaagaggagtcgcgaggtgaacagcaccgactgggacagcaagatgggcttctgggcgccgttggtgctgagccacagccgccgccagggggtggtgcgcctgcgtctgcgggacttgcaggaggcctttcagcgcaaggggagcgtcccgctggggctggccacggtgctgcaggacctgctgcgtcgaggggagctgcagcgggagtcagacttcatggccagtgtagacagcagctggatctcctggggggttggggtcttcctgctgaagcctctcaagtggactctttctaacatgctgggagataataaggttccagctgaggaggtccttgtcgctgtggagctgttgaaggaaaaggctgaggaggtgtatcgtctgtatcagaactcgcccctctcctcccaccccgtggtggccctgtcagagctcagcaccctctgtgctaactcctgcccagatgagaggaccttctacttggtgttgctgcagctgcagaaggagaagagggtcacagtcctcgagcagaacggggagaagattgtgaagtttgcccgagggccacgtgccaaggtctctccagtcaatgacgtagatgttggggtgtaccagctgatgcagagtgaacagcttctctcacgcaaagtggagtccttatcccaggaagcagagaggtgtaaagaagaagcccgccgggcatgccgagcaggaaagaagcagctggcactgaggtctctcaaggccaagcaacggacagagaagcgcatcgaggccttgcatgccaagctggacactgttcaaggcatcctggaccggatctatgcctcccagacagatcagatggtttttaacgcctaccaggctggggtaggagcactcaaactctccatgaaggatgtcacagtggagaaggcagagagcctcgtggatcagatccaagagctctgtgacacccaggatgaagtttctcagactctggctggtggggtaacaaatggcttagattttgacagtgaagaactggagaaggaattggacatcctccttcaggataccaccaaagaacctttggatctgcctgacaacccccgcaataggcattttaccaacagcgtgcctaaccctaggatctcagatgctgaacttgaagctgaacttgagaaactgtccttatcagagggaggtttggtcccaagcagtaaatctccaaaaaggcaattggaaccgactctaaagccattgtaggaccctcaagtgaaggaccctcatgtaaaagagagaccaggcttgctgggtgtgtacatagttatttaaacaagaaactctcagaatgtgtttggaagaggagaaaggagaaccactgattttatctggatgctactacttactacaggacagatagaatttctggaagcgatgctccaaaggcttgctcccagctgtatcatggacctgccttctcatctttatagtgccacgatttatacagtcctgtgtctgacctgtcatttcatagcctgcagttcttgccattggcacacttagatttgtcttcacccaccagcttcgttccagcccatgaaggggaaagatttgcagctttgccaaatctgaatcagttcccactcccccctgcggttttttagaggggtttatcctgtcgctaatgtcagttaaatagcttattcctgtcccacttgatttcactggtaagaatgagaatgaaatacttaaaggttttatttgaggggacttagctgccattttatggaaaaacatgtagtataaaaggtttttctccagggtcatttgacagaattctccaggtgttcagagaaagaggtcgccccaagtggcccagagtcgtgacagtccggaggggctggtgtggccggtgggcgctgcctcttcatctgatggagccaggaggtccgccggtgggggaaaggcttcttccgaggcttctgcactcgcctgggaagaggcccagtcactacaggaccctgagatgtccagaccccttatgcaaagaggcaaaattgttgccttggccccatcatggagtaacttaaaaatacatatctatcttccagaacaagccacacaacaacacacctcccctccttgagcctggcacacagcaccgggctctctgaatttggaaagtgtttcattttgtggcctactgctcggggcaggggggctgagccaggatgtgcaatagaagcaggggttgtcccctctgccagatctgtggggagctgcccaggccacccgtgcacctgtcctgcttcctgcagtccccactccctgcctcctttgagggactgggggacttggggtagggggacagacgggaaagggtctgaatgctctggcaatggcgagaagaagcgatcgccacactgccactctgctgcccagccaccaggatttctgcctggagccctttgccctgtccctgctgcctgaggtggcgtagagcagaggagaagaatttgctccaggccaggagcacaaatcgttgttcaatgaatttctctccaactcgaccttggtaaacggaaatgttgggggtgaagagaaacaatcactatttttttcttttttatctggtaaataattaaaagaaaaaccgaacactt

SEQ ID NO: 19 - protein, 451 aa, Mus musculus
MWSPEREAQAPTGGDPAGLLPPEWEEDEERMSFLFSAFKRSREVNSTDWDSKMGFWAPLVLSHSRRQGVVRLRLRDLQEAFQRKGSVPLGLATVLQDLLRRGELQRESDFMASVDSSWISWGVGVFLLKPLKWTLSNMLGDHKVPAEEVLVAVELLKEKAEEVYRLYQNSPLSSHPVVALSELSALCANSCPDERTFYLVLLQLQKEKRVTVLEQNGEKIVKFARGPHAKVSPVNDVDVGVYQLMQSEQLLSRKVESLSQESERCKEEARRACRAGKKQLALRSLKAKQRTEKRIEALHAKLDTVQGILDRIYASQTDQMVFNAYQAGVGALKLSMKDVTVEKAESLVDQIQELCDTQDEVSQTLAGGVTNGLDFDSEELEKELDILLQDTTTEPLSLLETPQETTLYTNSVPKPRILDAELEAELEKLSLSEGGLIPSSKSPKRQLEPTL

SEQ ID NO: 20 - DNA, 1356 bp, Mus musculus
  source 1..1356: mol_type = other DNA
atgtggtccccggagcgggaggcccaggccccgaccgggggagacccggcgggcctcctgccgcccgagtgggaggaggatgaggagcgcatgtcttttctgttctccgcctttaagaggagtcgcgaggtgaacagcaccgactgggacagcaagatgggcttctgggctccgttggtgctgagccacagccgccgccaaggggtggtgcgcctgcgactgcgggacttgcaggaggccttccagcgcaagggcagtgtcccgctggggctggctacggtgctgcaggacctgctgcgtcgaggcgagctgcagcgagagtcagacttcatggccagtgtggacagcagctggatctcctggggggtcggcgtcttcctgctgaagcccctcaagtggactctttctaacatgctgggggatcacaaagttccagcggaggaggtgctggtggctgtggagctgctgaaggaaaaggccgaggaggtgtatcgcctataccagaactcgcccctctcctcccaccctgtggtggccctgtcagagctgagtgctctgtgcgccaactcctgcccggatgagaggaccttctacctcgtgctgctgcagctgcagaaggagaagagagtcactgtcttggagcagaatggagagaagattgtgaagtttgccaggggaccacatgccaaggtatctcctgtgaacgacgtggatgttggggtctaccagctgatgcagagtgagcagctcctctctcggaaggtcgagtctttatcccaagagtctgagaggtgcaaagaggaagcccgccgggcatgccgagcaggaaagaagcagctggcactgaggtctctcaaggccaagcagaggacagagaaacgcattgaggccctgcatgccaagctggacactgttcaaggcatcctggatcgcatctatgcctcccagactgatcagatggtttttaatgcctaccaagctggtgtgggagccttgaaactctccatgaaggatgtcacagtggagaaagcagagagccttgtggatcagatccaggagctttgtgacacgcaagatgaagtttctcagactctggctggtggagtaaccaatggtttagattttgacagtgaggaactggagaaggagttggacatcctccttcaggacaccaccacagaacctttgagcctgctcgaaaccccccaggagacaacgctttataccaacagtgtgcctaagcctaggatcttggatgctgaacttgaagctgaacttgagaaattgtcattatcagaaggaggtttgatcccaagcagtaaatctccaaaaaggcaattggagccaactctctaa

SEQ ID NO: 21 - DNA, 1356 bp, synthetic construct
  misc_feature 1..1356: note = Synthetic
  source 1..1356: mol_type = other DNA
atgtggtcccctgagagagaagcccaagctcctacaggcggagatcctgctggactgttgcctcctgagtgggaagaggacgaggaacggatgagcttcctgttcagcgccttcaagagaagcagagaagtgaacagcaccgactgggacagcaagatgggcttctgggctcctctggtgctgagccactctagaaggcagggcgtcgtcagactgagactgagggatctgcaagaggccttccagagaaagggctctgtgcctctgggactcgctacagtgctgcaggacctgctgagaagaggcgagctgcagagggaaagcgacttcatggccagcgtggacagcagctggatctcttggggagtgggagtgttcctgctgaagcccctgaagtggaccctgagcaacatgctgggcgaccacaaggtgccagctgaagaggtgctggtggctgtggaactgctgaaagaaaaggccgaggaagtgtacaggctgtaccagaacagccctctgagcagccatcctgtggtggctctgtctgagctgtctgccctgtgtgccaacagctgcccagacgagagaaccttctacctggtgctgctgcagctccagaaagaaaaacgcgtgaccgtgctggaacagaacggcgagaagatcgtgaagttcgccagaggaccccacgccaaagtgtccccagtgaacgatgtggacgtgggcgtgtaccagctgatgcagtctgaacagctgctgagcagaaaggtggaaagcctgagccaagagtccgagagatgcaaagaggaagccagaagggcctgcagagccggcaaaaaacagctggccctgagaagcctgaaggccaagcagaggaccgagaagagaatcgaggccctgcacgccaagctggatacagtgcagggcatcctggacagaatctacgccagccagaccgaccagatggtgttcaacgcttaccaggctggcgtgggcgctctgaagctgagcatgaaggacgtgacagtcgagaaggccgagagcctggtggaccagatccaagagctgtgtgacacccaggacgaggtgtcccaaacacttgctggcggcgtgacaaacggcctggacttcgatagcgaggaactggaaaaagagctggacatcctgctccaggacaccaccacagaacctctgagcctgctggaaacccctcaagagacaaccctgtacaccaacagcgtgccaaagcctagaatcctggatgccgagctggaagccgaactcgagaagctgagtctgagcgaaggcggactgatccccagcagcaagagccctaagagacagctggaacccacactgtga

SEQ ID NO: 22 - DNA, 2706 bp, Mus musculus
  source 1..2706: mol_type = other DNA
gacggaagccgagagggacctggggcggaaggggacccgggccaggcttgtgttcggttccttggcggggctggggttccgatgtggtccccggagcgggaggcccaggccccgaccgggggagacccggcgggcctcctgccgcccgagtgggaggaggatgaggagcgcatgtcttttctgttctccgcctttaagaggagtcgcgaggtgaacagcaccgactgggacagcaagatgggcttctgggctccgttggtgctgagccacagccgccgccaaggggtggtgcgcctgcgactgcgggacttgcaggaggccttccagcgcaagggcagtgtcccgctggggctggctacggtgctgcaggacctgctgcgtcgaggcgagctgcagcgagagtcagacttcatggccagtgtggacagcagctggatctcctggggggtcggcgtcttcctgctgaagcccctcaagtggactctttctaacatgctgggggatcacaaagttccagcggaggaggtgctggtggctgtggagctgctgaaggaaaaggccgaggaggtgtatcgcctataccagaactcgcccctctcctcccaccctgtggtggccctgtcagagctgagtgctctgtgcgccaactcctgcccggatgagaggaccttctacctcgtgctgctgcagctgcagaaggagaagagagtcactgtcttggagcagaatggagagaagattgtgaagtttgccaggggaccacatgccaaggtatctcctgtgaacgacgtggatgttggggtctaccagctgatgcagagtgagcagctcctctctcggaaggtcgagtctttatcccaagagtctgagaggtgcaaagaggaagcccgccgggcatgccgagcaggaaagaagcagctggcactgaggtctctcaaggccaagcagaggacagagaaacgcattgaggccctgcatgccaagctggacactgttcaaggcatcctggatcgcatctatgcctcccagactgatcagatggtttttaatgcctaccaagctggtgtgggagccttgaaactctccatgaaggatgtcacagtggagaaagcagagagccttgtggatcagatccaggagctttgtgacacgcaagatgaagtttctcagactctggctggtggagtaaccaatggtttagattttgacagtgaggaactggagaaggagttggacatcctccttcaggacaccaccacagaacctttgagcctgctcgaaaccccccaggagacaacgctttataccaacagtgtgcctaagcctaggatcttggatgctgaacttgaagctgaacttgagaaattgtcattatcagaaggaggtttgatcccaagcagtaaatctccaaaaaggcaattggagccaactctctaaagccattgcaggaactcaagtgaaggaccctcatgtatcagagaggcagccttgcgcgtgtacatagttatttaatgagaaacactgagaagtcgttggaagaggagggaagaaccacttggatgtatctggatgctgctgctcacgccaagacagatacagcttctggaagccatgctatgaaggtgtgctcccagtgatgtcatggacctaccttctcgtctttatagtgccacaatttatacagttctgtgtttgacccgtcgtcttcatagcctgcagttcttgccattggctccgttaggtttgtcttcacccaccagcttcattccagccagtgaaggtgaaaggtttgcagctctgcccatctaagctagccctctcttccactcgtgaggtctttaaactgctcccctagcctgtgcttttctgggaatgggagtcaaacaagttttattcagagagacccaacagctgagtctggagaaacactgtccggaagatggttctctacattcttcgtcacagtctgggtattcgaggctctgaatggttcagggtcatggcagtttccagggtggcttatgtgattgggggccacaaccgcttccatcttagaagtagatctgctgatggagtgggggaaaggattaatctgccttttcaaggtgagcatgagaaacacaaaccctaggccccggggtgtgaaagaagctggattcatgatttggccccattataaaggatgtgttttctggagcaagccactgggcaccacatcaccctcaactggctccctccctgttaggatctggaaagtgtcttatctagtatccaccactcagggcaggggcctgagccaggatgtgcaataagaggcagggcgctgacctgccgcatctgctgcgtgctgcctcctgccggccagcacatctgtgactccaggaactgtaggacaggcagaagtagatctgaatgctctgggggcagcatggagaagagacatgacagccacactgccagtctgctgcccagggacggtggctgcctggacctctttgcactggcccttcctgcttgaggtgacttgagcagaggggagaactttgcctcaggccaggagcacgaatcattgttcaaagagtctctcccactcagccttggcaatggaaatagctgggggtgaagaaaaacaatcactattttttcttttttaatctggtaaataattaaaagagaaaaacaccaggttttgttgttgtctattct

SEQ ID NO: 23 - protein, 911 aa, Homo sapiens
MAAAAASAPQQLSDEELFSQLRRYGLSPGPVTESTRPVYLKKLKKLREEEQQQHRSGGRGNKTRNSNNNNTAAATVAAAGPAAAAAAGMGVRPVSGDLSYLRTPGGLCRISASGPESLLGGPGGASAAPAAGSKVLLGFSSDESDVEASPRDQAGGGGRKDRASLQYRGLKAPPAPLAASEVTNSNSAERRKPHSWWGARRPAGPELQTPPGKDGAVEDEEGEGEDGEERDPETEEPLWASRTVNGSRLVPYSCRENYSDSEEEDDDDVASSRQVLKDDSLSRHRPRRTHSKPLPPLTAKSAGGRLETSVQGGGGLAMNDRAAAAGSLDRSRNLEEAAAAEQGGGCDQVDSSPVPRYRVNAKKLTPLLPPPLTDMDSTLDSSTGSLLKTNNHIGGGAFSVDSPRIYSNSLPPSAAVAASSSLRINHANHTGSNHTYLKNTYNKPKLSEPEEELLQQFKREEVSPTGSFSAHYLSMFLLTAACLFFLILGLTYLGMRGTGVSEDGELSIENPFGETFGKIQESEKTLMMNTLYKLHDRLAQLAGDHECGSSSQRTLSVQEAAAYLKDLGPEYEGIFNTSLQWILENGKDVGIRCVGFGPEEELTNITDVQFLQSTRPLMSFWCRFRRAFVTVTHRLLLLCLGVVMVCVVLRYMKYRWTKEEEETRQMYDMVVKIIDVLRSHNEACQENKDLQPYMPIPHVRDSLIQPHDRKKMKKVWDRAVDFLAANESRVRTETRRIGGADFLVWRWIQPSASCDKILVIPSKVWQGQAFHLDRRNSPPNSLTPCLKIRNMFDPVMEIGDQWHLAIQEAILEKCSDNDGIVHIAVDKNSREGCVYVKCLSPEYAGKAFKALHGSWFDGKLVTVKYLRLDRYHHRFPQALTSNTPLKPSNKHMNSMSHLRLRTGLTNSQGSS

SEQ ID NO: 24 - DNA, 2736 bp, Homo sapiens
  source 1..2736: mol_type = other DNA
atggcggcggcagcagcttcggcgcctcagcagctctcggatgaggagcttttctctcagctccgccgttacggcctgtctcccggaccagtgacggagagcacccgcccggtctacctcaagaagctgaagaagcttcgagaggaagagcagcaacagcaccggtcagggggccgcggcaacaagacgcggaacagtaataacaataacacggcagccgccacggtcgcagccgcgggaccagcggcggcggcggccgcggggatgggggtccggccggtctcgggcgacctctcctacttacggactcctgggggcctgtgccgaatctcggcctctggcccagagagcctcctgggagggcccgggggcgcctccgccgcccccgcggctggcagcaaagtgctgctgggcttcagctcggacgagtcggacgtggaggccagtccccgggaccaggccggcggcggcgggaggaaagaccgggcttcgctccagtaccgcgggctcaaagcgccgccggcgcccctggccgccagcgaggtgactaacagcaactctgcagagcgaaggaagccccactcgtggtggggggccaggaggccggcgggccccgagctgcagaccccgccggggaaagatggagcagtggaggacgaggaaggggagggagaggacggtgaggagagggacccggagaccgaggagccgctctgggcgagccggaccgtgaatggcagccggcttgtcccctacagctgccgggaaaactattcggactcagaggaagaggacgacgacgacgtggcctccagcagacaggtattaaaggacgactccctttcccggcatcggcccagacgaacccatagtaagcctctccccccgctgactgctaaatcggccggcggcaggctggagacttcagttcagggagggggaggactcgcgatgaatgacagggcggcggctgccgggagtctagacaggagccgaaacctcgaagaggcggcggccgcggagcagggaggagggtgtgatcaagtggactccagccccgttcctagataccgtgttaacgctaagaaactgacccctctcctgcccccgccacttactgacatggactcaaccttggattcgtcaacaggctcccttctgaaaaccaataatcatattggcggtggggccttcagtgtggactcccccaggatttattctaacagtctccctcccagtgcggcggtggccgcctctagttcactcaggatcaatcacgccaatcatacgggctccaatcatacctacctgaaaaacacatacaacaaaccgaagctttccgaacccgaagaggaacttctccagcaatttaaacgggaggaggtgtccccaacagggagtttcagtgcccactacttgtcgatgtttctcttaactgctgcctgcttatttttcctaatactgggactgacttacctaggaatgagagggacaggagtatctgaggatggagaactcagcatagaaaacccctttggtgaaacatttggaaaaatacaagaaagtgaaaaaactcttatgatgaacacattatataagcttcatgatcgattggcacagcttgcaggagatcatgaatgtggcagttctagtcaaagaacgctttctgttcaagaggcagctgcgtatttaaaagatttaggtcctgaatatgaaggtatatttaacacttcattgcagtggatcttagaaaatggaaaagatgttggaataaggtgtgttggttttggccctgaggaagaattgacaaatataactgatgtgcagtttttacagtccacaagaccactgatgtctttttggtgtcgttttcgacgtgcttttgttactgtaactcacagattattgttgttatgcttaggtgtagtgatggtttgtgtcgttctgcgttacatgaaatatcgatggacaaaagaagaggaggaaacaaggcagatgtatgatatggtggtaaagattatagatgttttacgaagtcataatgaagcctgccaggaaaacaaagatttacaaccttacatgcctattccacatgtacgcgattccttaatacagcctcatgacaggaaaaaaatgaagaaagtctgggatagagctgttgacttccttgctgctaatgagtctagagttcgcacggaaacacgaagaataggtggtgcagattttctggtttggcggtggatccagccttctgcatcctgtgacaaaatattagttataccttctaaagtatggcaaggtcaagcatttcatttagatagaagaaattcaccaccaaatagtttgacaccgtgtctaaagattcggaatatgtttgatcccgttatggaaataggggatcagtggcatttggcaattcaagaagcaattttagaaaaatgcagtgataatgatggcattgttcacattgcagtagacaaaaattcacgtgagggttgtgtatatgttaaatgtctgtctccagaatatgctggaaaggcttttaaagcattgcatggctcttggtttgatgggaaattggttacagtaaaatatttacgactagatagataccaccatcgctttccccaggctctcacttccaacactccattgaagccatcaaataaacatatgaactccatgtctcatcttcgtcttcggactggcctaaccaattctcaaggaagttcctga

SEQ ID NO: 25 - DNA, 2736 bp, synthetic construct
  misc_feature 1..2736: note = Synthetic
  source 1..2736: mol_type = other DNA
atggctgctgctgcagcttctgctcctcagcagctgtccgacgaggaactgtttagccagctgcggagatacggactgagccctggacctgtgaccgagagcacaagacccgtgtacctgaagaagctgaaaaagctgcgcgaggaagaacagcagcaacacagatctggcggcagaggcaacaagaccagaaacagcaacaacaacaataccgccgctgccaccgttgccgctgctggacctgccgccgcagctgccgctggaatgggagttagaccagtgtctggcgacctgagctacctgagaacacctggcggcctgtgcagaatctctgcctctggacctgagtctctgctcggaggacctggcggagcttctgcagctccagctgctggatctaaggtgctgctgggctttagcagcgacgagagcgacgtggaagctagccctagagatcaagctggcggaggcggcagaaaggatagagccagcctgcagtacagaggcctgaaagctcctcctgctcctctggccgctagcgaagtgaccaatagcaatagcgccgagcggcggaagcctcactcttggtggggagctagaaggcctgcaggacccgaactgcaaacacctcctggaaaggatggcgccgtggaagatgaagaaggcgaaggcgaggacggcgaggaaagagatcctgagacagaggaacctctgtgggccagcagaaccgtgaatggctctagactggtgccctacagctgcagagagaactacagcgactccgaggaagaggacgacgacgacgtggcatctagcagacaggtgctgaaggacgacagcctgagcagacacagacccagaagaacccacagcaagcccctgcctccactgacagctaaatctgccggtggcagactggaaaccagcgttcaaggcggcggaggcctggccatgaatgatagagctgctgccgcaggcagcctggacagaagcagaaatctggaagaagccgctgccgccgaacaaggcggaggatgtgatcaggtggacagcagccctgtgcctcggtacagagtgaacgccaagaaactgacccctctgctgccacctcctctgaccgatatggatagcaccctggatagcagcaccggcagcctgctgaaaacaaacaaccacatcggcggaggggccttctccgtggatagcccaagaatctacagcaacagcctgccaccatctgccgccgtggctgcttcttctagcctgagaatcaaccacgccaaccacaccggctccaatcacacctatctgaagaacacctacaacaagcctaagctgagcgagcctgaagaggaactgctccagcagtttaagcgggaagaagtgtcccctaccggcagcttctctgcccactacctgagcatgtttctgctgaccgccgcctgcctgttctttctgatcctgggcctgacctacctgggcatgagaggaacaggcgtgtccgaggatggcgagctgagcatcgagaaccctttcggcgagacattcggcaagatccaagagtccgagaaaaccctgatgatgaacaccctgtacaagctgcacgaccggctggctcaactggccggcgatcatgagtgtggcagctctagccagagaaccctgagcgtgcaagaggctgccgcctacctgaaagatctgggccctgagtacgagggcatcttcaacacatccctgcagtggatcctggaaaacggcaaggacgtgggcatcagatgcgtcggctttggccccgaagaagaactgaccaatattaccgacgtgcagttcctgcagagcacccggcctctgatgagcttctggtgcagattcagacgggccttcgtgaccgtgacacacagattgctgctgctctgtctgggcgtcgtgatggtttgcgtggtgctgcggtacatgaagtaccggtggaccaaagaggaagaagagacacggcagatgtacgacatggtggtcaagatcatcgacgtgctgcggagccacaacgaggcctgccaagagaacaaggacctgcagccttacatgcccattcctcacgtgcgggactccctgatccagcctcacgaccggaagaaaatgaagaaagtgtgggacagagccgtggacttcctggccgccaatgaatccagagtgcggaccgagacaagacggattggcggagccgattttctcgtgtggcggtggattcagcctagcgccagctgtgacaagatcctggtcatccccagcaaagtgtggcaaggacaggccttccacctggaccgcagaaactccccacctaatagcctgacaccttgcctgaagatccgcaacatgttcgaccccgtgatggaaatcggcgaccagtggcacctggccatccaagaggccattctggaaaagtgcagcgacaacgacggcatcgtgcacattgccgtggacaagaactcccgcgagggctgcgtgtacgtgaaatgtctgagcccagagtacgccggcaaggcctttaaagccctgcacggcagttggttcgacggcaagctggtcaccgtgaagtacctgcggctggacagataccaccacagattccctcaggctctgaccagcaacacccctctgaagccctccaacaagcacatgaacagcatgagccacctgaggctgagaaccggcctgacaaatagccagggcagctcttga

SEQ ID NO: 26 - DNA, 4780 bp, Homo sapiens
  source 1..4780: mol_type = other DNA
accctggagagaaaatggcggcggcagcagcttcggcgcctcagcagctctcggatgaggagcttttctctcagctccgccgttacggcctgtctcccggaccagtgacggagagcacccgcccggtctacctcaagaagctgaagaagcttcgagaggaagagcagcaacagcaccggtcagggggccgcggcaacaagacgcggaacagtaataacaataacacggcagccgccacggtcgcagccgcgggaccagcggcggcggcggccgcggggatgggggtccggccggtctcgggcgacctctcctacttacggactcctgggggcctgtgccgaatctcggcctctggcccagagagcctcctgggagggcccgggggcgcctccgccgcccccgcggctggcagcaaagtgctgctgggcttcagctcggacgagtcggacgtggaggccagtccccgggaccaggccggcggcggcgggaggaaagaccgggcttcgctccagtaccgcgggctcaaagcgccgccggcgcccctggccgccagcgaggtgactaacagcaactctgcagagcgaaggaagccccactcgtggtggggggccaggaggccggcgggccccgagctgcagaccccgccggggaaagatggagcagtggaggacgaggaaggggagggagaggacggtgaggagagggacccggagaccgaggagccgctctgggcgagccggaccgtgaatggcagccggcttgtcccctacagctgccgggaaaactattcggactcagaggaagaggacgacgacgacgtggcctccagcagacaggtattaaaggacgactccctttcccggcatcggcccagacgaacccatagtaagcctctccccccgctgactgctaaatcggccggcggcaggctggagacttcagttcagggagggggaggactcgcgatgaatgacagggcggcggctgccgggagtctagacaggagccgaaacctcgaagaggcggcggccgcggagcagggaggagggtgtgatcaagtggactccagccccgttcctagataccgtgttaacgctaagaaactgacccctctcctgcccccgccacttactgacatggactcaaccttggattcgtcaacaggctcccttctgaaaaccaataatcatattggcggtggggccttcagtgtggactcccccaggatttattctaacagtctccctcccagtgcggcggtggccgcctctagttcactcaggatcaatcacgccaatcatacgggctccaatcatacctacctgaaaaacacatacaacaaaccgaagctttccgaacccgaagaggaacttctccagcaatttaaacgggaggaggtgtccccaacagggagtttcagtgcccactacttgtcgatgtttctcttaactgctgcctgcttatttttcctaatactgggactgacttacctaggaatgagagggacaggagtatctgaggatggagaactcagcatagaaaacccctttggtgaaacatttggaaaaatacaagaaagtgaaaaaactcttatgatgaacacattatataagcttcatgatcgattggcacagcttgcaggagatcatgaatgtggcagttctagtcaaagaacgctttctgttcaagaggcagctgcgtatttaaaagatttaggtcctgaatatgaaggtatatttaacacttcattgcagtggatcttagaaaatggaaaagatgttggaataaggtgtgttggttttggccctgaggaagaattgacaaatataactgatgtgcagtttttacagtccacaagaccactgatgtctttttggtgtcgttttcgacgtgcttttgttactgtaactcacagattattgttgttatgcttaggtgtagtgatggtttgtgtcgttctgcgttacatgaaatatcgatggacaaaagaagaggaggaaacaaggcagatgtatgatatggtggtaaagattatagatgttttacgaagtcataatgaagcctgccaggaaaacaaagatttacaaccttacatgcctattccacatgtacgcgattccttaatacagcctcatgacaggaaaaaaatgaagaaagtctgggatagagctgttgacttccttgctgctaatgagtctagagttcgcacggaaacacgaagaataggtggtgcagattttctggtttggcggtggatccagccttctgcatcctgtgacaaaatattagttataccttctaaagtatggcaaggtcaagcatttcatttagatagaagaaattcaccaccaaatagtttgacaccgtgtctaaagattcggaatatgtttgatcccgttatggaaataggggatcagtggcatttggcaattcaagaagcaattttagaaaaatgcagtgataatgatggcattgttcacattgcagtagacaaaaattcacgtgagggttgtgtatatgttaaatgtctgtctccagaatatgctggaaaggcttttaaagcattgcatggctcttggtttgatgggaaattggttacagtaaaatatttacgactagatagataccaccatcgctttccccaggctctcacttccaacactccattgaagccatcaaataaacatatgaactccatgtctcatcttcgtcttcggactggcctaaccaattctcaaggaagttcctgaaaagattttcttccatttctaagactgttatttacaataggaaaattcctgtttggctttttgtcttcctttttaaatgctttttgtatgtaatatttttattgatgaatacatctctgaacgttccagaagtcttaaggttccaaagggatttagcagtgaggcagcaatgctgagtaggtaggataattatttcattcagtttttggagctcagttaagccaatacatttaaagttttgcatgaggaacactgactttattaagcattttcagatgtggtggttgtatttttgccccaagaagtgtttggataaccacacaaaagcatgatgaaaaggcttcttgtagtcccataatttcttgtgaactaatgttgtgaatttttgtatacagtcacctgcatagttcctacaggctaatgtggtaaaactgttgatttcccaatttaaccttaggtttctgttgcttataagcgattcatttgctacggctggaatatgggaaaattaatttgggcttagtggttacaaatatgtgtaagtggatgtatatgtacttaaactggctttgtatatatgtataaatgctggtggtggtgaaagtagtcttgttttatgaggatgtctgcattaaagcagtaaaataagctttccattttattcataatctaatgtgtgtgtatatatgtatgtgtgtatgtgtgtgtatatatagatgtatatatatacacacacagagatatatacatatggctgtacttttgcatagatcaaacagccaaacacctggaagtattagatacaagtttaaaatatcttttataggttttatataaaaatgtctgagtatgattttgtgtgaaagttctgataccagttgtaatagagtcaaatttatgtgagcaataaagaagaaattggcagatattggaaaaatattttgtgaaaagctgtatttattataaatactagggccatgacatagtaccattggggttaagtcattttcccaatctatttataatttaatgatatgttagtgcctctgttatatgtcaagtttaaagcagggcacattgttgcagcaaaatgtgtatttgacaaattatacttgcaattttgggtcatgaaagtttgcaatatagtaaatgcacgattgactgttgctttgtgcctcagtatttaatttgtttcagtaaacttactttattactgtttatgatttcagataaaaatagttgctgagtaaattttacttgtaatctacaattggctttttaaaaataacctgttgatatataattgtcagtccaaatgaatatgtgaaacagctggaattgtacaaattttggttgtatttaaagctcagtttcctttttgttttattgtatgcgttgggtttgcagcatgaacttgcacagataatgcacgttttctggttaagtaaacatgatgcacactattctgtaacagaaagccctattgtgccttacctgtgtgcttttgtgggcaccttgtttatgaagaataaaaaatgatttgttatctgaagagaataaattttaaattctcagtttatgtctcagatgctaacgtgtgaaaatataaatatatataatatataaagtaaccagtcttcctgtattttatgtgcatcatagtgatttatctgagcttagtgacccccatcttgtaacctgttgcaagagtgaatgtaaaaaatagttgtggcattttaaaaggtcgcctttgatgcagatgcatctttttcttgcttctaaaacatatttcatgtaaacattgtacatttattattgtaatatatactattatgcagcttattttacctgaaactgttaagccgaccaagatccctccctgcaagacagatgggaatgtgtataataactaggtatttgagaagttctgaagtttgatgtaatctgttacttgtcctgttaaaaaaaggaaaaaattctaattaaaaatttattagtttttttttatgtgtcttggctttttaaaaaca

SEQ ID NO: 27 - protein, 921 aa, Mus musculus
MAAATAAAAPQQLSDEELFSQLRRYGLSPGPVTESTRPVYLKKLKKLREEEQQQQQQQQQQQHRAGGRGNKTRNSNNNNTATAMGGRPGSGDLAYLRSPAGLGRLSASAAESPVAGGSGGAAAVPAAGSKVLLGFSSDESDVEASPREQAGGGGGGGARRDRAALQYRGLRAPPAPPAAGEVTGGHPGERRKPHSWWGARRPAGPEPQPPAAGSDGAAEDADEELADGEDRDPEAEEPLWASRAVNGSRLLPYSSCREHYSDSEEEEEEGEEDGDVAPARQVLKDDSLARHRPRRSHSKPFSALTAKSGGSRQETSVQGGGALAMNDRAAAAGSLDRSRNLEEAAAEPGGGGGGGCGCDPVDSIPRYRAGAKKLAPLLSPPSPDGDSTLESPTGPLLKTNNHIGGGAFGVDSPGLYANSLPPGATAAAAPGTLRINHANHTGSNHTYLKTAYGKPKLCEPEEELLQQFKREEVSPTGSFSAHYLSMFLLTAACLFFLILGLTYLGMRGTGVPEDGGLIKNPFDETFGKIQESEKNLLMSTLYKLHDRLAQIAGDHECGSSSQRMLSVQEAAAYLKNLGPEYEDVFNTSLLWIFKNGKDVGIRCVGYGPEEDLTNITDVQFLQSTRPQMPFWCRFRRAFITVTHRLLLLCLGVVLVCVALRYMRYRWTKEEEETRQMYDMVVKIIDVLRSHNEACQETKDLQPYMPLPHVRDSLIQPQDRKKMKKVWDRAVDFLAANESRVRTETRRVGGADFLVWRWIQPSASCDKTLVIPSKVWQGQAFHLDRRNSPPNSLTPCLKIRNMFDPVMEIGDHWHLAIQEAILEKCSDNDGIVHIAVDRNSREGCVYVKCLSPEYAGKAFKALHGSWFDGKLVTVKYLRLDRYHHRFPQALTCNTPLKPANKHMNSLSHLRLRTGLANSQGSS

SEQ ID NO: 28 - DNA, 2766 bp, synthetic construct
  misc_feature 1..2766: note = Synthetic
  source 1..2766: mol_type = other DNA
atggctgctgctacagctgctgctgctcctcagcagctgagcgacgaggaactgttcagccagctgagaagatacggcctgtctcctggacctgtgaccgagagcacaagacccgtgtacctgaagaagctcaagaagctgagagaggaagaacagcagcaacaacaacagcaacagcaacaacagcacagagctggcggcagaggcaacaagaccagaaactctaacaacaacaataccgccaccgccatgggcggcagacctggatctggcgatctggcctatctgagatctcctgctggactgggcagactgtctgcttctgctgctgagtctcctgtggcaggcggttctggcggagctgctgcagttcctgctgctggatctaaggtgctgctgggcttcagcagcgacgagtctgacgtggaagcctctcctagagaacaagctggtggtggtggcggaggcggcgctagaagagatagagctgctctgcagtacagaggcctgagagcacctcctgctccaccagctgctggcgaagttacaggtggacaccctggcgagagaagaaagccccattcttggtggggagccagaaggccagctggacctgaacctcaacctccagccgctggatctgatggcgctgccgaagatgctgatgaggaactggctgacggcgaggacagagatcctgaggctgaagaacctctgtgggccagcagagctgtgaacggctctagactgctgccttacagcagctgcagagagcactacagcgactccgaggaagaagaggaagagggcgaagaggacggcgacgttgcaccagctagacaggtgctgaaggacgacagcctggccagacacagacccagaagaagccacagcaagcccttcagcgccctgacagctaagagcggaggcagcagacaagagacaagcgttcaaggcggcggagccctggccatgaatgatagagccgctgcagccggaagcctggacagatccagaaacctggaagaggccgcagctgaacctggcggcggaggtggcggtggatgtggatgtgatcctgtggacagcatccccagatacagagccggcgctaagaagctggctcctctgctgtctcctccatctcctgatggcgacagcaccctggaaagccctacaggacccctgctcaagaccaacaaccacatcggaggcggagctttcggcgtggactctccaggactgtacgccaactctcttcctcctggcgctactgccgctgctgcacctggaacactgagaatcaaccacgccaaccacaccggcagcaaccacacatacctgaaaaccgcctacggcaagcccaagctgtgcgagcctgaagaggaactcctgcagcagtttaagcgcgaggaagtgtctcctaccggcagcttctctgcccactacctgagcatgtttctgctgaccgccgcctgcctgttctttctgatcctgggcctgacctacctgggcatgagaggaacaggcgtgccagaagatggcggcctgatcaagaaccccttcgacgagacattcggcaagatccaagagagcgagaagaacctgctgatgtctaccctgtacaagctgcacgatagactggcccagatcgctggcgatcacgagtgtggaagcagctcccagagaatgctgagcgtgcaagaggctgccgcttatctgaagaatctgggccccgagtacgaggacgtgttcaacacatctctgctgtggattttcaagaacggcaaggacgtgggcatcagatgcgtcggctatggccctgaggaagatctgaccaatattaccgacgtgcagtttctgcagtccaccagacctcagatgcccttctggtgcagattcagacgggccttcatcaccgtgacacaccgcctgctgcttctctgtctgggagtcgtgcttgtgtgtgtggccctgcggtacatgagatacagatggaccaaagaagaagaagagacacggcagatgtacgacatggtggtcaagatcatcgacgtgctgagatcccacaacgaggcctgccaagagactaaggacctgcagccttacatgcccctgcctcacgttcgggacagcctgatccagccacaggaccggaagaaaatgaagaaagtgtgggacagagccgtggacttcctggccgctaacgagtctagagtgcggaccgagacaagaagagtcggaggcgccgattttctcgtttggagatggattcagcctagcgccagctgcgacaagacactggtcatccccagcaaagtgtggcagggccaagccttccacctggatagaagaaacagccctcctaacagcctgacaccttgcctgaagatccgcaacatgttcgaccccgtgatggaaatcggcgaccactggcacctggccatccaagaggctatcctggaaaagtgcagcgacaacgacggcatcgtgcatatcgccgtggacagaaactcccgcgagggctgtgtgtacgtgaagtgtctgagccctgagtacgccggcaaggcctttaaggctctgcacggcagttggttcgacggcaagctggtcacggtgaagtacctgagactggatagataccaccacaggttccctcaggctctgacctgcaacacccctctgaagcctgccaacaagcacatgaactccctgagccatctgagactgagaaccggcctggctaacagccagggcagttcttaa

SEQ ID NO: 29 - protein, 918 aa, Mus musculus
MAAATAAAAPQQLSDEELFSQLRRYGLSPGPVTESTRPVYLKKLKKLREEEQQQQQQQQQQQHRAGGRGNKTRNSNNNNTATAMGGRPGSGDLAYLRSPAGLGRLSASAAESPVAGGSGGAAAVPAAGSKVLLGFSSDESDVEASPREQAGGGGARRDRAALQYRGLRAPPAPPAAGEVTGGHPGERRKPHSWWGARRPAGPEPQPPAAGSDGAAEDADEELADGEDRDPEAEEPLWASRTVNGSRLLPYSSCREHYSDSEEEEEEGEEDGDVAPARQVLKDDSLARHRPRRSHSKPFSALTAKSGGSRQETSVQGGGALAMNDRAAAAGSLDRSRNLEEAAAEPGGGGGGGCGCDPVDSIPRYRAGAKKLAPLLSPPSPDGDSTLESPTGPLLKTNNHIGGGAFGVDSPGLYANSLPPGATAAAAPGTLRINHANHTGSNHTYLKTAYGKPKLCEPEEELLQQFKREEVSPTGSFSAHYLSMFLLTAACLFFLILGLTYLGMRGTGVPEDGGLIKNPFDETFGKIQESEKNLLMSTLYKLHDRLAQIAGDHECGSSSQRMLSVQEAAAYLKNLGPEYEDVFNTSLLWIFKNGKDVGIRCVGYGPEEDLTNITDVQFLQSTRPQMPFWCRFRRAFITVTHRLLLLCLGVVLVCVALRYMRYRWTKEEEETRQMYDMVVKIIDVLRSHNEACQETKDLQPYMPLPHVRDSLIQPQDRKKMKKVWDRAVDFLAANESRVRTETRRVGGADFLVWRWIQPSASCDKTLVIPSKVWQGQAFHLDRRNSPPNSLTPCLKIRNMFDPVMEIGDHWHLAIQEAILEKCSDNDGIVHIAVDRNSREGCVYVKCLSPEYAGKAFKALHGSWFDGKLVTVKYLRLDRYHHRFPQALTCNTPLKPANKHMNSLSHLRLRTGLANSQGSS

SEQ ID NO: 30 - DNA, 2757 bp, Mus musculus
  source 1..2757: mol_type = other DNA
atggcggcggcgacggcggcggcggcgcctcagcagctctcggatgaggagcttttctctcagctccgccgttacggcttatctccgggtcccgtgacggagagcacccggcccgtctacctcaagaagctgaagaagcttcgcgaggaagagcagcagcaacagcagcaacagcagcagcagcagcaccgggcggggggccgcggcaacaagacgcggaacagtaataacaataacacggcgacggcgatggggggccggccgggctccggggacctcgcgtacttacggagccccgcgggcctcggccgcctgtcggcctcggctgccgagagccccgtggcaggaggctccgggggcgcggcggccgtccccgcggcgggcagcaaagtgctgctgggcttcagctcggacgagtcggacgtggaggccagcccgcgggagcaggcgggcggcggcggcgcgcggagggaccgggctgcgctccagtaccgcgggctcagagccccaccggcgcccccggccgcgggcgaggtgacgggcggccacccgggcgagcggaggaagccgcactcgtggtggggggcgcggaggccggcgggccccgagccgcagcccccggcggcggggagcgacggggccgcggaggacgcggacgaggagctggcggacggtgaggaccgcgaccccgaggccgaggagccgctgtgggccagccgaacggtgaacggcagccggcttctcccctacagcagctgccgggagcactactcggactcggaggaggaggaggaggagggggaggaggacggcgacgtggccccggccagacaggtattaaaggacgactccctcgcccggcatcgacccagacggagccatagtaagccgttctcggcgctgactgctaaatctggcggcagccggcaggagacttcggttcagggagggggagcactcgcgatgaatgacagggcggcggccgcggggagtctagacaggagccgaaacctcgaggaggcggcggccgagccgggaggaggaggaggaggagggtgcgggtgcgatccagtggactccattcctcgataccgtgccggcgcgaagaagctggccccgctgctgtcgcctccgagtcccgatggggactccaccttggagtcgcccacggggcccctcctcaaaaccaataatcacataggcggcggggccttcggcgtggattcccccggtctgtatgccaacagcctccctcccggggccaccgccgcggccgcccctggcacactcaggatcaaccacgccaaccatactggctccaatcacacctacctgaaaaccgcctacggcaagccgaagctctgcgagcccgaagaggaacttctccagcagtttaaacgggaggaggtgtccccgacggggagtttcagcgctcactacttgtcgatgttcctcctaaccgctgcctgcttgtttttcctaatactgggactgacttacctaggaatgagagggacgggagtacccgaggacggaggactcataaaaaacccctttgatgaaacatttggaaaaatacaagaaagtgaaaaaaatcttttgatgagcacattgtacaaacttcatgatcgattggcccagattgcaggagaccatgaatgtggcagttctagtcaaaggatgctttctgtccaagaagcagctgcatatttaaaaaatttaggtcctgagtatgaagatgtatttaatacttcattgctgtggatttttaaaaatgggaaagatgttggaataaggtgtgttggttacgggcctgaggaagacttgacaaacataactgatgtgcagtttttacagtccaccaggccccagatgcccttctggtgccgtttccggcgtgctttcattactgtgacccataggttattgttgttatgtttaggtgtagtgctggtgtgcgttgctctccggtacatgagataccgctggactaaggaggaggaggagacgaggcagatgtatgacatggtggtaaagattatagatgttttgcgaagtcataatgaagcttgccaggagactaaagacttacagccttacatgcctcttccacatgtccgcgattccctaatacagccgcaggacaggaaaaagatgaagaaagtctgggatagagctgttgacttccttgctgctaacgaatctagagtccgtacggagactcgaagagtaggcggtgcagactttctagtctggcggtggatccagccctctgcgtcttgtgacaagacattagttataccctctaaagtatggcaaggtcaagcatttcatttagatagaagaaattcaccaccaaatagtttgactccatgtttaaagattcgaaatatgtttgacccggttatggaaataggagatcactggcacctggcgatccaagaagcgattttagagaagtgcagtgacaatgacggcatcgttcatattgccgtagacaggaactcgcgggagggctgtgtctatgttaaatgtttgtctccggaatatgctgggaaggcttttaaagcattgcatggctcttggtttgatggaaaattggttacagtaaaatacttacgactagatagataccaccatcgctttccccaggccctcacttgcaacactcccttgaagccagcaaataaacatatgaactctctgtctcacctgcgcctgcggactggcctagctaattctcaaggaagttcctga

SEQ ID NO: 31 - DNA, 4754 bp, Mus musculus
  source 1..4754: mol_type = other DNA
gaaaatggcggcggcgacggcggcggcggcgcctcagcagctctcggatgaggagcttttctctcagctccgccgttacggcttatctccgggtcccgtgacggagagcacccggcccgtctacctcaagaagctgaagaagcttcgcgaggaagagcagcagcaacagcagcaacagcagcagcagcagcaccgggcggggggccgcggcaacaagacgcggaacagtaataacaataacacggcgacggcgatggggggccggccgggctccggggacctcgcgtacttacggagccccgcgggcctcggccgcctgtcggcctcggctgccgagagccccgtggcaggaggctccgggggcgcggcggccgtccccgcggcgggcagcaaagtgctgctgggcttcagctcggacgagtcggacgtggaggccagcccgcgggagcaggcgggcggcggcggcgcgcggagggaccgggctgcgctccagtaccgcgggctcagagccccaccggcgcccccggccgcgggcgaggtgacgggcggccacccgggcgagcggaggaagccgcactcgtggtggggggcgcggaggccggcgggccccgagccgcagcccccggcggcggggagcgacggggccgcggaggacgcggacgaggagctggcggacggtgaggaccgcgaccccgaggccgaggagccgctgtgggccagccgaacggtgaacggcagccggcttctcccctacagcagctgccgggagcactactcggactcggaggaggaggaggaggagggggaggaggacggcgacgtggccccggccagacaggtattaaaggacgactccctcgcccggcatcgacccagacggagccatagtaagccgttctcggcgctgactgctaaatctggcggcagccggcaggagacttcggttcagggagggggagcactcgcgatgaatgacagggcggcggccgcggggagtctagacaggagccgaaacctcgaggaggcggcggccgagccgggaggaggaggaggaggagggtgcgggtgcgatccagtggactccattcctcgataccgtgccggcgcgaagaagctggccccgctgctgtcgcctccgagtcccgatggggactccaccttggagtcgcccacggggcccctcctcaaaaccaataatcacataggcggcggggccttcggcgtggattcccccggtctgtatgccaacagcctccctcccggggccaccgccgcggccgcccctggcacactcaggatcaaccacgccaaccatactggctccaatcacacctacctgaaaaccgcctacggcaagccgaagctctgcgagcccgaagaggaacttctccagcagtttaaacgggaggaggtgtccccgacggggagtttcagcgctcactacttgtcgatgttcctcctaaccgctgcctgcttgtttttcctaatactgggactgacttacctaggaatgagagggacgggagtacccgaggacggaggactcataaaaaacccctttgatgaaacatttggaaaaatacaagaaagtgaaaaaaatcttttgatgagcacattgtacaaacttcatgatcgattggcccagattgcaggagaccatgaatgtggcagttctagtcaaaggatgctttctgtccaagaagcagctgcatatttaaaaaatttaggtcctgagtatgaagatgtatttaatacttcattgctgtggatttttaaaaatgggaaagatgttggaataaggtgtgttggttacgggcctgaggaagacttgacaaacataactgatgtgcagtttttacagtccaccaggccccagatgcccttctggtgccgtttccggcgtgctttcattactgtgacccataggttattgttgttatgtttaggtgtagtgctggtgtgcgttgctctccggtacatgagataccgctggactaaggaggaggaggagacgaggcagatgtatgacatggtggtaaagattatagatgttttgcgaagtcataatgaagcttgccaggagactaaagacttacagccttacatgcctcttccacatgtccgcgattccctaatacagccgcaggacaggaaaaagatgaagaaagtctgggatagagctgttgacttccttgctgctaacgaatctagagtccgtacggagactcgaagagtaggcggtgcagactttctagtctggcggtggatccagccctctgcgtcttgtgacaagacattagttataccctctaaagtatggcaaggtcaagcatttcatttagatagaagaaattcaccaccaaatagtttgactccatgtttaaagattcgaaatatgtttgacccggttatggaaataggagatcactggcacctggcgatccaagaagcgattttagagaagtgcagtgacaatgacggcatcgttcatattgccgtagacaggaactcgcgggagggctgtgtctatgttaaatgtttgtctccggaatatgctgggaaggcttttaaagcattgcatggctcttggtttgatggaaaattggttacagtaaaatacttacgactagatagataccaccatcgctttccccaggccctcacttgcaacactcccttgaagccagcaaataaacatatgaactctctgtctcacctgcgcctgcggactggcctagctaattctcaaggaagttcctgaaaagatgtcttcactcctaggactgttaatttaaataggaaattcccatttggcttcctgtcttcctttgaaaatgctttttgtatgtaatatttttattgctgaagacagctgtttgagagttctgcaaatcttcaggttccgaaggctttggcagtgaggcaggcagtgcctaggaggcagagaaggacgtctcactcagtttgtgcggctcaggtatgccaggctgcacatgaggagcaccggcttgacagcaagcggttccgagatggtgcttgtaggcgtgcaccaagagtgttagagaaccacggacaagcatggcaaagcagcttgttttgtatttccataatttgtgaactaatgtgaatttttgtatacagtaagccgcatcgttccttacaggccactgtggtaaaaccattcacttcccactttaaccttaggttcctattgcttgtaaaaggttcatttgctgcagctggaatatggggaaatttatttggttttaatggttacatacatgtaagagggtgtacatgtacttacattggcttttgtatatatgtataaatgctgatgatagcgaaagtcttgttttgtgagaatgtctgcattagcagtaaaataagctttctattttactcataatctaatctgtatatatatacatatatatatatgtatatgtgcgtagctgtgtgtctatgtacatatgtagctgcacatatgtacatatccacacagatatatacatatggctgtacagtcatagatgatcaaacagccaaatacctggaagtattagatacatgtttaaaatatcttttatagattttatataaaaatatccgagtatggttttgtgtagaagttctggcaccagctgtaatgagttcaaatttatgtgagcaataaagaagcaattggcagatactggaaaatattttgtgaaaagctgtatttattataaatactgggactgtgacagaactaaagggattatgtcatttcccagtctgtttatagcttaaagaactgttagctcctgttctgtgtccagtgtaaaacagggtgcattgttgcagcaaaatgtactagacaaatttccttggaatgttgggccatgcaagtttgcagtgtagatcgtgcacgaatgaccattggtttgtgcctcagtgtttatttcagtatgatttcagatcagaatagttactgatcaaggtttacttgtaacctaaaaattggctttttgaaaaataacctgctggatagatcattgttggtccaagggaagatgtgaaacagctggaattacatgaatcttaatttttacgttaagttcagttttctttttgttgtgtatgtgtttggtttgcagcatgaacttgcacagataatgcacgttttctggttaagtaaacatgatgcacactattctgtaacagaaaacccttgtgccttacctgtgtgcttttgtgggcaccatgtttatgaagaataaatgatttgttatcagaagagaataaattttaaattctcaatctatgtctcagatgctaatgtgttaaaatatatatatataatatataaagtaaccaatctttctgtattttatgtgcatcatagtgatttatttgagcttagtgacacaatcttgtaacctgttgcaagagtgaatgtaaaataatatagttgtggcatttaaacaggtcaccttttgatgcagatgcatccttttcttgcttctaagatatatttcatgtaaacattgtacatttattattgtaatatatactattatgcagcttattttacctggaactcttaagccgaccaagagtcctccctgtaagacagatgggaatgtgtataataattaggtatttgaaaagttctgctttgatgtaatttgtttcttgttctgttttaaaaaaagaaggaaaacattctaattaaaaatttattaggtttttttaaaaacaaaaaaaaaaaa

SEQ ID NO: 32 - DNA, 6960 bp, synthetic construct
  misc_feature 1..6960: note = Synthetic
  misc_feature 3607..3876: note = hBANF1
  source 1..6960: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtggtaaccgccaccatgacaactagccagaagcaccgagattttgtcgccgagcccatgggtgaaaagcctgtagggagtttggcgggcatcggtgaagtgctgggaaagaaactggaggagaggggctttgataaggcgtatgttgtgctgggtcagtttctcgttctcaaaaaagatgaggatttgtttagggaatggttgaaagacacctgcggcgcgaacgctaaacaatcccgcgattgctttgggtgcctgcgcgaatggtgtgacgcatttttgtaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 33 - protein, 89 aa, Homo sapiens
MTTSQKHRDFVAEPMGEKPVGSLAGIGEVLGKKLEERGFDKAYVVLGQFLVLKKDEDLFREWLKDTCGANAKQSRDCFGCLREWCDAFL

SEQ ID NO: 34 - DNA, 8203 bp, synthetic construct
  misc_feature 1..8203: note = Synthetic
  misc_feature 3600..5111: note = hLEMD2
  source 1..8203: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatggctggactgtctgatctggaactgcggagagaactgcaggccctgggatttcagcctggacctatcaccgacaccaccagggacgtgtaccggaacaagctgagaaggctgagaggcgaggcccggctgagagatgaggaaagactgagagaggaagcccggcctagaggcgaagagagactgcgggaagaagccaggctgagggaagatgctcccctgagagctagacctgccgccgcttctcctagagctgaaccttggctgtctcagcctgccagcggaagcgcttatgctacacctggcgcctacggcgacattagaccatctgctgccagctgggtcggaagcagaggactggcttatcctgccagacctgctcagcttcggagaagggcctctgtgcggggaagctctgaagaggacgaggacgccagaacacccgacagagctacacaaggacctggactggctgctagacgttggtgggctgcttctcctgctcctgctagactgccttcttctctgctgggccccgatcctagaccaggactgagagcaacaagagccggacctgctggtgccgctagagccagaccagaagttggtagacggctggaacggtggctgagcagactgctgctttgggcttctctgggcctgctgctggtgtttctgggcatcctgtgggtcaagatgggcaagcctagcgctcctcaagaggccgaggataacatgaagctgctgcccgtggactgcgagagaaagaccgatgagttctgccaggccaagcagaaggccgctctgctggaactcctgcacgagctgtacaactttctggccatccaggccggcaacttcgagtgcggcaatcccgagaacctgaagtccaagtgcatccccgtgatggaagcccaagagtatatcgccaacgtgaccagcagctccagcgccaagtttgaggctgccctgacatggatcctgtccagcaacaaggacgtcggcatctggctgaagggcgaagatcagagcgagctggtcaccaccgtggacaaggtcgtgtgtctggaaagcgctcaccccagaatgggcgtgggctgtagactgtctagagcactgctgaccgccgtgaccaacgtgctgatcttcttctggtgcctggccttcctgtggggactgctgatcctgctgaagtacagatggcggaagctggaagaggaagaacaggctatgtacgagatggtcaagaaaatcatcgacgtggtgcaggaccactacgtggactgggagcaagacatggaaagatacccctacgtggggatcctgcatgtgcgggacagtctgatccctccacagagcaggcggcggatgaagagagtgtgggatagagccgtggaattcctggcctccaacgagagcagaatccagaccgagtctcacagagtggccggcgaggatatgctcgtttggagatggaccaagccaagcagcttctccgacagcgagagatgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 35 - DNA, 8226 bp, synthetic construct
  misc_feature 1..8226: note = Synthetic
  misc_feature 3607..5142: note = mLemd2
  source 1..8226: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtggtaaccgccaccatggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggattctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggaattcctggccagcaacgagtccagaatccagaccgagtctcacagagtggccggcgaggatatgctcgtttggagatggaccaagcctagcagcttctccgacagcgagagatgaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 36 - DNA, 7297 bp, synthetic construct
  misc_feature 1..7297: note = Synthetic
  misc_feature 3600..4205: note = hLEMD2iso2
  source 1..7297: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatggaagcccaagagtatatcgccaacgtgaccagcagcagctccgccaagtttgaggctgccctgacatggatcctgagcagcaacaaggacgtcggcatctggctgaaaggcgaggaccagtctgagctggtcaccaccgtggataaggtcgtgtgcctggaaagcgctcaccccagaatgggcgtgggctgtagactgtctagagcactgctgaccgccgtgaccaacgtgctgatcttcttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggcggatgaagagagtgtgggatagagccgtggaattcctggccagcaacgagagcagaatccagaccgagtctcacagagtggccggcgaggatatgctcgtttggagatggaccaagcctagcagcttcagcgacagcgagagatgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 37 - DNA, 9427 bp, synthetic construct
  misc_feature 1..9427: note = Synthetic
  misc_feature 3600..6335: note = hLEMD3
  source 1..9427: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatggctgctgctgcagcttctgctcctcagcagctgtccgacgaggaactgtttagccagctgcggagatacggactgagccctggacctgtgaccgagagcacaagacccgtgtacctgaagaagctgaaaaagctgcgcgaggaagaacagcagcaacacagatctggcggcagaggcaacaagaccagaaacagcaacaacaacaataccgccgctgccaccgttgccgctgctggacctgccgccgcagctgccgctggaatgggagttagaccagtgtctggcgacctgagctacctgagaacacctggcggcctgtgcagaatctctgcctctggacctgagtctctgctcggaggacctggcggagcttctgcagctccagctgctggatctaaggtgctgctgggctttagcagcgacgagagcgacgtggaagctagccctagagatcaagctggcggaggcggcagaaaggatagagccagcctgcagtacagaggcctgaaagctcctcctgctcctctggccgctagcgaagtgaccaatagcaatagcgccgagcggcggaagcctcactcttggtggggagctagaaggcctgcaggacccgaactgcaaacacctcctggaaaggatggcgccgtggaagatgaagaaggcgaaggcgaggacggcgaggaaagagatcctgagacagaggaacctctgtgggccagcagaaccgtgaatggctctagactggtgccctacagctgcagagagaactacagcgactccgaggaagaggacgacgacgacgtggcatctagcagacaggtgctgaaggacgacagcctgagcagacacagacccagaagaacccacagcaagcccctgcctccactgacagctaaatctgccggtggcagactggaaaccagcgttcaaggcggcggaggcctggccatgaatgatagagctgctgccgcaggcagcctggacagaagcagaaatctggaagaagccgctgccgccgaacaaggcggaggatgtgatcaggtggacagcagccctgtgcctcggtacagagtgaacgccaagaaactgacccctctgctgccacctcctctgaccgatatggatagcaccctggatagcagcaccggcagcctgctgaaaacaaacaaccacatcggcggaggggccttctccgtggatagcccaagaatctacagcaacagcctgccaccatctgccgccgtggctgcttcttctagcctgagaatcaaccacgccaaccacaccggctccaatcacacctatctgaagaacacctacaacaagcctaagctgagcgagcctgaagaggaactgctccagcagtttaagcgggaagaagtgtcccctaccggcagcttctctgcccactacctgagcatgtttctgctgaccgccgcctgcctgttctttctgatcctgggcctgacctacctgggcatgagaggaacaggcgtgtccgaggatggcgagctgagcatcgagaaccctttcggcgagacattcggcaagatccaagagtccgagaaaaccctgatgatgaacaccctgtacaagctgcacgaccggctggctcaactggccggcgatcatgagtgtggcagctctagccagagaaccctgagcgtgcaagaggctgccgcctacctgaaagatctgggccctgagtacgagggcatcttcaacacatccctgcagtggatcctggaaaacggcaaggacgtgggcatcagatgcgtcggctttggccccgaagaagaactgaccaatattaccgacgtgcagttcctgcagagcacccggcctctgatgagcttctggtgcagattcagacgggccttcgtgaccgtgacacacagattgctgctgctctgtctgggcgtcgtgatggtttgcgtggtgctgcggtacatgaagtaccggtggaccaaagaggaagaagagacacggcagatgtacgacatggtggtcaagatcatcgacgtgctgcggagccacaacgaggcctgccaagagaacaaggacctgcagccttacatgcccattcctcacgtgcgggactccctgatccagcctcacgaccggaagaaaatgaagaaagtgtgggacagagccgtggacttcctggccgccaatgaatccagagtgcggaccgagacaagacggattggcggagccgattttctcgtgtggcggtggattcagcctagcgccagctgtgacaagatcctggtcatccccagcaaagtgtggcaaggacaggccttccacctggaccgcagaaactccccacctaatagcctgacaccttgcctgaagatccgcaacatgttcgaccccgtgatggaaatcggcgaccagtggcacctggccatccaagaggccattctggaaaagtgcagcgacaacgacggcatcgtgcacattgccgtggacaagaactcccgcgagggctgcgtgtacgtgaaatgtctgagcccagagtacgccggcaaggcctttaaagccctgcacggcagttggttcgacggcaagctggtcaccgtgaagtacctgcggctggacagataccaccacagattccctcaggctctgaccagcaacacccctctgaagccctccaacaagcacatgaacagcatgagccacctgaggctgagaaccggcctgacaaatagccagggcagctcttgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 38 - DNA, 9456 bp, synthetic construct
  misc_feature 1..9456: note = Synthetic
  misc_feature 3607..6372: note = mLemd3
  source 1..9456: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtggtaaccgccaccatggctgctgctacagctgctgctgctcctcagcagctgagcgacgaggaactgttcagccagctgagaagatacggcctgtctcctggacctgtgaccgagagcacaagacccgtgtacctgaagaagctcaagaagctgagagaggaagaacagcagcaacaacaacagcaacagcaacaacagcacagagctggcggcagaggcaacaagaccagaaactctaacaacaacaataccgccaccgccatgggcggcagacctggatctggcgatctggcctatctgagatctcctgctggactgggcagactgtctgcttctgctgctgagtctcctgtggcaggcggttctggcggagctgctgcagttcctgctgctggatctaaggtgctgctgggcttcagcagcgacgagtctgacgtggaagcctctcctagagaacaagctggtggtggtggcggaggcggcgctagaagagatagagctgctctgcagtacagaggcctgagagcacctcctgctccaccagctgctggcgaagttacaggtggacaccctggcgagagaagaaagccccattcttggtggggagccagaaggccagctggacctgaacctcaacctccagccgctggatctgatggcgctgccgaagatgctgatgaggaactggctgacggcgaggacagagatcctgaggctgaagaacctctgtgggccagcagagctgtgaacggctctagactgctgccttacagcagctgcagagagcactacagcgactccgaggaagaagaggaagagggcgaagaggacggcgacgttgcaccagctagacaggtgctgaaggacgacagcctggccagacacagacccagaagaagccacagcaagcccttcagcgccctgacagctaagagcggaggcagcagacaagagacaagcgttcaaggcggcggagccctggccatgaatgatagagccgctgcagccggaagcctggacagatccagaaacctggaagaggccgcagctgaacctggcggcggaggtggcggtggatgtggatgtgatcctgtggacagcatccccagatacagagccggcgctaagaagctggctcctctgctgtctcctccatctcctgatggcgacagcaccctggaaagccctacaggacccctgctcaagaccaacaaccacatcggaggcggagctttcggcgtggactctccaggactgtacgccaactctcttcctcctggcgctactgccgctgctgcacctggaacactgagaatcaaccacgccaaccacaccggcagcaaccacacatacctgaaaaccgcctacggcaagcccaagctgtgcgagcctgaagaggaactcctgcagcagtttaagcgcgaggaagtgtctcctaccggcagcttctctgcccactacctgagcatgtttctgctgaccgccgcctgcctgttctttctgatcctgggcctgacctacctgggcatgagaggaacaggcgtgccagaagatggcggcctgatcaagaaccccttcgacgagacattcggcaagatccaagagagcgagaagaacctgctgatgtctaccctgtacaagctgcacgatagactggcccagatcgctggcgatcacgagtgtggaagcagctcccagagaatgctgagcgtgcaagaggctgccgcttatctgaagaatctgggccccgagtacgaggacgtgttcaacacatctctgctgtggattttcaagaacggcaaggacgtgggcatcagatgcgtcggctatggccctgaggaagatctgaccaatattaccgacgtgcagtttctgcagtccaccagacctcagatgcccttctggtgcagattcagacgggccttcatcaccgtgacacaccgcctgctgcttctctgtctgggagtcgtgcttgtgtgtgtggccctgcggtacatgagatacagatggaccaaagaagaagaagagacacggcagatgtacgacatggtggtcaagatcatcgacgtgctgagatcccacaacgaggcctgccaagagactaaggacctgcagccttacatgcccctgcctcacgttcgggacagcctgatccagccacaggaccggaagaaaatgaagaaagtgtgggacagagccgtggacttcctggccgctaacgagtctagagtgcggaccgagacaagaagagtcggaggcgccgattttctcgtttggagatggattcagcctagcgccagctgcgacaagacactggtcatccccagcaaagtgtggcagggccaagccttccacctggatagaagaaacagccctcctaacagcctgacaccttgcctgaagatccgcaacatgttcgaccccgtgatggaaatcggcgaccactggcacctggccatccaagaggctatcctggaaaagtgcagcgacaacgacggcatcgtgcatatcgccgtggacagaaactcccgcgagggctgtgtgtacgtgaagtgtctgagccctgagtacgccggcaaggcctttaaggctctgcacggcagttggttcgacggcaagctggtcacggtgaagtacctgagactggatagataccaccacaggttccctcaggctctgacctgcaacacccctctgaagcctgccaacaagcacatgaactccctgagccatctgagactgagaaccggcctggctaacagccagggcagttcttaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 39 - DNA, 8053 bp, synthetic construct
  misc_feature 1..8053: note = Synthetic
  misc_feature 3600..4961: note = hCHMP7
  source 1..8053: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtgccaccatgtggtcccccgagcgggaagctgaggcccctgccggaggagatcctgctggactgctgcctccagagtgggaggaagatgaggagagaatgtccttcctgttcagcgccttcaagagatccagagaggtgaactctacagactgggacagcaagatgggcttctgggcccctctggtgctgtctcacagccggcggcagggcgtggtgcggctcagactgcgggacctgcaagaggcttttcagcggaagggcagcgtgcccctcggcctggccaccgtgctgcaggacctgctgcggagaggagaactgcagagagaatctgacttcatggccagcgtggatagcagctggatcagctggggcgtgggcgtctttctgcttaagcctctgaagtggaccctatctaatatgctgggcgacaacaaggtgccagctgaagaggtgctggtggccgtggaactgctgaaggaaaaagccgaggaagtgtacagactgtaccaaaacagccctctgagcagtcatcctgtggtggccctgagcgagctgagcaccctgtgcgccaacagctgccccgacgagcggaccttctacctggtgctgctacaactgcagaaggaaaaacgcgtgaccgtgctggagcagaacggcgagaagatcgtgaagttcgccaggggccctagagccaaagtgtcccctgtaaacgacgtggacgtgggcgtttaccagctgatgcagagcgagcagctgctgagcagaaaggtggaaagcctgagccaggaggccgaacggtgcaaggaggaagctagaagagcctgcagagccggcaagaaacagctggctctgagatctctgaaagccaaacagcgcaccgagaagcggatcgaggccctgcacgcgaagctggatacagtgcagggcatcctggatagaatctacgcctctcaaacagatcagatggtgttcaacgcctatcaggccggcgtgggcgccctgaagctgagcatgaaggacgtgaccgtggagaaggccgagagcctggtcgaccagatccaggagctgtgtgacacccaggatgaggtgtcacagaccctggctggtggcgttacaaatggcctcgactttgacagcgaggaactggaaaaggaactggacatcctgctgcaagacaccaccaaggaacccctggacctgcctgataatcctagaaacagacacttcacaaactccgtgcctaaccccagaattagcgacgccgagctggaagccgagctggaaaaactgagcctgtctgagggcggcctggtccccagcagcaagtctcctaagagacagctggagccaacactgaagcccctgtgagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 40 - DNA, 8046 bp, synthetic construct
  misc_feature 1..8046: note = Synthetic
  misc_feature 3607..4962: note = mChmp7
  source 1..8046: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgcgctagctgtacaaaaaagcaggctttaaaggaaccaattgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcctcgagactagtggtaaccgccaccatgtggtcccctgagagagaagcccaagctcctacaggcggagatcctgctggactgttgcctcctgagtgggaagaggacgaggaacggatgagcttcctgttcagcgccttcaagagaagcagagaagtgaacagcaccgactgggacagcaagatgggcttctgggctcctctggtgctgagccactctagaaggcagggcgtcgtcagactgagactgagggatctgcaagaggccttccagagaaagggctctgtgcctctgggactcgctacagtgctgcaggacctgctgagaagaggcgagctgcagagggaaagcgacttcatggccagcgtggacagcagctggatctcttggggagtgggagtgttcctgctgaagcccctgaagtggaccctgagcaacatgctgggcgaccacaaggtgccagctgaagaggtgctggtggctgtggaactgctgaaagaaaaggccgaggaagtgtacaggctgtaccagaacagccctctgagcagccatcctgtggtggctctgtctgagctgtctgccctgtgtgccaacagctgcccagacgagagaaccttctacctggtgctgctgcagctccagaaagaaaaacgcgtgaccgtgctggaacagaacggcgagaagatcgtgaagttcgccagaggaccccacgccaaagtgtccccagtgaacgatgtggacgtgggcgtgtaccagctgatgcagtctgaacagctgctgagcagaaaggtggaaagcctgagccaagagtccgagagatgcaaagaggaagccagaagggcctgcagagccggcaaaaaacagctggccctgagaagcctgaaggccaagcagaggaccgagaagagaatcgaggccctgcacgccaagctggatacagtgcagggcatcctggacagaatctacgccagccagaccgaccagatggtgttcaacgcttaccaggctggcgtgggcgctctgaagctgagcatgaaggacgtgacagtcgagaaggccgagagcctggtggaccagatccaagagctgtgtgacacccaggacgaggtgtcccaaacacttgctggcggcgtgacaaacggcctggacttcgatagcgaggaactggaaaaagagctggacatcctgctccaggacaccaccacagaacctctgagcctgctggaaacccctcaagagacaaccctgtacaccaacagcgtgccaaagcctagaatcctggatgccgagctggaagccgaactcgagaagctgagtctgagcgaaggcggactgatccccagcagcaagagccctaagagacagctggaacccacactgtgaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 41 - DNA, 2247 bp, synthetic construct
  misc_feature 1..2247: note = Synthetic
  misc_feature 1146..1856: note = mCherry
  source 1..2247: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaggatccaagctttaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 2892 bp, synthetic construct
  misc_feature 1..2892: note = Synthetic
  misc_feature 1146..2501: note = Chmp7
  source 1..2892: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgtggtcccctgagagagaagcccaagctcctacaggcggagatcctgctggactgttgcctcctgagtgggaagaggacgaggaacggatgagcttcctgttcagcgccttcaagagaagcagagaagtgaacagcaccgactgggacagcaagatgggcttctgggctcctctggtgctgagccactctagaaggcagggcgtcgtcagactgagactgagggatctgcaagaggccttccagagaaagggctctgtgcctctgggactcgctacagtgctgcaggacctgctgagaagaggcgagctgcagagggaaagcgacttcatggccagcgtggacagcagctggatctcttggggagtgggagtgttcctgctgaagcccctgaagtggaccctgagcaacatgctgggcgaccacaaggtgccagctgaagaggtgctggtggctgtggaactgctgaaagaaaaggccgaggaagtgtacaggctgtaccagaacagccctctgagcagccatcctgtggtggctctgtctgagctgtctgccctgtgtgccaacagctgcccagacgagagaaccttctacctggtgctgctgcagctccagaaagaaaaacgcgtgaccgtgctggaacagaacggcgagaagatcgtgaagttcgccagaggaccccacgccaaagtgtccccagtgaacgatgtggacgtgggcgtgtaccagctgatgcagtctgaacagctgctgagcagaaaggtggaaagcctgagccaagagtccgagagatgcaaagaggaagccagaagggcctgcagagccggcaaaaaacagctggccctgagaagcctgaaggccaagcagaggaccgagaagagaatcgaggccctgcacgccaagctggatacagtgcagggcatcctggacagaatctacgccagccagaccgaccagatggtgttcaacgcttaccaggctggcgtgggcgctctgaagctgagcatgaaggacgtgacagtcgagaaggccgagagcctggtggaccagatccaagagctgtgtgacacccaggacgaggtgtcccaaacacttgctggcggcgtgacaaacggcctggacttcgatagcgaggaactggaaaaagagctggacatcctgctccaggacaccaccacagaacctctgagcctgctggaaacccctcaagagacaaccctgtacaccaacagcgtgccaaagcctagaatcctggatgccgagctggaagccgaactcgagaagctgagtctgagcgaaggcggactgatccccagcagcaagagccctaagagacagctggaacccacactgtgaggatccaagctttaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 3072 bp, synthetic construct
  misc_feature 1..3072: note = Synthetic
  misc_feature 1146..2681: note = Lemd2
  source 1..3072: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggatcctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggagttcctggccagcaacgagtccagaatccagaccgagtctcacagagtggccggcgaggatatgctcgtttggagatggaccaagcctagcagcttctccgacagcgagagatgaggatccaagctttaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 448 bp, synthetic construct
  misc_feature 1..448: note = Synthetic
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 45 - DNA, 19 bp, synthetic construct
  misc_feature 1..19: note = Synthetic
  source 1..19: mol_type = other DNA
gatccggttccttggtgtc

SEQ ID NO: 46 - DNA, 24 bp, synthetic construct
  misc_feature 1..24: note = Synthetic
  source 1..24: mol_type = other DNA
atccagccttagcaaaccagaggt

SEQ ID NO: 47 - DNA, 19 bp, synthetic construct
  misc_feature 1..19: note = Synthetic
  source 1..19: mol_type = other DNA
gcctcaaacctgggcaata

SEQ ID NO: 48 - DNA, 18 bp, synthetic construct
  misc_feature 1..18: note = Synthetic
  source 1..18: mol_type = other DNA
gatcgagggcgacctgaa

SEQ ID NO: 49 - DNA, 21 bp, synthetic construct
  misc_feature 1..21: note = Synthetic
  source 1..21: mol_type = other DNA
cccgacaacagcgacgtggac

SEQ ID NO: 50 - DNA, 20 bp, synthetic construct
  misc_feature 1..20: note = Synthetic
  source 1..20: mol_type = other DNA
tcctcgaacagctggttgta

SEQ ID NO: 51 - DNA, 23 bp, synthetic construct
  misc_feature 1..23: note = Synthetic
  source 1..23: mol_type = other DNA
ccgacgctaatctggacaaagtg

SEQ ID NO: 52 - DNA, 23 bp, synthetic construct
  misc_feature 1..23: note = Synthetic
  source 1..23: mol_type = other DNA
ctgtccgcctacaacaagcaccg

SEQ ID NO: 53 - DNA, 22 bp, synthetic construct
  misc_feature 1..22: note = Synthetic
  source 1..22: mol_type = other DNA
gtcagggtaaacaggtggatga

SEQ ID NO: 54 - DNA, 20 bp, synthetic construct
  misc_feature 1..20: note = Synthetic
  source 1..20: mol_type = other DNA
ggctgacgcattggacgatt

SEQ ID NO: 55 - DNA, 26 bp, synthetic construct
  misc_feature 1..26: note = Synthetic
  source 1..26: mol_type = other DNA
atctggatatgctgggaagtgacgcc

SEQ ID NO: 56 - DNA, 20 bp, synthetic construct
  misc_feature 1..20: note = Synthetic
  source 1..20: mol_type = other DNA
caagggcatccgaaccaagc

SEQ ID NO: 57 - DNA, 24 bp, synthetic construct
  misc_feature 1..24: note = Synthetic
  source 1..24: mol_type = other DNA
ggctccttctaatttcgctaatgg

SEQ ID NO: 58 - DNA, 21 bp, synthetic construct
  misc_feature 1..21: note = Synthetic
  source 1..21: mol_type = other DNA
tggcagagtggatcagctcca

SEQ ID NO: 59 - DNA, 20 bp, synthetic construct
  misc_feature 1..20: note = Synthetic
  source 1..20: mol_type = other DNA
ctgacgctgcatgtcacctt

SEQ ID NO: 60 - DNA, 22 bp, synthetic construct
  misc_feature 1..22: note = Synthetic
  source 1..22: mol_type = other DNA
agggcgtgtccatgtctcatag

SEQ ID NO: 61 - DNA, 23 bp, synthetic construct
  misc_feature 1..23: note = Synthetic
  source 1..23: mol_type = other DNA
acagccgaaccaatgctgatgga

SEQ ID NO: 62 - DNA, 19 bp, synthetic construct
  misc_feature 1..19: note = Synthetic
  source 1..19: mol_type = other DNA
ccagccgggtaatggcttc

SEQ ID NO: 63 - DNA, 18 bp, synthetic construct
  misc_feature 1..18: note = Synthetic
  source 1..18: mol_type = other DNA
caccagggaggagtggaa

SEQ ID NO: 64 - DNA, 24 bp, synthetic construct
  misc_feature 1..24: note = Synthetic
  source 1..24: mol_type = other DNA
ctggacactgctcagcagatcgtg

SEQ ID NO: 65 - DNA, 21 bp, synthetic construct
  misc_feature 1..21: note = Synthetic
  source 1..21: mol_type = other DNA
aacccaaggaaaccaggttct

SEQ ID NO: 66 - DNA, 20 bp, synthetic construct
  misc_feature 1..20: note = Synthetic
  source 1..20: mol_type = other DNA
cggaaagacgatgacggaaa

SEQ ID NO: 67 - DNA, 24 bp, synthetic construct
  misc_feature 1..24: note = Synthetic
  source 1..24: mol_type = other DNA
cgtggattacgtcgccagtcaagt

SEQ ID NO: 68 - DNA, 20 bp, synthetic construct
  misc_feature 1..20: note = Synthetic
  source 1..20: mol_type = other DNA
cggtacttcgtccacaaaca

SEQ ID NO: 69 - DNA, 21 bp, synthetic construct
  misc_feature 1..21: note = Synthetic
  source 1..21: mol_type = other DNA
ctgggaaagaaactggaggag

SEQ ID NO: 70 - DNA, 24 bp, synthetic construct
  misc_feature 1..24: note = Synthetic
  source 1..24: mol_type = other DNA
tgttgtgctgggtcagtttctcgt

SEQ ID NO: 71 - DNA, 24 bp, synthetic construct
  misc_feature 1..24: note = Synthetic
  source 1..24: mol_type = other DNA
gtgtctttcaaccattccctaaac

SEQ ID NO: 72 - DNA, 21 bp, synthetic construct
  misc_feature 1..21: note = Synthetic
  source 1..21: mol_type = other DNA
ggatttcagcctggacctatc

SEQ ID NO: 73 - DNA, 24 bp, synthetic construct
  misc_feature 1..24: note = Synthetic
  source 1..24: mol_type = other DNA
aacaagctgagaaggctgagaggc

SEQ ID NO: 74 - DNA, 21 bp, synthetic construct
  misc_feature 1..21: note = Synthetic
  source 1..21: mol_type = other DNA
ggcttcctctctcagtctttc

SEQ ID NO: 75 - DNA, 23 bp, synthetic construct
  misc_feature 1..23: note = Synthetic
  source 1..23: mol_type = other DNA
gggagcaagacatggaaagatac

SEQ ID NO: 76 - DNA, 24 bp, synthetic construct
  misc_feature 1..24: note = Synthetic
  source 1..24: mol_type = other DNA
acagtctgatccctccacagagca

SEQ ID NO: 77 - DNA, 22 bp, synthetic construct
  misc_feature 1..22: note = Synthetic
  source 1..22: mol_type = other DNA
gctctatcccacactctcttca

SEQ ID NO: 78 - DNA, 20 bp, synthetic construct
  misc_feature 1..20: note = Synthetic
  source 1..20: mol_type = other DNA
cagaggcaacaagaccagaa

SEQ ID NO: 79 - DNA, 24 bp, synthetic construct
  misc_feature 1..24: note = Synthetic
  source 1..24: mol_type = other DNA
acagcaacaacaacaataccgccg

SEQ ID NO: 80 - DNA, 22 bp, synthetic construct
  misc_feature 1..22: note = Synthetic
  source 1..22: mol_type = other DNA
gacactggtctaactcccattc

SEQ ID NO: 81 - DNA, 21 bp, synthetic construct
  misc_feature 1..21: note = Synthetic
  source 1..21: mol_type = other DNA
agaggagaactgcagagagaa

SEQ ID NO: 82 - DNA, 24 bp, synthetic construct
  misc_feature 1..24: note = Synthetic
  source 1..24: mol_type = other DNA
tgacttcatggccagcgtggatag

SEQ ID NO: 83 - DNA, 21 bp, synthetic construct
  misc_feature 1..21: note = Synthetic
  source 1..21: mol_type = other DNA
gttgtcgcccagcatattaga

SEQ ID NO: 84 - DNA, 41 bp, synthetic construct
  misc_feature 1..41: note = Synthetic
  source 1..41: mol_type = other DNA
aatggactatcatatgcttaccgtaacttgaaagtatttcg

SEQ ID NO: 85 - DNA, 44 bp, synthetic construct
  misc_feature 1..44: note = Synthetic
  source 1..44: mol_type = other DNA
ctttagtttgtatgtctgttgctattatgtctactattctttcc

SEQ ID NO: 86 - DNA, 83 bp, synthetic construct
  misc_feature 1..83: note = Synthetic
  source 1..83: mol_type = other DNA
aatgatacggcgaccaccgagatctacactctttccctacacgacgctcttccgatctttcttgtggaaaggacgaaacaccg

SEQ ID NO: 87 - DNA, 84 bp, synthetic construct
  misc_feature 1..84: note = Synthetic
  source 1..84: mol_type = other DNA
aatgatacggcgaccaccgagatctacactctttccctacacgacgctcttccgatctattcttgtggaaaggacgaaacaccg

SEQ ID NO: 88 - DNA, 85 bp, synthetic construct
  misc_feature 1..85: note = Synthetic
  source 1..85: mol_type = other DNA
aatgatacggcgaccaccgagatctacactctttccctacacgacgctcttccgatctgattcttgtggaaaggacgaaacaccg

SEQ ID NO: 89 - DNA, 86 bp, synthetic construct
  misc_feature 1..86: note = Synthetic
  source 1..86: mol_type = other DNA
aatgatacggcgaccaccgagatctacactctttccctacacgacgctcttccgatctcgattcttgtggaaaggacgaaacaccg

SEQ ID NO: 90 - DNA, 87 bp, synthetic construct
  misc_feature 1..87: note = Synthetic
  source 1..87: mol_type = other DNA
aatgatacggcgaccaccgagatctacactctttccctacacgacgctcttccgatcttcgattcttgtggaaaggacgaaacaccg

SEQ ID NO: 91 - DNA, 88 bp, synthetic construct
  misc_feature 1..88: note = Synthetic
  source 1..88: mol_type = other DNA
aatgatacggcgaccaccgagatctacactctttccctacacgacgctcttccgatctatcgattcttgtggaaaggacgaaacaccg

SEQ ID NO: 92 - DNA, 89 bp, synthetic construct
  misc_feature 1..89: note = Synthetic
  source 1..89: mol_type = other DNA
aatgatacggcgaccaccgagatctacactctttccctacacgacgctcttccgatctgatcgattcttgtggaaaggacgaaacaccg

SEQ ID NO: 93 - DNA, 90 bp, synthetic construct
  misc_feature 1..90: note = Synthetic
  source 1..90: mol_type = other DNA
aatgatacggcgaccaccgagatctacactctttccctacacgacgctcttccgatctcgatcgattcttgtggaaaggacgaaacaccg

SEQ ID NO: 94 - DNA, 91 bp, synthetic construct
  misc_feature 1..91: note = Synthetic
  source 1..91: mol_type = other DNA
aatgatacggcgaccaccgagatctacactctttccctacacgacgctcttccgatctacgatcgattcttgtggaaaggacgaaacaccg

SEQ ID NO: 95 - DNA, 83 bp, synthetic construct; identical to SEQ ID NO: 86
  misc_feature 1..83: note = Synthetic
  source 1..83: mol_type = other DNA

SEQ ID NO: 96 - DNA, 84 bp, synthetic construct; identical to SEQ ID NO: 87
  misc_feature 1..84: note = Synthetic
  source 1..84: mol_type = other DNA

SEQ ID NO: 97 - DNA, 85 bp, synthetic construct; identical to SEQ ID NO: 88
  misc_feature 1..85: note = Synthetic
  source 1..85: mol_type = other DNA

SEQ ID NO: 98 - DNA, 91 bp, synthetic construct
  misc_feature 1..91: note = Synthetic
  source 1..91: mol_type = other DNA
caagcagaagacggcatacgagataagtagaggtgactggagttcagacgtgtgctcttccgatcttctactattctttcccctgcactgt

SEQ ID NO: 99 - DNA, 91 bp, synthetic construct
  misc_feature 1..91: note = Synthetic
  source 1..91: mol_type = other DNA
caagcagaagacggcatacgagatacacgatcgtgactggagttcagacgtgtgctcttccgatcttctactattctttcccctgcactgt

SEQ ID NO: 100 - DNA, 91 bp, synthetic construct
  misc_feature 1..91: note = Synthetic
  source 1..91: mol_type = other DNA
caagcagaagacggcatacgagatcgcgcggtgtgactggagttcagacgtgtgctcttccgatcttctactattctttcccctgcactgt

SEQ ID NO: 101 - DNA, 91 bp, synthetic construct
  misc_feature 1..91: note = Synthetic
  source 1..91: mol_type = other DNA
caagcagaagacggcatacgagatcatgatcggtgactggagttcagacgtgtgctcttccgatcttctactattctttcccctgcactgt

SEQ ID NO: 102 - DNA, 91 bp, synthetic construct
  misc_feature 1..91: note = Synthetic
  source 1..91: mol_type = other DNA
caagcagaagacggcatacgagatcgttaccagtgactggagttcagacgtgtgctcttccgatcttctactattctttcccctgcactgt

SEQ ID NO: 103 - DNA, 91 bp, synthetic construct
  misc_feature 1..91: note = Synthetic
  source 1..91: mol_type = other DNA
caagcagaagacggcatacgagattccttggtgtgactggagttcagacgtgtgctcttccgatcttctactattctttcccctgcactgt

SEQ ID NO: 104 - DNA, 91 bp, synthetic construct
  misc_feature 1..91: note = Synthetic
  source 1..91: mol_type = other DNA
caagcagaagacggcatacgagataacgcattgtgactggagttcagacgtgtgctcttccgatcttctactattctttcccctgcactgt

SEQ ID NO: 105 - DNA, 91 bp, synthetic construct
  misc_feature 1..91: note = Synthetic
  source 1..91: mol_type = other DNA
caagcagaagacggcatacgagatacaggtatgtgactggagttcagacgtgtgctcttccgatcttctactattctttcccctgcactgt

SEQ ID NO: 106 - DNA, 91 bp, synthetic construct
  misc_feature 1..91: note = Synthetic
  source 1..91: mol_type = other DNA
caagcagaagacggcatacgagataggtaagggtgactggagttcagacgtgtgctcttccgatcttctactattctttcccctgcactgt

SEQ ID NO: 107 - DNA, 91 bp, synthetic construct
  misc_feature 1..91: note = Synthetic
  source 1..91: mol_type = other DNA
caagcagaagacggcatacgagataacaatgggtgactggagttcagacgtgtgctcttccgatcttctactattctttcccctgcactgt

SEQ ID NO: 108 - DNA, 91 bp, synthetic construct
  misc_feature 1..91: note = Synthetic
  source 1..91: mol_type = other DNA
caagcagaagacggcatacgagatactgtatcgtgactggagttcagacgtgtgctcttccgatcttctactattctttcccctgcactgt

SEQ ID NO: 109 - DNA, 91 bp, synthetic construct
  misc_feature 1..91: note = Synthetic
  source 1..91: mol_type = other DNA
caagcagaagacggcatacgagataggtcgcagtgactggagttcagacgtgtgctcttccgatcttctactattctttcccctgcactgt

SEQ ID NO: 110 - DNA, 100 bp, synthetic construct
  misc_feature 1..100: note = Synthetic
  source 1..100: mol_type = other DNA
aatgatacggcgaccaccgagatctacactctttccctacacgacgctcttccgatcttaagtagaggctttatatatcttgtggaaaggacgaaacacc

SEQ ID NO: 111 - DNA, 101 bp, synthetic construct
  misc_feature 1..101: note = Synthetic
  source 1..101: mol_type = other DNA
aatgatacggcgaccaccgagatctacactctttccctacacgacgctcttccgatctatcatgcttagctttatatatcttgtggaaaggacgaaacacc

SEQ ID NO: 112 - DNA, 102 bp, synthetic construct
  misc_feature 1..102: note = Synthetic
  source 1..102: mol_type = other DNA
aatgatacggcgaccaccgagatctacactctttccctacacgacgctcttccgatctgatgcacatctgctttatatatcttgtggaaaggacgaaacacc

SEQ ID NO: 113 - DNA, 103 bp, synthetic construct
  misc_feature 1..103: note = Synthetic
  source 1..103: mol_type = other DNA
aatgatacggcgaccaccgagatctacactctttccctacacgacgctcttccgatctcgattgctcgacgctttatatatcttgtggaaaggacgaaacacc

SEQ ID NO: 114 - DNA, 104 bp, synthetic construct
  misc_feature 1..104: note = Synthetic
  source 1..104: mol_type = other DNA
aatgatacggcgaccaccgagatctacactctttccctacacgacgctcttccgatcttcgatagcaattcgctttatatatcttgtggaaaggacgaaacacc

SEQ ID NO: 115 - DNA, 105 bp, synthetic construct
  misc_feature 1..105: note = Synthetic
  source 1..105: mol_type = other DNA
aatgatacggcgaccaccgagatctacactctttccctacacgacgctcttccgatctatcgatagttgcttgctttatatatcttgtggaaaggacgaaacacc

SEQ ID NO: 116 - DNA, 106 bp, synthetic construct
  misc_feature 1..106: note = Synthetic
  source 1..106: mol_type = other DNA
aatgatacggcgaccaccgagatctacactctttccctacacgacgctcttccgatctgatcgatccagttaggctttatatatcttgtggaaaggacgaaacacc

SEQ ID NO: 117 - DNA, 107 bp, synthetic construct
  misc_feature 1..107: note = Synthetic
  source 1..107: mol_type = other DNA
aatgatacggcgaccaccgagatctacactctttccctacacgacgctcttccgatctcgatcgatttgagcctgctttatatatcttgtggaaaggacgaaacacc

SEQ ID NO: 118 - DNA, 108 bp, synthetic construct
  misc_feature 1..108: note = Synthetic
  source 1..108: mol_type = other DNA
aatgatacggcgaccaccgagatctacactctttccctacacgacgctcttccgatctacgatcgatacacgatcgctttatatatcttgtggaaaggacgaaacacc

SEQ ID NO: 119 - DNA, 109 bp, synthetic construct
  misc_feature 1..109: note = Synthetic
  source 1..109: mol_type = other DNA
aatgatacggcgaccaccgagatctacactctttccctacacgacgctcttccgatcttacgatcgatggtccagagctttatatatcttgtggaaaggacgaaacacc

SEQ ID NO: 120 - DNA, 91 bp, synthetic construct
  misc_feature 1..91: note = Synthetic
  source 1..91: mol_type = other DNA
caagcagaagacggcatacgagattcgccttggtgactggagttcagacgtgtgctcttccgatctgccaagttgataacggactagcctt

SEQ ID NO: 121 - DNA, 91 bp, synthetic construct
  misc_feature 1..91: note = Synthetic
  source 1..91: mol_type = other DNA
caagcagaagacggcatacgagatatagcgtcgtgactggagttcagacgtgtgctcttccgatctgccaagttgataacggactagcctt

SEQ ID NO: 122 - DNA, 91 bp, synthetic construct
  misc_feature 1..91: note = Synthetic
  source 1..91: mol_type = other DNA
caagcagaagacggcatacgagatgaagaagtgtgactggagttcagacgtgtgctcttccgatctgccaagttgataacggactagcctt

SEQ ID NO: 123 - DNA, 91 bp, synthetic construct
  misc_feature 1..91: note = Synthetic
  source 1..91: mol_type = other DNA
caagcagaagacggcatacgagatattctagggtgactggagttcagacgtgtgctcttccgatctgccaagttgataacggactagcctt

SEQ ID NO: 124 - DNA, 91 bp, synthetic construct
  misc_feature 1..91: note = Synthetic
  source 1..91: mol_type = other DNA
caagcagaagacggcatacgagatcgttaccagtgactggagttcagacgtgtgctcttccgatctgccaagttgataacggactagcctt

SEQ ID NO: 125 - DNA, 91 bp, synthetic construct
  misc_feature 1..91: note = Synthetic
  source 1..91: mol_type = other DNA
caagcagaagacggcatacgagatgtctgatggtgactggagttcagacgtgtgctcttccgatctgccaagttgataacggactagcctt

SEQ ID NO: 126 - DNA, 91 bp, synthetic construct
  misc_feature 1..91: note = Synthetic
  source 1..91: mol_type = other DNA
caagcagaagacggcatacgagatttacgcacgtgactggagttcagacgtgtgctcttccgatctgccaagttgataacggactagcctt

SEQ ID NO: 127 - DNA, 91 bp, synthetic construct
  misc_feature 1..91: note = Synthetic
  source 1..91: mol_type = other DNA
caagcagaagacggcatacgagatttgaataggtgactggagttcagacgtgtgctcttccgatctgccaagttgataacggactagcctt

SEQ ID NO: 128 - DNA, 91 bp, synthetic construct
  misc_feature 1..91: note = Synthetic
  source 1..91: mol_type = other DNA
caagcagaagacggcatacgagattccttggtgtgactggagttcagacgtgtgctcttccgatctgccaagttgataacggactagcctt

SEQ ID NO: 129 - DNA, 91 bp, synthetic construct
  misc_feature 1..91: note = Synthetic
  source 1..91: mol_type = other DNA
caagcagaagacggcatacgagatacaggtatgtgactggagttcagacgtgtgctcttccgatctgccaagttgataacggactagcctt

SEQ ID NO: 130 - DNA, 91 bp, synthetic construct
  misc_feature 1..91: note = Synthetic
  source 1..91: mol_type = other DNA
caagcagaagacggcatacgagataggtaagggtgactggagttcagacgtgtgctcttccgatctgccaagttgataacggactagcctt

SEQ ID NO: 131 - DNA, 91 bp, synthetic construct
  misc_feature 1..91: note = Synthetic
  source 1..91: mol_type = other DNA
caagcagaagacggcatacgagataacaatgggtgactggagttcagacgtgtgctcttccgatctgccaagttgataacggactagcctt

SEQ ID NO: 132 - DNA, 91 bp, synthetic construct
  misc_feature 1..91: note = Synthetic
  source 1..91: mol_type = other DNA
caagcagaagacggcatacgagataggttatcgtgactggagttcagacgtgtgctcttccgatctgccaagttgataacggactagcctt

SEQ ID NO: 133 - DNA, 20 bp, synthetic construct
  misc_feature 1..20: note = Synthetic
  source 1..20: mol_type = other DNA
cgcctctcacgtgtaggctt

SEQ ID NO: 134 - DNA, 20 bp, synthetic construct
  misc_feature 1..20: note = Synthetic
  source 1..20: mol_type = other DNA
ccagaaccaattagatatcg

SEQ ID NO: 135 - DNA, 20 bp, synthetic construct
  misc_feature 1..20: note = Synthetic
  source 1..20: mol_type = other DNA
gcggggcgcgggcttctcgg

SEQ ID NO: 136 - DNA, 20 bp, synthetic construct
  misc_feature 1..20: note = Synthetic
  source 1..20: mol_type = other DNA
aaggagccgcccctgtacta

SEQ ID NO: 137 - DNA, 20 bp, synthetic construct
  misc_feature 1..20: note = Synthetic
  source 1..20: mol_type = other DNA
agaagaagctgaacgagtat

SEQ ID NO: 138 - DNA, 20 bp, synthetic construct
  misc_feature 1..20: note = Synthetic
  source 1..20: mol_type = other DNA
cttgaaagactcgagtgtgt

SEQ ID NO: 139 - DNA, 20 bp, synthetic construct
  misc_feature 1..20: note = Synthetic
  source 1..20: mol_type = other DNA
gaaatcaaaagagagaacgt

SEQ ID NO: 140 - DNA, 20 bp, synthetic construct
  misc_feature 1..20: note = Synthetic
  source 1..20: mol_type = other DNA
gcgtctcccttaccagatgt

SEQ ID NO: 141 - DNA, 20 bp, synthetic construct
  misc_feature 1..20: note = Synthetic
  source 1..20: mol_type = other DNA
ggaatgtctacattcccttc

SEQ ID NO: 142 - DNA, 20 bp, synthetic construct
  misc_feature 1..20: note = Synthetic
  source 1..20: mol_type = other DNA
agagtatctcgtcacattac

SEQ ID NO: 143 - DNA, 20 bp, synthetic construct
  misc_feature 1..20: note = Synthetic
  source 1..20: mol_type = other DNA
tctgcatacaataacctgaa

SEQ ID NO: 144 - DNA, 20 bp, synthetic construct
  misc_feature 1..20: note = Synthetic
  source 1..20: mol_type = other DNA
ttgcaggcctatgttgtcct

SEQ ID NO: 145 - DNA, 20 bp, synthetic construct
  misc_feature 1..20: note = Synthetic
  source 1..20: mol_type = other DNA
gcttcggatgccttcgagag

SEQ ID NO: 146 - DNA, 20 bp, synthetic construct
  misc_feature 1..20: note = Synthetic
  source 1..20: mol_type = other DNA
tttcctccagcttcttgccc

SEQ ID NO: 147 - DNA, 20 bp, synthetic construct
  misc_feature 1..20: note = Synthetic
  source 1..20: mol_type = other DNA
cgccaacgccaagcagtccc

SEQ ID NO: 148 - DNA, 20 bp, synthetic construct
  misc_feature 1..20: note = Synthetic
  source 1..20: mol_type = other DNA
ttggtgacgtcctgagcaag

SEQ ID NO: 149 - DNA, 20 bp, synthetic construct
  misc_feature 1..20: note = Synthetic
  source 1..20: mol_type = other DNA
gcttgaggtatccgcaggag

SEQ ID NO: 150 - DNA, 20 bp, synthetic construct
  misc_feature 1..20: note = Synthetic
  source 1..20: mol_type = other DNA
tcagcctcgtctagagagct

SEQ ID NO: 151 - DNA, 20 bp, synthetic construct
  misc_feature 1..20: note = Synthetic
  source 1..20: mol_type = other DNA
tcatcaacttcatcactgct

SEQ ID NO: 152 - DNA, 20 bp, synthetic construct
  misc_feature 1..20: note = Synthetic
  source 1..20: mol_type = other DNA
cagaccgaaccacctcctgt

SEQ ID NO: 153 - DNA, 20 bp, synthetic construct
  misc_feature 1..20: note = Synthetic
  source 1..20: mol_type = other DNA
gaagagccctcttctgggtc

SEQ ID NO: 154 - DNA, 20 bp, synthetic construct
  misc_feature 1..20: note = Synthetic
  source 1..20: mol_type = other DNA
acagcttctctcacgcaaag

SEQ ID NO: 155 - DNA, 20 bp, synthetic construct
  misc_feature 1..20: note = Synthetic
  source 1..20: mol_type = other DNA
gaggagcctgtgaggcgttc

SEQ ID NO: 156 - DNA, 20 bp, synthetic construct
  misc_feature 1..20: note = Synthetic
  source 1..20: mol_type = other DNA
ttcatgacccatctagaaag

SEQ ID NO: 157 - DNA, 20 bp, synthetic construct
  misc_feature 1..20: note = Synthetic
  source 1..20: mol_type = other DNA
tttaaggtcaggcgccttcc

SEQ ID NO: 158 - DNA, 20 bp, synthetic construct
  misc_feature 1..20: note = Synthetic
  source 1..20: mol_type = other DNA
gcttgtattgctgaaaagtt

SEQ ID NO: 159 - DNA, 20 bp, synthetic construct
  misc_feature 1..20: note = Synthetic
  source 1..20: mol_type = other DNA
gcaattaaaaagaaatttca

SEQ ID NO: 160 - DNA, 20 bp, synthetic construct
  misc_feature 1..20: note = Synthetic
  source 1..20: mol_type = other DNA
acagtccccgagaagttggg

SEQ ID NO: 161 - DNA, 20 bp, synthetic construct
  misc_feature 1..20: note = Synthetic
  source 1..20: mol_type = other DNA
gaaggtggcgcggggtaaac

SEQ ID NO: 162 - DNA, 20 bp, synthetic construct
  misc_feature 1..20: note = Synthetic
  source 1..20: mol_type = other DNA
ggaggggtcggcaattgaac

SEQ ID NO: 163 - DNA, 20 bp, synthetic construct
  misc_feature 1..20: note = Synthetic
  source 1..20: mol_type = other DNA
ccaccgctgtccaccgcctc

SEQ ID NO: 164 - DNA, 20 bp, synthetic construct
  misc_feature 1..20: note = Synthetic
  source 1..20: mol_type = other DNA
tccggccaggatcaactcgt

SEQ ID NO: 165 - DNA, 20 bp, synthetic construct
  misc_feature 1..20: note = Synthetic
  source 1..20: mol_type = other DNA
ggggaatggggggggttgca

SEQ ID NO: 166 - DNA, 20 bp, synthetic construct
  misc_feature 1..20: note = Synthetic
  source 1..20: mol_type = other DNA
tgggggcgcatgcgtagagg

SEQ ID NO: 167 - DNA, 20 bp, synthetic construct
  misc_feature 1..20: note = Synthetic
  source 1..20: mol_type = other DNA
aaaggggaaaagagtattgg

SEQ ID NO: 168 - DNA, 20 bp, synthetic construct
  misc_feature 1..20: note = Synthetic
  source 1..20: mol_type = other DNA
tgagataattctctggttca

SEQ ID NO: 169 - DNA, 20 bp, synthetic construct
  misc_feature 1..20: note = Synthetic
  source 1..20: mol_type = other DNA
tggtggaagcttcttagggg

SEQ ID NO: 170 - DNA, 20 bp, synthetic construct
  misc_feature 1..20: note = Synthetic
  source 1..20: mol_type = other DNA
tacttacggctatatattct

SEQ ID NO: 171 - DNA, 20 bp, synthetic construct
  misc_feature 1..20: note = Synthetic
  source 1..20: mol_type = other DNA
aagaacgctttctgttcaag

SEQ ID NO: 172 - DNA, 20 bp, synthetic construct
  misc_feature 1..20: note = Synthetic
  source 1..20: mol_type = other DNA
tccggtctctgacacctctg

SEQ ID NO: 173 - DNA, 20 bp, synthetic construct
  misc_feature 1..20: note = Synthetic
  source 1..20: mol_type = other DNA
tgtcccttctcagaaccttg

SEQ ID NO: 174 - DNA, 20 bp, synthetic construct
  misc_feature 1..20: note = Synthetic
  source 1..20: mol_type = other DNA
ttgtgggggagggccggagc

SEQ ID NO: 175 - DNA, 20 bp, synthetic construct
  misc_feature 1..20: note = Synthetic
  source 1..20: mol_type = other DNA
ctctgctctctgatccaagt

SEQ ID NO: 176 - DNA, 20 bp, synthetic construct
  misc_feature 1..20: note = Synthetic
  source 1..20: mol_type = other DNA
tctaagattgaagctttgtt

SEQ ID NO: 177 - DNA, 20 bp, synthetic construct
  misc_feature 1..20: note = Synthetic
  source 1..20: mol_type = other DNA
catggcgtggggagcgcgcg

SEQ ID NO: 178 - DNA, 20 bp, synthetic construct
  misc_feature 1..20: note = Synthetic
  source 1..20: mol_type = other DNA
agtacagtgtagccgtgatg

SEQ ID NO: 179 - DNA, 20 bp, synthetic construct
  misc_feature 1..20: note = Synthetic
  source 1..20: mol_type = other DNA
ctgcacgtcacctgcacaga

SEQ ID NO: 180 - DNA, 20 bp, synthetic construct
  misc_feature 1..20: note = Synthetic
  source 1..20: mol_type = other DNA
atccagccttagcaaaccag

SEQ ID NO: 181 - DNA, 20 bp, synthetic construct
  misc_feature 1..20: note = Synthetic
  source 1..20: mol_type = other DNA
ttcggaccactcaggagccg

SEQ ID NO: 182 - DNA, 20 bp, synthetic construct
  misc_feature 1..20: note = Synthetic
  source 1..20: mol_type = other DNA
gagctctagacaccaacgtg

SEQ ID NO: 183 - DNA, 20 bp, synthetic construct
  misc_feature 1..20: note = Synthetic
  source 1..20: mol_type = other DNA
caagcagctgtccgagtccc

SEQ ID NO: 184 - DNA, 20 bp, synthetic construct
  misc_feature 1..20: note = Synthetic
  source 1..20: mol_type = other DNA
acatcgaacctcttgaacgt

SEQ ID NO: 185 - DNA, 20 bp, synthetic construct
  misc_feature 1..20: note = Synthetic
  source 1..20: mol_type = other DNA
tagagttgtcatctttcaac

SEQ ID NO: 186 - DNA, 20 bp, synthetic construct
  misc_feature 1..20: note = Synthetic
  source 1..20: mol_type = other DNA
acgtacccacagcactatta

SEQ ID NO: 187 - DNA, 20 bp, synthetic construct
  misc_feature 1..20: note = Synthetic
  source 1..20: mol_type = other DNA
aggtctaaagccgtcacttc

SEQ ID NO: 188 - DNA, 20 bp, synthetic construct
  misc_feature 1..20: note = Synthetic
  source 1..20: mol_type = other DNA
aagaggagcagatcggtggt

SEQ ID NO: 189 - DNA, 20 bp, synthetic construct
  misc_feature 1..20: note = Synthetic
  source 1..20: mol_type = other DNA
aggccactccccaaatgtcc

SEQ ID NO: 190 - DNA, 20 bp, synthetic construct
  misc_feature 1..20: note = Synthetic
  source 1..20: mol_type = other DNA
catgggtcgggggcagtagg

SEQ ID NO: 191 - DNA, 20 bp, synthetic construct
  misc_feature 1..20: note = Synthetic
  source 1..20: mol_type = other DNA
actctgcccatgagccgcgc

SEQ ID NO: 192 - DNA, 20 bp, synthetic construct
  misc_feature 1..20: note = Synthetic
  source 1..20: mol_type = other DNA
ggccgggcgggccgggtaag

SEQ ID NO: 193 - DNA, 20 bp, synthetic construct
  misc_feature 1..20: note = Synthetic
  source 1..20: mol_type = other DNA
gggtggcctggcgggacctg

SEQ ID NO: 194 - DNA, 20 bp, synthetic construct
  misc_feature 1..20: note = Synthetic
  source 1..20: mol_type = other DNA
gcgatacgtgcgccatctgc

SEQ ID NO: 195 - DNA, 20 bp, synthetic construct
  misc_feature 1..20: note = Synthetic
  source 1..20: mol_type = other DNA
attgttctgtttcacccaca

SEQ ID NO: 196 - DNA, 20 bp, synthetic construct
  misc_feature 1..20: note = Synthetic
  source 1..20: mol_type = other DNA
gcatctgattctcttgtttg

SEQ ID NO: 197 - DNA, 20 bp, synthetic construct
  misc_feature 1..20: note = Synthetic
  source 1..20: mol_type = other DNA
ccaaggtttggttccagaac

SEQ ID NO: 198 - DNA, 20 bp, synthetic construct
  misc_feature 1..20: note = Synthetic
  source 1..20: mol_type = other DNA
gattgggcggctgctggggg

SEQ ID NO: 199 - DNA, 20 bp, synthetic construct
  misc_feature 1..20: note = Synthetic
  source 1..20: mol_type = other DNA
ggggcctctgcggatggggc

SEQ ID NO: 200 - DNA, 20 bp, synthetic construct
  misc_feature 1..20: note = Synthetic
  source 1..20: mol_type = other DNA
ggggcctctgctgatggggc

SEQ ID NO: 201 - DNA, 20 bp, synthetic construct
  misc_feature 1..20: note = Synthetic
  source 1..20: mol_type = other DNA
tgacggctcttccgacccgg

SEQ ID NO: 202 - DNA, 20 bp, synthetic construct
  misc_feature 1..20: note = Synthetic
  source 1..20: mol_type = other DNA
tcagctcttgacctcgtctt

SEQ ID NO: 203 - DNA, 20 bp, synthetic construct
  misc_feature 1..20: note = Synthetic
  source 1..20: mol_type = other DNA
tgccttaggaccctggtccg

SEQ ID NO: 204 - DNA, 20 bp, synthetic construct
  misc_feature 1..20: note = Synthetic
  source 1..20: mol_type = other DNA
ctaggtgaacagcctccaga

SEQ ID NO: 205 - DNA, 20 bp, synthetic construct
  misc_feature 1..20: note = Synthetic
  source 1..20: mol_type = other DNA
gtgaaatacggagtgaatcc

SEQ ID NO: 206 - DNA, 20 bp, synthetic construct
  misc_feature 1..20: note = Synthetic
  source 1..20: mol_type = other DNA
cgcccacccgcttccggttg

SEQ ID NO: 207 - DNA, 20 bp, synthetic construct
  misc_feature 1..20: note = Synthetic
  source 1..20: mol_type = other DNA
ctcaagtgactcggcgggcg

SEQ ID NO: 208 - DNA, 20 bp, synthetic construct
  misc_feature 1..20: note = Synthetic
  source 1..20: mol_type = other DNA
gcggacgcaaacacggggag

SEQ ID NO: 209 - DNA, 20 bp, synthetic construct
  misc_feature 1..20: note = Synthetic
  source 1..20: mol_type = other DNA
tggacgagagcctgttcgac

SEQ ID NO: 210 - DNA, 20 bp, synthetic construct
  misc_feature 1..20: note = Synthetic
  source 1..20: mol_type = other DNA
cagacatcagccgtcggaca

SEQ ID NO: 211 - DNA, 20 bp, synthetic construct
  misc_feature 1..20: note = Synthetic
  source 1..20: mol_type = other DNA
tttaaggaacccagtgacaa

SEQ ID NO: 212 - DNA, 20 bp, synthetic construct
  misc_feature 1..20: note = Synthetic
  source 1..20: mol_type = other DNA
actctctgcccggagccgcc

SEQ ID NO: 213 - DNA, 20 bp, synthetic construct
  misc_feature 1..20: note = Synthetic
  source 1..20: mol_type = other DNA
gcgcgggccaggtggctcca

SEQ ID NO: 214 - DNA, 20 bp, synthetic construct
  misc_feature 1..20: note = Synthetic
  source 1..20: mol_type = other DNA
ggccacgccctccctaaccc

SEQ ID NO: 215 - DNA, 22 bp, synthetic construct
  misc_feature 1..22: note = Synthetic
  source 1..22: mol_type = other DNA
gggagcaagacatggaaagata

SEQ ID NO: 216 - DNA, 24 bp, synthetic construct
  misc_feature 1..24: note = Synthetic
  source 1..24: mol_type = other DNA
acagtctgatccctccacagagca

SEQ ID NO: 217 - DNA, 22 bp, synthetic construct
  misc_feature 1..22: note = Synthetic
  source 1..22: mol_type = other DNA
ctgtcccacactctcttcattc

SEQ ID NO: 218 - DNA, 22 bp, synthetic construct
  misc_feature 1..22: note = Synthetic
  source 1..22: mol_type = other DNA
tctcgtttggagatggattcag

SEQ ID NO: 219 - DNA, 24 bp, synthetic construct
  misc_feature 1..24: note = Synthetic
  source 1..24: mol_type = other DNA
agctgcgacaagacactggtcatc

SEQ ID NO: 220 - DNA, 21 bp, synthetic construct
  misc_feature 1..21: note = Synthetic
  source 1..21: mol_type = other DNA
ctgttaggagggctgtttctt

SEQ ID NO: 221 - DNA, 20 bp, synthetic construct
  misc_feature 1..20: note = Synthetic
  source 1..20: mol_type = other DNA
gagatcctgctggactgttg

SEQ ID NO: 222 - DNA, 24 bp, synthetic construct
  misc_feature 1..24: note = Synthetic
  source 1..24: mol_type = other DNA
tcctgttcagcgccttcaagagaa

SEQ ID NO: 223 - DNA, 19 bp, synthetic construct
  misc_feature 1..19: note = Synthetic
  source 1..19: mol_type = other DNA
gtcggtgctgttcacttct

SEQ ID NO: 224 - DNA, 19 bp, synthetic construct
  misc_feature 1..19: note = Synthetic
  source 1..19: mol_type = other DNA
gtgttacaaccgactatca

SEQ ID NO: 225 - DNA, 20 bp, synthetic construct
  misc_feature 1..20: note = Synthetic
  source 1..20: mol_type = other DNA
gtcaacaacgtagaggatgc

SEQ ID NO: 226 - DNA, 20 bp, synthetic construct
  misc_feature 1..20: note = Synthetic
  source 1..20: mol_type = other DNA
ccattctcggaagaggtctt

SEQ ID NO: 227 - RNA, 20 nt, synthetic construct
  misc_feature 1..20: note = Synthetic
  source 1..20: mol_type = other RNA
ccagaaccaattagatatcg

SEQ ID NO: 228 - RNA, 20 nt, synthetic construct
  misc_feature 1..20: note = Synthetic
  source 1..20: mol_type = other RNA
ttggtgacgtcctgagcaag

SEQ ID NO: 229 - RNA, 20 nt, synthetic construct
  misc_feature 1..20: note = Synthetic
  source 1..20: mol_type = other RNA
acatcgaacctcttgaacgt

SEQ ID NO: 230 - DNA, 23 bp, synthetic construct
  misc_feature 1..23: note = Synthetic
  source 1..23: mol_type = other DNA
gggtgatgagatggaggggattg

SEQ ID NO: 231 - DNA, 23 bp, synthetic construct
  misc_feature 1..23: note = Synthetic
  source 1..23: mol_type = other DNA
agattttggttacagtgtgggcc

SEQ ID NO: 232 - DNA, 20 bp, synthetic construct
  misc_feature 1..20: note = Synthetic
  source 1..20: mol_type = other DNA
ctgcttcctgggcttgtgtg

SEQ ID NO: 233 - DNA, 22 bp, synthetic construct
  misc_feature 1..22: note = Synthetic
  source 1..22: mol_type = other DNA
gatgagggctggggattgagag

SEQ ID NO: 234 - DNA, 24 bp, synthetic construct
  misc_feature 1..24: note = Synthetic
  source 1..24: mol_type = other DNA
gtgtgcatctactgaaaaggctca

SEQ ID NO: 235 - DNA, 23 bp, synthetic construct
  misc_feature 1..23: note = Synthetic
  source 1..23: mol_type = other DNA
ccgtggtggttggggtatatctc

SEQ ID NO: 236 - DNA, 20 bp, synthetic construct
  misc_feature 1..20: note = Synthetic
  source 1..20: mol_type = other DNA
ccccgtacccaggacctcac

SEQ ID NO: 237 - DNA, 23 bp, synthetic construct
  misc_feature 1..23: note = Synthetic
  source 1..23: mol_type = other DNA
agagtctctaatgtcagctggca

SEQ ID NO: 238 - DNA, 22 bp, synthetic construct
  misc_feature 1..22: note = Synthetic
  source 1..22: mol_type = other DNA
gtaccacctgttgccaaccttc

SEQ ID NO: 239 - DNA, 23 bp, synthetic construct
  misc_feature 1..23: note = Synthetic
  source 1..23: mol_type = other DNA
atattgcccttgggacttgtggt

SEQ ID NO: 240 - DNA, 26 bp, synthetic construct
  misc_feature 1..26: note = Synthetic
  source 1..26: mol_type = other DNA
tgttagtgctgtttaatgctgaatgt

SEQ ID NO: 241 - DNA, 23 bp, synthetic construct
  misc_feature 1..23: note = Synthetic
  source 1..23: mol_type = other DNA
tcttacccagttcaaaaggccca

SEQ ID NO: 242 - RNA, 20 nt, synthetic construct
  misc_feature 1..20: note = Synthetic
  source 1..20: mol_type = other RNA
aaggagccgcccctgtacta

SEQ ID NO: 243 - RNA, 20 nt, synthetic construct
  misc_feature 1..20: note = Synthetic
  source 1..20: mol_type = other RNA
ttgcaggcctatgttgtcct

SEQ ID NO: 244 - RNA, 20 nt, synthetic construct
  misc_feature 1..20: note = Synthetic
  source 1..20: mol_type = other RNA
gagctctagacaccaacgtg

SEQ ID NO: 245 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 246 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 247 - DNA, 130 bp, synthetic construct
  misc_feature 1..130: note = Synthetic
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 248 - DNA, 130 bp, synthetic construct
  misc_feature 1..130: note = Synthetic
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 249 - DNA, 3373 bp, synthetic construct
  misc_feature 1..3373: note = Synthetic
  source 1..3373: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggattctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggagttcctggccagcaacgagtccagaatccagaccgagtctcacagagtggccggcgaggatatgctcgtttggagatggaccaagcctagcagcttctccgacagcgagagatgactcgaggatggggactgatagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 250 - DNA, 4604 bp, synthetic construct
  misc_feature 1..4604: note = Synthetic
  source 1..4604: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatggctgctgctacagctgctgctgctcctcagcagctgagcgacgaggaactgttcagccagctgagaagatacggcctgtctcctggacctgtgaccgagagcacaagacccgtgtacctgaagaagctcaagaagctgagagaggaagaacagcagcaacaacaacagcaacagcaacaacagcacagagctggcggcagaggcaacaagaccagaaactctaacaacaacaataccgccaccgccatgggcggcagacctggatctggcgatctggcctatctgagatctcctgctggactgggcagactgtctgcttctgctgctgagtctcctgtggcaggcggttctggcggagctgctgcagttcctgctgctggatctaaggtgctgctgggcttcagcagcgacgagtctgacgtggaagcctctcctagagaacaagctggtggtggtggcggaggcggcgctagaagagatagagctgctctgcagtacagaggcctgagagcacctcctgctccaccagctgctggcgaagttacaggtggacaccctggcgagagaagaaagccccattcttggtggggagccagaaggccagctggacctgaacctcaacctccagccgctggatctgatggcgctgccgaagatgctgatgaggaactggctgacggcgaggacagagatcctgaggctgaagaacctctgtgggccagcagagctgtgaacggctctagactgctgccttacagcagctgcagagagcactacagcgactccgaggaagaagaggaagagggcgaagaggacggcgacgttgcaccagctagacaggtgctgaaggacgacagcctggccagacacagacccagaagaagccacagcaagcccttcagcgccctgacagctaagagcggaggcagcagacaagagacaagcgttcaaggcggcggagccctggccatgaatgatagagccgctgcagccggaagcctggacagatccagaaacctggaagaggccgcagctgaacctggcggcggaggtggcggtggatgtggatgtgatcctgtggacagcatccccagatacagagccggcgctaagaagctggctcctctgctgtctcctccatctcctgatggcgacagcaccctggaaagccctacaggacccctgctcaagaccaacaaccacatcggaggcggagctttcggcgtggactctccaggactgtacgccaactctcttcctcctggcgctactgccgctgctgcacctggaacactgagaatcaaccacgccaaccacaccggcagcaaccacacatacctgaaaaccgcctacggcaagcccaagctgtgcgagcctgaagaggaactcctgcagcagtttaagcgcgaggaagtgtctcctaccggcagcttctctgcccactacctgagcatgtttctgctgaccgccgcctgcctgttctttctgatcctgggcctgacctacctgggcatgagaggaacaggcgtgccagaagatggcggcctgatcaagaaccccttcgacgagacattcggcaagatccaagagagcgagaagaacctgctgatgtctaccctgtacaagctgcacgatagactggcccagatcgctggcgatcacgagtgtggaagcagctcccagagaatgctgagcgtgcaagaggctgccgcttatctgaagaatctgggccccgagtacgaggacgtgttcaacacatctctgctgtggattttcaagaacggcaaggacgtgggcatcagatgcgtcggctatggccctgaggaagatctgaccaatattaccgacgtgcagtttctgcagtccaccagacctcagatgcccttctggtgcagattcagacgggccttcatcaccgtgacacaccgcctgctgcttctctgtctgggagtcgtgcttgtgtgtgtggccctgcggtacatgagatacagatggaccaaagaagaagaagagacacggcagatgtacgacatggtggtcaagatcatcgacgtgctgagatcccacaacgaggcctgccaagagactaaggacctgcagccttacatgcccctgcctcacgttcgggacagcctgatccagccacaggaccggaagaaaatgaagaaagtgtgggacagagccgtggacttcctggccgctaacgagtctagagtgcggaccgagacaagaagagtcggaggcgccgattttctcgtttggagatggattcagcctagcgccagctgcgacaagacactggtcatccccagcaaagtgtggcagggccaagccttccacctggatagaagaaacagccctcctaacagcctgacaccttgcctgaagatccgcaacatgttcgaccccgtgatggaaatcggcgaccactggcacctggccatccaagaggctatcctggaaaagtgcagcgacaacgacggcatcgtgcatatcgccgtggacagaaactcccgcgagggctgtgtgtacgtgaagtgtctgagccctgagtacgccggcaaggcctttaaggctctgcacggcagttggttcgacggcaagctggtcacggtgaagtacctgagactggatagataccaccacaggttccctcaggctctgacctgcaacacccctctgaagcctgccaacaagcacatgaactccctgagccatctgagactgagaaccggcctggctaacagccagggcagttcttaagctcgaggatggggactgatagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 251 - DNA, 3194 bp, synthetic construct
  misc_feature 1..3194: note = Synthetic
  source 1..3194: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggatcccgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgtggtcccctgagagagaagcccaagctcctacaggcggagatcctgctggactgttgcctcctgagtgggaagaggacgaggaacggatgagcttcctgttcagcgccttcaagagaagcagagaagtgaacagcaccgactgggacagcaagatgggcttctgggctcctctggtgctgagccactctagaaggcagggcgtcgtcagactgagactgagggatctgcaagaggccttccagagaaagggctctgtgcctctgggactcgctacagtgctgcaggacctgctgagaagaggcgagctgcagagggaaagcgacttcatggccagcgtggacagcagctggatctcttggggagtgggagtgttcctgctgaagcccctgaagtggaccctgagcaacatgctgggcgaccacaaggtgccagctgaagaggtgctggtggctgtggaactgctgaaagaaaaggccgaggaagtgtacaggctgtaccagaacagccctctgagcagccatcctgtggtggctctgtctgagctgtctgccctgtgtgccaacagctgcccagacgagagaaccttctacctggtgctgctgcagctccagaaagaaaaacgcgtgaccgtgctggaacagaacggcgagaagatcgtgaagttcgccagaggaccccacgccaaagtgtccccagtgaacgatgtggacgtgggcgtgtaccagctgatgcagtctgaacagctgctgagcagaaaggtggaaagcctgagccaagagtccgagagatgcaaagaggaagccagaagggcctgcagagccggcaaaaaacagctggccctgagaagcctgaaggccaagcagaggaccgagaagagaatcgaggccctgcacgccaagctggatacagtgcagggcatcctggacagaatctacgccagccagaccgaccagatggtgttcaacgcttaccaggctggcgtgggcgctctgaagctgagcatgaaggacgtgacagtcgagaaggccgagagcctggtggaccagatccaagagctgtgtgacacccaggacgaggtgtcccaaacacttgctggcggcgtgacaaacggcctggacttcgatagcgaggaactggaaaaagagctggacatcctgctccaggacaccaccacagaacctctgagcctgctggaaacccctcaagagacaaccctgtacaccaacagcgtgccaaagcctagaatcctggatgccgagctggaagccgaactcgagaagctgagtctgagcgaaggcggactgatccccagcagcaagagccctaagagacagctggaacccacactgtgagctcgaggatggggactgatagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 252 - DNA, 502 bp, synthetic construct
  misc_feature 1..502: note = Synthetic
  source 1..502: mol_type = other DNA
tgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 253 - DNA, 230 bp, synthetic construct
  misc_feature 1..230: note = Synthetic
  source 1..230: mol_type = other DNA
taatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtc

SEQ ID NO: 254 - DNA, 402 bp, synthetic construct
  misc_feature 1..402: note = Synthetic
  source 1..402: mol_type = other DNA
aaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgg

SEQ ID NO: 255 - DNA, 1536 bp, synthetic construct
  misc_feature 1..1536: note = Synthetic
  source 1..1536: mol_type = other DNA
atggctggcctgagcgacctggaactgagaagagaactgcaggccctgggcttccagcctggacctatcaccgacaccaccagaaacgtgtacagaaacaagctgagaaggctgagaggcgaggccagactgagagatgacgaacggctgagagaggacgctggccctagagaagatgccggacctagaggacccgagagacagagagaggaagcccgcctgagagaagaggctccccttagagctagacctgccgcctctgtgctgagatctgaaccttggcctctgagcccatctcctccagctccatctgctgcctctgatgcttctggcccctacggcaactttggcgcttctgcttctccttgggccgcttctagaggcctgtcttaccctcctcatgctggccctggacctcttcgtagaagggcttctgtcagaggctccagcgaggacgacgaggacacaagaacacccgatagacacgcccctggcagaggcagacattggtgggctccaccatctgccagcgccagacctcattctgctctgctgggagccgatgccagacctggactgaagggatctagaacaggatctgccggcgctggaagaaccagacctgaagtcggaagatggctggaaagatgcctgagcagactgctgctgtgggcttctctgggactgctcctgggctttctggctatcctgtgggtcaagatgggcaagcccagcgctcctcaagaggccgaggataacatgaagctgctgcccgtggactgcgagagaaagaccgacgagttctgccaggccaagcagaaggctgcactgctggaactgctgcacgagctgtacaactttctggccattcaggccggcaacttcgagtgcggaaaccccgagaagctgaagtccaagtgcatccccgtgctggaagctcaagagtatatcgccaacgtgacaagcagccctagcagcagattcaaggccgctctgacctggattctgagcagcaacaaggacgtcggcatctggctgaagggcgaagatccttctgagctggccaccaccgtggacaaggtcgtgtgtctggaaagcgccaggcctagaatgggcatcggatgcagactgagcagagccctgctgacagctgtgacccacgtgctgatctttttctggtgcctggccttcctgtggggcctgctgatcctgctgaagtacagatggcggaagctggaagaggaagaacaggctatgtacgagatggtcaagaaaatcatcgacgtggtgcaggaccactacgtggactgggagcaagacatggaaagatacccctacgtgggcatcctgcatgtgcgggacagtctgatccctccacagagcaggcggagaatgaagagagtgtgggacagagccgtggagttcctggccagcaacgagtccagaatccagaccgagtctcacagagtggccggcgaggatatgctcgtttggagatggaccaagcctagcagcttctccgacagcgagagatga

(19) United States
(12) Patent Application Publication
(10) Pub. No.: US 2023/0293727 A1
(43) Pub. Date: Sep. 21, 2023

(54) OVEREXPRESSION OF LEMD2, LEMD3, OR CHMP7 AS A THERAPEUTIC MODALITY FOR TAUOPATHY

(71) Applicant: REGENERON PHARMACEUTICALS, INC., TARRYTOWN, NY (US)

(72) Inventors: Marine Prissette, Brooklyn, NY (US); Wen Fury, New York, NY (US); Daria Fedorova, Bardonia, NY (US); Claudia Racioppi, New York, NY (US); Brian Zambrowicz, Sleepy Hollow, NY (US)

(21) Appl. No.: 18/049,834

(22) Filed: Oct. 26, 2022

Related U.S. Application Data
(60) Provisional application No. 63/369,557, filed on Jul. 27, 2022.
(60) Provisional application No. 63/271,839, filed on Oct. 26, 2021.

(51) Int. Cl. A61K 48/00; C12N 15/86; A61K 38/17; A61P 25/28

(52) U.S. Cl. CPC A61K 48/0058; A61K 38/1709; A61P 25/28; C12N 15/86; C12N 2740/15043; C12N 2740/15071; C12N 2750/14143; C12N 2750/14171

(57) ABSTRACT

Provided herein are methods of inhibiting tau aggregation in a cell or a subject, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell or the subject. Also provided herein are methods of treating or preventing a tauopathy in a subject, comprising administering LEMD2, CHMP7, or LEMD3 or a nucleic acid encoding the LEMD2, the CHMP7, or the LEMD3 to the subject, wherein the LEMD2, the CHMP7, or the LEMD3 inhibits tau aggregation in a cell in the subject. Also provided are nucleic acids encoding LEMD2, CHMP7, or LEMD3 (e.g., in an expression construct and operably linked to a heterologous promoter).

### CROSS-REFERENCE TO RELATED APPLICATIONS

This application claims the benefit of U.S. Application No. 63/271,839, filed Oct. 26, 2021, and U.S. Application No. 63/369,557, filed Jul. 27, 2022, each of which is herein incorporated by reference in its entirety for all purposes.

### REFERENCE TO A SEQUENCE LISTING SUBMITTED AS A TEXT FILE VIA EFS WEB

The Sequence Listing written in file 057766-586576.xml is 452 kilobytes, was created on Oct. 26, 2022, and is hereby incorporated by reference.

### BACKGROUND

Tau is as a member of a large family of microtubule-associated proteins that are enriched in the brain. Tau is predominantly found in neurons of the central nervous system and is restricted to axons, where it functions to enhance microtubule stability. There are six major isoforms of tau, ranging from 352 to 441 amino acids in length, that are produced by alternative splicing of transcripts from the MAPT gene, located on chromosome 17 in humans. Like other microtubule associated proteins, such as MAP2, each tau isoform contains a series of three or four tandem repeat units (3RD and 4RD) responsible for microtubule binding. Most interest in tau has focused on its roles in cytoskeletal microtubule dynamics, but recent evidence points to a nuclear function in RNA metabolism and pre-mRNA splicing. Despite decades of investigation, many questions remain with respect to tau’s normal function in neurons and the ways its dysfunction promotes neurodegenerative disease.

### SUMMARY

Provided herein are compositions and methods for inhibiting tau aggregation in a cell or a subject, methods of reducing tau phosphorylation in a cell or subject, compositions and methods for of treating or preventing a tauopathy in a subject, nucleic acids encoding a LEM domain-containing protein 2 (LEMD2), a charged multivesicular body protein 7 (CHMP7), or an inner nuclear membrane protein Man 1 (LEMD3), and expression constructs encoding LEMD2, CHMP7, or LEMD3. Also provided herein are compositions and methods for reducing serum neurofilament light chain (sNfL) or preventing accumulation of serum neurofilament light chain (sNfL) in a subject.

In one aspect, provided are methods of inhibiting or reducing tau aggregation, inhibiting or reducing tau phosphorylation, or inhibiting or reducing accumulation of insoluble tau in a cell or subject. Likewise, provided are methods of reducing serum neurofilament light chain (sNfL) or preventing accumulation of serum neurofilament light chain (sNfL) in a subject. Some such methods are for inhibiting or reducing tau aggregation in a cell or subject. Some such methods are for inhibiting tau aggregation in a cell or subject. Some such methods are for inhibiting tau aggregation in a cell or subject. Some such methods are for inhibiting or reducing tau phosphorylation in a cell or subject. Some such methods are for inhibiting tau phosphorylation in a cell or subject. Some such methods are for reducing tau phosphorylation in a cell or subject. In some such methods, the tau phosphorylation that is inhibited or reduced is phosphorylation of tau on serine 356. In some such methods, levels of phosphorylated tau (e.g., phospho-tau-Ser356) are decreased in the soma, in the perinuclear region, and/or in the nucleoplasm. In some such methods, levels of phosphorylated tau (e.g., phospho-tau-Ser356) are decreased in the soma. In some such methods, levels of phosphorylated tau (e.g., phospho-tau-Ser356) are decreased in the perinuclear region. In some such methods, levels of phosphorylated tau (e.g., phospho-tau-Ser356) are decreased in the nucleoplasm. Some such methods are for inhibiting or reducing accumulation of insoluble tau in a cell or subject. Some such methods are for inhibiting accumulation of insoluble tau in a cell or subject. Some such methods are for reducing accumulation of insoluble tau in a cell or subject. Some such methods comprise administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell or subject.

In one aspect, provided are methods of inhibiting tau aggregation or reducing tau phosphorylation in a cell or subject. Some such methods are for inhibiting tau aggregation in a cell or subject. Some such methods are for reducing tau phosphorylation in a cell or subject. Some such methods comprise administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell or subject.

Some such methods comprise administering the LEMD2 or the nucleic acid encoding the LEMD2 to the cell or subject. In some such methods, the LEMD2 is a human LEMD2. Optionally, the LEMD2 comprises SEQ ID NO: 1 or 5. Optionally, the nucleic acid encoding the LEMD2 comprises SEQ ID NO: 2, 3, 6, or 7. In some such methods, the LEMD2 is a mouse LEMD2. Optionally, the LEMD2 comprises SEQ ID NO: 10. Optionally, the nucleic acid encoding the LEMD2 comprises SEQ ID NO: 11, 12, or 13. Optionally, the nucleic acid encoding the LEMD2 comprises SEQ ID NO: 255.

Some such methods comprise administering the CHMP7 or the nucleic acid encoding the CHMP7 to the cell or subject. In some such methods, the CHMP7 is a human CHMP7. Optionally, the CHMP7 comprises SEQ ID NO: 15. Optionally, the nucleic acid encoding the CHMP7 comprises SEQ ID NO: 16 or 17. In some such methods, the CHMP7 is a mouse CHMP7. Optionally, the CHMP7 comprises SEQ ID NO: 19. Optionally, the nucleic acid encoding the CHMP7 comprises SEQ ID NO: 20 or 21.

Some such methods comprise administering the LEMD3 or the nucleic acid encoding the LEMD3 to the cell or subject. In some such methods, the LEMD3 is a human LEMD3. Optionally, the LEMD3 comprises SEQ ID NO: 23. Optionally, the nucleic acid encoding the LEMD3 comprises SEQ ID NO: 24 or 25. In some such methods, the LEMD3 is a mouse LEMD3. Optionally, the LEMD3 comprises SEQ ID NO: 27 or 29. Optionally, the nucleic acid encoding the LEMD3 comprises SEQ ID NO: 29 or 30.

In some such methods, the nucleic acid encoding the LEMD2, the CHMP7, or the LEMD3 is administered to the cell or subject. Optionally, the nucleic acid is codon-optimized for expression in human cells or mouse cells. In some such methods, the nucleic acid comprises a complementary DNA encoding the LEMD2, the CHMP7, or the LEMD3. In some such methods, the nucleic acid comprises a messenger RNA encoding the LEMD2, the CHMP7, or the LEMD3.

In some such methods, the method comprises administering an expression construct comprising the nucleic acid encoding the LEMD2, the CHMP7, or the LEMD3 operably linked to a promoter. In some such methods, the promoter is a heterologous promoter. In some such methods, the promoter is a constitutive promoter, a tissue-specific promoter, or an inducible promoter. In some such methods, the promoter is a neuron-specific promoter. Optionally, the promoter is a synapsin-1 promoter. Optionally, the promoter is a human synapsin-1 promoter.

In some such methods, the nucleic acid is in a vector. In some such methods, the vector is a viral vector. In some such methods, the viral vector is a lentivirus vector or an adeno-associated virus (AAV) vector. In some such methods, the vector is the AAV vector. Optionally, the AAV vector is an AAV-PHP.eB vector.

In some such methods, the cell is a mammalian cell or the subject is a mammal. In some such methods, the mammalian cell is a human cell, a rodent cell, a mouse cell, or a rat cell or the subject is a human, a rodent, a mouse, or a rat. In some such methods, the cell is the human cell or the subject is a human.

In some such methods, the cell is a neuron. In some such methods, the cell is in vivo in a subject. In some such methods, the cell is a neuron in the brain of the subject. In some such methods, the LEMD2, the CHMP7, or the LEMD3 or the nucleic acid encoding the LEMD2, the CHMP7, or the LEMD3 is administered to the subject via intracerebroventricular injection, intracranial injection, or intrathecal injection. In some such methods, the LEMD2, the CHMP7, or the LEMD3 or the nucleic acid encoding the LEMD2, the CHMP7, or the LEMD3 is administered to the subject via intraperitoneal injection.

Some such methods further comprise assessing one or more signs or symptoms of tauopathy or tau aggregation in the cell or subject. Some such methods further comprise assessing phospho-tau levels in the cell or subject. Some such methods further comprise assessing serum neurofilament light chain (sNfL) levels in the subject.

Some such methods reduce the amount of new tau aggregate formation in the cell or subject. Some such methods reduce the amount of preexisting tau aggregate formation in the cell or subject.

In another aspect, provided are methods of treating or preventing a tauopathy in a subject. Some such methods or for treating a tauopathy in a subject. Some such methods are for preventing a tauopathy in a subject. Some such methods comprise administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the subject, wherein the LEMD2, the CHMP7, or the LEMD3 inhibits tau aggregation in a cell in the subject.

Some such methods comprise administering the LEMD2 or the nucleic acid encoding the LEMD2 to the subject. In some such methods, the LEMD2 is a human LEMD2. Optionally, the LEMD2 comprises SEQ ID NO: 1 or 5. Optionally, the nucleic acid encoding the LEMD2 comprises SEQ ID NO: 2, 3, 6, or 7. In some such methods, the LEMD2 is a mouse LEMD2. Optionally, the LEMD2 comprises SEQ ID NO: 10. Optionally, the nucleic acid encoding the LEMD2 comprises SEQ ID NO: 11, 12, or 13. Optionally, the nucleic acid encoding the LEMD2 comprises SEQ ID NO: 255.

Some such methods comprise administering the CHMP7 or the nucleic acid encoding the CHMP7 to the subject. In some such methods, the CHMP7 is a human CHMP7. Optionally, the CHMP7 comprises SEQ ID NO: 15. Optionally, the nucleic acid encoding the CHMP7 comprises SEQ ID NO: 16 or 17. In some such methods, the CHMP7 is a mouse CHMP7. Optionally, the CHMP7 comprises SEQ ID NO: 19. Optionally, the nucleic acid encoding the CHMP7 comprises SEQ ID NO: 20 or 21.

Some such methods comprise administering the LEMD3 or the nucleic acid encoding the LEMD3 to the subject. In some such methods, the LEMD3 is a human LEMD3. Optionally, the LEMD3 comprises SEQ ID NO: 23. Optionally, the nucleic acid encoding the LEMD3 comprises SEQ ID NO: 24 or 25. In some such methods, the LEMD3 is a mouse LEMD3. Optionally, the LEMD3 comprises SEQ ID NO: 27 or 29. Optionally, the nucleic acid encoding the LEMD3 comprises SEQ ID NO: 29 or 30.

In some such methods, the nucleic acid encoding the LEMD2, the CHMP7, or the LEMD3 is administered to the subject. Optionally, the nucleic acid is codon-optimized for expression in human cells or mouse cells. In some such methods, the nucleic acid comprises a complementary DNA encoding the LEMD2, the CHMP7, or the LEMD3. In some such methods, the nucleic acid comprises a messenger RNA encoding the LEMD2, the CHMP7, or the LEMD3.

In some such methods, the method comprises administering an expression construct comprising the nucleic acid encoding the LEMD2, the CHMP7, or the LEMD3 operably linked to a promoter. In some such methods, the promoter is a heterologous promoter. In some such methods, the promoter is a constitutive promoter, a tissue-specific promoter, or an inducible promoter. In some such methods, the promoter is a neuron-specific promoter. Optionally, the promoter is a synapsin-1 promoter. Optionally, the promoter is a human synapsin-1 promoter.

In some such methods, the nucleic acid is in a vector. In some such methods, the vector is a viral vector. In some such methods, the viral vector is a lentivirus vector or an adeno-associated virus (AAV) vector. In some such methods, the vector is the AAV vector. Optionally, the AAV vector is an AAV-PHP.eB vector.

In some such methods, the subject is a mammal. In some such methods, the subject is a human, a rodent, a mouse, or a rat. In some such methods, the subject is the human.

In some such methods, the cell is a neuron. In some such methods, the neuron is in the brain of the subject. In some such methods, the LEMD2, the CHMP7, or the LEMD3 or the nucleic acid encoding the LEMD2, the CHMP7, or the LEMD3 is administered to the subject via intracerebroventricular injection, intracranial injection, or intrathecal injection. In some such methods, the LEMD2, the CHMP7, or the LEMD3 or the nucleic acid encoding the LEMD2, the CHMP7, or the LEMD3 is administered to the subject via intraperitoneal injection.

Some such methods further comprise assessing one or more signs or symptoms of tauopathy or tau aggregation in the cell or the subject. Some such methods further comprise assessing phospho-tau levels in the cell or the subject. Some such methods further comprise assessing serum neurofilament light chain (sNfL) levels in the subject.

Some such methods reduce the amount of new tau aggregate formation in the cell or the subject. Some such methods reduce the amount of preexisting tau aggregate formation in the cell or the subject.

In another aspect, provided are expression constructs comprising a nucleic acid encoding a LEM domain-containing protein 2 (LEMD2), a charged multivesicular body protein 7 (CHMP7), or an inner nuclear membrane protein Man 1 (LEMD3) operably linked to a heterologous promoter.

Some such expression constructs comprise the nucleic acid encoding the LEMD2. In some such expression constructs, the LEMD2 is a human LEMD2. Optionally, the LEMD2 comprises SEQ ID NO: 1 or 5. Optionally, the nucleic acid encoding the LEMD2 comprises SEQ ID NO: 2, 3, 6, or 7. In some such expression constructs, the LEMD2 is a mouse LEMD2. Optionally, the LEMD2 comprises SEQ ID NO: 10. Optionally, the nucleic acid encoding the LEMD2 comprises SEQ ID NO: 11, 12, or 13. Optionally, the nucleic acid encoding the LEMD2 comprises SEQ ID NO: 255.

Some such expression constructs comprise the nucleic acid encoding the CHMP7. In some such expression constructs, the CHMP7 is a human CHMP7. Optionally, the CHMP7 comprises SEQ ID NO: 15. Optionally, the nucleic acid encoding the CHMP7 comprises SEQ ID NO: 16 or 17. In some such expression constructs, the CHMP7 is a mouse CHMP7. Optionally, the CHMP7 comprises SEQ ID NO: 19. Optionally, the nucleic acid encoding the CHMP7 comprises SEQ ID NO: 20 or 21.

Some such expression constructs comprise the nucleic acid encoding the LEMD3. In some such expression constructs, the LEMD3 is a human LEMD3. Optionally, the LEMD3 comprises SEQ ID NO: 23. Optionally, the nucleic acid encoding the LEMD3 comprises SEQ ID NO: 24 or 25. In some such expression constructs, the LEMD3 is a mouse LEMD3. Optionally, the LEMD3 comprises SEQ ID NO: 27 or 29. Optionally, the nucleic acid encoding the LEMD3 comprises SEQ ID NO: 29 or 30.

In some such expression constructs, the nucleic acid encoding the LEMD2, the CHMP7, or the LEMD3 is codon-optimized for expression in human cells or mouse cells. In some such expression constructs, the nucleic acid comprises a complementary DNA encoding the LEMD2, the CHMP7, or the LEMD3.

In some such expression constructs, the promoter is a constitutive promoter, a tissue-specific promoter, or an inducible promoter. In some such expression constructs, the promoter is a neuron-specific promoter. Optionally, the promoter is a synapsin-1 promoter. Optionally, the promoter is a human synapsin-1 promoter.

In some such expression constructs, the nucleic acid is in a vector. In some such expression constructs, the vector is a viral vector. In some such expression constructs, the viral vector is a lentivirus vector or an adeno-associated virus (AAV) vector. In some such expression constructs, the vector is the AAV vector. Optionally, the AAV vector is an AAV-PHP.eB vector.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D show a screen for mutations that enhance tau aggregation. FIG. 1A shows a schematic illustrating the detection of tau aggregation by the induction of FRET. Tau biosensor cells treated with conditioned medium from cells lacking tau aggregates, tau-YFP Agg- (IF image on top left), do not produce a FRET signal after three days in culture (flow cytometry plot on top right). Biosensor cells treated with conditioned medium from cells containing tau aggregates, tau-YFP Agg+ (IF image on bottom left), induce a detectable FRET signal in 0.1% of cells (flow cytometry plot on bottom right). FIG. 1B shows a schematic of the CRISPR screening timeline. Lentivirus-packaged CRISPR gRNA libraries were transduced into Cas9-expressing biosensor cells on day 0. Differently colored nuclei indicate that each cell has received a different gRNA-expressing transgene. Cells were sampled on day 3 and day 6, and FACS was performed on day 10 to sort and collect FRET+ cells. gRNA representation was determined by PCR amplification and sequencing of amplicons. FIG. 1C shows a plot of gene-centric enrichment relative to p-value in day 10 FRET+ cells for gRNAs targeting the same gene. BANF1 is indicated with circles marked with an “X” and PPP2CA is indicated by white circles. FIG. 1D shows secondary screening of 14 primary screen candidate genes for enhancement of tau aggregation in biosensor cells seeded with tau-YFP Agg+ conditioned medium, measured as FRET induction (calculated as integrated FRET density), after inactivation by individual lentiviral gRNAs expression vectors (four gRNAs for BANF1 and two for each of the 13 other genes). The bar heights represent the mean ± SEM of all gRNAs analyzed. Genes targeted by the gRNAs are indicated on the X-axis. Large enhancements of FRET induction for gRNAs targeting BANF1 or PPP2CA confirm these genes as hits in the screen for enhanced tau aggregation. Controls (first two bars) were mock transduction without virus and transduction with a lentiviral vector expressing a non-targeting control gRNA. See also FIGS. 5A-5E and 6A-6G.

FIGS. 2A-2E show targeted modulation of genes encoding proteins in a nuclear envelope-related BANF1-interacting network can promote or inhibit tau aggregation. FIG. 2A shows that a String database query for BANF1 returns a functional interaction network of proteins that participate in the maintenance of nuclear envelope integrity. FIG. 2B shows that the members of the BANF1 interacting network reveal ANKLE2 as a gene whose gRNA-targeted inactivation enhances tau aggregation in biosensor cells with tau-YFP Agg+ conditioned medium, measured as FRET induction (calculated as integrated FRET density, mean ± std. dev.). Cas9-expressing biosensor cells were transduced with lentiviral vectors expressing targeting gRNAs, LV-gRNAs. Controls (first two bars) were mock transduced without virus and transduced with a lentiviral vector expressing a control gRNA. Inactivation of ANKLE2, but not other BANF1-interacting proteins, enhances FRET induction. FIG. 2C shows confirmation that inactivation of BANF1, PPP2CA, or ANKLE2, but not LEMD2 or LEMD3, with individual LV-gRNAs enhances tau aggregation measured as FRET induction by three different sources of tau seeding activity: sonicated whole cell lysate from tau-YFP Agg+ cells; purified recombinant tau fibrils; and spinal cord lysate from 9-month-old tau P301S transgenic mice. Cas9-expressing biosensor cells were transduced with individual LV-gRNAs. Controls were transduced with a control gRNA (first three bars) and gRNAs targeting LEMD2 and LEMD3, two genes not confirmed in the screen of network members (last six bars). FIG. 2D shows an illustration of the nuclear envelope with the associated LEM domain-containing proteins ANKLE2, LEMD2, short isoform LEMD2iso2, and LEMD3. Also shown is the repair factor CHMP7. ER, endoplasmic reticulum; NPC, nuclear pore complex; INM, inner nuclear membrane; ONM; outer nuclear membrane. FIG. 2E shows expression in biosensor cells of cDNAs that encode LEMD2, LEMD2i2, LEMD3, and CHMP7 proteins reduces FRET induced by a strong seeding agent (tau-YFP Agg+ whole cell lysate with LIPOFECTAMINE™) compared with control cells expressing the firefly luciferase. Biosensor cells were transduced with lentiviral vectors expressing cDNAs, LV-cDNAs. Bars represent the mean integrated FRET density ± std. dev. for four replicate samples. Significance was determined using an unpaired two-tailed t test, *p<0.01, **p<0.0001.

FIGS. 3A-3H show identification of genes whose overexpression rescues tau aggregation. FIGS. 3A and 3B show rescue of FRET induction phenotype through over-expression of LEMD2, LEMD2i2, LEMD3 and CHMP7 nuclear envelope components. dCas9-KRAB-expressing biosensor cells were transduced with LV-gRNAs targeting BANF1 (pink) or ANKLE2 (blue) that transcriptionally repress their gene targets, or with a control gRNA (gray), and with LV-cDNAs that encode LEMD2, LEMD2i2, LEMD3, and CHMP7 proteins. FRET was induced by seeding with spinal cord lysate from a 9-month-old tau P301S transgenic mouse (FIG. 3A) or with tau-YFP Agg+ whole cell lysate (FIG. 3B). Bars represent the mean integrated FRET density for three replicate samples. Graphs show the mean ± SEM. FIGS. 3C-3D show western blots detecting total tau protein or tau phosphorylated on Serine 356. dCas9-KRAB expressing tau biosensor cells transduced with gRNAs targeting BANF1 or ANKLE2 were treated with whole cell lysate from tau-YFP Agg- cells (left) or tau-YFP Agg+ cells (right), and co-transduced with LV-cDNAs for luciferase (FIG. 3C) or LEMD2 (FIG. 3D). Only cells treated with the tau-YFP Agg+ whole cell lysate exhibit increases in total tau and P-tau-Ser356 in the insoluble fraction (FIG. 3C, red box). This increase is prevented by transduction of LEMD2 cDNA (FIG. 3D, red box). As these Western blots were performed on denaturing gels, monomeric and multimeric species of tau cannot be distinguished. FIG. 3E shows representative confocal microscopy images of immunofluorescence detection of SRRM2 (yellow) and tau phosphorylated on serine 356 (magenta). Biosensor cells were treated with tau-YFP Agg+ whole cell lysate. In cells with tau aggregates, SRRM2 is mislocalized from the nucleus (indicated by blue DAPI staining) and co-localizes with cytoplasmic tau aggregates positive for P-tau-Ser356. FIG. 3F shows representative confocal microscopy images of immunofluorescence detection of SRRM2 (yellow) in dCas9-KRAB-expressing biosensor cells transduced with a control gRNA or with gRNAs targeting BANF1 or ANKLE2 and treated with the tau-YFP Agg+ whole cell lysate. In cells depleted for BANF1 and ANKLE2, some SRRM2 is detected as cytoplasmic foci close to the nucleus. DAPI staining (blue) identifies nuclei. Scale bar = 20 µm. FIG. 3G shows histogram plots showing the proportion of cells with only nuclear SRRM2 (solid color) and cells with nuclear as well as mislocalized cytoplasmic SRRM2 (cross hatching). FIG. 3H shows quantification of the percentage of total SRRM2 immunofluorescence intensity in the nucleus of each cell. Quantifications for biosensor cells depleted for BANF1 are shown in pink, for ANKLE2 in blue and for control cells in gray. Fluorescence images of individual cells were quantified by Harmony software (Perkin-Elmer). The bar heights represent the mean ± SEM of all cells analyzed (n = 113). Significance was determined using an unpaired two-tailed t test, **p<0.0001.

FIGS. 4A-4I show that disruption of Ankle2, Banf1, or Ppp2ca in primary mouse cortical neurons enhances the production of tau phosphorylated on Serine 356 and impairs nuclear envelope integrity. FIG. 4A shows representative confocal microscopy images of immunofluorescence staining for P-tau-Ser356 (yellow) and microtubule-associated protein 2 (MAP2, red) on wildtype primary mouse cortical neurons 14 days after transduction with lentiviral vectors co-expressing Cas9 with either a control gRNA or a gRNA targeting Banf1. DAPI staining (blue) identifies nuclei. Scale bar = 50 µm. FIG. 4B shows quantification of DAPI+ cells per culture well. FIG. 4C shows quantification of P-tau-Ser356 in the soma, FIG. 4D shows quantification of P-tau-Ser356 in the nucleoplasm, and FIG. 4E shows quantification of P-tau-Ser356 in the perinuclear domain of control and Ankle2, Banf1, and Ppp2ca mutant cells. Primary mouse cortical neurons transduced with LV-Cas9-gRNA were immunostained with antibodies to MAP2 or P-tau-Ser356. FIG. 4F shows representative microscopy images of live primary mouse cortical neurons expressing EF1a-nls::mCherry, treated for 8 days with ASOs delivered gymnotically. Depicted are merged images of bright-field and mCherry views (left, red color) and mCherry alone (right, white color). Scale bar = 10 µm. FIG. 4G and FIG. 4H show Banf1 and Ankle2 relative expression, respectively, assessed by TaqMan qRT-PCR and normalized to control treatment. Each value represents the average ± STDEV of two replicates. Gapdh expression used as a reference gene. FIG. 4I shows quantification of mCherry fluorescence intensity. Three equivalent areas were measured in the soma per cell. Graphs show the mean ± SEM (n = 12 for FIGS. 4B-4E and n = 7 for FIG. 4I) analyzed by the Mann-Whitney U test, two-tailed exact significance, *p<0.01, **p<0.007, ***p<0.0001, n.s.=not significant. See also FIGS. 8A-8K and 9A-9G.

FIGS. 5A-5H show development of a tau biosensor cell-based screening platform. FIG. 5A shows a negative stain TEM micrograph of recombinant tau Q244-E372; P301L, V337M (tau 244-372 LM tau) fibrils. FIG. 5B shows that static Thioflavin T (ThT) fluorescence of tau 244-372 LM monomer and fibrils, showing that fibrils bind the amyloid specific dye ThT, while monomer does not. FIGS. 5C-5D show fibril formation kinetics of tau 244-372 LM (FIG. 5C) with heparin or (FIG. 5D) without heparin monitored by ThT fluorescence. Tau 244-372 LM was incubated at 37° C. with 700 rpm double orbital shaking, with or without heparin at a 4:1 tau to heparin ratio. With heparin all three concentrations tested (50, 25, and 10 µM) display a rapid increase in ThT intensity, corresponding to T_{½} ~0.5 hrs. Tau 244-372 LM incubated without heparin displayed a concentration dependent increase in ThT intensity, with 50 µM tau 244-372 LM showing a T_{½} ~14 hours, 25 µM tau 244-372 LM showing a T_{½} ~31 hours, while 10 µM tau 244-372 LM did not display any increase in ThT intensity within the 120 hours measured. FIG. 5E shows tau biosensor cells are HEK293T cells with two transgenes expressing the four Repeat Domain (4RD) of human protein tau, containing the P301S pathogenic mutation, and fused to a CFP or YFP fluorescent reporter. Upon treatment with a source of tau seeding activity, biosensor cells will form visible tau aggregates that are phosphorylated on Ser356. Biosensor cells will produce a FRET signal upon tau aggregation. FIG. 5F shows representative confocal microscopy images of biosensor cells seeded with whole cell lysate from tau-YFP Agg+ cells, showing immunofluorescence detection of P-tau-Ser356 (magenta), and aggregated tau protein (tau-YFP, visualized in yellow). DAPI staining (blue) indicates nuclei. Scale bar= 20 µm. FIG. 5G shows evaluation of tau biosensor Cas9 clones. Expression of the Cas9 transgene was assessed by TaqMan qRT-PCR and cutting efficiency evaluated by digital PCR (dPCR) at 3 and 7 days after transduction of a gRNA targeting PERK. FIG. 5H shows a plot of gRNA enrichment relative to p-value in day 10 FRET+ cells as compared with day 6 cells. gRNAs targeting BANF1 indicated by circles marked with an “X”.

FIGS. 6A-6G shows modulation of nuclear envelope-associated genes affects tau aggregation and related phenotypes. FIG. 6A shows treatment with tau-YFP Agg+ Conditioned Medium is required to produce FRET signal, and to detect enhancement of FRET signal resulting from disruption of tau modifier genes. Treatment with fresh medium resulted in no FRET signal, even after disruption of BANF1 or PPP2CA. FIG. 6B shows western blots showing specific reduction in protein levels in Cas9-expressing biosensor cells after transduction with LV-gRNAs targeting BANF1 or PPP2CA. FIG. 6C shows that Cas9-expressing biosensor cells transduced with LV-gRNAs targeting BANF1 or PPP2CA were used to isolate single-cell knockdown clones. These clones exhibit increased FRET induction only after treatment with tau-YFP Agg+ Conditioned Medium, which correlates with the percent of gene editing at the target locus, as assessed by NGS. In FIG. 6C, FRET induction is calculated as Integrated FRET Density, and each value represents the average ± STDEV of at least two replicates. FIG. 6D shows co-transduction of multiple LV-gRNAs into biosensor cells at high MOI. Co-transduction of LV-gRNAs targeting BANF1, ANKLE2, and PPP2CA in different combinations does not result in increased FRET induction as compared to targeting ANKLE2 alone. FIG. 6E shows validation of the CRISPRa SAM activation system in biosensor cells. dCas9-SAM-expressing tau biosensor cells were transduced with LV-gRNAs, each targeting one of 11 genes. TaqMan expression analysis revealed that the SAM-mediated transcriptional activation inversely correlates with the basal transcript level (as RPKM, a normalized unit of transcript expression). FIGS. 6F and 6G show cDNA expression analysis of potential tau modifier genes. Biosensor cells were transduced with individual lentivirus-packaged cDNAs, LV-cDNAs, that induce high expression of the encoded protein. Transduced cells were selected and collected for expression analysis using TaqMan assays designed to amplify specifically these codon-optimized (FIG. 6F) and MAPT-4RD (FIG. 6G) cDNAs. ΔCt values are indicated for each specific cDNA-transduced sample. Each value represents the average ± STDEV of four replicates (shown individually as symbols).

FIGS. 7A-7F show genetic interactions of nuclear envelope components modifying tau aggregation. FIG. 7A shows an illustration of the recently uncovered roles of BANF1 and LEMD2 to facilitate the sealing of the nuclear envelope after damage. Mechanical stress imposed on the nucleus may lead to ruptures of the nuclear envelope, which are repaired by the recruitment of the Endosomal Sorting Complex Required for Transport-III, ESCRT-III, complex. At rupture sites, cytosolic BANF1 coats the exposed chromatin and recruits membranes through its interaction with LEMD2. Local increased concentration of LEMD2 allows the recruitment of CHMP7 to the Inner Nuclear Membrane, which promotes ESCRT-III nucleation. Finally, ESCRT-III complex with ATPase VPS4 constricts the rupture and promotes sealing of the nuclear envelope, reviewed by Zhen et al. (2021) EMBO J. 40:e106922, herein incorporated by reference in its entirety for all purposes. FIG. 7B shows relative expression of BANF1, FIG. 7C shows relative expression of ANKLE2, and FIG. 7D shows relative expression of MAPT-4RD, in dCas9-KRAB and cDNA expressing biosensor cells. Relative expression assessed by TaqMan qRT-PCR and normalized to control treatment. Each value represents the average ± STDEV of at least two replicates. GAPDH expression was used as a reference gene. FIG. 7E shows rescue of FRET induction phenotype through over-expression of nuclear envelope components. Over-expression of BANF1 cDNA specifically abolishes the increased FRET induction by tau-YFP Agg+ cell lysate resulting from BANF1 knockdown, but not that resulting from ANKLE2 knockdown, in dCas9-KRAB expressing biosensor cells. FIG. 7F shows a western blot detecting BANF1 protein in dCas9-KRAB expressing biosensor cells transduced with LV-gRNAs targeting BANF1 or ANKLE2, and seeded with whole cell lysate from tau-YFP Agg- cells (left) or tau-YFP Agg+ cells (right), and co-transduced with cDNA expressing Luciferase (top) or BANF1 (bottom). Disruption of ANKLE2 causes a mislocalization of BANF1 protein from the chromatin bound fraction into the cytoplasmic fractions, which is not rescued by overexpression of BANF1 cDNA.

FIGS. 8A-8K show that disruption of Ankle2, Banf1, or Ppp2ca in primary mouse cortical neurons affects tau phosphorylation and sub-cellular localization, but does not have strong effects on total tau protein. FIG. 8A shows confocal images of primary mouse cortical neurons, highlighting the compartments of interest as segmented by the Harmony software: nucleoplasm (left), perinuclear domain (middle), and soma (right). FIG. 8B shows percent of gene editing at the target cutting site, as assessed by NGS. FIG. 8C shows relative gene expression of primary mouse cortical neurons transduced with LV-Cas9-gRNA, as assessed by TaqMan qRT-PCR assays. Data are normalized to control cells. Each value represents the average ± STDEV of four replicates. Gapdh expression was used as a reference gene. FIG. 8D shows representative confocal microscopy images of immunofluorescence staining for P-tau-Ser356 (yellow) and microtubule-associated protein 2 (MAP2, red) on primary cortical neurons 14 days after transduction with LV-Cas9-gRNAs. DAPI staining (blue) identifies nuclei. Scale bar= 50 µm . FIG. 8E shows quantification of MAP2 protein in the soma that reveals significant reduction in signal associated with disruption of Ankle2, Banf1, and Ppp2ca, with the latter being the most severe. FIG. 8F shows representative confocal images of primary mouse cortical neurons immunostained for MAP2 (red) and total tau protein (yellow), following disruption of Ankle2, Banf1, or Ppp2ca. FIG. 8G shows quantification of DAPI+ cells per culture well, showing apparent toxicity associated with loss of Ppp2ca. FIG. 8H shows quantification of MAP2 protein in the soma of primary mouse cortical neurons. FIGS. 8I-8K show quantification of total tau in the soma, perinuclear domain and nucleoplasm of primary mouse cortical neurons transduced with LV-Cas9-gRNA and immunostained with antibodies to MAP2 or total tau. Graphs show the mean ± SEM (n = 12) analyzed by the Mann-Whitney U test, two-tailed exact significance, *p<0.01, n.s.=not significant.

FIGS. 9A-9G show Lemd2, Lemd3 and Chmp7 cDNAs can rescue increased Phospho-tau-Ser356 in Banf1 or Ankle2 targeted knockdown in primary mouse cortical neurons. Neurons were treated for 10 days with individual ASO via gymnotic delivery. ASO were replenished at each medium change. LV-hSyn1-cDNAs were transduced 4 days after initial ASO treatment. Quantification of DAPI+ cells per replicate well, of MAP2 protein in the soma of neurons, of P-tau-Ser356 in the soma, perinuclear domain and nucleoplasm domains is shown in FIGS. 9A-9D. Neurons were transduced with LV- hSyn1-cDNAs encoding Luciferase (FIG. 9A), Lemd2 (FIG. 9B), Lemd3 (FIG. 9C) and Chmp7 (FIG. 9D). At Day 14, Banf1 (FIG. 9E) or Ankle2 (FIG. 9F) relative expression of ASOs treated neurons was assessed by target specific TaqMan qRT-PCR assays, as well as codon-optimized cDNAs specific TaqMan assays (FIG. 9G). Graphs show the mean ± SEM (n = 12) analyzed by the Mann-Whitney U test, two-tailed exact significance, *p<0.05, **p<0.005, *** p<0.0001, n.s.=not significant.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous LEMD2 sequence of an animal refers to a native LEMD2 sequence that naturally occurs at the LEMD2 locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of′ means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values ± 5% of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p ≤0.05.

### DETAILED DESCRIPTION

### I. Overview

The microtubule-associated protein tau is an abundant component of neurons of the central nervous system, where it functions to maintain microtubule stability and promote axonal growth. In Alzheimer’s disease and other neurodegenerative tauopathies, tau is found hyperphosphorylated and aggregated in neurofibrillary tangles. To obtain a better understanding of the cellular perturbations that initiate tau pathogenesis, we performed a CRISPR-Cas9 genetic screen for mutations that enhance tau aggregation. This screen yielded three genes, BANF1, ANKLE2, and PPP2CA, whose inactivation promoted the accumulation of tau in a phosphorylated and insoluble form. In a complementary screen, we identified three additional genes, LEMD2, LEMD3, and CHMP7, that when overexpressed provided protection against tau aggregation. The proteins encoded by the identified genes participate in the maintenance and repair of the nuclear envelope. These results implicate disruption of nuclear envelope integrity as a possible initiating event in tauopathies and reveal new targets for therapeutic intervention.

Provided herein are methods of inhibiting tau aggregation in a cell or a subject, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell or the subject. Also provided herein are methods of reducing tau phosphorylation (e.g., phosphorylation on serine 356) in a cell or a subject, comprising administering LEMD2, CHMP7, or LEMD3 or a nucleic acid encoding the LEMD2, the CHMP7, or the LEMD3 to the cell or the subject. Also provided are methods of reducing serum neurofilament light chain (sNfL) or preventing accumulation of serum neurofilament light chain (sNfL) in a subject, comprising administering LEMD2, CHMP7, or LEMD3 or a nucleic acid encoding the LEMD2, the CHMP7, or the LEMD3 to the subject. Also provided herein are methods of treating a tauopathy in a subject, comprising administering LEMD2, CHMP7, or LEMD3 or a nucleic acid encoding the LEMD2, the CHMP7, or the LEMD3 to the subject, wherein the LEMD2, the CHMP7, or the LEMD3 inhibits tau aggregation in a cell in the subject. Also provided are nucleic acids encoding LEMD2, CHMP7, or LEMD3 (e.g., in an expression construct and operably linked to a heterologous promoter), constructs comprising the nucleic acids, vectors comprising the nucleic acid or constructs, lipid nanoparticles comprising the nucleic acids, constructs, or vectors, and cells or subjects (e.g., animals) comprising the nucleic acids, constructs, vectors, or lipid nanoparticles.

### II. Methods of Inhibiting Tau Aggregation and Methods of Treating or Preventing Tauopathies

Provided herein are methods of inhibiting tau aggregation in a cell or a subject. Such methods can comprise administering a LEM domain-containing protein 2 (LEMD2), a charged multivesicular body protein 7 (CHMP7), or an inner nuclear membrane protein Man 1 (LEMD3) to the cell (i.e., an exogenous LEMD2, CHMP7, or LEMD3), or such methods can comprise administering a nucleic acid encoding the LEMD2, the CHMP7, or the LEMD3 to the cell (i.e., an exogenous nucleic acid encoding the LEMD2, CHMP7, or LEMD3) or the subject such that the LEMD2, the CHMP7, or the LEMD3 is expressed. Also provided herein are methods of reducing tau phosphorylation (e.g., phosphorylation on serine 356) in a cell or a subject, comprising administering LEMD2, CHMP7, or LEMD3 or a nucleic acid encoding the LEMD2, the CHMP7, or the LEMD3 (i.e., an exogenous nucleic acid encoding the LEMD2, CHMP7, or LEMD3) to the cell or the subject. The phospho-tau can be, for example, phospho-tau (S356) or phospho-tau AT8 (S202, T205). Also provided are methods of reducing serum neurofilament light chain (sNfL) or preventing accumulation of serum neurofilament light chain (sNfL) in a subject. Such methods can comprise administering LEMD2, CHMP7, or LEMD3 or a nucleic acid encoding the LEMD2, the CHMP7, or the LEMD3 to the subject. Also provided are methods of treating a tauopathy in a subject. Such methods can comprise administering LEMD2, CHMP7, or LEMD3 to the subject (i.e., an exogenous LEMD2, CHMP7, or LEMD3), or such methods can comprise administering a nucleic acid encoding the LEMD2, the CHMP7, or the LEMD3 to the subject (i.e., an exogenous nucleic acid encoding the LEMD2, CHMP7, or LEMD3) such that the LEMD2, the CHMP7, or the LEMD3 is expressed. The LEMD2, the CHMP7, or the LEMD3 then inhibits tau aggregation in the subject (e.g., in a cell in the subject or in one or more cells of the subject). Also provided are methods of preventing a tauopathy in a subject. Such methods can comprise administering LEMD2, CHMP7, or LEMD3 to the subject (i.e., an exogenous LEMD2, CHMP7, or LEMD3), or such methods can comprise administering a nucleic acid encoding the LEMD2, the CHMP7, or the LEMD3 to the subject (i.e., an exogenous nucleic acid encoding the LEMD2, CHMP7, or LEMD3) such that the LEMD2, the CHMP7, or the LEMD3 is expressed. The LEMD2, the CHMP7, or the LEMD3 then inhibits tau aggregation in the subject (e.g., in a cell in the subject or in one or more cells of the subject).

Microtubule-associated protein tau (also called neurofibrillary tangle protein, paired helical filament-tau (PHF-tau), or tau) is a protein that promotes microtubule assembly and stability and is predominantly expressed in neurons, where it is preferentially localized to the axonal compartment. Tau is encoded by the MAPT gene (also called MAPTL, MTBT1, TAU, or MTAPT). Tau has a role in stabilizing neuronal microtubules and thus in promoting axonal outgrowth. In humans, it appears as a set of six isoforms which are differentially spliced from transcripts of a single gene located on chromosome 17. Each tau isoform contains a series of ¾ tandem repeat units (depending on the isoform) that bind to microtubules and serve to stabilize them. The microtubule-binding repeat region of tau is flanked by serine/threonine-rich regions which can be phosphorylated by a variety of kinases and that are associated with tau hyperphosphorylation in a family of related neurodegenerative diseases called tauopathies.

The tau proteins are the products of alternate splicing from a single gene that in humans is designated MAPT (microtubule-associated protein tau). The tau repeat domain carries the sequence motifs responsible for aggregation (i.e., it is the aggregation-prone domain from tau). Depending on splicing, the repeat domain of the tau protein has either three or four repeat regions that constitute the aggregation-prone core of the protein, which is often termed the repeat domain (RD). Specifically, the repeat domain of tau represents the core of the microtubule-binding region and harbors the hexapeptide motifs in R2 and R3 that are responsible for Tau aggregation. In the human brain, there are six tau isoforms ranging from 352 to 441 amino acids in length. These isoforms vary at the carboxyl terminal according to the presence of either three repeat or four repeat domains (R1-R4), in addition to the presence or absence of one or two insert domains at the amino-terminus. The repeat domains, located at the carboxyl-terminal half of tau, are believed to be important for microtubule binding as well as for the pathological aggregation of tau into paired helical filaments (PHFs), which are the core constituents of the neurofibrillary tangles found in tauopathies.

In some of the above methods for inhibiting tau aggregation, reducing tau phosphorylation, treating a tauopathy, or preventing a tauopathy, the methods comprise administering the LEMD2 or the nucleic acid encoding the LEMD2 to the cell or the subject. In some of the above methods for reducing serum neurofilament light chain (sNfL) or preventing accumulation of serum neurofilament light chain (sNfL), the methods comprise administering the LEMD2 or the nucleic acid encoding the LEMD2 to the subject. For example, the LEMD2 can be a wild type LEMD2. LEMD2 is involved in nuclear structure organization and is required for maintaining the integrity of the nuclear envelope.

In one example the LEMD2 is a human LEMD2. Human LEMD2 (also called LEM domain-containing protein 2, hLEM2, or LEM domain nuclear envelope protein 2) is assigned UniProt reference number Q8NC56. The human gene encoding LEMD2 (LEMD2, or LEM domain nuclear envelope protein 2) is assigned NCBI GeneID 221496 and is found at location 6p21.31 on chromosome 6 (assembly: GRCh38.p13 (GCF_000001405.39); location: NC_000006.12 (33771213..33794274, complement)). At least two isoforms of human LEMD2 are known. The first isoform is 503 amino acids and is assigned UniProt reference number Q8NC56-1 and NCBI reference number NP_851853.1 (SEQ ID NO: 1). An exemplary coding sequence is assigned reference number CCDS4785.1 (SEQ ID NO: 2), and an exemplary mRNA (cDNA) sequence is assigned reference number NM_181336.4 (SEQ ID NO: 4). A codon-optimized coding sequence to distinguish from the native CDS is set forth in SEQ ID NO: 3. The second isoform is 201 amino acids and is assigned UniProt reference number Q8NC56-2 and NCBI reference numbers NP_001137416.1 and NP_001335638.1 (SEQ ID NO: 5). An exemplary coding sequence is assigned reference number CCDS47411.1 (SEQ ID NO: 6), and exemplary mRNA (cDNA) sequences are assigned reference numbers NM_001143944.1 and NM_001348709.2 (SEQ ID NOS: 8 and 9, respectively). A codon-optimized coding sequence to distinguish from the native CDS is set forth in SEQ ID NO: 7.

In another example the LEMD2 is a mouse LEMD2. Mouse LEMD2 is assigned UniProt reference number Q6DVA0. The mouse gene encoding LEMD2 (Lemd2) is assigned NCBI GeneID 224640 and is found at location 17; 17 A3.3 on chromosome 17 (assembly: GRCm39 (GCF_000001635.27); Location: NC_000083.7 (27408574..27426228, complement)). An exemplary mouse LEMD2 is 511 amino acids and is assigned UniProt reference number Q6DVA0-1 and NCBI reference number NP_666187.2 (SEQ ID NO: 10). An exemplary coding sequence is assigned reference number CCDS50043.1 (SEQ ID NO: 11), and an exemplary mRNA (cDNA) sequence is assigned reference number NM_146075.2 (SEQ ID NO: 14). Codon-optimized coding sequences to distinguish from the native CDS are set forth in SEQ ID NOS: 12 and 13. Another codon-optimized coding sequence to distinguish from the native CDS is set forth in SEQ ID NO: 255.

In some of the above methods for inhibiting tau aggregation, reducing tau phosphorylation, treating a tauopathy, or preventing a tauopathy, the methods comprise administering the CHMP7 or the nucleic acid encoding the CHMP7 to the cell or the subject. In some of the above methods for reducing serum neurofilament light chain (sNfL) or preventing accumulation of serum neurofilament light chain (sNfL), the methods comprise administering the CHMP7 or the nucleic acid encoding the CHMP7 to the subject. For example, the CHMP7 can be a wild type CHMP7. CHMP7 is an ESCRT-III-like protein required to recruit the ESCRT-III complex to the nuclear envelope during late anaphase. Together with SPAST, the ESCRT-III complex promotes nuclear envelope sealing and mitotic spindle disassembly during late anaphase. CHMP7 also plays a role in the endosomal sorting pathway.

In one example the CHMP7 is a human CHMP7. Human CHMP7 (also called charged multivesicular body protein 7 or chromatin-modifying protein 7) is assigned UniProt reference number Q8WUX9. The human gene encoding CHMP7 (CHMP7) is assigned NCBI GeneID 91782 and is found at location 8p21.3 on chromosome 8 (assembly: GRCh38.p13 (GCF_000001405.39); Location: NC_000008.11 (23243637..23262000)). An exemplary CHMP7 protein is 453 amino acids and is assigned UniProt reference number Q8WUX9-1 and NCBI reference number NP_689485.1 (SEQ ID NO: 15). An exemplary coding sequence is assigned reference number CCDS6040.1 (SEQ ID NO: 16), and an exemplary mRNA (cDNA) sequence is assigned reference number NM_152272.5 (SEQ ID NO: 18). A codon-optimized coding sequence to distinguish from the native CDS is set forth in SEQ ID NO: 17.

In another example the CHMP7 is a mouse CHMP7. Mouse CHMP7 is assigned UniProt reference number Q8R1T1. The mouse gene encoding CHMP7 (Chmp7) is assigned NCBI GeneID 105513 and is found at location 14; 14 D2 on chromosome 14 (assembly: GRCm39 (GCF_000001635.27); Location: NC_000080.7 (69954428..69970019, complement)). An exemplary mouse CHMP7 is 451 amino acids and is assigned UniProt reference number Q8R1T1-1 and NCBI reference number NP_598839.2 (SEQ ID NO: 19). An exemplary coding sequence is assigned reference number CCDS27242.1 (SEQ ID NO: 20), and an exemplary mRNA (cDNA) sequence is assigned reference number NM_134078.4 (SEQ ID NO: 22). A codon-optimized coding sequence to distinguish from the native CDS is set forth in SEQ ID NO: 21.

In some of the above methods for inhibiting tau aggregation, reducing tau phosphorylation, treating a tauopathy, or preventing a tauopathy, the methods comprise administering the LEMD3 or the nucleic acid encoding the LEMD3 to the cell or the subject. In some of the above methods for reducing serum neurofilament light chain (sNfL) or preventing accumulation of serum neurofilament light chain (sNfL), the methods comprise administering the LEMD3 or the nucleic acid encoding the LEMD3 to the subject. For example, the LEMD3 can be a wild type LEMD3.

In one example the LEMD3 is a human LEMD3. Human LEMD3 (also called inner nuclear membrane protein Man1 or LEM domain-containing protein 3) is assigned UniProt reference number Q9Y2U8. The human gene encoding LEMD3 (LEMD3, MAN1, or LEM domain containing 3) is assigned NCBI GeneID 23592 and is found at location 12q14.3 on chromosome 12 (assembly: GRCh38.p13 (GCF_000001405.39); Location: NC_000012.12 (65169583..65248355)). An exemplary LEMD3 protein is 911 amino acids and is assigned UniProt reference number Q9Y2U8-1 and NCBI reference number NP-055134.2 (SEQ ID NO: 23). An exemplary coding sequence is assigned reference number CCDS8972.1 (SEQ ID NO: 24), and an exemplary mRNA (cDNA) sequence is assigned reference number NM_014319.5 (SEQ ID NO: 26). A codon-optimized coding sequence to distinguish from the native CDS is set forth in SEQ ID NO: 25.

In another example the LEMD3 is a mouse LEMD3. Mouse LEMD3 is assigned UniProt reference number Q9WU40. The mouse gene encoding LEMD3 (Lemd3) is assigned NCBI GeneID 380664 and is found at location 10; 10 D2 on chromosome 10 (assembly: GRCm39 (GCF_000001635.27); Location: NC_000076.7 (120759316..120815491, complement)). An exemplary mouse LEMD3 is 921 amino acids and is assigned UniProt reference number Q9WU40-1 (SEQ ID NO: 27). A codon-optimized coding sequence to distinguish from the native CDS is set forth in SEQ ID NO: 28. Another exemplary mouse LEMD3 is 918 amino acids in length and is assigned NCBI reference number NP_001074662.2 (SEQ ID NO: 29). An exemplary coding sequence is assigned reference number CCDS48703.1 (SEQ ID NO: 30), and an exemplary mRNA (cDNA) sequence is assigned reference number NM_001081193.2 (SEQ ID NO: 31).

Some such methods comprise administering the nucleic acid encoding the LEMD2, the nucleic acid encoding the CHMP7, or the nucleic acid encoding the LEMD3 to the cell or the subject. The nucleic acid can be a nucleic acid construct described in more detail elsewhere herein. In some cases, the nucleic acid encoding the LEMD2, CHMP7, or LEMD3 can be a native coding sequence. In other cases, it can be codon-optimized (e.g., codon-optimized for expression in a human or expression in a mouse). For example, the nucleic acid can be modified to substitute codons having a higher frequency of usage in a human cell, a non-human cell, a mammalian cell, a rodent cell, a mouse cell, a rat cell, or any other host cell of interest.

The nucleic acid encoding the LEMD2, the CHMP7, or the LEMD3 can be DNA or RNA. The nucleic acid in some cases can be a messenger RNA (mRNA) encoding the LEMD2, the CHMP7, or the LEMD3. The nucleic acid in some cases can be a complementary DNA (cDNA) encoding the LEMD2, the CHMP7, or the LEMD3. For example, such nucleic acids may contain only coding sequence without any intervening introns. In other cases, the nucleic acid can comprise one or more introns separating exons in the LEMD2, CHMP7, or LEMD3 coding sequence. For example, the nucleic acid can comprise LEMD2, CHMP7, or LEMD3 genomic sequence including both exons and introns.

In some methods, the nucleic acid is in an expression construct comprising the nucleic acid encoding the LEMD2, the CHMP7, or the LEMD3 operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In some cases, the promoter is a heterologous promoter (i.e., a promoter to which the LEMD2, the CHMP7, or the LEMD3 nucleic acid is not naturally operably linked). In other cases, the promoter can be an endogenous promoter (i.e., LEMD2 nucleic acid operably linked to a LEMD2 promoter, CHMP7 nucleic acid operably linked to a CHMP7 promoter, or LEMD3 nucleic acid operably linked to a LEMD3 promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter that is very specific with a low level of expression is a synapsin-1 promoter (e.g., a human synapsin-1 promoter, such as the promoter set forth in SEQ ID NO: 44). In some embodiments, the synapsin-1 promoter can be used together with a hemoglobin subunit beta (HBB) intron 2 (e.g., downstream of the synapsin-1 promoter) such as the one set forth in SEQ ID NO: 254. Inclusion of this element can enhance gene expression.

The nucleic acids and expression constructs disclosed herein can also comprise post-transcriptional regulatory elements, such as the woodchuck hepatitis virus post-transcriptional regulatory element.

The nucleic acids and expression constructs can further comprise one or more polyadenylation signal sequences. For example, the nucleic acid construct can comprise a polyadenylation signal sequence located 3′ of the LEMD2, CHMP7, or LEMD3 coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. Examples of suitable polyadenylation signals include, for example, those set forth in SEQ ID NO: 252 and 253.

The nucleic acids and expression constructs can also optionally comprise a polyadenylation signal sequence upstream of the LEMD2, CHMP7, or LEMD3 coding sequence. The polyadenylation signal sequence upstream of the LEMD2, CHMP7, or LEMD3 coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the LEMD2, CHMP7, or LEMD3 encoded by the nucleic acid or expression constructs can be expressed in a neuron-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., US 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

The nucleic acids or expression constructs can be in a vector, such as a viral vector. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The nucleic acids or expression constructs can be in a vector, such as a viral vector. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include about 10¹², about 10¹³, about 10¹⁴, about 10¹⁵, and about 10¹⁶ vector genomes/mL. Other exemplary viral titers (e.g., AAV titers) include about 10¹², about 10¹³, about 10¹⁴, about 10¹⁵, and about 10¹⁶ vector genomes(vg)/kg of body weight.

In one example, the nucleic acid or expression construct is in an AAV vector. The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell’s DNA replication machinery to synthesize the complementary strand of the AAV’s single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In some methods, the LEMD2, CHMP7, or LEMD3 or the nucleic acid encoding the LEMD2, CHMP7, or LEMD3 is associated with a lipid nanoparticle. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

The LEMD2, the CHMP7, the LEMD3, or the nucleic acid encoding the LEMD2, the CHMP7, or the LEMD3 can be administered to the cell or the subject by any suitable means. Various methods and compositions are provided herein to allow for introduction of molecule (e.g., a nucleic acid or protein) into a cell or subject.

The methods provided herein do not depend on a particular method for introducing a nucleic acid or protein into the cell, only that the nucleic acid or protein gains access to the interior of the cell. Methods for introducing nucleic acids and proteins into various cell types are known in the art and include, for example, stable transfection methods, transient transfection methods, and virus-mediated methods.

Transfection protocols as well as protocols for introducing molecules (e.g., nucleic acids or proteins) into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4): 1590-4, and Kriegler, M (1991). Transfer and Expression: A Laboratory Manual. New York: W. H. Freeman and Company. pp. 96-97, each of which is herein incorporated by reference in its entirety for all purposes); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28, herein incorporated by reference in its entirety for all purposes). Viral methods can also be used for transfection.

Introduction of molecules (e.g., nucleic acids or proteins) into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell can also be accomplished by microinjection. Microinjection of an mRNA is preferably into the cytoplasm (e.g., to deliver mRNA directly to the translation machinery), while microinjection of a protein or a DNA encoding a protein is preferably into the nucleus. Alternatively, microinjection can be carried out by injection into both the nucleus and the cytoplasm: a needle can first be introduced into the nucleus and a first amount can be injected, and while removing the needle from the cell a second amount can be injected into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acids or proteins) into a cell can include, for example, vector delivery, particle-mediated delivery, exosome-mediated delivery, lipid-nanoparticle-mediated delivery, cell-penetrating-peptide-mediated delivery, or implantable-device-mediated delivery. Methods of administering nucleic acids or proteins to a subject to modify cells in vivo are disclosed elsewhere herein. As specific examples, a molecule (e.g., nucleic acid or protein) can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., lentivirus-mediated delivery or adeno-associated virus (AAV)-mediated delivery), and lipid-nanoparticle-mediated delivery.

In one example, the LEMD2, the CHMP7, the LEMD3, or the nucleic acid encoding the LEMD2, the CHMP7, or the LEMD3 can be administered via viral transduction such as lentiviral transduction or adeno-associated viral transduction. In another example, the LEMD2, the CHMP7, the LEMD3, or the nucleic acid encoding the LEMD2, the CHMP7, or the LEMD3 can be administered via lipid nanoparticle (LNP)-mediated delivery.

Administration in vivo can be by any suitable route such that the LEMD2, the CHMP7, the LEMD3, or the nucleic acid encoding the LEMD2, the CHMP7, or the LEMD3 reaches the intended target cell(s) (e.g., neurons in the brain of the subject) or target tissue (e.g., brain). Examples of routes of administration include parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. For example, the LEMD2, the CHMP7, the LEMD3, or the nucleic acid encoding the LEMD2, the CHMP7, or the LEMD3 may be administered directly to the brain of a subject or to neurons in the brain of a subject. In a specific example, administration to a subject is by intrathecal injection or by intracranial injection (e.g., stereotactic surgery for injection in the hippocampus and other brain regions, or intracerebroventricular injection). In a specific example, administration to a subject is by intracerebroventricular injection. In another specific example, administration to a subject is by intracranial injection. In another specific example, administration to a subject is by intrathecal injection.

The frequency of administration and the number of dosages can depend on the half-life of the composition being administered and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

The cells or subjects in the methods can be, for example, mammalian, non-human mammalian, and human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans. In a specific example, the cells or subjects are human.

The cells can be isolated cells (e.g., in vitro) or can be in vivo within a subject (e.g., animal or mammal). Cells can also be any type of undifferentiated or differentiated state. In one example, the cells are neurons.

The cells provided herein can be normal, healthy cells, or can be diseased cells comprising tau aggregates. The cells can be, for example, prone to tau aggregation, or they can have preexisting tau aggregation.

In one example, the cell is a human cell, a rodent cell, a mouse cell, or a rat cell such as a human neuron, a rodent neuron, a mouse neuron, or a rat number. In a specific example, the cell is a human neuron. In a specific example, the cell is in vivo in a subject (e.g., a neuron in the brain of a subject). For example, such methods can be methods of inhibiting tau aggregation or methods of reducing tau phosphorylation in a cell of a subject (e.g., a neuron in the brain of the subject).

Such methods can further comprise screening the cells or subjects to confirm the presence of the LEMD2, the CHMP7, the LEMD3, or the nucleic acid encoding the LEMD2, the CHMP7, or the LEMD3. Screening the cells or subjects for the LEMD2, the CHMP7, the LEMD3, or the nucleic acid encoding the LEMD2, the CHMP7, or the LEMD3 can be performed by any known means. Such methods can further comprise screening the cells or subjects to confirm expression of the nucleic acid encoding the LEMD2, the CHMP7, or the LEMD3. Screening the cells or subjects for expression of the nucleic acid encoding the LEMD2, the CHMP7, or the LEMD3 can be performed by any known means. For example, methods for measuring protein expression and for measuring expression of mRNA encoded by a coding sequence are well-known.

One example of an assay that can be used is the BASESCOPE™ RNA in situ hybridization (ISH) assay, which a method that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a 1 ZZ probe and it can differentially detect single nucleotide edits and mutations in intact fixed tissue.

As another example, reporter genes can be used for screening. For example, the nucleic acid encoding the LEMD2, the CHMP7, or the LEMD3 can encode a LEMD2, CHMP7, or LEMD3 fused to a reporter gene such as a fluorescent protein. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase.

As another example, selection markers can be used to screen for cells that have the LEMD2, the CHMP7, the LEMD3, or the nucleic acid encoding the LEMD2, the CHMP7, or the LEMD3. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k).

The methods can further comprise assessing one or more signs or symptoms of tauopathy or tau aggregation by any suitable means. Examples of such signs and symptoms are discussed in more detail elsewhere herein and include, for example, tau hyperphosphorylation or tau aggregation. Other signs and symptoms can include, for example, increased tau and/or phospho-tau in an insoluble fraction following cell fractionation, increased phospho-tau, increased phospho-tau in the somatodendritic compartment of neurons, increased phospho-tau in the perinuclear region of neurons, decreased nuclear pore complex protein Nup98-Nup96 (Nup98) nuclear-to-cytoplasmic ration in neurons, decreased GTP-binding nuclear protein Ran (Ran) nuclear-to-cytoplasmic ratio in neurons, or decreased Ran GTPase-activating protein 1 (RanGAP1) nuclear-to-cytoplasmic ratio in neurons. The phospho-tau can be, for example, phospho-tau (S356) or phospho-tau AT8 (S202, T205). Other signs and symptoms can include, for example, serum neurofilament light chain (sNfL). This can be done, for example, about 1 day, about 2 days, about 3 days, about 4 days, about 5 days, about 6 days, about 7 days, about 1 week, about 2 weeks, about 3 weeks, about 4 weeks, about 5 weeks, about 6 weeks, or longer after introducing the LEMD2, the CHMP7, the LEMD3, or the nucleic acid encoding the LEMD2, the CHMP7, or the LEMD3. For example, the assessing can be done about 2 weeks to about 6 weeks or about 3 weeks to about 5 weeks after introducing the LEMD2, the CHMP7, the LEMD3, or the nucleic acid encoding the LEMD2, the CHMP7, or the LEMD3.

The methods described herein can, for example, reduce the amount of new tau aggregate formation (new tau aggregation) in a cell or a subject and/or can reduce the amount of existing tau aggregate formation (preexisting tau aggregation) in a cell or a subject. For example, the methods described herein can prevent new tau aggregate formation and/or can reverse existing tau aggregate formation. The methods described herein can also, for example, reduce the levels of phospho-tau (e.g., phospho-tau (S356) or phospho-tau AT8 (S202, T205) in a cell or a subject.

The methods described herein can, for example, reduce the amount of new serum neurofilament light chain (sNfL) accumulation in a subject and/or can reduce the amount of existing serum neurofilament light chain (sNfL) levels in a subject. For example, the methods described herein can prevent new tau serum neurofilament light chain (sNfL) accumulation and/or can reverse existing serum neurofilament light chain (sNfL) accumulation.

In some of the methods described herein, the methods are for treating or preventing tauopathies in a subject. In some methods (e.g., methods for treating), the subject has one or more signs or symptoms of a tauopathy. For example, the subject can have preexisting tau aggregate formation in one or more cells. Tauopathies are a class of diseases caused by misfolding of the tau protein. They are a group of progressive neurodegenerative disorders that are pathologically defined by the presence of tau protein aggregates in the brain. Tauopathies are a group of heterogeneous neurodegenerative conditions characterized by deposition of abnormal tau in the brain. These include, for example, Alzheimer’s disease (AD), Down’s syndrome, Pick’s disease, progressive supranuclear palsy (PSP), corticobasal degeneration (CBD), and frontotemporal dementia with parkinsonism linked to chromosome 17 (FTDP-17). In AD and other tauopathies, tau protein is abnormally hyperphosphorylated and aggregated into bundles of filaments (paired helical filaments), which manifest as neurofibrillary tangles.

In some methods described herein, the methods are for treating or preventing a primary tauopathy. The primary tauopathies are a group of neurodegenerative diseases in which tau is believed to be the major contributing factor of the neurodegenerative process. In the primary tauopathies, there is a disassociation between tau, a microtubule associated protein, and microtubules, as a result of tau hyperphosphorylation. This disassociation between tau and microtubules results in tau fibrillization and inclusion formation, and microtubule dysfunction. All diseases that are considered primary tauopathies have in common the abnormal deposition of aggregated tau in the brain. There are other diseases, in which tau deposition can be observed, but for one reason or another, tau either co-exists with another protein, or tau is not considered the primary neurodegenerative process. Diseases in this latter category include: Alzheimer’s disease in which beta-amyloid is also present, Lewy body disease in which alpha-synuclein is also present, myotonic dystrophy, subacute sclerosing panencephalitis, Down’s syndrome, and Niemann-Pick disease-type C.

There are six isoforms of tau that are expressed in the adult brain. These six isoforms are derived from alternative splicing of three N-terminal exons in the tau gene: exon 2, exon 3 and exon 10. Three of the six isoforms are due to the splicing in of exon 10, while the other three isoforms are a result of the splicing out of exon 10. The splicing in of exon 10 results in isoforms with four repeated microtubule binding domains, while the splicing out of exon 10 results in isoforms with three repeated microtubule binding domains. This is important, because although the healthy human brain consists of equal amounts of tau with three and four repeated binding domains, some primary tauopathies are characterized by a predominance of isoforms with four repeated binding domains (4R tauopathies), some by a predominance of isoforms with three repeated binding domains (3R tauopathies), and some by an approximately equal mix of isoforms with three and four repeated binding domains (3R + 4R tauopathies). See Table 1.

TABLE 1

Primary Tauopathies.

| | |
|---|---|
| Tauopathy | Type |
| Pick’s disease | 3R tauopathy |
| Progressive supranuclear palsy | 4R tauopathy |
| Corticobasal degeneration | 4R tauopathy |
| Argyrophilic grain disease | 4R tauopathy |
| Globular glial tauopathies | 4R tauopathy |
| Aging-related tau astrogliopathy | 4R tauopathy |
| Chronic traumatic encephalopathy | 3R+4R tauopathy |
| Primary age-related tauopathy (PART) | 3R+4R tauopathy |
| Parkinsonism-Dementia complex of Guam | 3R+4R tauopathy |
| Postencephalitic Parkinsonism | 3R+4R tauopathy |
| Atypical Parkinsonism of Guadeloupe | 3R+4R tauopathy |
| Diffuse neurofilament tangles with calcification | 3R+4R tauopathy |
| Frontotemporal dementia & Parkinsonism liked to Chromosome 17 | 3R, 4R, or 3R+4R tauopathy |

There are several tau pathogenic mutations, such as pro-aggregation mutations, that are associated with (e.g., segregate with) or cause a tauopathy. Pathogenic tau mutations, which can be either exonic or intronic, generally alter the relative production of tau isoforms and can lead to changes in microtubule assembly and/or the propensity of tau to aggregate. As one example, such a mutation can be an aggregation-sensitizing mutation that sensitizes tau to seeding but does not result in tau readily aggregating on its own. For example, the mutation can be the disease-associated P301S mutation. By P301S mutation is meant the human tau P301S mutation or a corresponding mutation in another tau protein when optimally aligned with the human tau protein. Other pathogenic tau mutations include, for example, A152T, G272V, K280del, P301L, S320F, V337M, R406W, P301L/V337M, K280del/I227P/I308P, G272V/P301L/R406W, and A152T/P301L/S320F. See alzforum.org/mutations/mapt, Brandt et al. (2005) Biochim. Biophys. Acta 1739:331-354, and Wolfe (2009) J. Biol. Chem. 284(10):6021-6025, each of which is herein incorporated by reference in its entirety for all purposes.

The methods described herein can alleviate one or more signs and symptoms of tauopathy in a cell or subject. Some examples of signs and symptoms of tauopathy at the cellular level include tau hyperphosphorylation (e.g., in the somatodendritic compartment of a neuron because although generally considered an axonal protein, tau is found in the dendritic compartment of degenerating neurons, and this redistribution is thought to be a trigger of neurodegeneration in Alzheimer’s disease), tau aggregation, abnormal shape of nuclear lamina, and impaired nucleocytoplasmic transport. Other signs and symptoms at an organism level can include neurofibrillary tangles (e.g., in the neocortex, amygdala, hippocampus, brain stem, or spinal cord), neuron loss (e.g., in the hippocampus, amygdala, or neocortex), microgliosis, synaptic loss, cognitive impairment, or motor deficits. Other signs and symptoms can include, for example, increased tau and/or phospho-tau in an insoluble fraction following cell fractionation, increased phospho-tau in the somatodendritic compartment of neurons, increased phospho-tau in the perinuclear region of neurons, decreased nuclear pore complex protein Nup98-Nup96 (Nup98) nuclear-to-cytoplasmic ration in neurons, decreased GTP-binding nuclear protein Ran (Ran) nuclear-to-cytoplasmic ratio in neurons, or decreased Ran GTPase-activating protein 1 (RanGAP1) nuclear-to-cytoplasmic ratio in neurons. The phospho-tau can be, for example, phospho-tau (S356) or phospho-tau AT8 (S202, T205). Other signs and symptoms can include, for example, serum neurofilament light chain (sNfL), a biomarker of neuronal damage that is a strong indicator of neurodegenerative processes. Neurofilament light chain (NfL) is a cytoskeletal protein component whose release into blood is indicative of neuronal damage and is a well-known biomarker of many neurodegenerative diseases. See, e.g., Rubsamen et al. (2021) BMC Medicine 19:38 and Loeffler et al. (2020) Front. Neurosci. 14:579, each of which herein incorporated by reference in its entirety for all purposes. Neurofilament light (NF-L) is a 68 kDa cytoskeletal intermediate filament protein that is expressed in neurons. It associates with the 125 kDa Neurofilament medium (NF-M) and the 200 kDa Neurofilament heavy (NF-H) to form neurofilaments. They are major components of the neuronal cytoskeleton, and are believed to function primarily to provide structural support for the axon and to regulate axon diameter. Neurofilaments can be released in significant quantity following axonal damage or neuronal degeneration. NF-L has been shown to associate with traumatic brain injury, multiple sclerosis, frontotemporal dementia, and other neurodegenerative diseases.

### III Nucleic Acids Encoding LEMD2, CHMP7, or LEMD3

Provided herein are nucleic acids or nucleic acid constructs encoding LEMD2, CHMP7, or LEMD3 (i.e., an exogenous nucleic acid encoding the LEMD2, CHMP7, or LEMD3). The nucleic acids or nucleic acid constructs can be isolated nucleic acid constructs.

Some nucleic acids or nucleic acid constructs described herein comprise a nucleic acid encoding LEMD2. For example, the LEMD2 can be a wild type LEMD2. In one example the LEMD2 is a human LEMD2. Human LEMD2 (also called LEM domain-containing protein 2, hLEM2, or LEM domain nuclear envelope protein 2) is assigned UniProt reference number Q8NC56. The human gene encoding LEMD2 (LEMD2, or LEM domain nuclear envelope protein 2) is assigned NCBI GeneID 221496 and is found at location 6p21.31 on chromosome 6 (assembly: GRCh38.p13 (GCF_000001405.39); location: NC_000006.12 (33771213..33794274, complement)). At least two isoforms of human LEMD2 are known. The first isoform is 503 amino acids and is assigned UniProt reference number Q8NC56-1 and NCBI reference number NP_851853.1 (SEQ ID NO: 1). An exemplary coding sequence is assigned reference number CCDS4785.1 (SEQ ID NO: 2), and an exemplary mRNA (cDNA) sequence is assigned reference number NM_181336.4 (SEQ ID NO: 4). A codon-optimized coding sequence to distinguish from the native CDS is set forth in SEQ ID NO: 3. The second isoform is 201 amino acids and is assigned UniProt reference number Q8NC56-2 and NCBI reference numbers NP_001137416.1 and NP_001335638.1 (SEQ ID NO: 5). An exemplary coding sequence is assigned reference number CCDS47411.1 (SEQ ID NO: 6), and exemplary mRNA (cDNA) sequences are assigned reference numbers NM_001143944.1 and NM_001348709.2 (SEQ ID NOS: 8 and 9, respectively). A codon-optimized coding sequence to distinguish from the native CDS is set forth in SEQ ID NO: 7.

In another example the LEMD2 is a mouse LEMD2. Mouse LEMD2 is assigned UniProt reference number Q6DVA0. The mouse gene encoding LEMD2 (Lemd2) is assigned NCBI GeneID 224640 and is found at location 17; 17 A3.3 on chromosome 17 (assembly: GRCm39 (GCF_000001635.27); Location: NC_000083.7 (27408574..27426228, complement)). An exemplary mouse LEMD2 is 511 amino acids and is assigned UniProt reference number Q6DVA0-1 and NCBI reference number NP_666187.2 (SEQ ID NO: 10). An exemplary coding sequence is assigned reference number CCDS50043.1 (SEQ ID NO: 11), and an exemplary mRNA (cDNA) sequence is assigned reference number NM_146075.2 (SEQ ID NO: 14). Codon-optimized coding sequences to distinguish from the native CDS are set forth in SEQ ID NOS: 12 and 13. Another codon-optimized coding sequence to distinguish from the native CDS is set forth in SEQ ID NO: 255.

Some nucleic acids or nucleic acid constructs described herein comprise a nucleic acid encoding CHMP7. For example, the CHMP7 can be a wild type CHMP7. In one example the CHMP7 is a human CHMP7. Human CHMP7 (also called charged multivesicular body protein 7 or chromatin-modifying protein 7) is assigned UniProt reference number Q8WUX9. The human gene encoding CHMP7 (CHMP7) is assigned NCBI GeneID 91782 and is found at location 8p21.3 on chromosome 8 (assembly: GRCh38.p13 (GCF_000001405.39); Location: NC_000008.11 (23243637..23262000)). An exemplary CHMP7 protein is 453 amino acids and is assigned UniProt reference number Q8WUX9-1 and NCBI reference number NP_689485.1 (SEQ ID NO: 15). An exemplary coding sequence is assigned reference number CCDS6040.1 (SEQ ID NO: 16), and an exemplary mRNA (cDNA) sequence is assigned reference number NM_152272.5 (SEQ ID NO: 18). A codon-optimized coding sequence to distinguish from the native CDS is set forth in SEQ ID NO: 17.

In another example the CHMP7 is a mouse CHMP7. Mouse CHMP7 is assigned UniProt reference number Q8R1T1. The mouse gene encoding CHMP7 (Chmp7) is assigned NCBI GeneID 105513 and is found at location 14; 14 D2 on chromosome 14 (assembly: GRCm39 (GCF_000001635.27); Location: NC_000080.7 (69954428..69970019, complement)). An exemplary mouse CHMP7 is 451 amino acids and is assigned UniProt reference number Q8R1T1-1 and NCBI reference number NP_598839.2 (SEQ ID NO: 19). An exemplary coding sequence is assigned reference number CCDS27242.1 (SEQ ID NO: 20), and an exemplary mRNA (cDNA) sequence is assigned reference number NM_134078.4 (SEQ ID NO: 22). A codon-optimized coding sequence to distinguish from the native CDS is set forth in SEQ ID NO: 21.

Some nucleic acids or nucleic acid constructs described herein comprise a nucleic acid encoding LEMD3. For example, the LEMD3 can be a wild type LEMD3. In one example the LEMD3 is a human LEMD3. Human LEMD3 (also called inner nuclear membrane protein Man1 or LEM domain-containing protein 3) is assigned UniProt reference number Q9Y2U8. The human gene encoding LEMD3 (LEMD3, MAN1, or LEM domain containing 3) is assigned NCBI GeneID 23592 and is found at location 12q14.3 on chromosome 12 (assembly: GRCh38.p13 (GCF_000001405.39); Location: NC_000012.12 (65169583..65248355)). An exemplary LEMD3 protein is 911 amino acids and is assigned UniProt reference number Q9Y2U8-1 and NCBI reference number NP-055134.2 (SEQ ID NO: 23). An exemplary coding sequence is assigned reference number CCDS8972.1 (SEQ ID NO: 24), and an exemplary mRNA (cDNA) sequence is assigned reference number NM_014319.5 (SEQ ID NO: 26). A codon-optimized coding sequence to distinguish from the native CDS is set forth in SEQ ID NO: 25.

In another example the LEMD3 is a mouse LEMD3. Mouse LEMD3 is assigned UniProt reference number Q9WU40. The mouse gene encoding LEMD3 (Lemd3) is assigned NCBI GeneID 380664 and is found at location 10; 10 D2 on chromosome 10 (assembly: GRCm39 (GCF_000001635.27); Location: NC_000076.7 (120759316..120815491, complement)). An exemplary mouse LEMD3 is 921 amino acids and is assigned UniProt reference number Q9WU40-1 (SEQ ID NO: 27). A codon-optimized coding sequence to distinguish from the native CDS is set forth in SEQ ID NO: 28. Another exemplary mouse LEMD3 is 918 amino acids in length and is assigned NCBI reference number NP_001074662.2 (SEQ ID NO: 29). An exemplary coding sequence is assigned reference number CCDS48703.1 (SEQ ID NO: 30), and an exemplary mRNA (cDNA) sequence is assigned reference number NM_001081193.2 (SEQ ID NO: 31).

In some cases, the nucleic acid encoding the LEMD2, CHMP7, or LEMD3 can be a native coding sequence. In other cases, it can be codon-optimized (e.g., codon-optimized for expression in a human or expression in a mouse). For example, the nucleic acid can be modified to substitute codons having a higher frequency of usage in a human cell, a non-human cell, a mammalian cell, a rodent cell, a mouse cell, a rat cell, or any other host cell of interest.

The nucleic acid encoding the LEMD2, the CHMP7, or the LEMD3 can be DNA or RNA. The nucleic acid in some cases can be a messenger RNA (mRNA) encoding the LEMD2, the CHMP7, or the LEMD3. The nucleic acid in some cases can be a complementary DNA (cDNA) encoding the LEMD2, the CHMP7, or the LEMD3. For example, such nucleic acids may contain only coding sequence without any intervening introns. In other cases, the nucleic acid can comprise one or more introns separating exons in the LEMD2, CHMP7, or LEMD3 coding sequence. For example, the nucleic acid can comprise genomic sequence including both exons and introns.

In some cases, the nucleic acid is in an expression construct comprising the nucleic acid encoding the LEMD2, the CHMP7, or the LEMD3 operably linked to a promoter. The promoter can be any suitable promoter for expression in vivo within an animal or in vitro within an isolated cell. The promoter can be a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. In a specific example, the promoter is active in a neuron. In some cases, the promoter is a heterologous promoter (i.e., a promoter to which the LEMD2, the CHMP7, or the LEMD3 nucleic acid is not operably linked naturally). In other cases, the promoter can be an endogenous promoter (i.e., LEMD2 nucleic acid operably linked to a LEMD2 promoter, CHMP7 nucleic acid operably linked to a CHMP7 promoter, or LEMD3 nucleic acid operably linked to a LEMD3 promoter). The heterologous promoter can be any type of promoter as disclosed elsewhere herein. For example, the promoter can be a constitutive promoter, such as an EF1 alpha promoter. Alternatively, the promoter can be a tissue-specific promoter or an inducible promoter. For example, the promoter can be a neuron-specific promoter. One example of a suitable neuron-specific promoter is a synapsin-1 promoter (e.g., a human synapsin-1 promoter, such as the promoter set forth in SEQ ID NO: 44). In some embodiments, the synapsin-1 promoter can be used together with a hemoglobin subunit beta (HBB) intron 2 (e.g., downstream of the synapsin-1 promoter) such as the one set forth in SEQ ID NO: 254. Inclusion of this element can enhance gene expression.

The nucleic acids and expression constructs disclosed herein can also comprise post-transcriptional regulatory elements, such as the woodchuck hepatitis virus post-transcriptional regulatory element.

The nucleic acids and expression constructs can further comprise one or more polyadenylation signal sequences. For example, the nucleic acid construct can comprise a polyadenylation signal sequence located 3′ of the LEMD2, CHMP7, or LEMD3 coding sequence. Any suitable polyadenylation signal sequence can be used. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. Examples of suitable polyadenylation signals include, for example, those set forth in SEQ ID NO: 252 and 253.

The nucleic acids and expression constructs can also comprise a polyadenylation signal sequence upstream of the LEMD2, CHMP7, or LEMD3 coding sequence. The polyadenylation signal sequence upstream of the LEMD2, CHMP7, or LEMD3 coding sequence can be flanked by recombinase recognition sites recognized by a site-specific recombinase. In some constructs, the recombinase recognition sites also flank a selection cassette comprising, for example, the coding sequence for a drug resistance protein. In other constructs, the recombinase recognition sites do not flank a selection cassette. The polyadenylation signal sequence prevents transcription and expression of the protein or RNA encoded by the coding sequence. However, upon exposure to the site-specific recombinase, the polyadenylation signal sequence will be excised, and the protein or RNA can be expressed.

Such a configuration can enable tissue-specific expression or developmental-stage-specific expression if the polyadenylation signal sequence is excised in a tissue-specific or developmental-stage-specific manner. Excision of the polyadenylation signal sequence in a tissue-specific or developmental-stage-specific manner can be achieved if an animal comprising the nucleic acid or expression constructs further comprises a coding sequence for the site-specific recombinase operably linked to a tissue-specific or developmental-stage-specific promoter. The polyadenylation signal sequence will then be excised only in those tissues or at those developmental stages, enabling tissue-specific expression or developmental-stage-specific expression. In one example, the LEMD2, CHMP7, or LEMD3 encoded by the nucleic acid or expression constructs can be expressed in a neuron-specific manner.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The nucleic acids disclosed herein can comprise deoxyribonucleic acid (DNA) or ribonucleic acid (RNA), they can be single-stranded or double-stranded, and they can be in linear or circular form. The nucleic acid constructs can be naked nucleic acids or can be delivered by vectors, such as AAV vectors, as described elsewhere herein. If in linear form, the ends of the nucleic acid can be protected (e.g., from exonucleolytic degradation) by well-known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. The nucleic acids or expression constructs can, in some cases, comprise one or more of the following terminal structures: hairpin, loop, inverted terminal repeat (ITR), or toroid. For example, the nucleic acids or expression constructs can comprise ITRs.

The nucleic acids or expression constructs can include modifications or sequences that provide for additional desirable features (e.g., modified or regulated stability; tracking or detecting with a fluorescent label; a binding site for a protein or protein complex; and so forth). Nucleic acid constructs can comprise one or more fluorescent labels, purification tags, epitope tags, or a combination thereof. For example, a nucleic acid construct can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and-6)-carboxytetramethylrhodamine (TAMRA), and Cy7. A wide range of fluorescent dyes are available commercially for labeling oligonucleotides (e.g., from Integrated DNA Technologies). The label or tag can be at the 5′ end, the 3′ end, or internally within the nucleic acid construct. For example, a nucleic acid construct can be conjugated at 5′ end with the IR700 fluorophore from Integrated DNA Technologies (5′IRDYE®700).

The nucleic acids and expression constructs can also comprise a conditional allele. The conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

Nucleic acids and expression constructs can also comprise a polynucleotide encoding a selection marker. Alternatively, the nucleic acids and expression constructs can lack a polynucleotide encoding a selection marker. The selection marker can be contained in a selection cassette. Optionally, the selection cassette can be a self-deleting cassette. See, e.g., US 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. Exemplary selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), or herpes simplex virus thymidine kinase (HSV-k), or a combination thereof. The polynucleotide encoding the selection marker can be operably linked to a promoter active in a cell being targeted. Examples of promoters are described elsewhere herein.

The nucleic acids or expression constructs can also comprise a reporter gene. Exemplary reporter genes include those encoding luciferase, β-galactosidase, green fluorescent protein (GFP), enhanced green fluorescent protein (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, ZsGreen, MmGFP, mPlum, mCherry, tdTomato, mStrawberry, J-Red, mOrange, mKO, mCitrine, Venus, YPet, Emerald, CyPet, Cerulean, T-Sapphire, and alkaline phosphatase. Such reporter genes can be operably linked to a promoter active in a cell being targeted. Examples of promoters are described elsewhere herein.

### IV. Vectors

Also provided herein are vectors comprising the nucleic acids, nucleic acid constructs, or expression constructs encoding LEMD2, CHMP7, or LEMD3. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The nucleic acids or expression constructs can be in a vector, such as a viral vector. The viral vector can be, for example, an adeno-associated virus (AAV) vector or a lentivirus (LV) vector (i.e., a recombinant AAV vector or a recombinant LV vector). Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging. Exemplary viral titers (e.g., AAV titers) include about 10¹², about 10¹³, about 10¹⁴, about 10¹⁵, and about 10¹⁶ vector genomes/mL. Other exemplary viral titers (e.g., AAV titers) include about 10¹², about 10¹³, about 10¹⁴, about 10¹⁵, and about 10¹⁶ vector genomes(vg)/kg of body weight. In one example, the viral titer is between about 10¹³ to about 10¹⁴ vg/mL or vg/kg.

In one example, the nucleic acid or expression construct is in an AAV vector. The AAV may be any suitable serotype and may be a single-stranded AAV (ssAAV) or a self-complementary AAV (scAAV). The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediated AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 245, SEQ ID NO: 246, or SEQ ID NO: 247 or 248. Other examples of ITRs comprise one or more mutations compared to SEQ ID NO: 245, SEQ ID NO: 246, or SEQ ID NO: 247 or 248 and can be at least 90%, at least 91%, at least 92%, at least 93%, at least 94%, at least 95%, at least 96%, at least 97%, at least 98%, or at least 99% identical to SEQ ID NO: 245, SEQ ID NO: 246, or SEQ ID NO: 247 or 248. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end). In one example, the ITR on each end can comprise, consist essentially of, or consist of SEQ ID NO: 245. In another example, the ITR on each end can comprise, consist essentially of, or consist of SEQ ID NO: 246. In one example, the ITR on at least one end comprises, consists essentially of, or consists of SEQ ID NO: 247 or 248. In one example, the ITR on the 5′ end comprises, consists essentially of, or consists of SEQ ID NO: 247 or 248. In one example, the ITR on the 3′ end comprises, consists essentially of, or consists of SEQ ID NO: 247 or 248. In one example, the ITR on each end can comprise, consist essentially of, or consist of SEQ ID NO: 247 or 248. In one example, the ITR on at least one end comprises, consists essentially of, or consists of SEQ ID NO: 245. In one example, the ITR on the 5′ end comprises, consists essentially of, or consists of SEQ ID NO: 245. In one example, the ITR on the 3′ end comprises, consists essentially of, or consists of SEQ ID NO: 245. In one example, the ITR on each end can comprise, consist essentially of, or consist of SEQ ID NO: 245. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 245, and the ITR on the other end comprises, consists essentially of, or consists of SEQ ID NO: 246. In another example, the ITR on one end comprises, consists essentially of, or consists of SEQ ID NO: 245, and the ITR on the other end comprises, consists essentially of, or consists of SEQ ID NO: 247 or 248. In one example, the ITR on one end comprises, consists essentially of, or consists of SEQ ID NO: 246, and the ITR on the other end comprises, consists essentially of, or consists of SEQ ID NO: 247 or 248.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Selectivity of AAV serotypes for gene delivery in neurons is discussed, for example, in Hammond et al. (2017) PLoS One 12(12):e0188830, herein incorporated by reference in its entirety for all purposes. In a specific example, an AAV-PHP.eB vector is used. The AAV-PHP.eB vector shows high ability to cross the blood-brain barrier, increasing its CNS transduction efficiency. In another specific example, an AAV9 vector is used.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell’s DNA replication machinery to synthesize the complementary strand of the AAV’s single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### IV. Lipid Nanoparticles

Also provided herein are lipid nanoparticles comprising the LEMD2, CHMP7, or LEMD3 or the nucleic acids, nucleic acid constructs, expression constructs, or vectors encoding the LEMD2, CHMP7, or LEMD3.

Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

### V. Compositions

Also provided herein are compositions comprising the LEMD2, CHMP7, or LEMD3 or the nucleic acids, nucleic acid constructs, expression constructs, vectors, or lipid nanoparticle disclosed herein. Such compositions can be, for example, for use in administering LEMD2, CHMP7, or LEMD3 into a cell or subject or for use in expressing LEMD2, CHMP7, or LEMD3 in a cell or subject. Such compositions can be, for example, for use in inhibiting tau aggregation in a cell or subject. Such compositions can be, for example, for use in reducing tau phosphorylation in a cell or a subject. Such compositions can be, for example, for use in reducing serum neurofilament light chain (sNfL) or preventing accumulation of serum neurofilament light chain (sNfL) in a subject. Such compositions can be, for example, for use in treating a tauopathy in a subject. Such compositions can be, for example, for use in preventing a tauopathy in a subject.

### VI. Cells or Animals

Cells or subjects (e.g., animals) comprising the LEMD2, CHMP7, or LEMD3 or the nucleic acids, nucleic acid constructs, expression constructs, vectors, or lipid nanoparticles disclosed herein are also provided. The cells or subjects can express the LEMD2, CHMP7, or LEMD3.

The cells or subjects can be, for example, mammalian, non-human mammalian, and human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans.

The cells can be isolated cells (e.g., in vitro) or can be in vivo within a subject (e.g., animal or mammal). Cells can also be any type of undifferentiated or differentiated state. In one example, the cells are neurons.

The cells provided herein can be normal, healthy cells, or can be diseased cells comprising tau aggregates. The cells can be, for example, prone to tau aggregation, or they can have preexisting tau aggregation.

In one example, the cell is a human cell, a rodent cell, a mouse cell, or a rat cell such as a human neuron, a rodent neuron, a mouse neuron, or a rat number. In a specific example, the cell is a human neuron. In a specific example, the cell is in vivo in a subject (e.g., a neuron in the brain of a subject).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2

Description of Sequences

| SEQ ID NO | Type | Description |
|---|---|---|
| 1 | Protein | Human LEMD2 (Q8NC56-1; NP_851853.1) |
| 2 | DNA | Human LEMD2 CDS (CCDS4785.1) |
| 3 | DNA | Human LEMD2 CDS codon optimized (LV) |
| 4 | DNA | Human LEMD2 mRNA (NM_181336.4) |
| 5 | Protein | Human LEMD2 Isoform 2 (Q8NC56-2; NP_001137416.1; NP_001335638.1) |
| 6 | DNA | Human LEMD2 Isoform 2 CDS (CCDS47411.1) |
| 7 | DNA | Human LEMD2 Isoform 2 CDS codon optimized (LV) |
| 8 | DNA | Human LEMD2 Isoform 2 mRNA v1 (NM_001143944.1) |
| 9 | DNA | Human LEMD2 Isoform 2 mRNA v2 (NM_001348709.2) |
| 10 | Protein | Mouse LEMD2 (Q6DVA0-1; NP_666187.2) |
| 11 | DNA | Mouse Lemd2 CDS (CCDS50043.1) |
| 12 | DNA | Mouse Lemd2 CDS codon optimized (LV) |
| 13 | DNA | Mouse Lemd2 CDS codon optimized (AAV) |
| 14 | DNA | Mouse Lemd2 mRNA (NM_146075.2) |
| 15 | Protein | Human CHMP7 (Q8WUX9-1; NP_689485.1) |
| 16 | DNA | Human CHMP7 CDS (CCDS6040.1) |
| 17 | DNA | Human CHMP7 CDS codon optimized (LV) |
| 18 | DNA | Human CHMP7 mRNA (NM_152272.5) |
| 19 | Protein | Mouse CHMP7 (Q8R1T1-1; NP_598839.2) |
| 20 | DNA | Mouse Chmp7 CDS (CCDS27242.1) |
| 21 | DNA | Mouse Chmp7 CDS codon optimized (AAV, LV) |
| 22 | DNA | Mouse Chmp7 mRNA (NM_134078.4) |
| 23 | Protein | Human LEMD3 (Q9Y2U8-1; NP-055134.2) |
| 24 | DNA | Human LEMD3 CDS (CCDS8972.1) |
| 25 | DNA | Human LEMD3 CDS codon optimized (LV) |
| 26 | DNA | Human LEMD3 mRNA (NM_014319.5) |
| 27 | Protein | Mouse LEMD3 (Q9WU40-1) |
| 28 | DNA | Mouse Lemd3 CDS codon optimized (LV) |
| 29 | Protein | Mouse LEMD3 v2 (NP_001074662.2) |
| 30 | DNA | Mouse Lemd3 v2 CDS (CCDS48703.1) |
| 31 | DNA | Mouse Lemd3 v2 mRNA (NM_001081193.2) |
| 32 | DNA | LV716 hBANF1.WT |
| 33 | Protein | BANF1 |
| 34 | DNA | LV603 hLEMD2 |
| 35 | DNA | LV687 mLEMD2 |
| 36 | DNA | LV602 hLEMD2iso2 |
| 37 | DNA | LV604 hLEMD3 |
| 38 | DNA | LV689 mLEMD3 |
| 39 | DNA | LV611 hCHMP7 |
| 40 | DNA | LV680 mCHMP7 |
| 41 | DNA | AAV699 - mCherry |
| 42 | DNA | AAV698 - mCHMP7 |
| 43 | DNA | AAV697 - mLEMD2 |
| 44 | DNA | hSynapsin-1 promoter |
| 45-83 | DNA | Primers and Probes for TAQMAN Assays |
| 84-132 | DNA | Primers for gRNA Library Sample Preparation |
| 133-214 | DNA | gRNAs used for the study [Cas9, SAM and KRAB] |
| 215-223 | DNA | Primers and Probes for TAQMAN Assays |
| 224-226 | RNA | ASOs |
| 227-229 | RNA | gRNA DNA-Targeting Segments |
| 230-241 | DNA | Primers |
| 242-244 | RNA | gRNA DNA-Targeting Segments |
| 245 | DNA | ITR 145 |
| 246 | DNA | ITR 141 |
| 247 | DNA | ITR 130 |
| 248 | DNA | ITR 130 v2 |
| 249 | DNA | AAV-mmLemd2 |
| 250 | DNA | AAV-mmLemd3 |
| 251 | DNA | AAV-mmChmp7 |
| 252 | DNA | PolyA v1 |
| 253 | DNA | PolyA v2 |
| 254 | DNA | Hemoglobin Subunit Beta (HBB) Intron 2 |
| 255 | DNA | Mouse Lemd2 CDS codon optimized (AAV) v2 |

### EXAMPLES

### Example 1. Loss of Nuclear Envelope Integrity Promotes Aggregation of Microtubule Associated Protein Tau

In Alzheimer’s disease and other tauopathies, tau is abnormally hyperphosphorylated and aggregated into bundles of paired helical filaments, which manifest as neurofibrillary tangles. The fibrillization of tau into insoluble aggregates is not only a hallmark of the disease but has also been implicated as a causative factor of neurotoxicity. Neurodegenerative diseases with tau pathology are characterized by propagation of tau aggregates through the central nervous system following stereotypical patterns, a process that correlates with disease progression. This progressive pathology proceeds along neuroanatomical circuits and is proposed to occur by a prion-like mechanism of cell-to-cell transmission of misfolded tau. Although mutations in the MAPT gene have been linked to frontal temporal dementia and other tauopathies, providing a mechanism for disease causation in these instances, in Alzheimer’s disease and other tauopathies that are not associated with MAPT mutations, the etiology of tau pathology is poorly understood. Most interest in tau has focused on its roles in cytoskeletal microtubule dynamics, but recent evidence that nuclear components can be associated with tau aggregates in the cytoplasm may provide insight into the mechanisms by which tau becomes misfolded and aggregated in the disease.

To discover perturbations that can trigger tau aggregation and reveal normal processes that protect cells from tau pathology, we undertook a series of genetic screens for mutations that promote or inhibit tau aggregation in a human embryonic kidney (HEK293T) biosensor cell line. See, e.g., Holmes et al. (2014) Proc. Natl. Acad. Sci. U.S.A.111:E4376-4385 and Sanders et al. (2014) Neuron 82:1271-1288, each of which is herein incorporated by reference in its entirety for all purposes. The biosensor cells stably express two transgenes, each encoding tau’s 4RD complete microtubule binding domain, including a pathogenic mutation of proline 301 to serine, fused to either cyan fluorescent protein (tau-CFP) or yellow fluorescent protein (tau-YFP). Aggregation of the tau fragments is detected when the two tau fusion proteins come into a close and favorable orientation to induce Förster resonance energy transfer (FRET) between CFP and YFP. Aggregation of tau can be induced in the biosensor cells by treatment with tau seeding agent, such as extracts from cells and tissues that have pathogenic tau aggregates or misfolded fibrils of wild type or mutant tau. The tau aggregates in the biosensor cells can be visualized by fluorescence microscopy, while FRET enables quantification of the number of cells with aggregates by flow cytometry and their purification by fluorescence-activated cell sorting (FACS). Tau aggregates can also be visualized by fluorescence microscopy.

To facilitate genetic screening, we expressed Cas9 in the tau biosensor cell line and then introduced a library of lentivirus-expressed CRISPR guide RNAs (gRNAs) that direct specific Cas9 cleavage at nearly all the protein-coding genes of the genome, producing a large collection of cells, each of which carries an integrated gene-specific mutagen. Cells with a desired property, such as gain or loss of FRET, can be isolated by FACS, and the integrated gRNAs they carry can be discovered by DNA sequencing, thereby identifying genes that, when mutated, produce the desired property. As a complementary screening strategy, we modified the tau biosensor cell line with the Synergistic Activation Mediator (SAM) CRISPR platform for gRNA-targeted gene activation. From genetic screens with the two complementary approaches, we identified genes that when inactivated promoted or enhanced tau aggregation and when overexpressed prevented or inhibited tau aggregation. These genes shared a common biological function-maintenance of nuclear envelope integrity-a process that, prior to our study, had not been implicated in tau pathology.

### Results

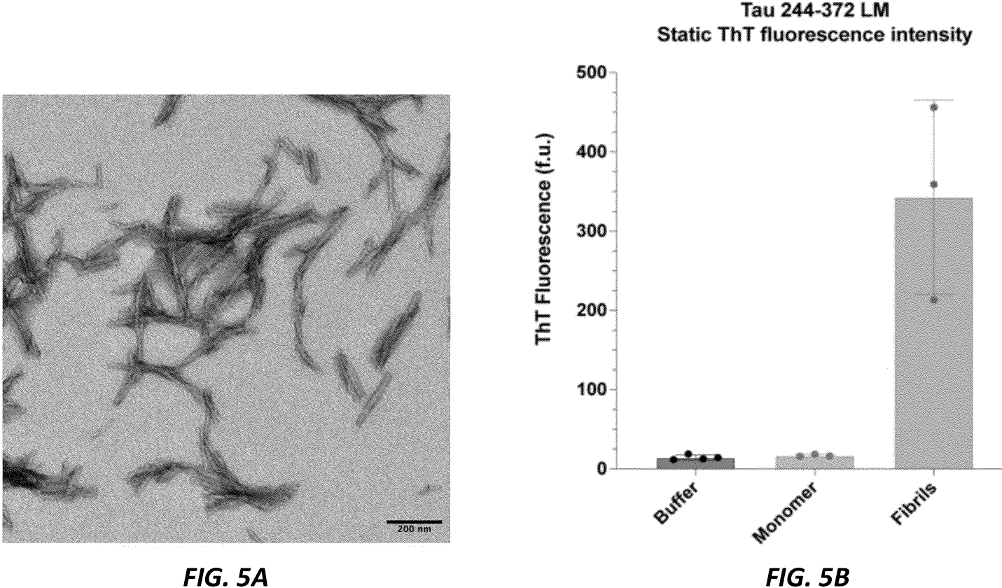

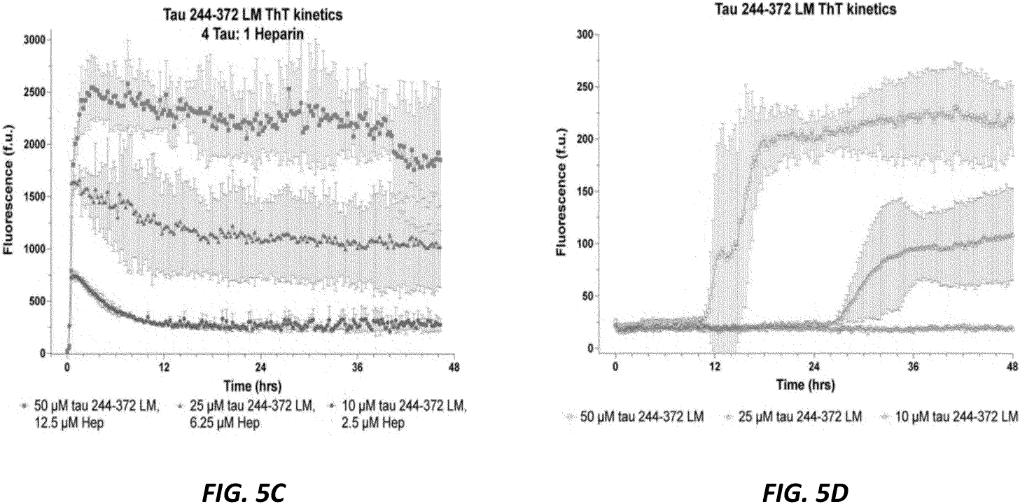

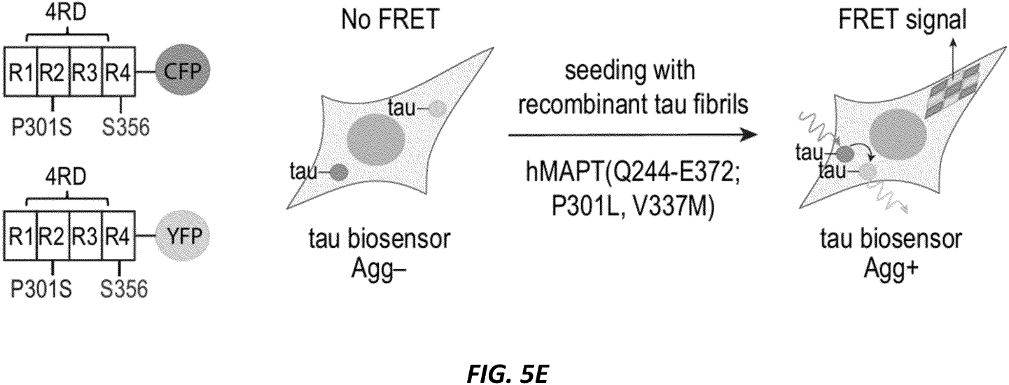

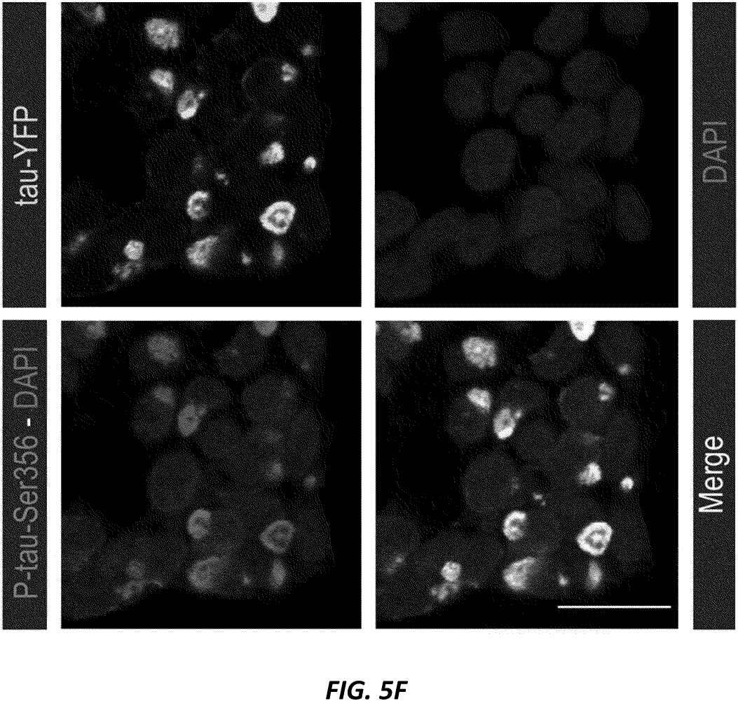

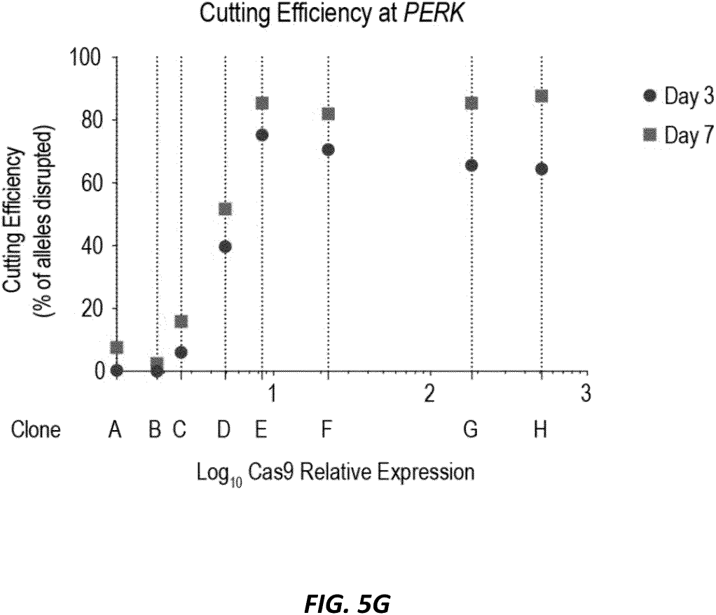

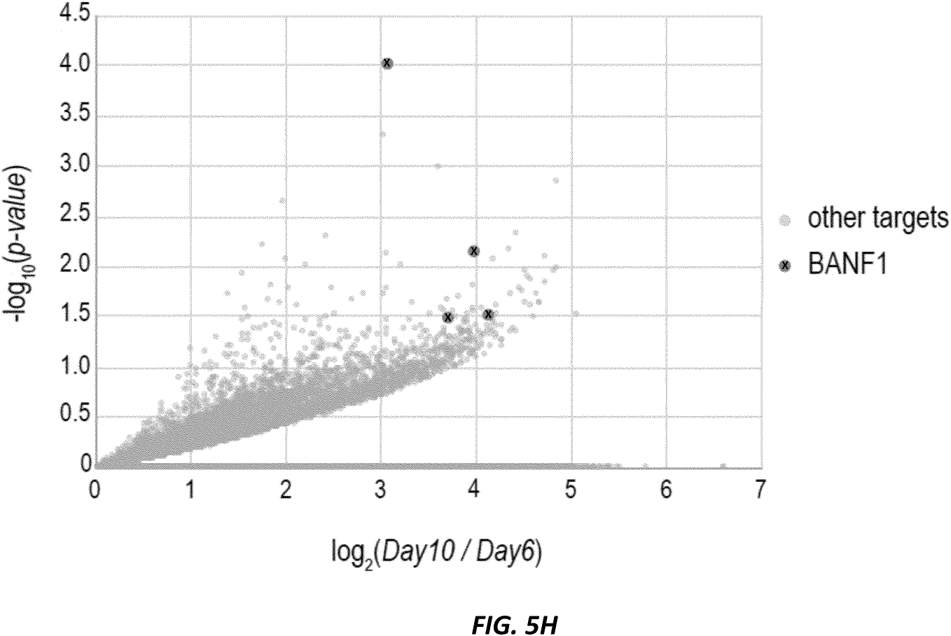

Use of the tau biosensor cell line for CRISPR-Cas9 genetic screens. The tau biosensor cells express the tau-CFP and tau-YFP reporter fusion proteins in a stable, soluble state with no visible fluorescent aggregates or FRET signal. Treating the biosensor cells with a strong seeding agent, such as purified recombinant tau fibrils complexed with a transfection reagent, induces visible fluorescent aggregates and FRET in most of the cells (FIGS. 5A-5F). The intracellular aggregates are detected by an antibody that recognizes tau phosphorylated at Serine 356 (P-tau-Ser356, FIG. 5F), a hallmark for pathological tau aggregation in the human brain that correlates with the seeding activity that promotes cell-to-cell spread. To ready the tau biosensor cell line for CRISPR-Cas9 genetic screening, we transduced it with a lentiviral vector that expresses Streptococcus pyogenes Cas9 and selected clones that were evaluated for Cas9 mRNA expression and mutagenic efficiency with a gene-specific gRNA. We chose a clone (clone E, FIG. 5G) with the lowest Cas9 expression level sufficient to induce the maximum gene disruption activity (>80% of alleles mutated) as the biosensor cell line for CRISPR-Cas9 gene inactivation screens.

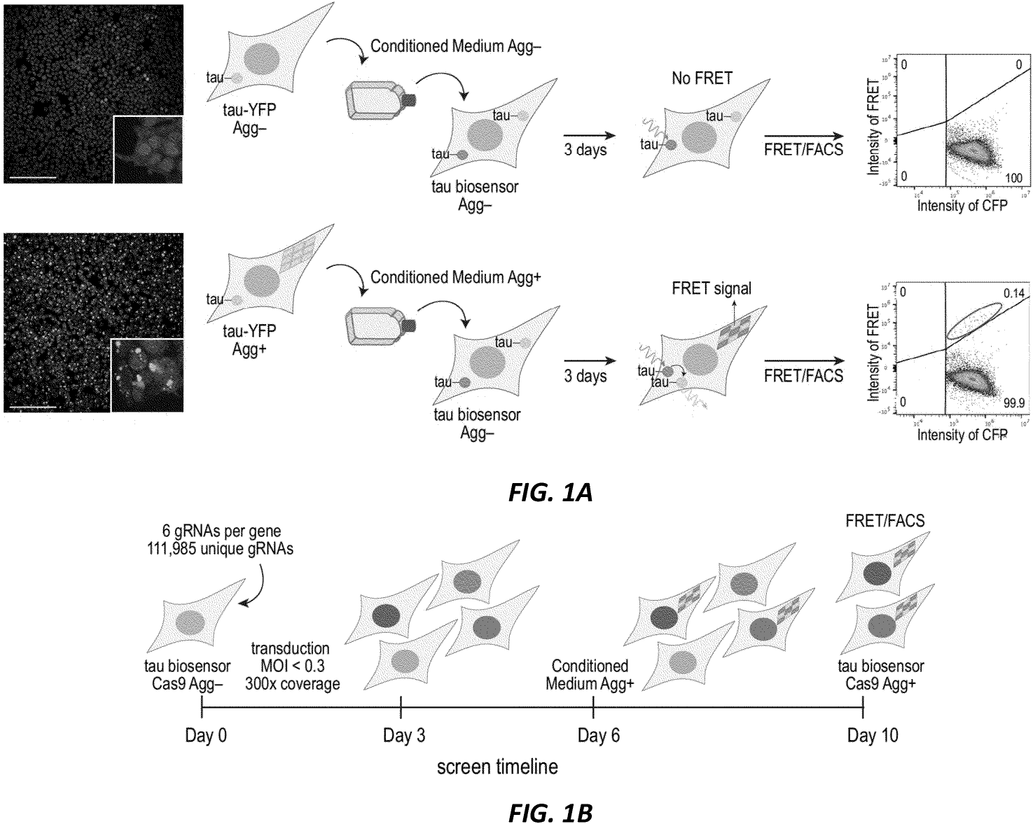

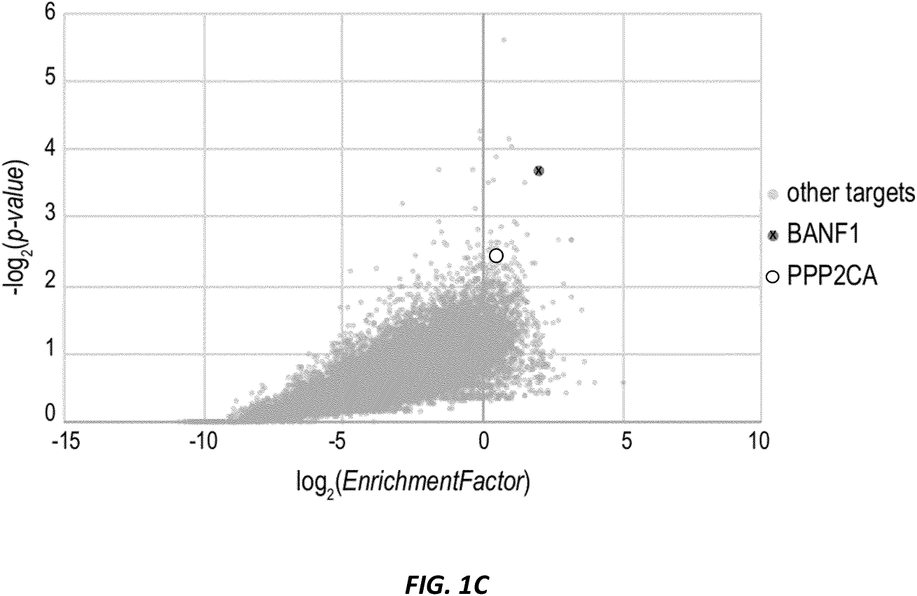

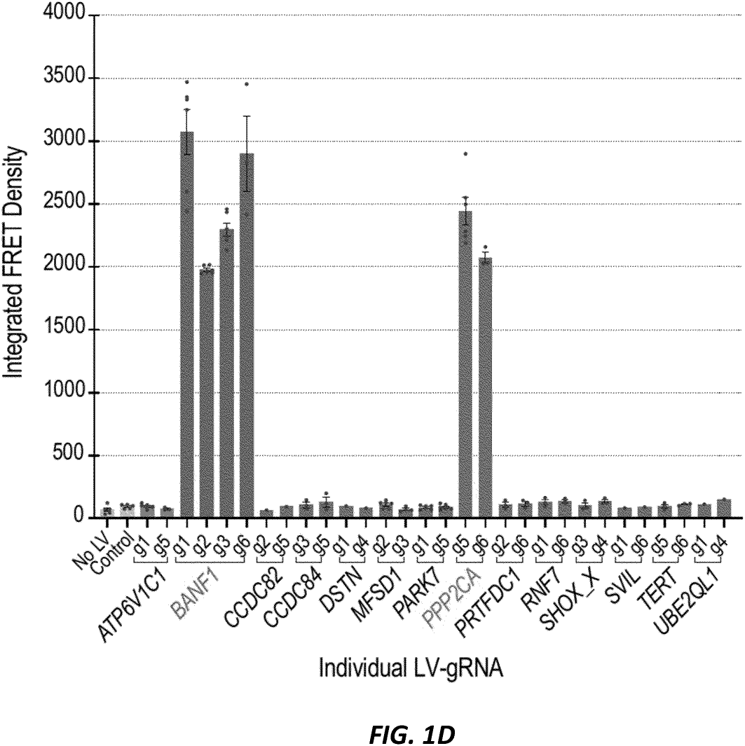

Visible fluorescent tau aggregates can also be induced in HEK293T cells that stably express only one of the biosensor fluorescent fusion proteins (FIG. 1A), but these cells do not produce a FRET signal. To establish an aggregation-positive (Agg+) cell line, we treated tau-YFP cells with recombinant tau fibrils complexed with LIPOFECTAMINE™ and isolated cell clones that stably propagate and maintain tau-YFP aggregates through many cell divisions. Treatment of the tau biosensor cells with cell-free conditioned medium from tau-YFP Agg+ cells induced a low level of tau aggregation indicated by a FRET signal in about 0.1% of the cells (FRET+ cells) analyzed by flow cytometry (FIG. 1A). In contrast, conditioned medium from the tau-YFP Agg- parental cell line failed to induce FRET. The weak induction of tau aggregation by conditioned medium from Agg+ cells may represent a more natural seeding agent and model for cell-to-cell spread in tauopathies than purified tau fibrils or tissue extracts complexed with transfection reagents. For these reasons, we decided to use conditioned medium as the seeding modality in our screens for mutations that promote tau aggregation.

A screen for mutations that enhance tau aggregation. FIG. 1B shows the protocol for the screen to discover mutations that enhance tau aggregation. We transduced the Cas9-expressing subclone of the tau biosensor cell line with the human Genome-scale CRISPR-Cas9 Knock Out (GeCKO) libraries packaged in a lentiviral expression vector. The hGeCKO-A and B half-libraries combined comprise 111,985 unique single gRNAs targeting 19,050 genes in the human genome (6 gRNAs per gene) and 1,000 non-targeting gRNAs as negative controls. We performed the transduction of the libraries at a low multiplicity of infection (MOI) to ensure that most cells would receive at most one gRNA construct at a coverage of 300 cells per gRNA and selected for viral vector integration events in the transduced cells by growth in the presence of puromycin. Six days after transduction of the GeCKO library, we introduced the tau aggregation seeding agent by transferring cells to new medium consisting of a 3:1 mixture of Agg+ conditioned medium: fresh growth medium. We sampled the transduced cell cultures at three days after transduction and at six days just before the addition of the conditioned medium seeding agent. After four additional days of growth in the presence of the seeding agent, we harvested the culture and purified FRET+ cells by FACS. We performed five replicate paired screens with the GeCKO A and B libraries separately, which generated samples at the day 3 and day 6 time points and the FACS-purified cells at day 10.

As a control, we performed identical paired screens but without the addition of the seeding agent. FACS analysis of these screens detected no FRET+ cells, identical to seeding with conditioned medium from an Agg- cell line (FIG. 1A). These results indicated that without a seeding agent, CRISPR-Cas9 mutagenesis alone was incapable of inducing spontaneous tau aggregation as measured by FRET. Either single-gene mutations are insufficient to induce tau aggregation, or such mutations are too rare to be detected with the number of cells analyzed. Even in the experimental screens with the Agg+ conditioned medium seeding agent, terminal FACS analysis at day 10 revealed that CRISPR-Cas9 mutagenesis did not enhance the proportion of FRET+ cells above the approximately 0.1% level produced in the absence of mutagenesis (FIG. 1A). These results suggested that either our CRISPR-Cas9 screening protocol failed to discover any mutations that could enhance seed-induced tau aggregation or that such mutations were too rare to produce a discernable increase in the low number of FRET+ cells produced by the weak seeding agent.

To search for evidence of mutations that might have promoted tau aggregation, we isolated DNA from the 10 pools of FRET+ cells and performed quantitative ILLUMINA® sequencing on PCR amplicons derived from the gRNA expression cassettes. We employed two strategies to identify potential gRNA-directed mutations. First, we used DESeq2 (Love et al. (2014) Genome Biol. 15:550, herein incorporated by reference in its entirety for all purposes) to identify gRNAs whose sequence reads were enriched in the FRET+ day 10 samples as compared to day 3 or day 6 samples, but not enriched in the day 6 samples as compared to day 3. Using an enrichment factor of ≥ 1.5 and a negative binomial Wald testp-value < 0.05 as cutoff thresholds, we found 104 gRNAs associated with 100 genes to be significantly enriched in the FRET+ day 10 samples compared with both day 3 and day 6 samples. Of the 100 genes identified, only one, Barrier-to-autointegration factor 1 (BANF1), was represented by 4 significantly enriched gRNAs (circles marked with an “X” in FIG. 5H). The remaining 99 genes were represented by single significantly enriched gRNAs. As the read count data generated from the CRISPR screening may not follow the data distribution assumed by the Wald test used by DESeq2, we developed an alternative gene-centric enrichment analysis algorithm that does not require any assumption of data distribution across samples. As in DESeq2, our alternative algorithm has 2 components: an enrichment factor (referred to as fold change in DESeq2) and an enrichment p-value. The enrichment factor is similar to that used in DESeq2 except that it is summarized at the gene level by averaging the enrichment of all gRNAs targeting the same gene. Different from existing CRISPR data analysis methods, in our method, we used a hypergeometric distribution-based p-value calculation applied only to the enrichment of the different gRNAs targeting a gene in each of the FRET+ day 10 samples. The final enrichment p-value of a gene is the average of the p-values obtained from each of the 10 FRET+ day 10 samples. Our alternative algorithm revealed BANF1 as a top hit (pink highlighted dot in FIG. 1C), confirming the DESeq2 results. From a visual inspection of the data quality among the genes ranked by p-value and enrichment from DESeq2 and our gene-centric method, we selected BANF1 and 13 other genes that had at least two active gRNAs that exhibited enrichment for experimental validation.

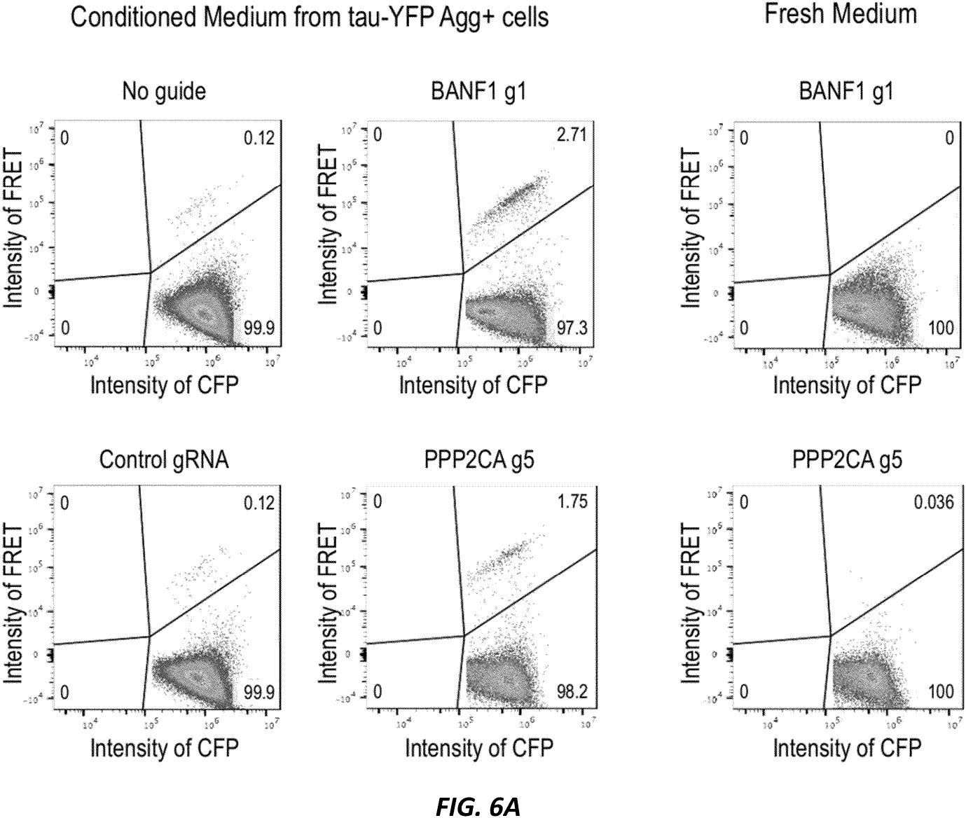

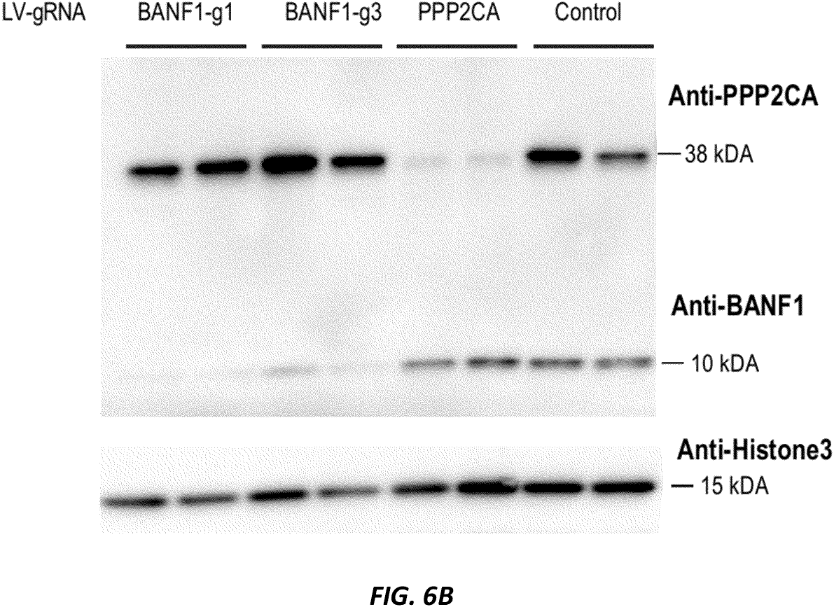

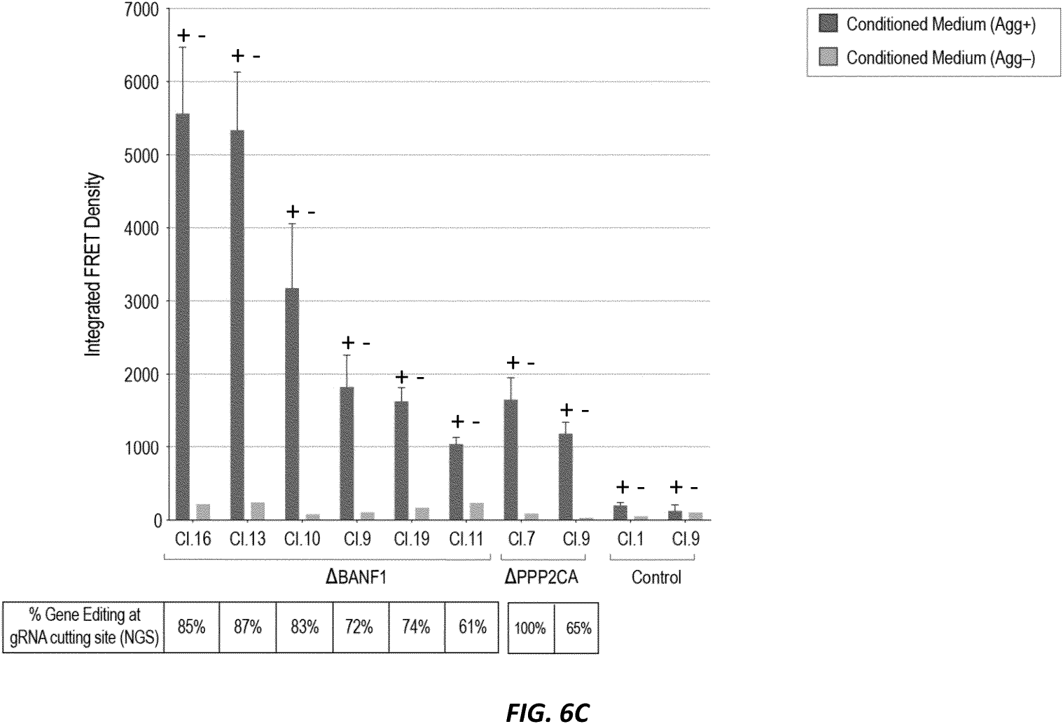

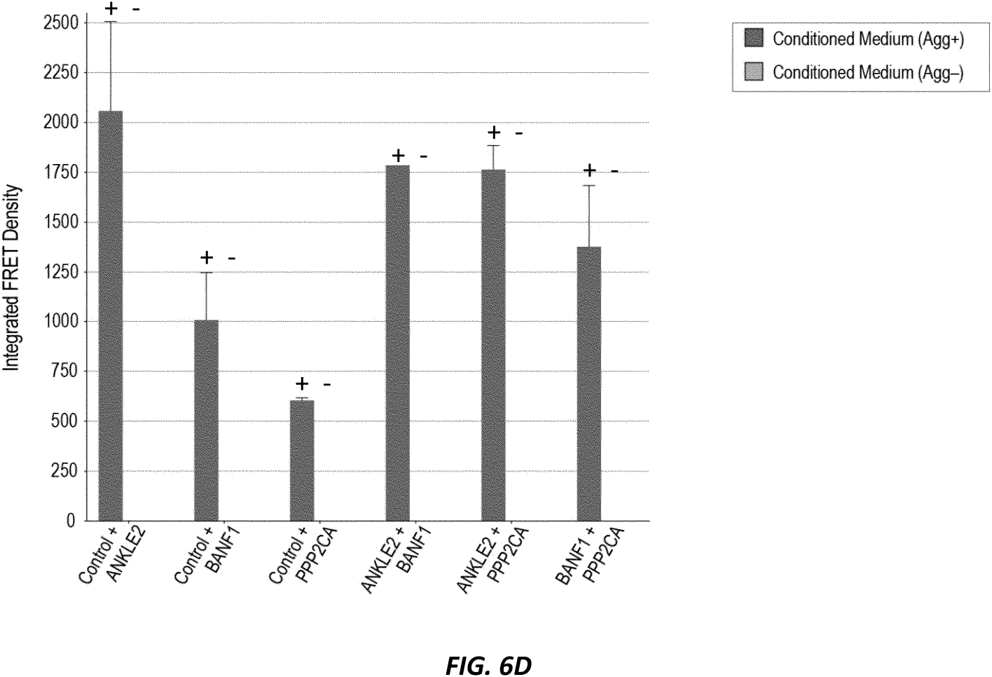

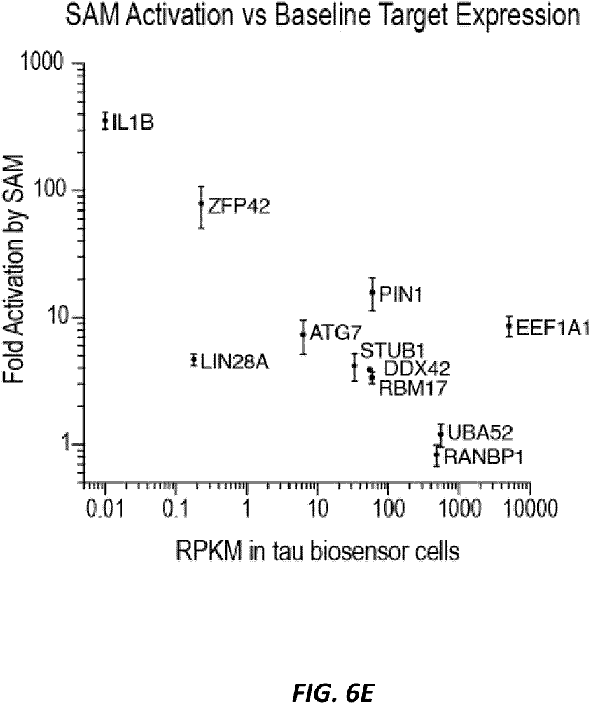

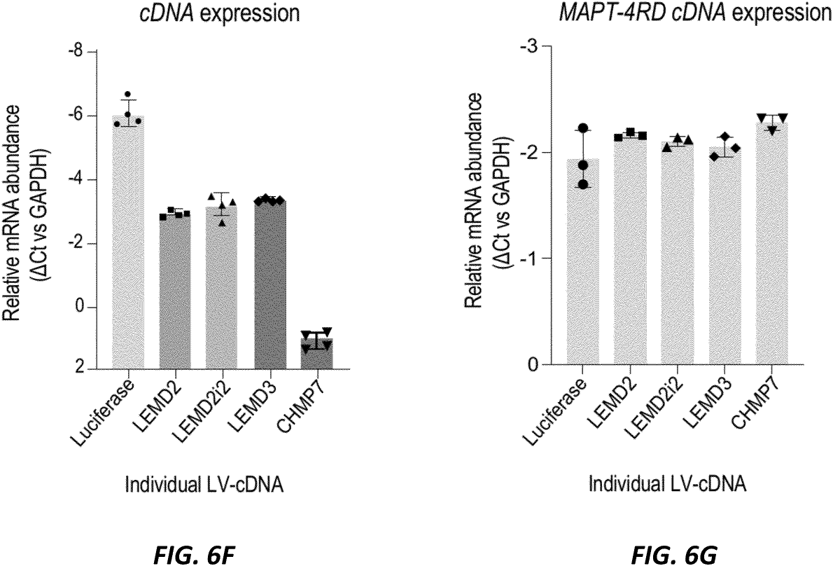

To confirm the candidate genes, we tested 30 individual GeCKO library lentiviral gRNA expression vectors, four for BANF1 and two each for the 13 other genes, for their ability to enhance FRET induced by Agg+ conditioned medium. Transduction of the tau biosensor cells with each of the four BANF1-targeting gRNAs (pink bars in FIG. 1D) and both gRNAs targeting the PPP2CA gene (blue bars in FIG. 1D and blue dot in FIG. 1C) increased the induction of FRET signal by a factor of 15-20 compared with no gRNA or control gRNA (FIG. 1D). None of the gRNAs for the other candidate genes promoted enhanced FRET. CRISPR-Cas9 disruption of BANF1 and PPP2CA did not induce FRET when fresh medium was used in place of conditioned medium (FIG. 6A). Western blotting assays confirmed that gRNAs targeting BANF1 and PPP2CA greatly reduced protein produced from the targeted genes (FIG. 6B). Moreover, individual isolated BANF1 and PPP2CA knockdown clones revealed a positive correlation between the extent of gene editing and the enhancement of FRET induced by Agg+ conditioned medium (FIG. 6C). Combined inactivation of BANF1 and PPP2CA produced an apparent additive effect on FRET induction (FIG. 6D), suggesting that these two genes participate in a common function.

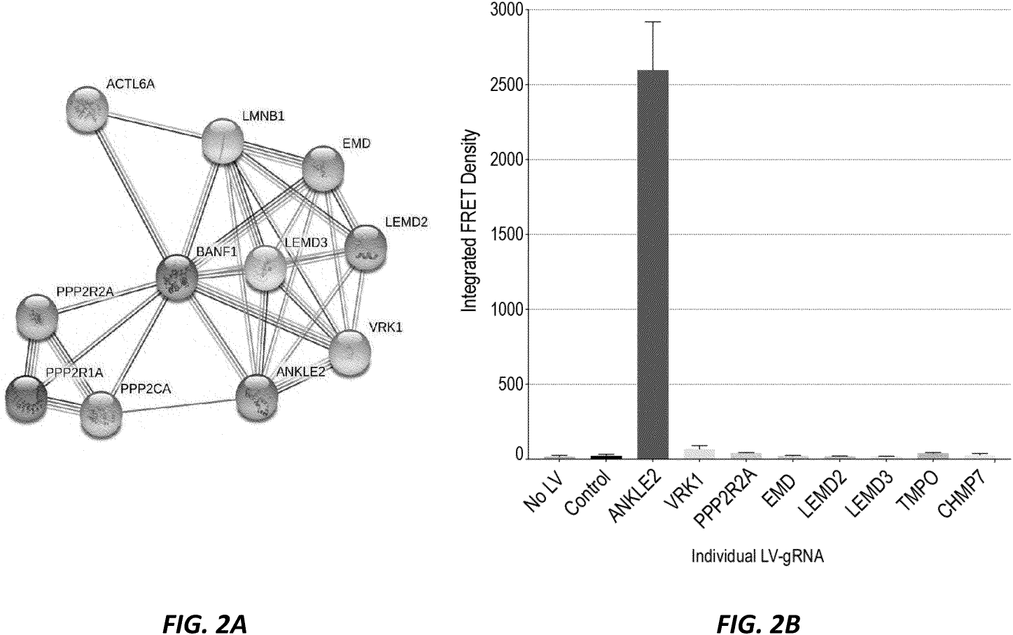

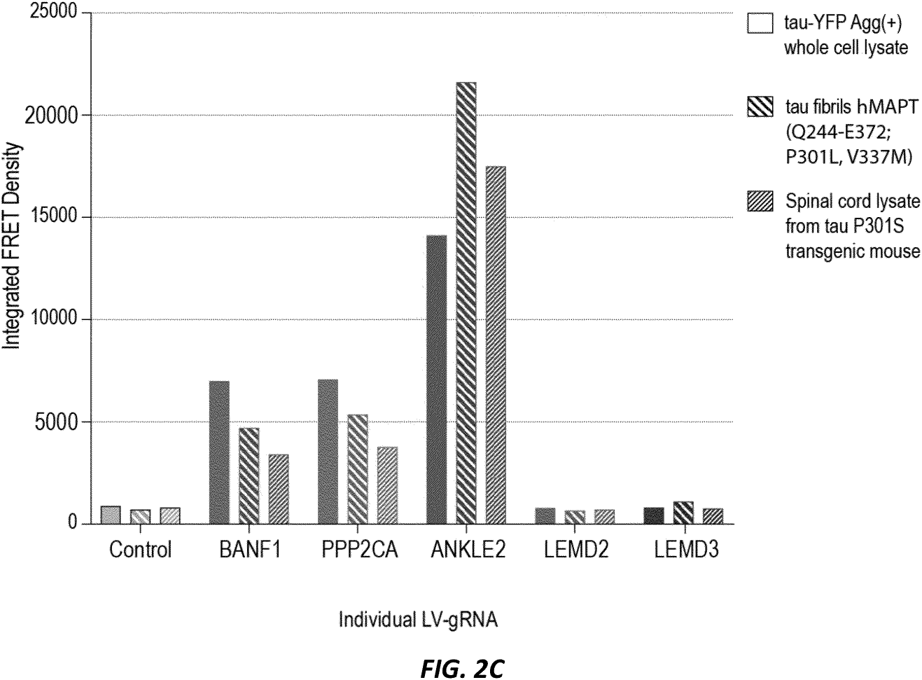

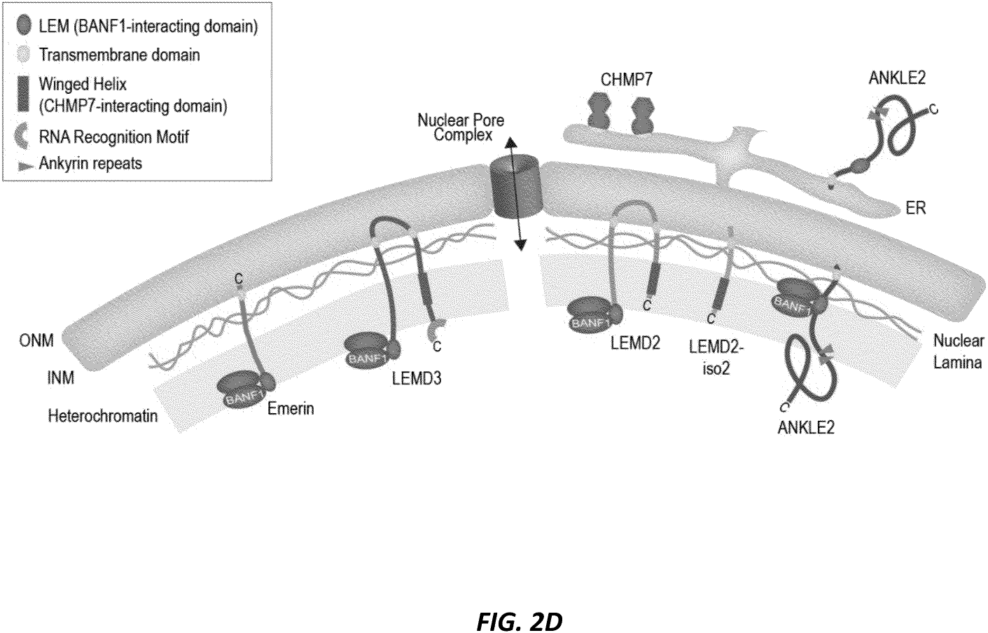

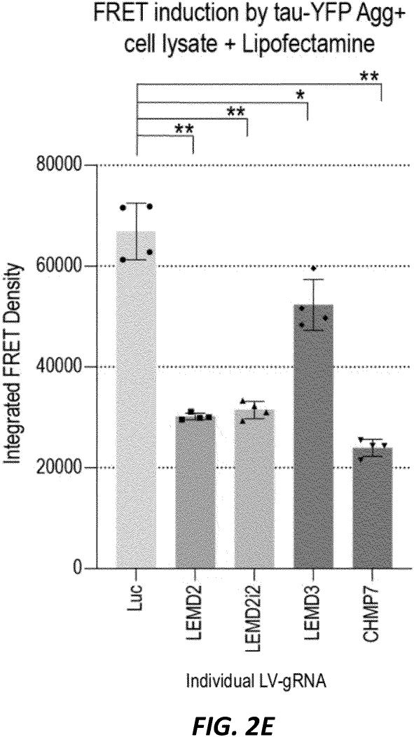

Validated gene hits from the gene disruption screen encode proteins that are part of a functional network of components that maintain the nuclear envelope. To better understand the biology of the confirmed hits from the screen, we used the top hit, BANF1, to search for its protein-protein association network in String. Szklarczyk et al. (2019) Nucleic Acids Res. 47:D607-D613, herein incorporated by reference in its entirety for all purposes. The network revealed that BANF1 directly interacts with PPP2CA, the second confirmed hit, in a catalytic relationship (FIG. 2A). In multiple functional enrichment analyses of the network, nuclear envelope stands out as the most significant feature. To test whether mutations in other components of the BANF1 interaction network might promote enhanced tau aggregation, we tested gRNAs directed against ANKLE2, VRK1, PPP2R2A, EMD, LEMD2, LEMD3, and TMPO (FIG. 2A) for their ability to induce enhanced FRET. We also included CHMP7 because of its role in the maintenance of nuclear envelope integrity. Only the gRNA targeting ANKLE2 was able to induce a large enhancement of FRET compared with the controls (FIG. 2B). We confirmed that disruption of BANF1, PPP2CA, and ANKLE2 produced enhancement of tau aggregation, indicated by FRET, with three different seeding modalities: sonicated whole cell lysate from tau-YFP Agg+ cells, purified misfolded recombinant tau fibrils (tau 244-372 LM, FIGS. 5A-5D), and a spinal cord extract from tau P301S transgenic mice (FIG. 2C). See, e.g., Yoshiyama et al. (2007) Neuron 53:337-351, herein incorporated by reference in its entirety for all purposes. Inactivation of ANKLE2 induces a stronger FRET signal than loss of either BANF1 or PPP2CA, and combining ANKLE2 inactivation with that for BANF1 or PPP2CA produced no additive effect (FIG. 6D). The results of our primary screen, the secondary validation, and the tertiary screen for biologically related genes point to an unexpected link between microtubule associated protein tau and the biological processes that maintain the nuclear envelope (FIG. 2D), suggesting that defects in nuclear envelope integrity could initiate pathogenesis or promote its spread in tauopathies.

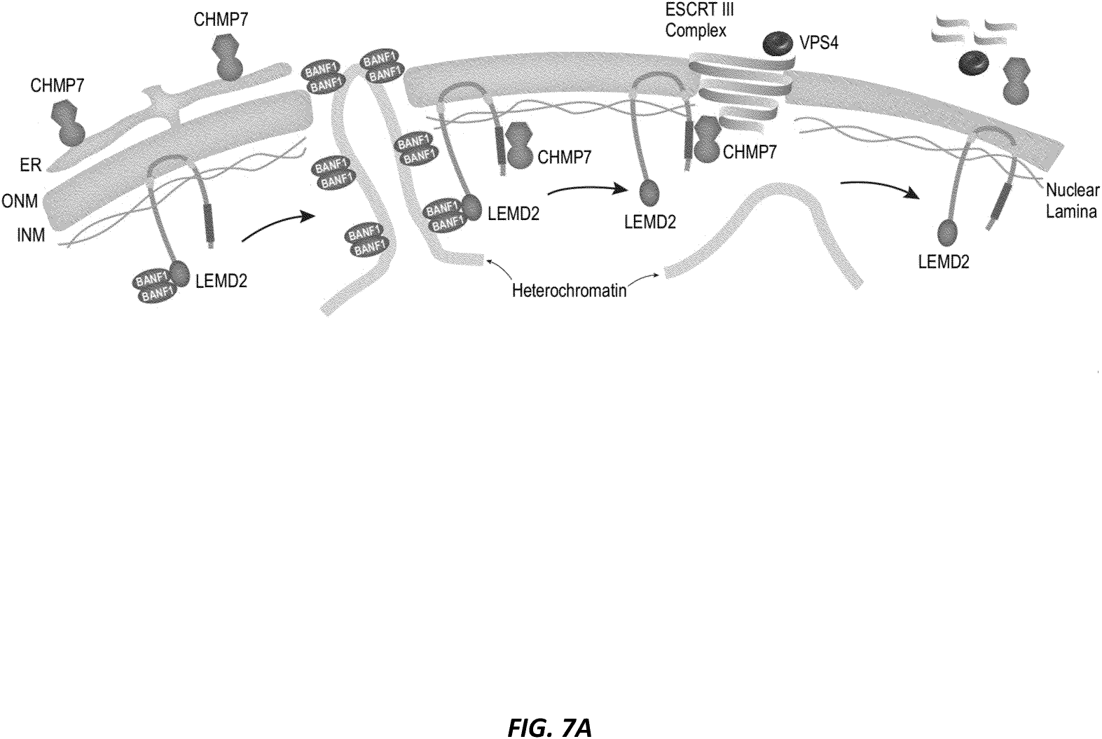

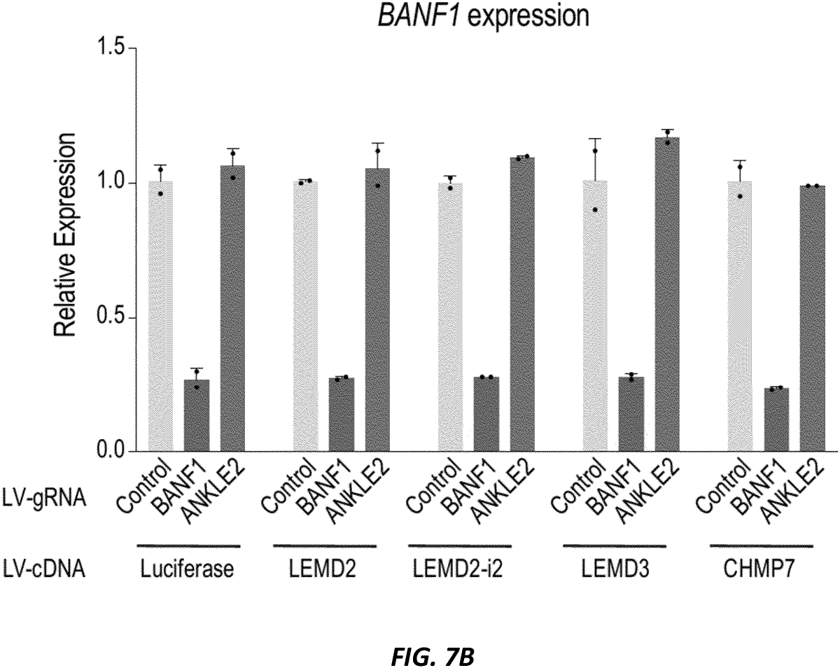

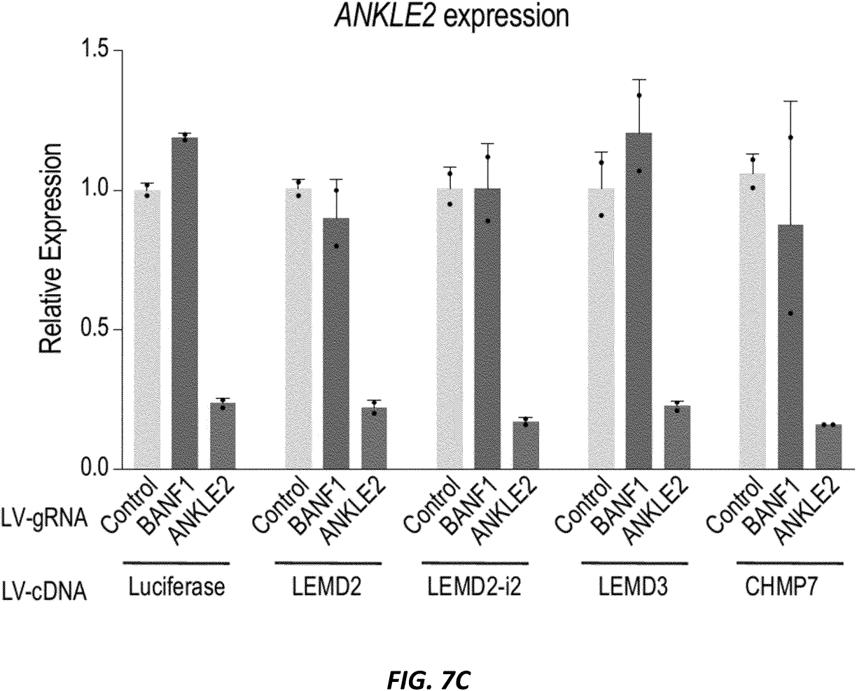

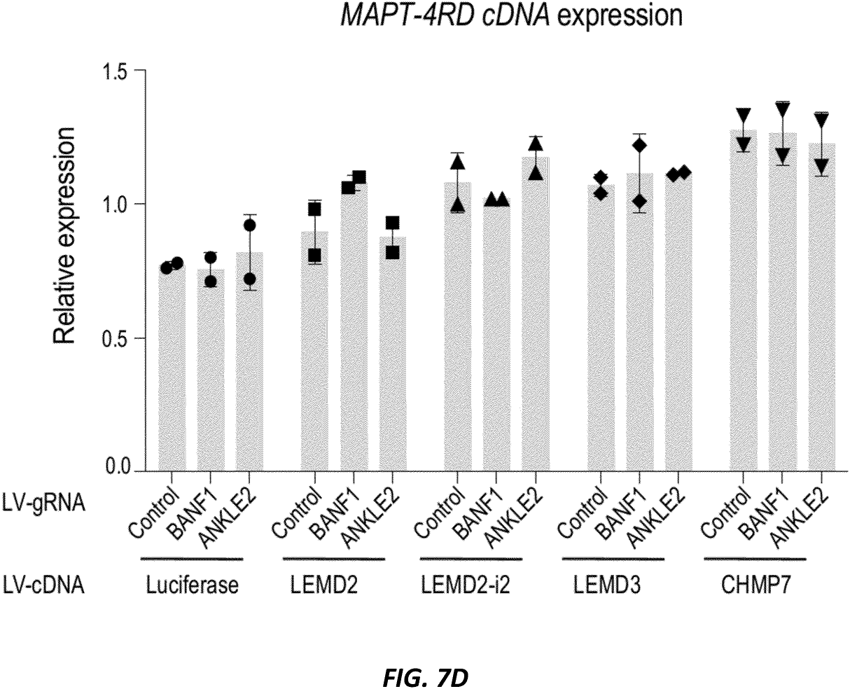

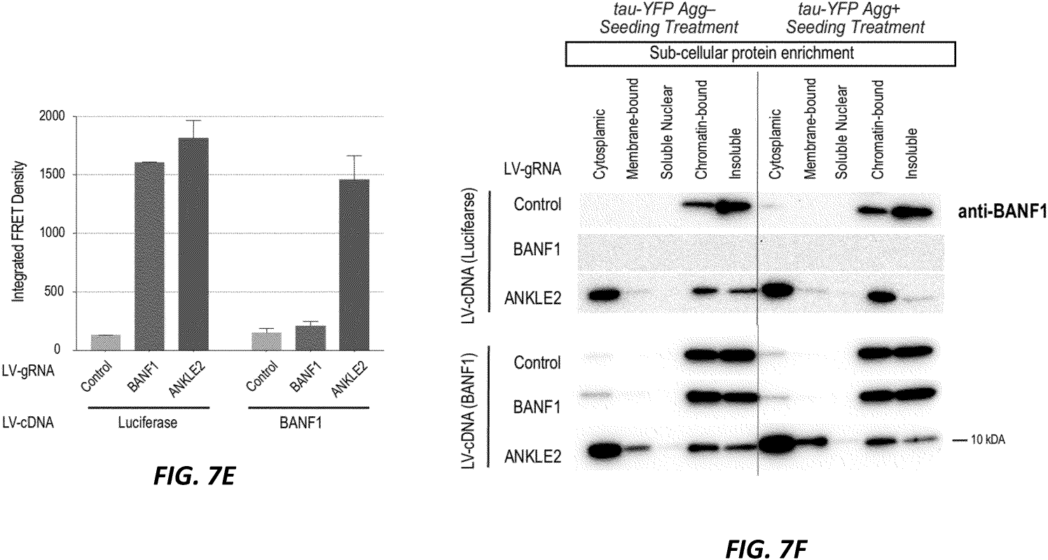

A screen for genes that when overexpressed inhibit or prevent tau aggregation. As disruption of BANF1, PPP2CA, or ANKLE2 enhances tau aggregation, the normal functioning of these genes might serve to protect cells from tau pathogenesis. To discover additional protective genes whose expression might reverse or prevent tau aggregation, we reversed the logic of our CRISPR-Cas9 gene disruption screen by employing the SAM system for gRNA-targeted gene activation to screen for genes whose activation or enhanced expression would abolish FRET induction in the tau biosensor cells. As with the gene disruption screen, we first established a tau biosensor subclone that stably expressed the components of the SAM system and confirmed that we could induce or enhance gene expression with specific SAM gRNAs (FIG. 6E). We then performed five replicate screens by transduction of the SAM-ready biosensor cell line with a human SAM gRNA library as depicted in FIG. 1B except that we replaced the weak conditioned medium seeding agent with a strong seeding agent, a sonicated lysate of tau-YFP Agg+ cells complexed with Lipofectamine™. This method of seeding routinely produces ~60% FRET+ cells compared with 0.1% for the conditioned medium. For the overexpression screen, we sampled cells on day 7 and day 10, when the seeding agent was added. At day 13, FRET+ and FRET- cells were collected by FACS. For gRNA sequence enrichment analysis we used the DESeq2 algorithm to compare the FRET- population with both the FRET+ cells and with cells sampled at day7 and day 10. These analyses revealed two gRNAs for the gene, LEMD2, that were significantly enriched in the FRET- cells. String analysis (FIG. 2A) identified LEMD2 within the nuclear envelope functional network, wherein it binds to BANF1 and, like ANKLE2, is an integral component of the inner nuclear membrane of the nuclear envelope (FIG. 2D). For secondary confirmation of LEMD2, we used cDNA expression rather than dCas9-SAM transcriptional enhancement. We tested cDNAs that express two isoforms of LEMD2 and the closely related LEMD3 protein and a cDNA for CHMP7 because it binds LEMD2 and participates in the repair of nuclear envelope ruptures to promote membrane sealing (FIG. 7A). Sequences for the lentiviral cDNA constructs are set forth in SEQ ID NOS: 34, 36, 37, and 39. Expression of all four cDNAs in biosensor cells reduced FRET induction by a strong seeding agent (FIGS. 2E and 6F), and didn’t affect the expression of the MAPT-4RD transgenes (FIG. 6G). The LEMD2 isoforms and CHMP7 showed stronger rescue than LEMD3.

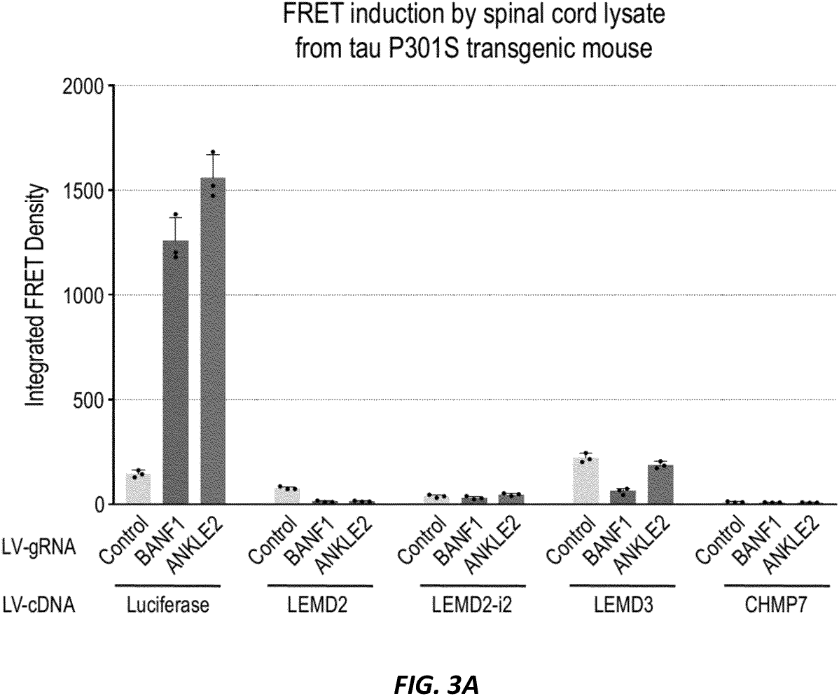

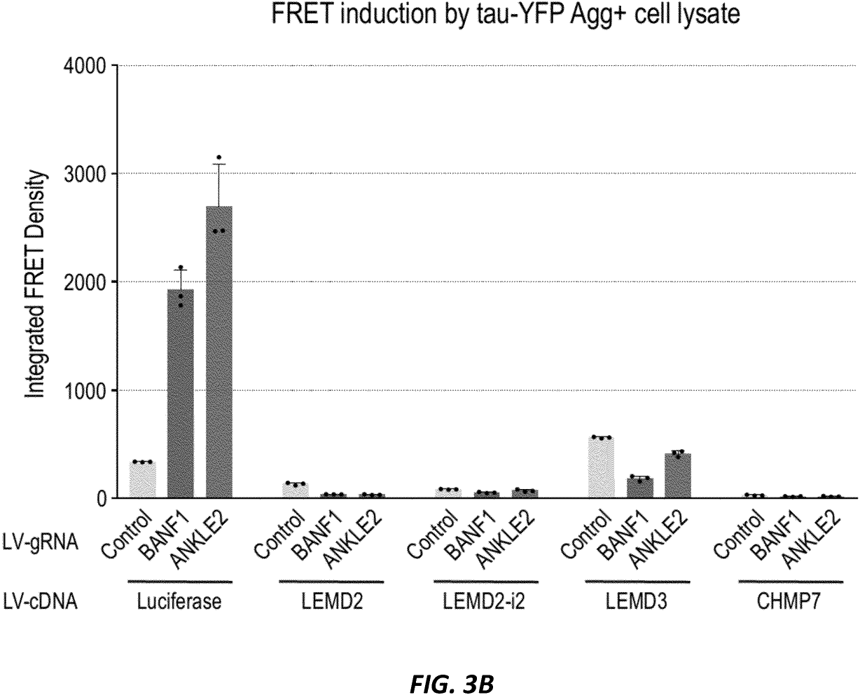

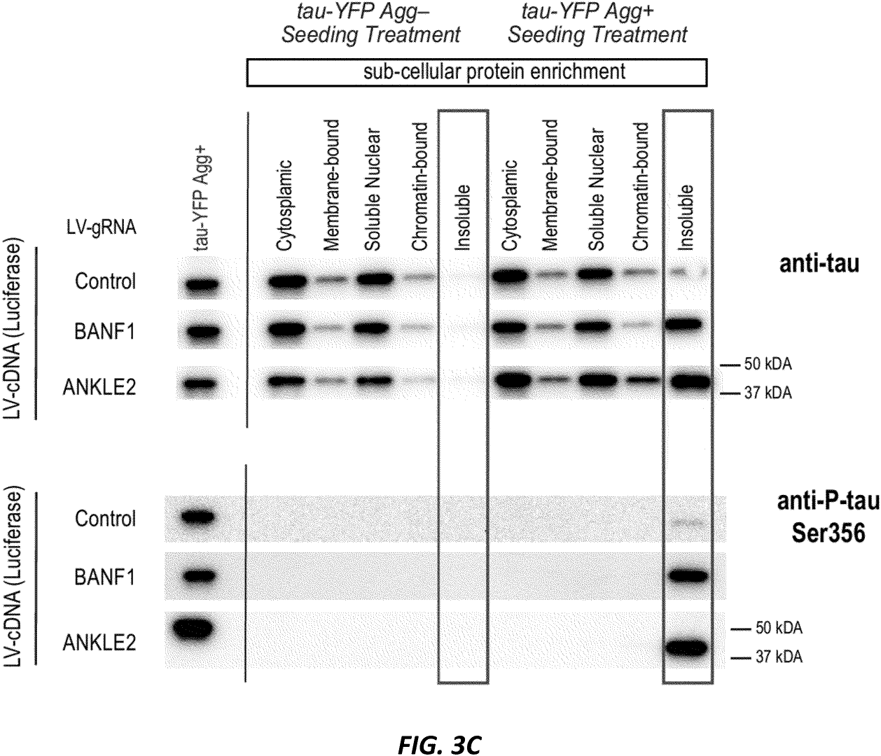

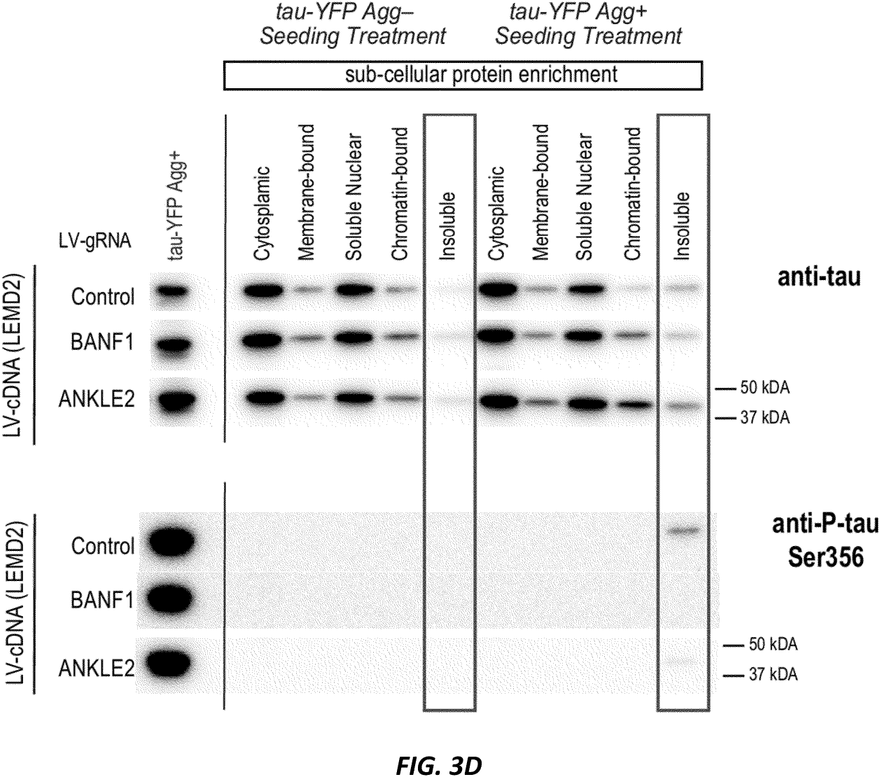

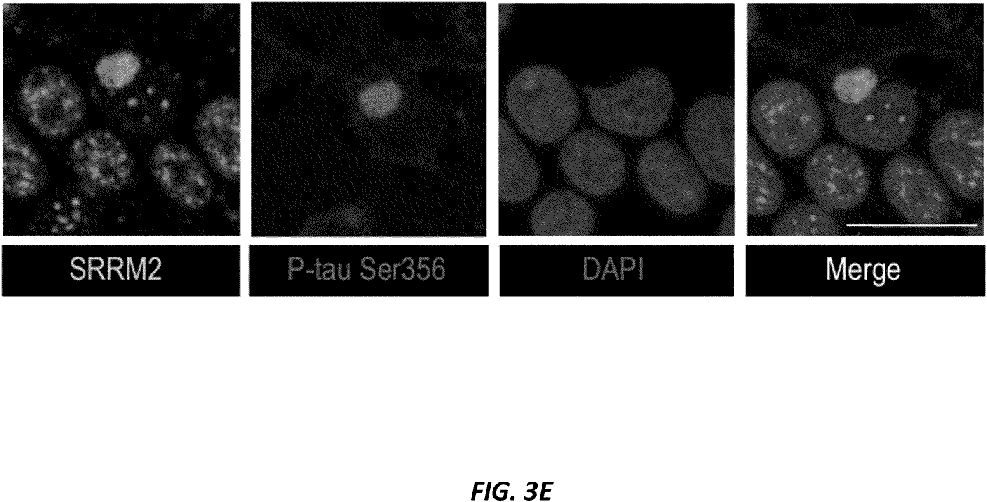

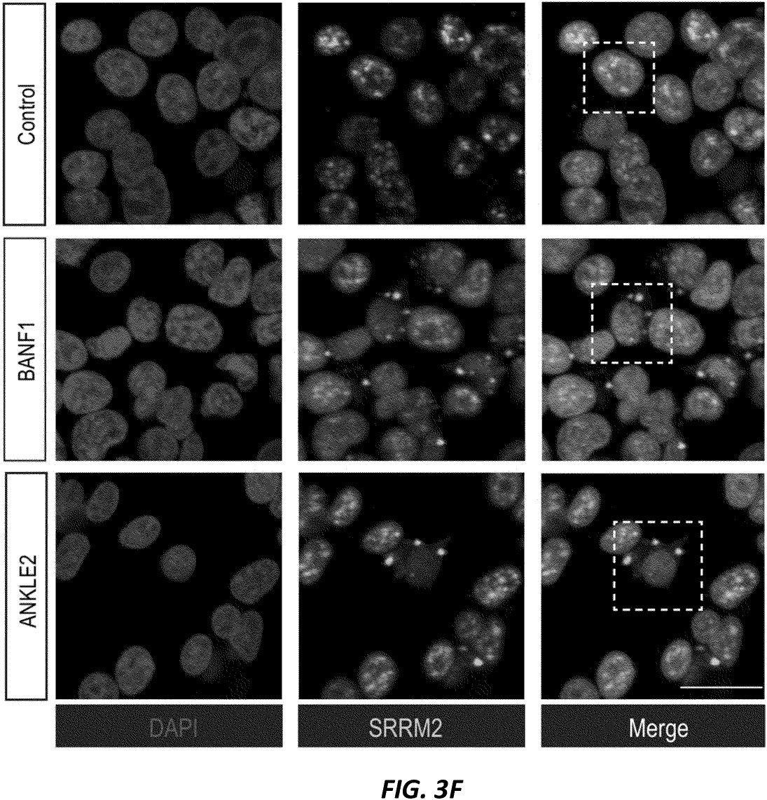

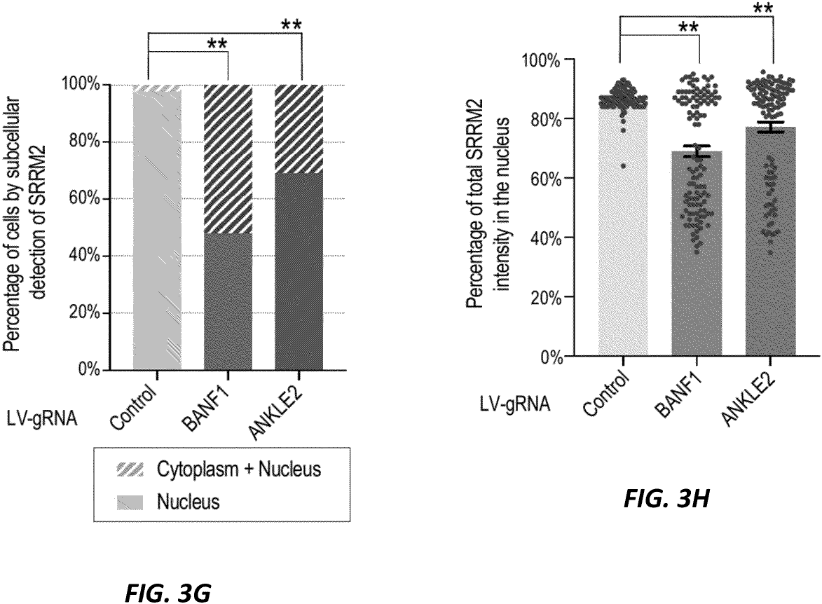

To further investigate the protective properties of LEMD2, LEMD3, and CHMP7, we tested the ability of cDNA overexpression of these proteins to rescue tau aggregation induced by loss of BANF1 and ANKLE2. To set up these screens, we first established a tau biosensor subclone that stably expresses a catalytically dead form of Cas9 fused to a Krüppel associated box transcriptional repression domain (dCas9-KRAB). We then transduced promoter-targeting lentiviral gRNA expression vectors to establish stable knockdowns of BANF1 and ANKLE2 gene expression. Seeding agents induced tau aggregation as indicated by a strong FRET signal in the BANF1 and ANKLE2 knockdown biosensor cells compared with control cells that stably express a control gRNA, gNT303 (FIGS. 3A and 3B). As a positive control, expression of a BANF1 cDNA abolished seed-induced FRET in the BANF1 knockdown cell line but not in cells with knockdown of ANKLE2 (FIG. 7E). Expression of cDNAs for both isoforms of LEMD2 and for LEMD3 and CHMP7 all abolished or significantly reduced FRET induction by two different seeding agents (FIGS. 3A and 3B). Expression of all four cDNAs did not reverse the dCas9-KRAB transcriptional repression of BANF1 and ANKLE2 (FIGS. 7B and 7C). Although we did not assess tau-4RD protein levels, expression of these cDNAs did not affect expression of MAPT-4RD cDNA transgenes (FIG. 7D) assessed by TAQMAN qRT-PCR, indicating that the rescue of FRET induction by these cDNAs was a direct effect. Sequences for the lentiviral cDNA constructs are set forth in SEQ ID NOS: 32, 34, 36, 37, and 39.

To examine how LEMD2 expression affects the biochemical behavior of tau, we analyzed subcellular fractions of the BANF1 and ANKLE2 knockdown cell lines with and without LEMD2 overexpression. In the absence of a seeding agent, we found that most of the tau protein was distributed between the cytoplasmic and soluble nuclear fractions in control dCas9-KRAB biosensor cells expressing a control gRNA (upper left quadrants in FIGS. 3C and 3D). This pattern was not altered by knockdown of BANF1 or ANKLE2; however, treatment with the tau-YFP Agg+ cell lysate caused tau to accumulate in the insoluble fractions in the BANF1 or ANKLE2 knockdown cells but not in the control cells (FIG. 3C, upper right quadrant). The insoluble tau was phosphorylated on serine 356 (FIG. 3C, lower right quadrant), which is a marker for tau aggregates in seeded tau biosensor cells and correlates with the seeding activity in the tau-YFP Agg+ cells (FIG. 5F). Consistent with its ability to prevent tau aggregation assessed by FRET (FIGS. 2E, 3A, and 3B), expression of the LEMD2 cDNA prevented accumulation of tau in a seed-induced insoluble and phosphorylated biochemical isoform (FIG. 3D).

Loss of BANF1 or ANKLE2 induces mislocalization of nuclear speckle component SRRM2. Our results demonstrate that loss of the nuclear envelope components BANF1 and ANKLE2 enhances tau aggregation in the tau biosensor cells and promotes the accumulation of P-tau-Ser356 in insoluble subcellular fractions (FIGS. 3A-3D), but these changes in tau’s biochemical properties require induction with a seeding agent. Our biochemical fractionation indicates that tau is an abundant constituent of the nucleus. Might reduction of BANF1 and ANKLE2 cause a disruption in a nuclear function that promotes susceptibility to tau seeding? Lester et al. showed that small nuclear and nucleolar RNAs and components of nuclear speckles, sites of pre-mRNA splicing, were associated with tau aggregates induced by a strong seeding agent in the tau biosensor cells. Lester et al. (2021) Neuron 109(10):1675-1691, herein incorporated by reference in its entirety for all purposes. In particular, the nuclear speckle component SRRM2 was colocalized with tau in large cytoplasmic aggregates. We reproduced this result with our tau biosensor cells and the tau-YFP Agg+ whole cell lysate as tau seeding agent (FIG. 3E). But even without tau seeding, we found that biosensor cells with dCas9-KRAB-induced reductions in BANF1 or ANKLE2 showed mislocalization of SRRM2 from the nucleus to the cytoplasm (FIG. 3F), yet these cells did not show any signs of tau aggregation or phosphorylation (see the biochemical analysis of similar cells in FIG. 3C). In normal tau biosensor cells, immunofluorescent detection of SRRM2 is nearly completely confined to the nucleus, while in the BANF1- and ANKLE2-deficient cells, some SRRM2 is clearly seen in perinuclear foci (FIG. 3F), indicating escape of the protein in a form that appears to be locally concentrated. Visual counting of immunofluorescent cells revealed that approximately half of the BANF1-deficient cells had lost exclusive nuclear retention of SRRM2 and showed a pattern of both nuclear and cytoplasmic localization, while nearly 30% of ANKLE2-deficient cells showed both nuclear and cytoplasmic SRRM2 detection (FIG. 3G). Using a different type of quantitative analysis, we determined the percentage of SRRM2 immunofluorescence associated with the nucleus in each cell examined. Most of the control cells had about 90% of their SRRM2 signal in the nucleus. In the BANF1- and ANKLE2-deficient cells, we observed two populations: one that looked like the control, with 90% of the SRRM2 retained in the nucleus; and a second group that showed approximately equal (40-60%) distribution of SRRM2 between the nucleus and the cytoplasm (FIG. 3H). We observed a similar effect for BANF1. Reduction of ANKLE2 expression causes BANF1 protein accumulation in the cytoplasm and its loss from the chromatin-bound fraction (FIG. 7F). These results indicate that loss of nuclear envelope integrity in the absence of overt tau aggregation could be an initiating event that enables leakage of SRRM2, tau, and perhaps other nuclear components into the cytoplasm, which eventually triggers their aggregation.

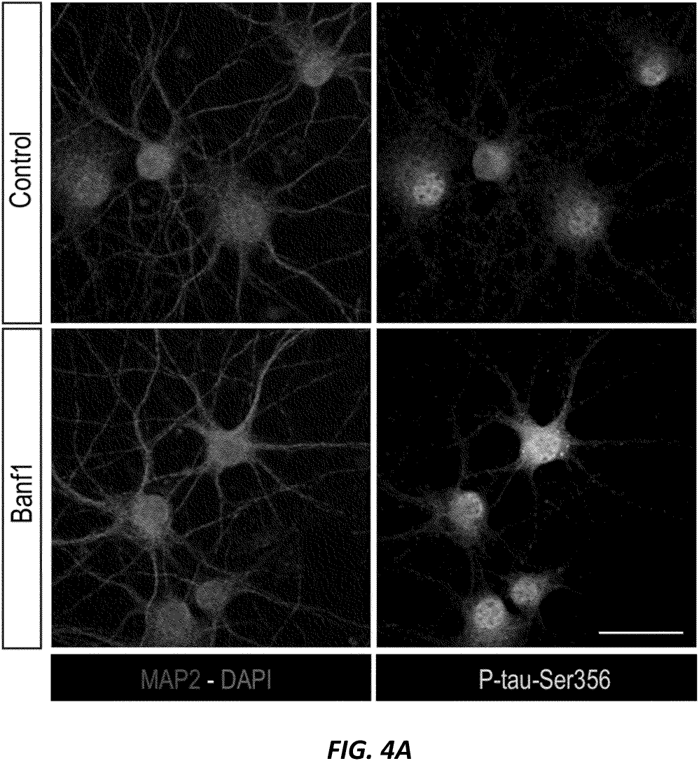

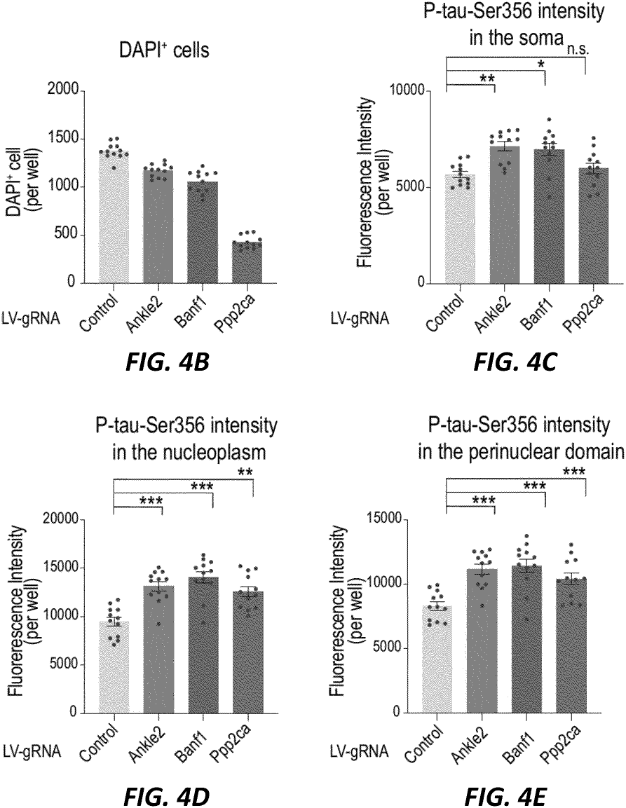

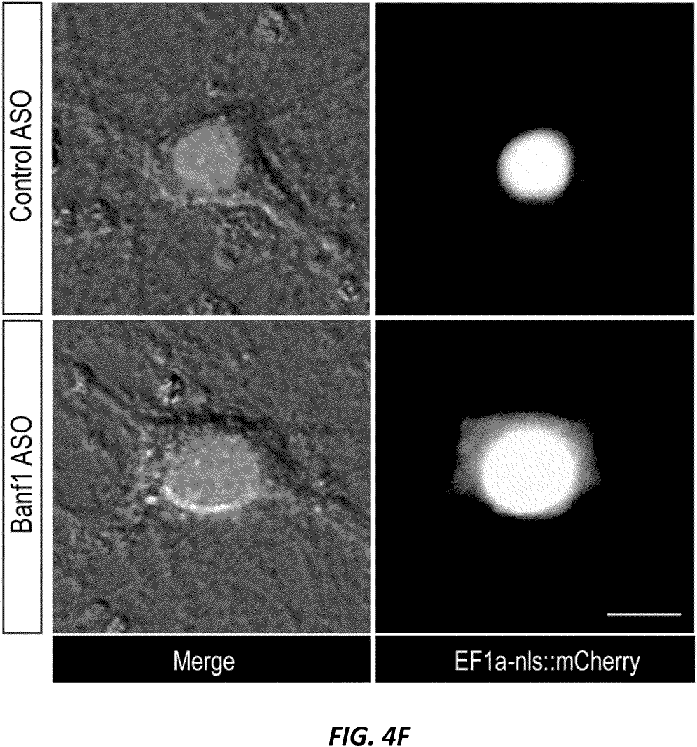

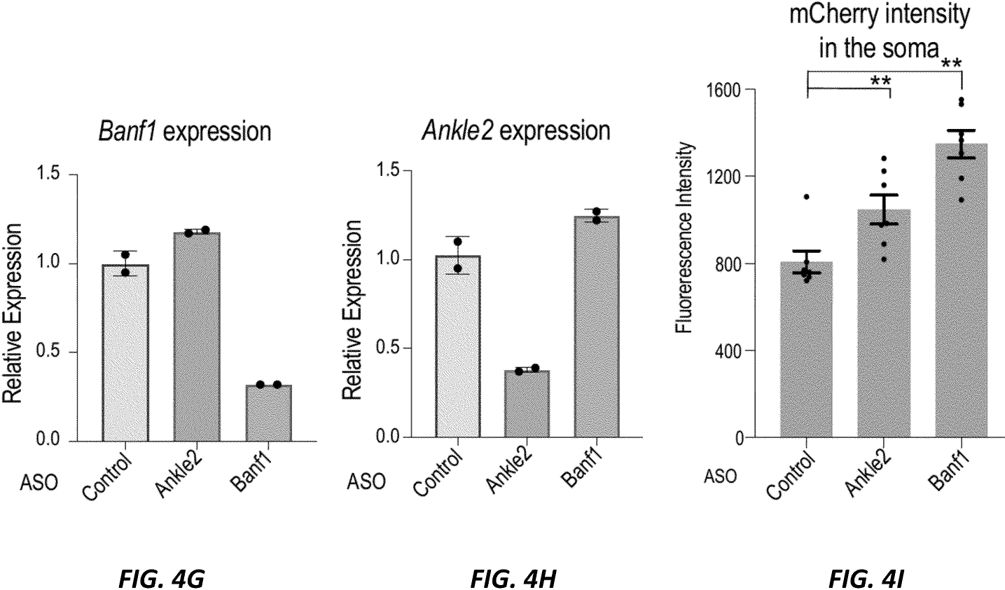

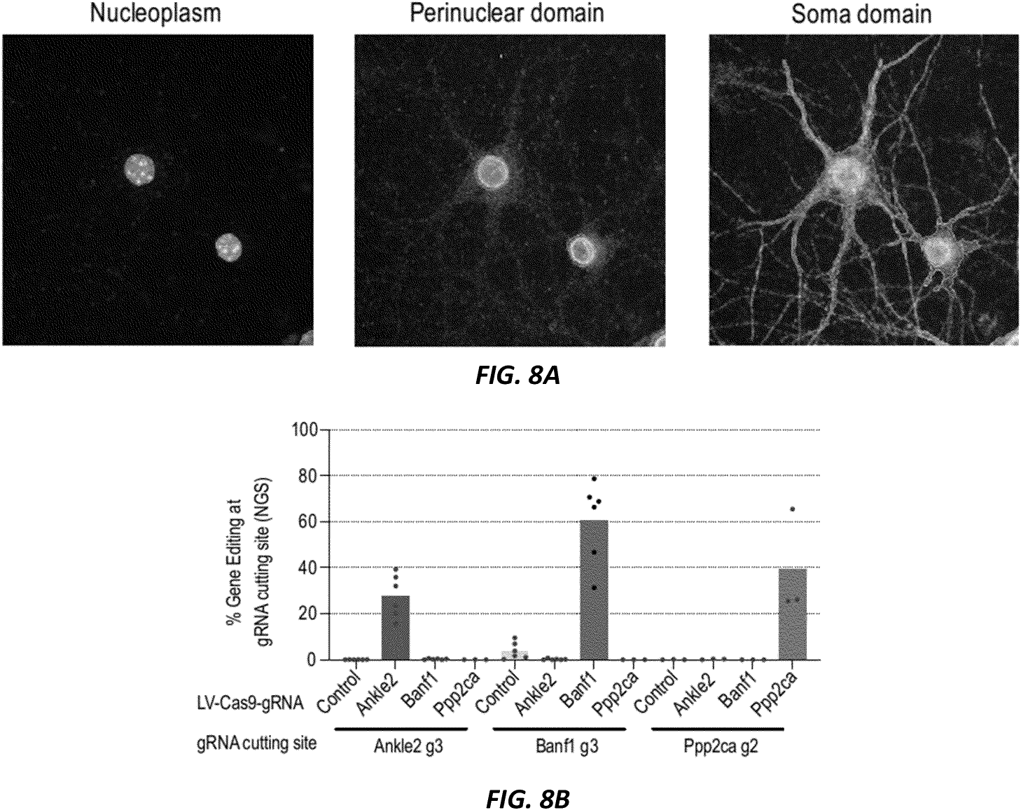

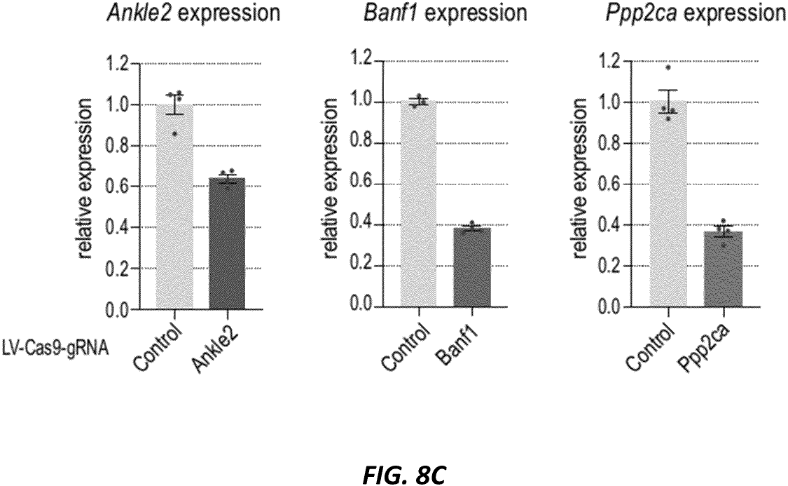

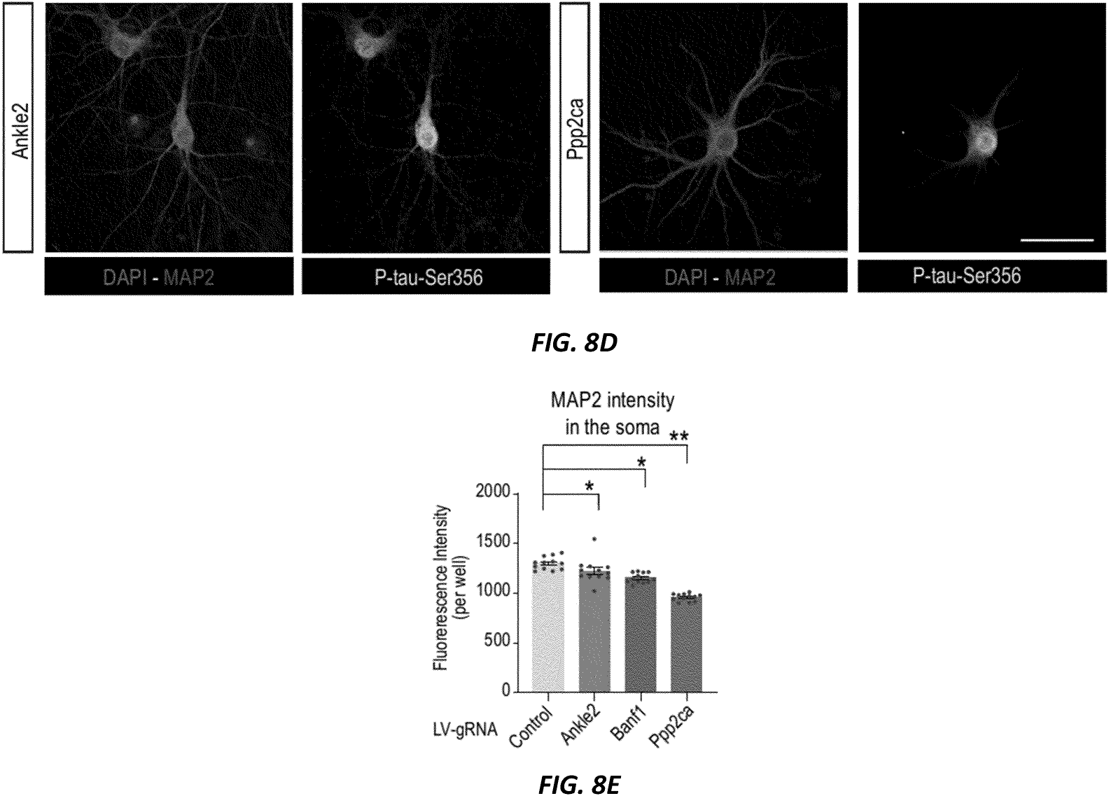

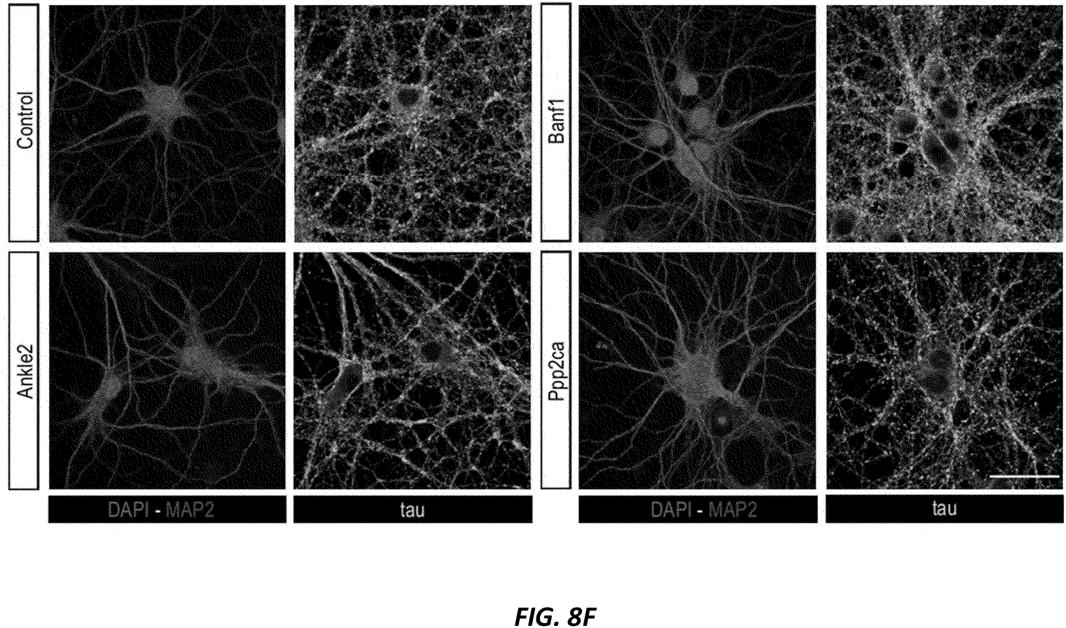

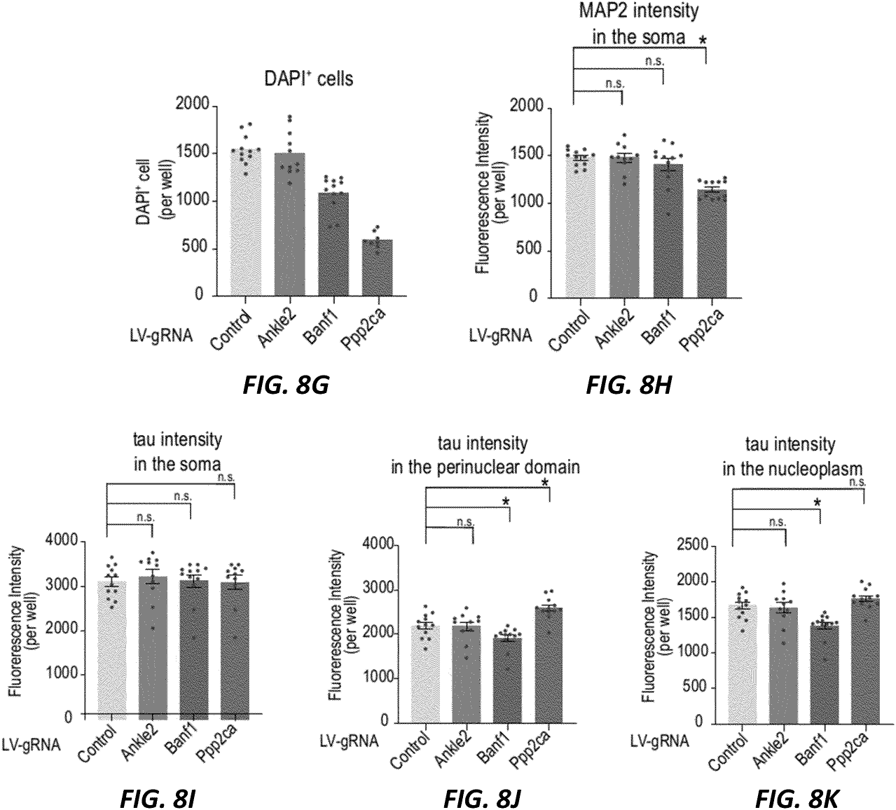

Reduced expression of Banf1, Ankle2, and Ppp2ca causes increased phosphorylation of tau on serine 356 and impaired nuclear envelope integrity in mouse cortical neurons. Our genetic screens were performed in highly proliferative HEK293T cells, which undergo constant cycles of nuclear breakdown and reformation. To examine the consequences of perturbation of the nuclear envelope in a post-mitotic cell more relevant to tau pathology, we transduced wild type primary mouse cortical neurons with lentiviruses that co-expressed Cas9 with either a control gRNA or a gRNA targeting either Banf1, Ankle2, or Ppp2ca or a control gRNA. We confirmed gene disruption by NGS and TAQMAN qRT-PCR (FIGS. 8B-8C). Disruption of Banf1, Ankle2, or Ppp2ca caused a significant increase in P-tau-Ser356 in the perinuclear domain and the nucleoplasm (FIGS. 4A, 4D, 4E, 8D). P-tau-Ser356 signal was enhanced in the soma upon knockdown of Banf1 or Ankle2 but not Ppp2ca (FIG. 4C). Disruption of any of the three genes had no effect on total tau in the soma (FIGS. 8F, 8I). Disruption of Banf1 caused a slight loss of tau detection in the perinuclear domain and in the nucleoplasm (FIGS. 8J-8K), while knockdown of Ppp2ca produced a slight increase in tau detection in the perinuclear domain (FIG. 8J). Disruption of Ppp2ca appeared to cause some toxicity(FIGS. 4B, 8E, 8G, 8H).

We next wanted to assess the effect of the depletion of Banf1 and Ankle2 upon the nuclear envelope integrity of primary mouse cortical neurons. To this end, we made use of a fluorescent nuclear reporter (NLS::mCherry), and ASOs targeting mouse Banf1, Ankle2, or a scrambled ASO sequence as control. The ASOs were introduced to the mouse cortical neurons via gymnotic delivery, and qRT-PCR analysis confirmed specific and significantly reduced expression of Banf1 and Ankle2 (FIGS. 4G and 4H). Live-cell imaging studies revealed increased detection of mCherry in the soma of mouse cortical neurons treated with Banf1 or Ankle2 ASO as compared to control ASO, as evident in FIG. 4F. Quantification of mCherry fluorescence intensity revealed significantly increased mCherry in the soma of Ankle2- or Banf1-depleted neurons, possibly resulting from nuclear leakage.

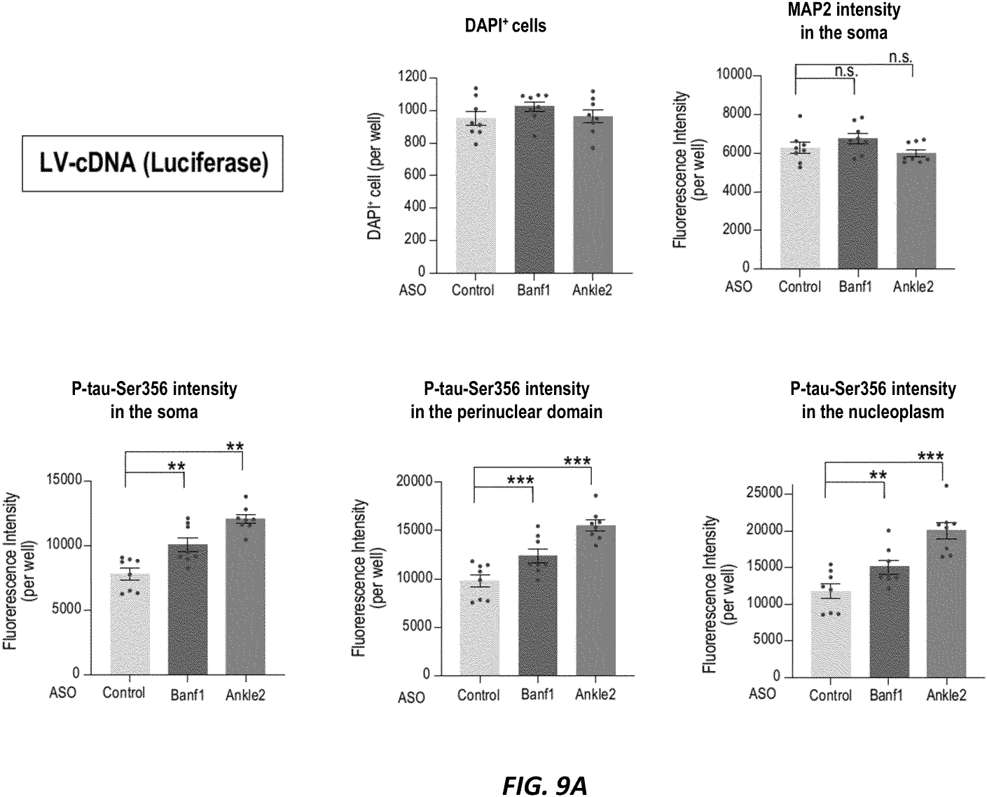

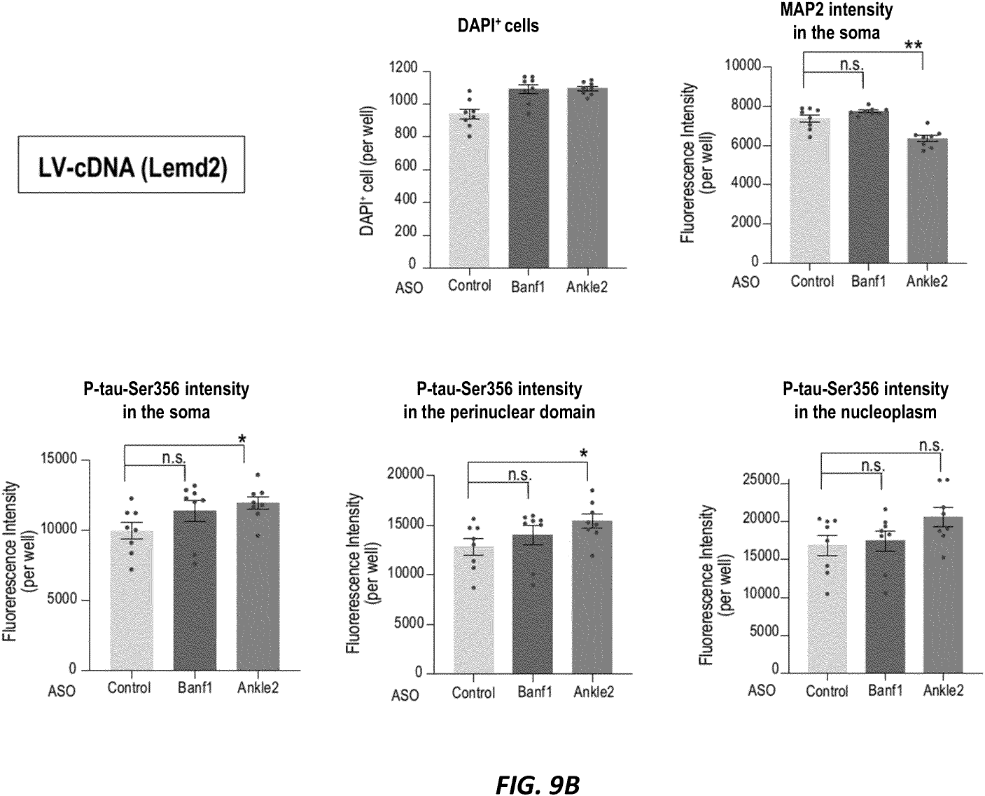

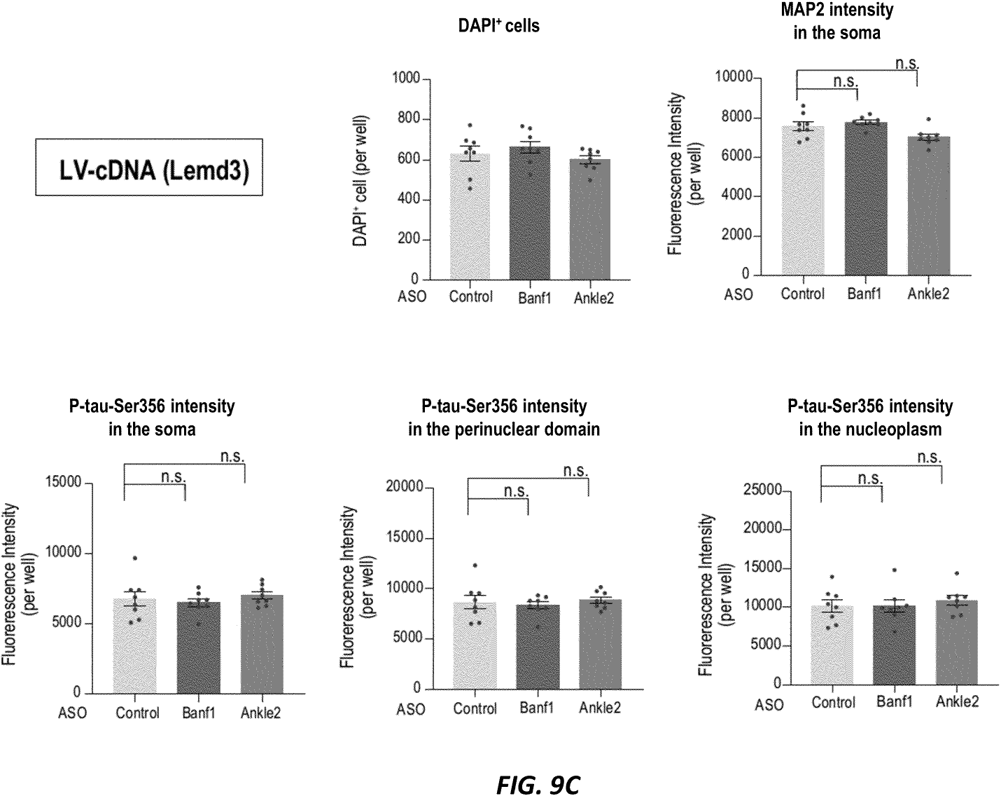

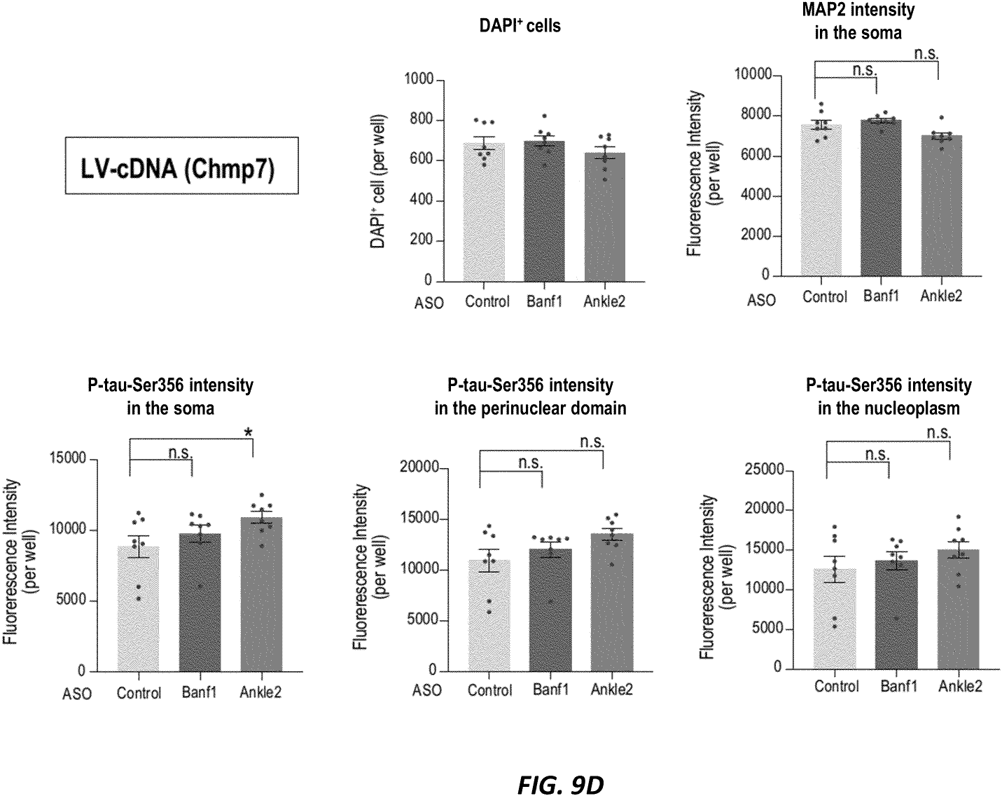

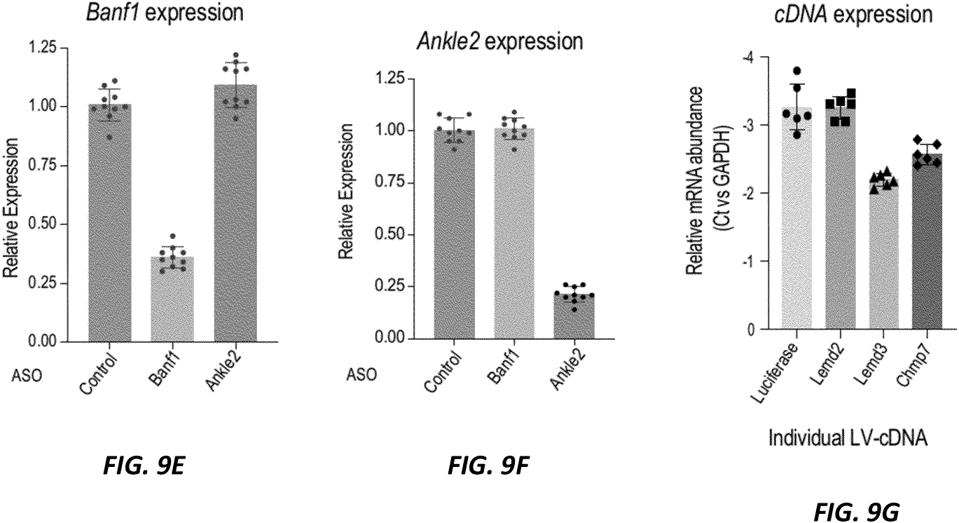

To further confirm the specific, nuclear envelope-related effects of ASO-mediated depletion of Ankle2 and Banf1 in mouse cortical neurons, we recapitulated the rescue experiment conducted in biosensor cells (FIGS. 3A-3D). We treated mouse cortical neurons with control ASO or ASOs targeting Banf1 or Ankle2, together with LV-cDNAs encoding Lemd2, Lemd3, Chmp7, or Luciferase as a control. Depletion of Ankle2 or Banf1 in mouse cortical neurons increases P-tau-Ser356 in the soma, perinuclear domain, and nucleoplasm domains (FIG. 9A). This effect is significantly reduced or eliminated by the concomitant expression of cDNAs encoding Lemd2, Lemd3, or Chmp7 (FIGS. 9B-9D). Knockdown expression of Banf1 or Ankle2 was confirmed by qRT-PCR as well as cDNA expression (FIGS. 9E-9G). Taken together, these results provide strong evidence of an association between nuclear envelope leakage and increased phosphorylation of tau on serine 356, in both biosensor cells and primary cortical neurons. P-tau-Ser356 is a biomarker for tau aggregation, insolubility, and seeding activity (FIGS. 5D and 3C), yet the cortical neurons in these experiments were not treated with a tau seeding agent. Unlike the proliferating HEK293T biosensor cells, which require a seeding event to induce phosphorylation of tau at serine 356 in response to the loss of Banf1, Ankle2, or Ppp2ca, in the post-mitotic cortical neurons, loss of the nuclear envelope components is sufficient to cause a biochemical change in tau that could be a harbinger of pathogenesis.

### Discussion

Most of the studies on tauopathy have focused on the damage to cells and tissues caused by extensive misfolding and aggregation of tau. Cellular and mouse models of disease often employ strong overexpression of mutant tau proteins or the application of high doses of misfolded amyloid fibrils derived from the mutant forms. See, e.g., Allen et al. (2002) J. Neurosci. 22:9340-9351, Frank et al. (2008) Acta Neuropathol. 115:39-53, and Yoshiyama et al. (2007) Neuron 53:337-351, each of which is herein incorporated by reference in its entirety for all purposes. While these types of investigations can model the ravages associated with the late stages of tauopathy disease, they do not necessarily address the root causes. In this study our questions were different. We wanted to get a better understanding of the early events that initiate tau pathology by asking what types of normal cellular processes, pathways, and functions, that when disrupted, could promote tau misfolding and aggregation.

Our first genetic screen employed CRISPR-Cas9 mutagenesis to discover mutations that enhance tau aggregation in a tau biosensor cell line that is exquisitely sensitive to tau seeding agents. See, e.g., Holmes et al. (2014) Proc. Natl. Acad. Sci. U.S.A. 111:E4376-4385 and Sanders et al. (2014) Neuron 82:1271-1288, each of which is herein incorporated by reference in its entirety for all purposes. We found that diluted conditioned medium from cells with stable tau aggregates could, without the assistance of a transfection reagent, induce FRET in a small proportion of cells. When the biosensor cells were mutated in the absence of a seeding agent, we did not find any FRET positive cells. This result implied that, even in a system that is primed for tau aggregation, mutations in single genes that cause spontaneous aggregation are exceedingly rare or nonexistent. When we challenged the mutagenized cells with the conditioned medium weak seeding agent, however, we discovered two genes that when disrupted promoted tau aggregation as indicated by enhanced FRET. One gene, PPP2CA, encodes a catalytic subunit for the 2A family of serine/threonine protein phosphatases that is the main tau phosphatase and has been linked to Alzheimer’s disease. The other gene, BANF1, encodes barrier-to-autointegration factor, a small, abundant, highly conserved DNA-binding protein that has been associated with several key cellular processes. In addition, a homozygous missense mutation in the BANF1 gene has been found in patients diagnosed with progeroid syndrome in which cells exhibited morphological abnormalities of the nuclear envelope. PPP2CA and BANF1 are part of a functional network involved in nuclear envelope maintenance, cycling, and repair (FIGS. 2A-2E, 3A-3H, and 7A-7F). Systematic testing of other members of this network identified a third gene, ANKLE2 (ankyrin repeat and LEM domain-containing protein 2), that when disrupted promotes enhanced FRET in biosensor cells.

BANF1 connects chromatin to the inner membrane of the nuclear envelope and interacts with the nuclear lamina (FIG. 2D). It also binds to the LEM (LAP2/Emerin/MAN1) domains of other protein components of the inner nuclear membrane. In dividing cells, phosphorylation of BANF1 by the VRK1 kinase, a member of BANF1′s interaction network (FIG. 2A), breaks its links with chromatin and the LEM proteins to promote nuclear envelope dissolution prior to entry into mitosis. After the completion of mitosis, ANKLE2 has two functions during nuclear envelope reformation: it both inhibits VRK1 kinase activity and enhances PPP2CA phosphatase activity to promote dephosphorylation of BANF1 so that it can once again connect chromatin to the nuclear envelope through its association with LEM proteins. Besides its documented function in dephosphorylating tau, PPP2CA’s association with Alzheimer’s disease could, in part, be through its involvement with nuclear envelope maintenance and recycling. During interphase, BANF1 facilitates the repair of nuclear envelope ruptures, and coats nuclear DNA at rupture sites to prevent the activation of the cyclic GMP-AMP synthetase-stimulator of interferon genes (cGAS-STING) innate immune pathway.

We confirmed a connection between tau and the nuclear envelope by demonstrating that seed-induced enhancement of tau aggregation upon loss of BANF1 and ANKLE2 could be rescued by overexpression of LEMD2, its related protein LEMD3, or CHMP7, a protein that participates in the repair of nuclear envelope ruptures. We demonstrated that overexpression of LEMD2 not only abolished enhanced tau aggregation as measured by FRET, but also prevented the biochemical accumulation of insoluble tau phosphorylated at serine 356. That LEMD2, LEMD3, and CHMP7 were not hits in the loss-of-function screen for enhanced tau aggregation suggests that their roles in nuclear envelope maintenance might be redundant. Their discovery in the overexpression screen, particularly given CHMP7′s role in nuclear envelope repair, points to more prominent functions for these three proteins in repair of nuclear envelope damage that would otherwise promote tau aggregation. Previous studies have shown connections between tau misfolding and defects in nuclear function. Pathogenic tau aggregation has been linked to disruption of nucleocytoplasmic transport through the nuclear pore complex, the regulatory gatekeeper of traffic into and out of the nucleus. In contrast, the genes identified by our CRISPR-Cas9 screens encode proteins that maintain the basic barrier function of the nuclear envelope. Our biochemical fractionation and immunofluorescence analyses indicate that tau is abundant in the nucleus (FIGS. 3A-3H and 8A-8K), implying an important function in this cellular compartment. Lester et al. also showed that tau is an abundant constituent of the nucleus and that when induced to aggregate, it associates with small nuclear and nucleolar RNAs and with components of nuclear speckles, one being the RNA binding protein SRRM2, which is found mislocalized in the cytoplasm. Lester et al. (2021) Neuron 109(10):1675-1691, herein incorporated by reference in its entirety for all purposes. We reproduced the mislocalization of SRRM2 by reducing the expression of BANF1 or ANKLE2, but in our case, we employed no seeding agents and detected no tau aggregation. Loss of nuclear envelope components alone initiated an event associated with tau pathogenesis in Alzheimer’s disease. Similarly, in primary mouse cortical neurons, a cell type more relevant to tauopathy, we found that disruption of Banf1, Ankle2, or Ppp2ca caused enhanced production of P-tau-Ser356, particularly in the perinuclear and nuclear domains. The induction of this biomarker of tau aggregation, insolubility, and seeding activity in mouse neurons that were not treated with a seeding agent strongly implicated the loss of nuclear envelope integrity as an initiating event for the promotion of tau aggregation and the onset of tau pathogenesis. Abrogation of nuclear envelope function could change the biochemical properties of tau and disrupt retention of nuclear constituents whose appearance in the cytoplasm might nucleate tau aggregation. Post-mitotic neurons, which do not undergo continuous cycles of nuclear envelope disintegration and reassembly, might be more sensitive to events that compromise nuclear envelope integrity.

Most of the interest in tau in neurodegenerative disease has focused on its role in promoting the stability of axonal microtubules. What could be the connection between axonal microtubules and the nuclear envelope? As axons lengthen during normal neuronal growth, there might be communication of this lengthening through the cytoskeleton to the nucleus, causing deformation of the nuclear envelope. Structural modification of the nuclear envelope could alter the connection of BANF1 to chromatin and promote adaptive responses in gene expression. This same type of communication between the axonal microtubules and the nucleus might also cause transient ruptures of the nuclear envelope that release some of the nuclear contents into the cytoplasm. One such constituent could be tau, which upon release from its nuclear depot, could bind to unoccupied sites on the growing microtubules. Both tau’s nucleic acid binding partners in the nucleus and microtubules in the cytoplasm are polyanions that could be bound by tau’s positively charged microtubule binding domain, implying an exchange of binding partners from the nucleus to the cytoplasm. Alternatively, if tau is released from the nucleus as the result of compromised nuclear envelope integrity in the absence of axonal microtubule synthesis, tau would enter the cytoplasm without an available binding partner. This could promote tau misfolding, insolubility, and aggregation. Subsequent axonal microtubule growth could stimulate the release of tau from the nucleus, but it would be met by the previously aggregated form that could seed further tau aggregation and prevent its binding to the newly made microtubules, thereby compromising their stability. Thus, an initial insult to the nuclear envelope could tip the balance between nuclear and cytoplasmic tau toward a dead-end cytoplasmic aggregated form, leading to loss of neuron health and function. Such a scenario could also be imagined for the RNA binding protein TDP-43, a predominantly nuclear component that is found in cytoplasmic aggregates at the end stage of disease in many instances of amyotrophic lateral sclerosis and frontal temporal dementia not associated with mutations in TDP-43. In these cases, like tau, TDP-43 might not have appropriate cytoplasmic RNA binding partners to prevent its misfolding and aggregation.

Our results point to nuclear envelope integrity as a promising new area of investigation in efforts to understand the origins of tauopathy disease and to develop novel therapeutic modalities.

### Materials and Methods

Tau FRET biosensor cell culture. HEK293T tau-CFP/tau-YFP (tau) biosensor cells expressing transgenes encoding tau’s 4RD microtubule binding domains fused to fluorescent reporters CFP or YFP were grown in DMEM culture medium containing DMEM (GIBCO, Cat. 11971-025) with 10% Fetal Bovine Serum (GIBCO, Cat. 16000-036) and with 1% Penicillin/Streptomycin (GIBCO, Cat. 15140-122), and maintained at 37° C. with 5% CO₂.

Primary mouse cortical neuron culture. Wild type primary mouse cortical neurons (MCNs) were purchased from ThermoFisher (GIBCO, Cat. A15586) and following manufacturer’s user guide, cells were plated in Neurobasal Plus Medium (GIBCO, Cat. A35829-01) + 1% B-27 Plus Supplement (GIBCO, Cat. A35828-01) + 1X GlutaMAX Supplement (GIBCO, Cat. 35050-061) at a density of ~20,000 neurons in a volume of 100 µL per well in Poly-D-Lysine treated 96-well plates (Greiner Bio-One, Cat. 655946), and maintained at 37° C. with 5% CO₂. Three days after plating, neurons were transduced with (Cas9 + gRNA) constructs, as mouse targeting gRNAs Banf1_gRNA3, Ankle2_gRNA3, Ppp2ca_gRNA2, or Control gRNA non targeting 303, gNT303, cloned into the pLentiCRISPR-v2 Cas9 expression vector and packaged in lentiviruses (GenScript). After 6 hours, half of Neurobasal medium volume was replaced and replenished every 3-4 days. For ASO treatment, 3 days after plating, MCNs were treated via gymnotic delivery with ASOs targeting Banf1 or Ankle2 or scrambled control ASO (synthesized by IDT). MCNs were maintained in culture for two weeks before expression analysis and fixation for analysis by immunofluorescence.

Production of lentiviral particles. Lentiviral particles are produced following standard LIPOFECTAMINE™-mediated co-transfection of HEK293T cells with the transfer plasmid encoding the gRNA library or the individual gRNA or the Cas9 components (expression vectors, GenScript) with a second-generation packaging plasmid encoding the gag, pol and rev genes and a third plasmid encoding the VSV-G envelope. 24 hours before transfection, HEK293T cells were plated at a density of 10 × 10⁶ cells / plate in 150 mm cell culture dishes in DMEM medium containing DMEM (GIBCO, Cat. 11971-025) with 10% Fetal Bovine Serum (GIBCO, Cat. 16000-036) and with 1% Penicillin/Streptomycin (GIBCO, Cat. 15140-122). On the day of transfection, DMEM medium was replaced with Opti-MEM medium (GIBCO, Cat. 31985-070) supplemented with 25 nM chloroquine (Sigma-Aldrich, Cat. C6628-25G). The DNA mix was prepared by mixing 20 µg of transfer DNA with 20 µg of packaging DNA and 10 µg of envelope DNA in 1.5 mL of Opti-MEM with 60 µL of PLUS™ Reagent (GIBCO, Cat. 11514015). In parallel, 100 µL of LIPOFECTAMINE™ LTX (Life Technologies, Cat. 15338500) was diluted into 1.5 mL of OptiMEM medium. Both mixes were combined for 20 min before addition to cells. The culture medium was changed 6 hours after transfection. Cells were incubated at 37° C. in an incubator with 5% CO₂ atmosphere. After 48 hours, the culture medium containing the lentiviral particles was centrifuged to remove debris and filtered. The supernatant was treated with DNAse to remove residual DNA. The lentiviral batch was concentrated by ultracentrifugation and resuspended in Phosphate Buffered Saline solution (PBS, GIBCO, Cat. 14040-133) overnight. Viral particles were finally aliquoted and stored at -80° C. Lentiviral vectors were titrated using the NucleoSpin RNA Virus kit (Takara, Cat. 740956.250) and the Lenti-X qRT-PCR titration kit (Takara, Cat. 631235). Lentiviral gRNA library particle titers were determined by limiting dilution (adapted protocol from SIGMA Mission RNAi).

Expression and purification of recombinant human MAPT (Q244-E372; P301L, V337M) protein. Recombinant human tau (Q244-E372; P301L, V337M) protein (tau 244-372 LM) was expressed in BL21 (DE3) competent E. coli. Lysis and purification of tau 244-372 LM protein was carried out similar to published reports. Supernatant from the cell lysate was passed over HiTrap SP-Sepharose HP resin to isolate the tau 244-372 LM protein. Peak fractions from this step were then applied to Superdex 75 26/600 column (Cytiva) & eluted in 50 mM HEPES pH 7.0, 50 mM NaCl, 1 mM EDTA, 2.5% glycerol, 10 mM dithiothreitol. SDS-Page analysis showed that the protein was purified to ≥90% homogeneity. The concentration of this material was determined against a standard curve in a colorimetric BCA assay kit from Pierce.

Preparation of recombinant human MAPT (Q244-E372; P301L, V337M) fibrils. A stock concentration of low molecular weight heparin (United States Pharmacopeia catalog #1235820; average molecular weight 4,370 Daltons) was prepared by dissolving Enoxaparin Sodium in Milli-Q water. Recombinant tau 244-372 LM protein at a concentration of 73 µM was mixed with freshly prepared 1 mM dithiothreitol and 18 µM heparin and then transferred to a small polycarbonate container. A 10 mM magnetic Teflon stir bar was added to the vessel. The solution was incubated at 37° C. for 4 days with constant stirring at 750 rpm. Fibrils were harvested by ultracentrifugation at 150,000 rcf for 30 minutes in a Beckman T-55 bio-contained rotor. Following the initial spin, excess heparin & soluble protein were removed from the fibril pellet by carrying out a wash step three times. Briefly, the supernatant was discarded, the fibril pellet was resuspended in several milliliters of 50 mM HEPES, 25 mM NaCl buffer, and then was centrifuged again. Prior to aliquoting, the fibril pellet was resuspended in the same buffer as above and then sonicated 30 seconds at 50% amplitude in a Qsonica cup horn. This step helped to evenly disperse the fibrils in solution and break up any visual clumps.

Characterization of tau aggregation by Thioflavin T fluorescence. Aggregation of the purified, recombinant tau 244-372 LM monomer was monitored by measuring the fluorescence of the amyloid specific dye, Thioflavin T (ThT) at 485 nm (excitation 443 nm). Samples of tau 244-372 LM monomer (50 µM, 25 µM, and 10 µM) were prepared from a 73 µM stock by dilution in buffer (50 mM HEPES pH 7.4, 30 mM NaCl). Each concentration of tau was transferred in triplicate to a black, 96-well microplate along with freshly prepared ThT in twofold molar excess over tau 244-372 LM. Heparin was added to three wells at a molar ratio 1:4 heparin to tau at each concentration (heparin at 12.5 µM, 6.25 µM, and 2.5 µM). Fluorescence was collected over 120 hours in a BMG ClarioStar plate reader. The plate was maintained at a constant 37° C. & underwent constant double-orbital shaking at 700 rpm. Measurements were recorded every 15 minutes through the bottom of the plate.

Transmission electron microscopy of recombinant tau 244-372 LM fibrils. Tau 244-372 LM fibrils were diluted 1:5 in buffer (50 mM HEPES pH 7.4, 25 mM NaCl). Approximately 8-10 µL of sample was applied to the center of 300 mesh formvar-coated copper grids and incubated for 2 minutes undisturbed. Subsequently, remaining liquid was blotted away using filter paper while 10 µL of 1% uranyl acetate stain was applied at the same time. Filter paper was then used again to wick away any excess stain. Images were acquired on a JEOL 1200 EX electron microscope at 80 kV.

Development of stable tau aggregate cell clone as source of tau seeding material. HEK293T tau-YFP cells were plated in a 6-well dish at 500,000 cells per well in DMEM medium. The next day, cells were treated with purified tau 244-372 LM fibrils. Tau fibrils (1 µg) were incubated with LIPOFECTAMINE™ 2000 (Invitrogen, Cat. 11668-019) in Opti-MEM medium for 20 min at room temperature and then added to the well. The following day, cells were passaged with serial dilution into 96-well plates such that each column of the plate received a 2-fold dilution compared to the previous column. Plates were expanded and visually inspected to identify wells containing single colonies. Single cell derived clones were further inspected by fluorescence microscopy to identify clones that contained tau-YFP aggregates, Agg+, in all cells, and which maintained those aggregates over the course of several passages. Three clones were validated to assess their ability to produce tau seeding activity. Briefly, aggregate containing cells were grown to confluency in T175 flasks, medium was changed to fresh DMEM medium, and that medium was then collected after 4 days incubation. This conditioned medium was then centrifuged at 800 rpm for 5 minutes to remove debris, aliquoted, and stored at -80° C. To test tau seeding activity, HEK293T tau-CFP/tau-YFP (tau) biosensor cells were incubated in a mixture of 75% conditioned medium: 25% DMEM medium for 3 days, and then collected for analytical flow cytometry using the CytoFLEX LX (Beckman Coulter) to measure the FRET signal in seeded cells. Tau-YFP Agg+ clone 18 was selected for further expansion based on its retention of aggregates in all cells over many passages, and the ability of clone 18 conditioned medium to consistently induce FRET signal in ~0.1% of tau biosensor cells, that we defined as a minimum tau seeding treatment.

As another source of tau seeding activity, whole cell lysate was collected from tau-YFP Agg+ clone 18. Cells were expanded in six T175 flasks. When reaching confluency, cells were collected by scraping into cold PBS. Total cell pellet was resuspended in 4 ml of fresh PBS with 40 µL of EDTA (ThermoFisher, Cat. 1861283), 40 µL of HALT Protease and Phosphatase Inhibitor Cocktail (ThermoFisher, Cat. 78446) and sonicated for 3 min at 51 Amp using the Qsonica Q500 sonicator. After a final centrifugation, supernatant containing the whole cell lysate was collected, and the protein concentration determined before aliquoting and storage at -80° C.

Selection of Cas9 expressing tau biosensor clones. HEK293T tau-CFP/tau-YFP (tau) biosensor cells were grown in DMEM medium and transduced in 24-well dishes at high MOI (Multiplicity Of Infection) in the presence of Polybrene at 8 µg/mL (Millipore, Cat.TR-1003-G) with the pLentiCas9-Blast vector (GenScript) packaged in lentivirus. After 24 hours, medium was replaced with DMEM medium with 10 µg/mL blasticidin (Invivogen, Cat. ant-bl-1), and cells were grown under selection. At day 3 post-transduction, cells were passaged with serial dilution. Eight expanded single-cell clones were evaluated for both their Cas9 expression level and their cleavage activity. The level of Cas9 mRNA expression was evaluated by isolating RNA with the Quick RNA 96 kit (Zymo Research, Cat. R1053) followed by TaqMan qRT-PCR using the Quantinova One-Step RT-PCR kit (Qiagen, Cat. 208352) with the VG_Cas9U2 TaqMan assay and the reference assay β2M. Samples were run on the QuantStudio real-time PCR system (ThermoFisher). The Cas9 cleavage activity was determined as the percentage of indel alleles after transduction of the PERK gRNA6 at the PERK gene locus. This gRNA was cloned into the pLentiGuide-Puro vector (GenScript), packaged in lentivirus and transduced into the Cas9 clones with three replicate wells. After 24 hours the medium was replaced with DMEM medium with 1.5 µg/mL puromycin (Invivogen, Cat. ant-pr-1). Cells were grown under puromycin selection. Transduced cells were collected at day 3 and day 7, and genomic DNA was extracted using the Blood & Cell Culture DNA Mini Kit (Qiagen, Cat. 13323) for digital PCR analysis (dPCR). The dPCR was performed using the QuantStudio 3D Digital PCR master mix V2 (ThermoFisher, Cat. A26358) with the VIC-labeled Copy number reference assay, human TERT (ThermoFisher, Cat. 4403315) and a FAM-labeled assay targeting the PERK_gRNA6 cutting site. The dPCR reaction was loaded on a QuantStudio 3D Digital PCR 20K Chip v2 (ThermoFisher, Cat. A26316) and carried out using the ProFlex 2X Flat PCR System (ThermoFisher, Cat. 4484078). The emissions of FAM and VIC dyes were analyzed by the QuantStudio 3D Analysis Suite software. The PERK gRNA6 cutting efficiency was determined as a percentage of the FAM/VIC ratio. Clone E was expanded to conduct genome wide CRISPRn screens. See Tables 4-9.

Selection of dCas9-SAM expressing tau biosensor clones. Tau biosensor cells were transduced (as described above) with pLentidCas9-VP64-Blast and pLentiMS2-P65-HSF1-Hyg vectors (GenScript) packaged in lentiviruses. Cells were grown under 5 µg/mL blasticidin and 100 µg/mL hygromycin (Invivogen, Cat. ant-hg-1) selection. At day 3 post-transduction, cells were passaged with serial dilution. Nine single cell expanded clones were evaluated for both their transgene expression levels and gene activation activity. The levels of dCas9, VP64, MS2 and P65 mRNA expression were evaluated by TaqMan qRT-PCR using the assays: Cas9D_VG_SAM, VP64_VG_SAM, MS2_VG_SAM, p65_VG_SAM with the reference β2M. Based on its high expression of both components of the dCas9-SAM system, using the ΔCt method, clone DC11 was expanded for further validation. See Table 8 and Table 9.

Tau biosensor dCas9-SAM clone DC11 was transduced with gRNAs targeting 11 genes (Il1B, LIN28A, UBA52, RANBP1, EEF1A1, ZFP42, PIN1, ATG7, RBM17, DDX42, and STUB1) (FIG. 6E). These genes were selected based on their basal transcription levels in tau biosensor cells, as determined by RNAseq transcriptional profiling analysis, and as shown in a previous report that SAM-mediated fold activation inversely correlate with the basal transcript level. See Konermann et al. (2015) Nature 517:583-588, herein incorporated by reference in its entirety for all purposes. gRNAs were cloned into the pLenti_sgRNA(MS2)_zeo vector (GenScript), and packaged in lentiviruses. gRNAs 1-3 for each gene target were pooled and transduced into DC11 cells, with three replicate wells per target. After 24 hours, the media were replaced with DMEM medium with 600 µg/mL zeocin (Invivogen, Cat. ant-zn-1). At day 7, transduced cells were collected, and mRNA expression analysis performed by TaqMan qRT-PCR using TaqMan Gene Expression Assays (ThermoFisher) with the reference assay β2M. Relative expression was calculated based on ΔΔCt method, and for each assay normalized to the average of the samples transduced with gRNAs targeting the other genes, as a non-targeting control. See Tables 4-9.

Design and cloning of a dCas9-KRAB expression vector. The Streptococcus pyogenes Cas9 nuclease sequence was obtained from National Center for Biotechnology Information (NCBI, accession NP_269215). The nucleic acid sequence was modified to include a Kozak signal, N-terminus nuclear localization signal (NLS), and a C-terminus NLS linker fused to the KRAB domain of human Zinc Finger Protein 10 (accession CAA36558). The nucleic acid sequence was codon optimized using MacVector 18.1.5 and the nuclease cleavage domains were inactivated by incorporating D10A and N863A amino acid substitutions. A 2A peptide was incorporated to support co-expression of dCas9-KRAB and blasticidin-S deaminase from an EF1α promoter. The full sequence was synthesized by GenScript and cloned into a Lentiviral backbone for packaging.

Selection of dCas9-KRAB expressing tau biosensor clones. Tau biosensor cells were transduced with pLentidCas9-KRAB-Blast packaged in lentivirus. Cells were grown with 5 µg/mL blasticidin selection. At day 2 post-transduction, cells were passaged with serial dilution. Ten single cell expanded clones were evaluated for transgene expression. The level of dCas9-KRAB mRNA expression was evaluated by TaqMan qRT-PCR using the VG_Cas9D and 2629_KRAB.P assays and the reference assay β2M. Transcriptional repression activity was determined in three clones exhibiting the highest level of dCas9-KRAB expression. Activity was determined by measuring mRNA levels of four target genes (EGFR, HGF, HSPA8, and NEDD4) after transduction of gRNAs targeting the specific sequences located within 200bp of the transcriptional start sites of these genes. The gRNAs were cloned into the pLentiGuide-Puro vector (GenScript), packaged in lentiviruses, and transduced into the dCas9-KRAB clones. Cells were grown under 1.5 µg/mL puromycin selection and collected at day 7 after transduction for target mRNA expression analysis by TaqMan qRT-PCR using TaqMan Gene Expression Assays (ThermoFisher) and the reference assay β2M. Clone TK-B4 was determined to have the highest level of dCas9-KRAB expression, to trigger the strongest transcriptional repression and was chosen for subsequent validation experiments. See Tables 4-9.

Genome wide CRISPRn screen using the hGeCKO library combined with minimum tau seeding. The hGeCKO-A and B half-libraries combined comprise 111,985 unique single gRNAs targeting 19,050 genes in the human genome (6 gRNAs per gene) and 1,000 non-targeting gRNAs as negative controls. We performed the transduction of the libraries at a low multiplicity of infection (0.3, MOI) to ensure that most cells would receive at most one gRNA construct at a coverage of 300 cells per gRNA and selected for viral vector integration events in the transduced cells by growth in the presence of puromycin. We sampled the transduced cell cultures at three and six days after transduction of the GeCKO library, and then induced tau aggregation by transferring cells to new medium consisting of a 3:1 mixture of Agg+ conditioned medium: fresh growth medium. After four additional days of growth, we harvested the culture and purified FRET+ cells by FACS (FIG. 1B). Cas9-expressing Clone E tau biosensor cells were expanded under blasticidin selection and plated in four T175 flasks at a density of 15 × 10⁶ cells / flask in DMEM medium. At day 0, cells were transduced in DMEM medium in the presence of Polybrene at 8 µg/m with the hGeCKO-A or the hGeCKO-B lentiviral packaged gRNA library at a MOI of 0.3 with a coverage of 300 cells transduced per unique gRNA in the library. After 24 hours, medium was replaced with DMEM medium with 1.5 µg/mL puromycin and 10 µg/mL blasticidin and cells were grown under both blasticidin and puromycin selections. At day 3 after transduction, flasks were washed with PBS and cells detached using a solution of 0.05% Trypsin-EDTA (GIBCO, Cat. 25300-054) neutralized with DMEM medium. Cell suspensions from four T175 flasks were combined and transferred into three T175 flasks with a 1:4 dilution factor in DMEM medium. Remaining cells were counted and ~20 × 10⁶ cells were pelleted for genomic DNA isolation and NGS analysis. At day 6 after transduction, cells were transferred into two T175 flasks with a 1:4 dilution factor (no selection). In each T175 flask, 30 mL of tau-YFP Agg+ Conditioned medium was added to 10 mL of cell suspension. ~20 × 10⁶ cells were also collected for genomic DNA isolation and NGS analysis. At day 10 post transduction, cells were collected for flow cytometry with the MoFlo Astrios Cell Sorter (Beckman Coulter). Cells were pelleted and resuspended in Hanks’ Balanced Salt Solution (HBSS, GIBCO, Cat. 14175-079) with 2% FBS at a concentration of 10 × 10⁶ cells/mL and filtered through a cell-strainer cap tube (Falcon, Cat. 352235). Collection tubes were coated with 80 % FBS and 20 % DMEM medium. FRET positive cells, FRET+, were pelleted for genomic DNA (gDNA) isolation and NGS analysis. The CRISPRn screen was replicated 5 times with both hGeCKO sub-libraries, for a total of 10 genome wide CRISPRn screens.

Next Generation Sequencing. the gRNA libraries were multiplexed and sequenced on NextSeq 500 (Illumina) to generate 1 × 80-base pair (bp) single-end reads. After demultiplexing using bcl2fastq (Illumina), reads were screened for the 16-bp rival vector sequence leading up to the gRNA, and the downstream 20-bp gRNA reads were extracted for the gRNA count. DESeq2 analysis was performed on OmicSoft Studio software version 10.0.1.118 (Qiagen).

Gene-centric enrichment analysis. Fold change for each gene was obtained by calculating the arithmetic mean of the log2 transformed FRET+ day 10 vs day 3 or day 6 ratio generated for corresponding gRNAs by DESEq2. Love et al. (2014) Genome Biol. 15:550, herein incorporated by reference in its entirety for all purposes. The enrichment p value for each gene was calculated as the following. First, in each FRET+ day 10 sample, a gRNA is considered to be present if its DESeq2 method normalized read count is equal or over 30, as we considered read counts below 30 as background noise. The enrichment of the presence of gRNAs corresponding to a gene in a FRET+ day 10 sample is calculated using hypergeometric distribution-based function phyper(x, m, n, k, lower.tail=FALSE) in R (https://www.R-project.org/). The number of present gRNA corresponding to the gene minus 1 is x, the number of all gRNAs corresponding to the gene in the library is m, the total number of gRNA in the library minus m is n, and the total number of gRNA present in the FRET+ sample is k. The p value was then log 10 transformed and averaged across all 10 FRET+ day 10 samples.

Genome wide CRISPRa screen using the hSAM Library combined with maximum tau seeding. dCas9-SAM Clone DC11 tau biosensor cells were expanded under blasticidin and hygromycin selection and plated into four T175 flasks at a density of 23 × 10⁶ cells per flask. At day 0, cells were transduced in DMEM medium in the presence of polybrene at 8 µg/mL with the hSAM lentiviral packaged gRNA library at a MOI of 0.3 with a coverage of 300 cells transduced per unique gRNA in the library. After 24 hours, medium was replaced with DMEM medium with 600 µg/mL zeocin and cells were grown under triple zeocin, blasticidin and hygromycin selections. At day 3 and day 7 after transduction, cells were detached, and cell suspensions from four T175 flasks were combined and transferred into three T175 flasks with a 1:4 dilution factor in DMEM medium. At day 10, cells were detached and transferred into two T175 flasks with a 1:4 dilution factor in DMEM medium (no selection). Maximum seeding treatment consisting of a mix of LIPOFECTAMINE™ 2000 (4 µL/mL of medium) combined with tau-YFP Agg+ whole cell lysate (5 µg/mL of medium) was added directly to the cells. At day 7 and day 10, cell samples were also collected for genomic DNA isolation and NGS analysis. At day 13, cells were collected for flow cytometry with the MoFlo Astrios Cell Sorter to isolate FRET+ and FRET- cells, which were pelleted for DNA isolation and NGS analysis. The genome wide CRISPRa screen was replicated 5 times.

Next Generation Sequencing. Performed on NextSeq (Illumina) by multiplexed single-read run with 80 cycles. Data generated were de-multiplexed using unique index reads. gRNA counts were determined based on perfectly matched sequencing reads of both leader and gRNA sequence.

Differential gRNA read count analysis. For a comparison of gRNA read count between two groups of samples, we used DESeq2 Generalized Linear Model (GLM) and tested for differential abundance using Wald test based on negative binomial distribution in ArrayStudio (OmicSoft). The terms experiment and FRET status were used to build the model for FRET+ vs FRET- comparison among the time point day 13 samples across 5 experiments. gRNAs with significant read count quantity difference between the comparison groups were identified using the criteria of i) fold change ≥ 1.5 in either direction, and ii) p-value < 0.05. The gRNAs enriched in FRET-samples were further defined using these 3 criteria: i) significantly increased in FRET- compared to FRET+ among the day 13 samples, ii) significantly increased in FRET- of day 13 samples compared to day 10 samples, iii) not significantly increased (either no significant difference or significantly decreased) in FRET+ day 13 samples compared to day 10 samples. We paid particular attention to the gene targets that had multiple gRNAs enriched in FRET- samples.

Preparation for NGS of cell samples transduced with -hGeCKO or -hSAM library. Genomic DNAs from cell pellets were extracted using with Blood & Cell Culture DNA Midi kit (Qiagen, Cat. 13343) or the QIAamp DNA mini kit (Qiagen, Cat. 51304). For the hGeCKO library, genomic DNA from day 3 and day 6 cell samples were prepared using a two-step nested PCR strategy. For the first PCR, 130 µg of genomic DNA was amplified per sample to achieve 300 times the coverage of the total unique gRNAs of the library. For each sample, thirteen 100 µL PCR reaction with 10 µg of genomic DNA in each reaction using NEB Next High-Fidelity PCR Master Mix (New England BioLabs, Cat. M0541S) were performed and combined. For the second nested PCR, ten PCR reactions to attach Illumina adaptors and to barcode samples for multiplexing of the NGS runs were performed. Day 10 FRET+ samples (~50,000 cells per sample) were amplified from entire cell population based on 10 µg of DNA amplified per 100 µL first PCR reaction. 5 µL of the first PCR reaction was used for the second PCR, as well as a combination of 9 different forward primers in equimolar amount and a reverse primer with a unique barcode (Table 3). Primers were all used at a final concentration of 0.5 µM. PCR cycles began with initial denaturation at 98° C. for 30 sec; followed by 18 cycles for PCR1 and 15 cycles for PCR2 of denaturation at 98° C. for 10 sec, annealing at 60° C. for 30 sec and extension at 72° C. for 30 sec; and a final extension of 72° C. for 5 min. PCR products were combined and concentrated using DNA Clean & Concentrator kit (Zymo Research, Cat. D4034) and purified on Pippin Prep instrument (DNA Size Selection System, Sage Science) prior submission for NGS. For the hSAM library, 140 µg of genomic DNA of the day 7 and day 10 cell samples were amplified for a 300 times coverage of the SAM gRNA library. Both day 13 FRET+ and FRET- samples were amplified from the entire cell population. Only one step PCR was performed using 2.5 µg of DNA per 100 µL reaction, 10 different forward primers, and a reverse primer with a unique barcode. PCR cycles began with initial denaturation at 98° C. for 30 sec; followed by 26 cycles of denaturation at 98° C. for 10 sec, annealing at 60° C. for 30 sec and extension at 72° C. for 30 sec; and a final extension of 72° C. for 5 min. Combined PCR products were purified prior submission for NGS. See Table 3.

Validation of individual tau modifier targets with minimum tau seeding treatment. Based on the analysis of the CRISPRn screen with the hGeCKO library, 30 gRNAs targeting 14 different candidate genes as well as the control gRNA were purchased (GenScript) and packaged in lentiviruses. Individual gRNAs were transduced into the Cas9 Clone E tau biosensor cells and grown under puromycin selection for 6 days. Three replicate wells were transduced for each gRNA. At day 7, conditioned medium from tau-YFP Agg+ cells or control fresh DMEM medium were added to transduced cells at 75% Conditioned Medium vs 25% DMEM medium. At day 10, cells were collected for analytical flow cytometry using the CytoFLEX LX in order to evaluate the percentage of cells positive for FRET signal. Briefly, cells were washed with PBS and trypsinized to form single-cell suspensions, centrifuged at 800 rpm for 5 minutes and resuspended in 200 µL HBSS + 2% FBS. Cells were filtered with cell-strainer tubes, transferred to a 96-well round-bottom plate, and stained with 0.5 µL of 7-Aminoactinomycin D (7-AAD, ThermoFisher, Cat. A1310) to assess cell viability. Percent FRET+ cells was assessed as the percentage of live, single, CFP+/YFP+ cells that were FRET+. Integrated FRET Density (IFD) was calculated as the product resulting from multiplying the percentage of FRET+ cells by the Median Fluorescence Intensity (MFI) of FRET+ cells. See Tables 4-9.

Determination of gene editing at gRNA cutting sites of BANF1, ANKLE2 and PPP2CA. Cas9 Clone E tau biosensor cells were transduced with BANF1 gRNA1, PPP2CA gRNA5 and control gRNA, and grown under puromycin selection. At day 3 post-transduction, cells were passaged with serial dilution and expanded. Nine single cell derived clones (7 for BANF1 and 2 for PPP2CA) were evaluated for FRET signal in response to seeding by conditioned media from tau-YFP Agg+ and tau-YFP Agg- cells and for the level of gene editing at BANF1 and PPP2CA gRNA cutting sites. This was evaluated by amplifying the region around the cutting sites and performing NGS to characterize the sequence and prevalence of indel alleles. The NGS reads from each clone featuring an indel, as a percentage of total reads, was determined to be the percent of gene editing.

NGS amplicon library prep. Target specific oligos were designed (21-27 base pairs, bp) to generate a maximum amplicon size of 350bp with primer melting temperature (Tm) of 60-65° C. degrees. Barcode adapter sequences were added to the target specific oligo (Table 8 and Table 9) and the full sequence was ordered from Integrated DNA Technologies (IDT). PCR was completed on each DNA sample. Briefly, in each reaction, 4 ng of DNA was combined with IDT oligos, Q5 polymerase (New England Biolabs, Cat. M0491), 10 µM dNTPs, buffer, and water per manufacturer’s specifications. Next, the amplification products were diluted 1:100 and used for the PCR barcoding reaction to create the final sequencing library. Each barcoding reaction contained a single amplified target with a forward and reverse primer containing a unique barcode and index. Each plate of PCRs was pooled in equal volumes and then purified in a single tube using AMPure XP reagent (Beckmann-Coulter, Cat.A63881), per the manufacturer’s instructions. Final library concentration was measured using the Qubit fluorometer (Invitrogen, Cat.Q32866). Four nanomoles of the prepared library was loaded onto the Illumina MiSeq according to the manufacturer’s instruction utilizing the 2×300 read kit (Illumina, Cat.MS-102-3003).

Sequence mapping and characterization. Barcoded samples were de-multiplexed to individual reads (FASTQ format). Forward and reverse reads of each FASTQ file were then merged using PEAR (ncbi.nlm.nih.gov/pmc/articles/PMC3933873/). Merged reads were mapped to the Mus Musculus genome version 9 (mm9) using Bowtie2 (ncbi.nlm.nih.gov/pmc/articles/PMC3322381/). Each sample was sequenced with a minimum of 20,000 merged reads across the expected guide cleavage location. Finally, characterization of barcoded samples was performed using a custom perl script. Briefly, all insertions, deletions, or base changes (INDEL) within a window of 20 bases upstream and downstream of the expected cut site were considered to be CRISPR/Cas9 induced modifications. The number of reads containing an INDEL was compared to the number of reads with wild type sequence to determine the percent editing per animal and tissue.

STRING database search. BANF1 was used as input in the String database search (https://string-db.org). The settings are: minimum required interaction score 0.4 and max number of interaction of first shell <=10.

Gene expression analysis by qRT-PCR. For all other gene expression analysis in tau biosensor cells, RNA was extracted with Zymo Quick RNA 96 kit and samples were diluted to 10 ng/µL. Gene expression analysis was performed by multiplex TaqMan qRT-PCR using the QuantiNova Pathogen + IC Kit (Qiagen, Cat. 208654), in 384-well PCR plates run on QuantStudio thermocyclers (ThermoFisher). TaqMan Gene Expression Assays (ThermoFisher or custom design) were labeled with FAM and the reference assay, GAPDH, was labeled with VIC and could serve as an internal control in each well. See Table 8 and Table 9.

Modifier cDNA cloning into a lentiviral backbone. Based on the analysis of the CRISPRa screen with the hSAM library, cDNA sequences encoding full-length LEMD2, short-isoform LEMD2 (LEMDi2), LEMD3, CHMP7, or Luciferase (Luc), as control cDNA, were cloned in an expression vector and packaged into lentivirus, LV-cDNA. The cDNA and protein sequences were obtained from Ensembl and confirmed in Uniprot. The expression plasmids for the expression of cDNA sequences were generated by synthesizing (GenScript) and subcloning nucleic acid fragments into the pLVX-pEF1α-IRES-Hyg expression vector for the human and control Luciferase cDNAs, and into the pLVX-phSynapsin1-IRES-Hyg expression vector for the mouse and control Luciferase cDNAs (expression vectors developed at Regeneron). The cDNA fragments were inserted downstream of the EF1α promoter by using Spe-I and Not-I restriction sites (GenScript). The nucleic acid sequences were obtained by reverse translation of the protein sequences (Reverse translator tool, MacVector 18.1.5) and codon optimized for expression in human and murine cells. Human protein accession numbers: BANF1 (075531), LEMD2 (Q8NC56), LEMD2-isoform 2 (Q8NC56-2), LEMD3 (Q9Y2U8), CHMP7 (Q8WUX9). Mouse protein accession numbers: Lemd2 (Q6DVA0), Lemd3 (Q9WU40), Chmp7 (Q8R1T1). As control cDNA, the coding sequence of the reporter gene Luciferase was cloned in the same expression vectors. For live-cell imaging studies, NLS::mCherry cDNA was cloned in the same expression vector upstream EF1α promoter. Plasmids were sequence confirmed by Sanger Sequencing, before lentiviral packaging.

Expression of tau modifier genes by transduced cDNAs. To test the effects of LEMD2, LEMDi2, LEMD3, CHMP7, or Luciferase cDNA overexpression on tau aggregation, packaged lentiviruses were transduced into tau biosensor cells. Sequences for the lentiviral constructs are set forth in SEQ ID NOS: 32 and 34-40. After 24 hours the media was replaced with DMEM medium with 50 µg/mL hygromycin, and cells were grown under hygromycin selection. At day 3 post-transduction, transduced cells were seeded with maximum tau seeding treatment as LIPOFECTAMINE™ 2000 (4 µL/mL) and tau-YFP Agg+ cell lysate (5 µg/mL). At day 4, cells were collected for analytical flow cytometry using the CytoFLEX LX to measure the FRET signal in seeded cells. Transduced cells were also collected for cDNA expression analysis by TaqMan qRT-PCR using TaqMan assays designed to amplify specifically the codon optimized cDNAs and the MAPT-4RD transgenes. See FIGS. 6F, 6G, Table 8, and Table 9.

To test the effect of overexpression of these modifier targets on biosensor cells with increased propensity to tau aggregation, cDNAs encoding LEMD2, LEMDi2, LEMD3, CHMP7, or Luciferase were transduced into tau biosensor cells with reduced expression of BANF1 or ANKLE2. Briefly, dCas9-KRAB clone TK-B4 tau biosensor cells were plated in 6-well dishes and transduced, with gRNAs targeting BANF1 (kBANF1_gRNA6), ANKLE2 (kANKLE2_gRNA2) or control gRNA, non-targeting 303, control gNT303, cloned into the pLentiGuide-Puro expression vector and packaged in lentiviruses (GenScript). Cells were grown under both puromycin and blasticidin selection and passaged at Day 3 after transduction into 12-well plates, with each sample duplicated into two wells. At Day 4, cells were transduced with LEMD2, LEMDi2, LEMD3, CHMP7, or Luciferase LV-cDNAs as described above. After 24 hours, the media was replaced with DMEM medium + 1.5 µg/mL puromycin + 50 µg/mL hygromycin. At day 7, cells were passaged into three sets of 24-well plates as well as a set of 6-well for protein cell fractionation (for Luc and LEMD2 cDNA expressing cells). At day 9, DMEM medium was replaced, and minimum tau seeding was added to the wells, as follows: the first set of plates received 1 µg/mL of spinal cord lysate from 9-month-old P301S Het transgenic mouse in each well; the second set of plates received 1 µg/mL of tau-YFP Agg+ whole cell lysate in each well. Confluent cells in 6-well format were treated with 10 µg/mL of cell lysates from tau-YFP Agg+ cells or from tau-YFP Agg- cells. At day 11, cells from the first two sets of plates were collected for analytical flow cytometry using the CytoFLEX LX to measure the FRET signal in seeded cells, and the cells from the third set of plates were collected for extraction of RNA, to assess the expression of transduced cDNA constructs and knockdown of KRAB gRNA target genes by TaqMan qRT-PCR. Cells in 6-well format were collected for protein cell fractionation.

Subcellular protein fractionation combined with Western Blot analysis. Whole cell lysates were prepared from confluent cells on a 6-well plate scraped into 1 mL of ice-cold PBS and centrifuged at 3000 g for 5 minutes. Pellets were resuspended with 150 µL Novex tris-glycine SDS sample buffer (ThermoFisher, Cat. LC2676) and 150 µL of PBS, homogenized with 23G needle and heated at 95° C. for 5 minutes. Protein was quantified with RC DC Protein Assay Kit II (Bio-Rad, Cat. 5000122). Cellular fractionation was performed from 6-well confluent cells using the Subcellular Protein Fractionation Kit for Cultured Cells (ThermoFisher, Cat. 78840), which contains 4 extraction buffers. Following the manufacturer’s protocol, the first buffer added to the cell pellet caused selective membrane permeabilization and released soluble cytoplasmic proteins. The second buffer dissolved plasma, mitochondria and ER-Golgi membranes but did not solubilize the nuclear membranes. After recovering intact nuclei by centrifugation, the third buffer extracted soluble nuclear proteins. An additional nuclear extraction with micrococcal nuclease was performed to release chromatin-bound nuclear proteins. The recovered insoluble pellet was resuspended with the final buffer. The fractions were quantified using the Qubit Protein Assay kit (ThermoFisher, Cat. Q33212). For the Western Blot analysis, Novex tris-glycine SDS sample buffer (2X) was added to each fraction and heated at 95° C. for 5 minutes. 10 µL of protein extract was migrated on a 4-20% Novex tris-glycine wedge well protein gel, and dry transferred onto a nitrocellulose membrane using the Invitrogen iBlot2 system (ThermoFisher). Membranes were blocked with 5% milk in Tris Buffer Saline with 0.05% Tween 20 (TBST) at room temperature for 1 hour. Primary antibodies were incubated overnight at 4° C. on a rocker. Primary antibodies used: tau (Dako, A0024, 1:250,000), phospho-tau Serine 356 (Abcam, ab92682 or ab75603, 1:10,000), BANF1 (Abcam, ab129184, 1:1000), PPP2CA (Proteintech, 13482-1-AP, 1:1000). HRP conjugated Rabbit secondary antibody was added and imaged using SuperSignal West Pico PLUS Chemiluminescent Substrate (ThermoFisher, Cat. 34580) and recorded on a FluorChem M imaging system (ProteinSimple).

Antisense Oligonucleotides. 25 ASOs targeting mouse Banf1 and 96 ASOs targeting mouse Ankle 2 mRNA transcripts (Banf1 transcript ID NM_011793.3, Ankle2 transcript id NM_001253814.1) were designed in silico by scanning through the entire mature mRNA transcripts. All the ASOs were designed using 5-10-5 “gapmer” format where the 5nt “wings” had 2′Methoxy Ethyl (MOE) modifications and the 10nt core had DNA bases to facilitate RNaseH mediated knockdown. Additionally, the ASOs had phosphorothioate (PS) linkages all throughout. To assay for mRNA knockdown, the ASOs were individually transfected at 100 nM in NSC34 cells using LIPOFECTAMINE RNAiMAx as per manufacturer’s recommendation. mRNA knock-down was measured after 72 hours using transcript specific TAQMAN qRT-PCR assays.

6 potent ASOs targeting mouse Banf1 and Ankle2 identified from these screenings were secondary screened in primary mouse cortical neurons to determine the best hits. Gymnotic delivery was obtained by diluting the 1 mM ASO stock solutions (synthesized by IDT) with Neurobasal culture medium to a final ASO concentration of 2.5 µM.

For the NLS::mCherry experiment, neurons were treated with individual ASOs 3 days after plating in a 96-well format (gymnotic delivery). Half of Neurobasal medium volume was replaced 4 days later replenishing ASOs. At day 8 after initial ASO treatment, neurons were transduced with the “EF1α-NLS::mCherry” LV-cDNA construct. Neurons were maintained in culture for 2 more days before live-cell imaging recording and sample collection for expression analysis by TaqMan qRT-PCR.

For the cDNA rescue experiment, neurons were treated 3 days after plating with individual ASOs (gymnotic delivery) at a final concentration of 2.5 µM. 4 days later, neurons were transduced with LV- hSyn1-cDNAs constructs encoding Lemd2, Lemd3, Chmp7, or Luciferase as a control cDNA. After 6 hours, Neurobasal medium was replaced and ASOs replenished. Half of Neurobasal medium volume was replaced 4 days. At day 10, neurons were fixed for immunofluorescence studies. Samples were also collected for expression analysis by TaqMan qRT-PCR.

Culture of mouse cortical neurons. Primary mouse cortical neurons were purchased from ThermoFisher (GIBCO, Cat. A15586) and following manufacturer’s user guide, cells were plated in Neurobasal Plus Medium (GIBCO, Cat. A35829-01) + 1% B-27 Plus Supplement (GIBCO, Cat. A35828-01) + 1X GlutaMAX Supplement (GIBCO, Cat. 35050-061) at a density of ~20,000 neurons in a volume of 100 µL per well in Poly-D-Lysine treated 96-well plates (Greiner Bio-One, Cat.655946). Three days after plating, Neurons were transduced with (Cas9 + gRNA) constructs, as mouse targeting gRNAs Banf1_gRNA3, Ankle2_gRNA3, Ppp2ca_gRNA2, or control non-targeted gRNA NT303, cloned into the pLentiCRISPR-v2 Cas9 expression vector and packaged in lentiviruses (GenScript). After 6 hours, half of Neurobasal medium volume was replaced and replenished every 3-4 days. Neurons were maintained in culture for two weeks before fixation for analysis by Immuno-fluorescence.

Immunofluorescence staining. Primary mouse cortical neurons plated in 96 well plates were prefixed with 2% paraformaldehyde (PFA) (Electron Microscopy Sciences, Cat. 15714) in PBS solution on ice for 5 min. After gentle aspiration, fixation solution was added as 100 µL of cold 4% PFA in PBS for 15 min on ice. Fixation solution was washed three times with 150 µL of cold PBS. Finally, 100 µL of cell permeabilization and blocking solution as 10% Donkey normal serum (DNS) (Sigma-Aldrich, Cat. S30-100ML) in TBS-Triton-0.2% solution, (TBST, Millipore, Cat. 807423) was added for 1 hour at room temperature. Immunostaining was performed in blocking solution with following antibodies: rabbit anti-phospho-tau Ser356 1:1,000 (Abcam, Cat. ab92682), rabbit anti-tau 1:10,000 (Dako, A0024), and chicken anti-MAP2 1:20,000 (Abcam, Cat. ab5392) overnight at 4° C. Next day, each well was washed 3 times with 150 µL of TBST and incubated with 100 µL of secondary antibody solution: Alexa Fluor568 donkey anti-rabbit IgG 1:1000 (Invitrogen, Cat. A10042), Alexa Fluor647 goat anti-chicken IgG 1:1,000 (Invitrogen, Cat. A21449) and DAPI (ThermoFisher, Cat. 62248) in TBST for 1 hour at room temperature. Finally, 3 washes with 150 µL of TBST were performed and 100 µL of PBS was finally added per well. Plates were kept at 4° C. until imaging.

Image analysis. Unbiased image analysis and calculations were performed using the analysis software Harmony 4.9 (PerkinElmer). Immuno-fluorescent imaging was performed using the Opera Phenix High-Content Screening System (PerkinElmer) with a 40x water objective to capture ~40 fields per 96-well. To quantify the percentage of SRRM2 in the nucleus of cells, Harmony was used. DAPI staining was used to label the nucleus, whereas YFP immunostaining to label the cytoplasm. Nuclei were identified using Method C with an area higher than 30 µm² for HEK293T cells. Cytoplasm was defined based on the nuclei (Harmony Method A). The percentage of total SRRM2 intensity (Abcam, Cat. ab11826) in the nucleus was calculated by dividing the nuclear SRRM2 intensity by the sum of the nuclear and cytosolic intensities per image. Significance was determined using an unpaired two-tailed t test.

To quantify phospho-tau Ser356 in cultured mouse cortical neurons, DAPI staining was used to label the nucleus, whereas MAP2 immunostaining to label the soma. Nuclei Detection method C was chosen for robustness with respect to size and fluorescence signal contrast variation of nuclei. Cytoplasm was defined based on the nuclei (Harmony Method A). The perinuclear and soma regions were defined as rings surrounding the DAPI⁺ nuclei cells. For quantification, a minimum of 1,000 cells were analyzed for each condition.

For live-cell imaging recording, we used a Zeiss Axio Observer 7 microscope equipped with incubation components, a colibir 7 LED lightsource and Axiocam 703 Mono sCMOS camera. mCherry fluorescence intensity measurements were performed in Zen Blue (Zeiss) by calculating the mean intensity of three regions of interest with the same area, within the soma in each individual cell.

Statistical analysis of NGS data from CRISPR screens. Gene-centric enrichment analysis. As in DESeq2, our alternative algorithm has 2 components: an enrichment factor (referred to as fold change in DESeq2) and an enrichment p-value. The enrichment factor is similar to that used in DESeq2 except that it is summarized at the gene level by averaging the enrichment of all gRNAs targeting the same gene. Different from existing CRISPR data analysis methods, in our method we used a hypergeometric distribution-based p-value calculation applied only to the enrichment of the different gRNAs targeting a gene in each of the FRET+ day 10 samples. The final enrichment p-value of a gene is the average of the p-values obtained from each of the 10 FRET+ day 10 samples. Fold change for each gene was obtained by calculating the arithmetic mean of the log2 transformed FRET+ day 10 vs day 3 or day 6 ratio generated for corresponding gRNAs by DESEq2 (Love et al., 2014). The enrichment p value for each gene was calculated as the following. First, in each FRET+ day 10 sample, a gRNA is considered to be present if its DESeq2 method normalized read count is equal or over 30, as we considered read counts below 30 as background noise. The enrichment of the presence of gRNAs corresponding to a gene in a FRET+ day 10 sample is calculated using hypergeometric distribution-based function phyper(x, m, n, k, lower.tail=FALSE) in R (R-project.org/). The number of present gRNA corresponding to the gene minus 1 is x, the number of all gRNAs corresponding to the gene in the library is m, the total number of gRNA in the library minus m is n, and the total number of gRNA present in the FRET+ sample is k. The p value was then log 10 transformed and averaged across all 10 FRET+ day 10 samples.

Differential gRNA read count analysis. For a comparison of gRNA read count between two groups of samples, we used DESeq2 Generalized Linear Model (GLM) and tested for differential abundance using Wald test based on negative binomial distribution in ArrayStudio (OmicSoft). The terms experiment, and FRET status were used to build the model for FRET+ vs FRET- comparison among the time point day 13 samples across 5 experiments. gRNAs with significant read count quantity difference between the comparison groups were identified using the criteria of i) fold change ≥ 1.5 in either direction, and ii) p-value < 0.05. The gRNAs enriched in FRET-samples were further defined using these 3 criteria: i) significantly increased in FRET- compared to FRET+ among the day 13 samples, ii) significantly increased in FRET- of day 13 samples compared to day 10 samples, iii) not significantly increased (either no significant difference or significantly decreased) in FRET+ day 13 samples compared to day 10 samples. We paid particular attention to the gene targets that had multiple gRNAs enriched in FRET- samples.

Statistical analysis of immunofluorescence. Statistical analysis was performed in GraphPad Prism 8. Specifically, Mann-Whitney U (two-tailed test) was used to determine significance for immunofluorescence staining and NLS::mCherry fluorescence intensity on the primary mouse cortical neurons. Quantifications are reported as the mean ± SEM (error bars).

TABLE 3

Primers for gRNA Library Sample Preparation.

| Primer (hGecko) | SEQ ID | Primer (hSAM) | SEQ ID |
|---|---|---|---|
| Fwd - 1^{st} Round | 84 | F1 | 110 |
| Rev - 1^{st} Round | 85 | F2 | 111 |
| F01 | 86 | F3 | 112 |
| F02 | 87 | F4 | 113 |
| F03 | 88 | F5 | 114 |
| F04 | 89 | F6 | 115 |
| F05 | 90 | F7 | 116 |
| F06 | 91 | F8 | 117 |
| F07 | 92 | F9 | 118 |
| F08 | 93 | F10 | 119 |
| F09 | 94 | R1 | 120 |
| F10 | 95 | R2 | 121 |
| F11 | 96 | R3 | 122 |
| F12 | 97 | R4 | 123 |
| R01 | 98 | R5 | 124 |
| R02 | 99 | R6 | 125 |
| R03 | 100 | R7 | 126 |
| R04 | 101 | R8 | 127 |
| R05 | 102 | R9 | 128 |
| R06 | 103 | R10 | 129 |
| R07 | 104 | R11 | 130 |
| R08 | 105 | R12 | 131 |
| R09 | 106 | R13 | 132 |
| R10 | 107 | | |
| R11 | 108 | | |
| R12 | 109 | | |

TABLE 4

gRNAs used for the study [Cas9, SAM and KRAB.

| Gene | CRISPR Platform | Targeting | gRNA ID | gRNA Target Sequence | SEQ ID |
|---|---|---|---|---|---|
| Non Targeting | Cas9/dCas9SAM/ dCas9KRAB | Non Targeted | gNT303 | CGCCTCTCACGTGTAGGCTT | 133 |
| Ankle2 | Cas9 | Mouse | gRNA3 | CCAGAACCAATTAGATATCG | 134 |
| ANKLE2 | dCas9KRAB | Human | gRNA2 | GCGGGGCGCGGGCTTCTCGG | 135 |
| ANKLE2 | Cas9 | Human | gRNA3 | AAGGAGCCGCCCCTGTACTA | 136 |
| ATG7 | Cas9 | Human | gRNA1 | AGAAGAAGCTGAACGAGTAT | 137 |
| ATG7 | Cas9 | Human | gRNA2 | CTTGAAAGACTCGAGTGTGT | 138 |
| ATG7 | dCas9SAM | Human | gRNA1 | GAAATCAAAAGAGAGAACGT | 139 |
| ATG7 | dCas9SAM | Human | gRNA2 | GCGTCTCCCTTACCAGATGT | 140 |
| ATG7 | dCas9SAM | Human | gRNA3 | GGAATGTCTACATTCCCTTC | 141 |
| ATP6V1C1 | Cas9 | Human | gRNA1 | AGAGTATCTCGTCACATTAC | 142 |
| ATP6V1C1 | Cas9 | Human | gRNA5 | TCTGCATACAATAACCTGAA | 143 |
| BANF1 | Cas9 | Human | gRNA1 | TTGCAGGCCTATGTTGTCCT | 144 |
| BANF1 | Cas9 | Human | gRNA2 | GCTTCGGATGCCTTCGAGAG | 145 |
| BANF1 | Cas9 | Human | gRNA3 | TTTCCTCCAGCTTCTTGCCC | 146 |
| BANF1 | Cas9 | Human | gRNA6 | CGCCAACGCCAAGCAGTCCC | 147 |
| Banf1 | Cas9 | Mouse | gRNA3 | TTGGTGACGTCCTGAGCAAG | 148 |
| BANF1 | dCas9KRAB | Human | gRNA6 | GCTTGAGGTATCCGCAGGAG | 149 |
| CCDC82 | Cas9 | Human | gRNA2 | TCAGCCTCGTCTAGAGAGCT | 150 |
| CCDC82 | Cas9 | Human | gRNA5 | TCATCAACTTCATCACTGCT | 151 |
| CCDC84 | Cas9 | Human | gRNA3 | CAGACCGAACCACCTCCTGT | 152 |
| CCDC84 | Cas9 | Human | gRNA5 | GAAGAGCCCTCTTCTGGGTC | 153 |
| CHMP7 | Cas9 | Human | gRNA2 | ACAGCTTCTCTCACGCAAAG | 154 |
| DDX42 | dCas9SAM | Human | gRNA1 | GAGGAGCCTGTGAGGCGTTC | 155 |
| DDX42 | dCas9SAM | Human | gRNA2 | TTCATGACCCATCTAGAAAG | 156 |
| DDX42 | dCas9SAM | Human | gRNA3 | TTTAAGGTCAGGCGCCTTCC | 157 |
| DSTN | Cas9 | Human | gRNA1 | GCTTGTATTGCTGAAAAGTT | 158 |
| DSTN | Cas9 | Human | gRNA4 | GCAATTAAAAAGAAATTTCA | 159 |
| EEF1A1 | dCas9SAM | Human | gRNA1 | ACAGTCCCCGAGAAGTTGGG | 160 |
| EEF1A1 | dCas9SAM | Human | gRNA2 | GAAGGTGGCGCGGGGTAAAC | 161 |
| EEF1A1 | dCas9SAM | Human | gRNA3 | GGAGGGGTCGGCAATTGAAC | 162 |
| EGFR | dCas9SAM | Human | gRNA1 | CCACCGCTGTCCACCGCCTC | 163 |
| EMD | Cas9 | Human | gRNA2 | TCCGGCCAGGATCAACTCGT | 164 |
| HGF | dCas9SAM | Human | gRNA3 | GGGGAATGGGGGGGGTTGCA | 165 |
| HSPA8 | dCas9SAM | Human | gRNA3 | TGGGGGCGCATGCGTAGAGG | 166 |
| Il1B | dCas9SAM | Human | gRNA1 | AAAGGGGAAAAGAGTATTGG | 167 |
| Il1B | dCas9SAM | Human | gRNA2 | TGAGATAATTCTCTGGTTCA | 168 |
| Il1B | dCas9SAM | Human | gRNA3 | TGGTGGAAGCTTCTTAGGGG | 169 |
| LEMD2 | Cas9 | Human | gRNA3 | TACTTACGGCTATATATTCT | 170 |
| LEMD3 | Cas9 | Human | gRNA1 | AAGAACGCTTTCTGTTCAAG | 171 |
| LIN28A | dCas9SAM | Human | gRNA1 | TCCGGTCTCTGACACCTCTG | 172 |
| LIN28A | dCas9SAM | Human | gRNA2 | TGTCCCTTCTCAGAACCTTG | 173 |
| LIN28A | dCas9SAM | Human | gRNA3 | TTGTGGGGGAGGGCCGGAGC | 174 |
| MFSD1 | Cas9 | Human | gRNA2 | CTCTGCTCTCTGATCCAAGT | 175 |
| MFSD1 | Cas9 | Human | gRNA3 | TCTAAGATTGAAGCTTTGTT | 176 |
| NEDD4 | dCas9SAM | Human | gRNA1 | CATGGCGTGGGGAGCGCGCG | 177 |
| PARK7 | Cas9 | Human | gRNA1 | AGTACAGTGTAGCCGTGATG | 178 |
| PARK7 | Cas9 | Human | gRNA5 | CTGCACGTCACCTGCACAGA | 179 |
| PERK | Cas9 | Human | gRNA6 | ATCCAGCCTTAGCAAACCAG | 180 |
| PIN1 | dCas9SAM | Human | gRNA3 | TTCGGACCACTCAGGAGCCG | 181 |
| PPP2CA | Cas9 | Human | gRNA5 | GAGCTCTAGACACCAACGTG | 182 |
| PPP2CA | Cas9 | Human | gRNA6 | CAAGCAGCTGTCCGAGTCCC | 183 |
| Ppp2ca | Cas9 | Mouse | gRNA2 | ACATCGAACCTCTTGAACGT | 184 |
| PPP2R2A | Cas9 | Human | gRNA1 | TAGAGTTGTCATCTTTCAAC | 185 |
| PRTFDC1 | Cas9 | Human | gRNA2 | ACGTACCCACAGCACTATTA | 186 |
| PRTFDC1 | Cas9 | Human | gRNA6 | AGGTCTAAAGCCGTCACTTC | 187 |
| RANBP1 | dCas9SAM | Human | gRNA1 | AAGAGGAGCAGATCGGTGGT | 188 |
| RANBP1 | dCas9SAM | Human | gRNA2 | AGGCCACTCCCCAAATGTCC | 189 |
| RANBP1 | dCas9SAM | Human | gRNA3 | CATGGGTCGGGGGCAGTAGG | 190 |
| RBM17 | dCas9SAM | Human | gRNA1 | ACTCTGCCCATGAGCCGCGC | 191 |
| RBM17 | dCas9SAM | Human | gRNA2 | GGCCGGGCGGGCCGGGTAAG | 192 |
| RBM17 | dCas9SAM | Human | gRNA3 | GGGTGGCCTGGCGGGACCTG | 193 |
| RNF7 | Cas9 | Human | gRNA1 | GCGATACGTGCGCCATCTGC | 194 |
| RNF7 | Cas9 | Human | gRNA6 | ATTGTTCTGTTTCACCCACA | 195 |
| SHOX X | Cas9 | Human | gRNA3 | GCATCTGATTCTCTTGTTTG | 196 |
| SHOX X | Cas9 | Human | gRNA4 | CCAAGGTTTGGTTCCAGAAC | 197 |
| STUB1 | dCas9SAM | Human | gRNA1 | GATTGGGCGGCTGCTGGGGG | 198 |
| STUB1 | dCas9SAM | Human | gRNA2 | GGGGCCTCTGCGGATGGGGC | 199 |
| STUB1 | dCas9SAM | Human | gRNA3 | GGGGCCTCTGCTGATGGGGC | 200 |
| SVIL | Cas9 | Human | gRNA1 | TGACGGCTCTTCCGACCCGG | 201 |
| SVIL | Cas9 | Human | gRNA6 | TCAGCTCTTGACCTCGTCTT | 202 |
| TERT | Cas9 | Human | gRNA5 | TGCCTTAGGACCCTGGTCCG | 203 |
| TERT | Cas9 | Human | gRNA6 | CTAGGTGAACAGCCTCCAGA | 204 |
| TMPO | Cas9 | Human | gRNA5 | GTGAAATACGGAGTGAATCC | 205 |
| UBA52 | dCas9SAM | Human | gRNA1 | CGCCCACCCGCTTCCGGTTG | 206 |
| UBA52 | dCas9SAM | Human | gRNA2 | CTCAAGTGACTCGGCGGGCG | 207 |
| UBA52 | dCas9SAM | Human | gRNA3 | GCGGACGCAAACACGGGGAG | 208 |
| UBE2QL1 | Cas9 | Human | gRNA1 | TGGACGAGAGCCTGTTCGAC | 209 |
| UBE2QL1 | Cas9 | Human | gRNA4 | CAGACATCAGCCGTCGGACA | 210 |
| VRK1 | Cas9 | Human | gRNA3 | TTTAAGGAACCCAGTGACAA | 211 |
| ZFP42 | dCas9SAM | Human | gRNA1 | ACTCTCTGCCCGGAGCCGCC | 212 |
| ZFP42 | dCas9SAM | Human | gRNA2 | GCGCGGGCCAGGTGGCTCCA | 213 |
| ZFP42 | dCas9SAM | Human | gRNA3 | GGCCACGCCCTCCCTAACCC | 214 |

TABLE 5

ASOs used for the study

| ASO | SEQ ID NO |
|---|---|
| Control (i.e., scrambled) | 224 |
| Ankle2 | 225 |
| Banf1 | 226 |

TABLE 6

NGS Amplicon Primer Sequences

| Guides | Forward Primer SEQ ID NO | Reverse Primer SEQ ID NO |
|---|---|---|
| hs_ANKLE2_g3 | 230 | 231 |
| hs_BANF1_g1 | 232 | 233 |
| hs_PPP2CA_g5 | 234 | 235 |
| mm_Ankle2_g3 | 236 | 237 |
| mm_Banf1_g3 | 238 | 239 |
| mm_Ppp2ca_g2 | 240 | 241 |

TABLE 7

Guide RNA Locations for NGS

| Gene | Name | Assembly | Chr | Cut Coordinate | Sequence | PAM |
|---|---|---|---|---|---|---|
| ANKLE2 | hs_ANKLE2_g3 | hg19 | chr12 | 133331305 | AAGGAGCCGCCCCUG UACUA (SEQ ID NO: 242) | TGG |
| BANF1 | hs_BANF1_g1 | hg19 | chr11 | 65771107 | UUGCAGGCCUAUGUU GUCCU (SEQ ID NO: 243) | TGG |
| PPP2CA | hs_PPP2CA_g5 | hg19 | chr5 | 133536059 | GAGCUCUAGACACCA ACGUG (SEQ ID NO: 244) | AGG |
| Ankle2 | mm_Ankle2_g3 | mm9 | chr5 | 110666832 | CCAGAACCAAUUAGA UAUCG (SEQ ID NO: 227) | AGG |
| Banf1 | mm_Banf1_g3 | mm9 | chr19 | 5365848 | UUGGUGACGUCCUGA GCAAG (SEQ ID NO: 228) | AGG |
| Ppp2ca | mm_Ppp2ca_g2 | mm9 | chr11 | 51926653 | ACAUCGAACCUCUUG AACGU (SEQ ID NO: 229) | TGG |

TABLE 8

TaqMan Expression Assays.

| cDNA Target | Assay name | Forward Primer SEQ ID NO | Probe SEQ ID NO | Reverse Primer SEQ ID NO |
|---|---|---|---|---|
| PERK | PERK_gRNA6 | 45 | 46 | 47 |
| Cas9 | VG_Cas9U2 | 48 | 49 | 50 |
| \\dCas9-VP64 | Cas9D_VG_SAM | 51 | 52 | 53 |
| dCas9-VP64 | VP64_VG_SAM | 54 | 55 | 56 |
| MS2-P65-HSF1 | MS2_VG_SAM | 57 | 58 | 59 |
| MS2-P65-HSF1 | P65_VG_SAM | 60 | 61 | 62 |
| dCas9-KRAB | 2629_KRAB.P | 63 | 64 | 65 |
| Luciferase | Luc_cDNA_opt | 66 | 67 | 68 |
| human BANF1 | Banf1_opt2 | 69 | 70 | 71 |
| human LEMD2 | hLEMD2_opt2 | 72 | 73 | 74 |
| human LEMD2i2 | hLEMD2_i2_opt2 | 75 | 76 | 77 |
| human LEMD3 | hLEMD3_opt | 78 | 79 | 80 |
| human CHMP7 | CHMP7_opt2 | 81 | 82 | 83 |
| mouse Lemd2 | mLemd2_opt | 215 | 216 | 217 |
| mouse Lemd3 | mLemd3_opt2 | 218 | 219 | 220 |
| mouse Chmp7 iso1 | mChemp7_iso1 | 221 | 222 | 223 |

TABLE 9

ThermoFisher Expression Assays.

| | | |
|---|---|---|
| Gene | ThermoFisher Scientific Catalog ID | |
| MAPT 4RD | hs00902312_m1 | |
| BANF1 | Hs01849026_sl;Hs00427805 g1 | Mm07294182_g1 |
| ANKLE2 | Hs01103914_m1 | Mm01205802_m1 |
| PPP2CA | Hs00427260_m1 | Mm00479816_m1 |
| LEMD2 | Hs04936219_m1 | Mm00524829_m1 |
| LEMD3 | Hs00929680_m1 | Mm03024122_m1 |
| TMPO | Hs07287287_g1 | |
| EMD | Hs02560738_s1 | |
| CHMP7 | Hs01061798_m1 | Mm00513649_m1 |
| VRK1 | Hs00177470_m1 | |
| PPP2R2A | Hs00953658_m1 | |
| β2M | Hs99999907_m1 | |
| IL1B | Hs01555410_m1 | |
| LIN28A | Hs00702808_s1 | |
| UBA52 | Hs03004332_g1 | |
| RANBP1 | Hs01597912_g1 | |
| EEF1A1 | Hs00265885_g1 | |
| ZFP42 | Hs00399279_m1 | |
| PIN1 | Hs01598308_m1 | |
| ATG7 | Hs00197348_m1 | |
| RBM17 | Hs00998713_m1 | |
| DDX42 | Hs00201296_m1 | |
| STUB1 | Hs01071598_g1 | |
| EGFR | Hs01076090_m1 | |
| HGF | Hs00300159_m1 | |
| HSPA8 | Hs03044880_gH | |
| NEDD4 | Hs00406454_m1 | |
| DROSHA | Hs00203008_m1 | |
| GAPDH | Hs99999905_m1 | Mm99999915_g1 |

### Example 2. Overexpression of LEMD2, LEMD3, or CHMP7 as a Therapeutic Modality for Diseases of Tauopathy

We have identified LEMD2, LEMD3 and CHMP7 as modifiers of tau aggregation (see Example 1). We further validated these modifiers using cDNAs that we designed to allow for overexpression in tau biosensor cells. These targets are further validated ex vivo in mouse cortical neurons as well as in vivo using a mouse model of tauopathy.

### Mouse Cortical Neurons

Experiments are performed to assess if the overexpression of mouse LEMD2, LEMD3, or CHMP7 can reduce the hyper-phosphorylation of tau at Ser356, as detected by immunofluorescence in the nuclear and peri-nuclear regions of mouse cortical neurons, treated either with ASOs targeting Banf1 or Ankle2, which induce greater hyper-phosphorylation of tau, or with a non-targeting scrambled ASO control.

Mouse cortical neurons are treated with ASOs targeting Banf1 or Ankle2 or a non-targeting scrambled ASO control and are transduced with AAV (5000 VG/neuron) or lentivirus (LV) (10000 VG/neuron) encoding codon-optimized mouse LEMD2, LEMD3, or CHMP7. Sequences for the AAV constructs are set forth in SEQ ID NOS: 41-43. Sequences for the LV constructs are set forth in SEQ ID NOS: 35, 38, and 40. Expression of mouse LEMD2, LEMD3, or CHMP7 is confirmed by codon-optimized TAQMAN assays specific to the codon-optimized cDNA as compared to the endogenous gene. Phospho-tau is analyzed by immunofluorescence and western blot combined with cell fractionation. Neurons are assessed for evidence of misfolded tau by combining neuron cell lysate with LIPOFECTAMINE™ 2000 and adding treating tau biosensor cells (as a seeding agent) for qualitative FRET analysis.

### Mouse Model of Tauopathy

AAVs encoding mouse LEMD2 or CHMP7 or a mCherry control are injected by intracerebroventricular (ICV) injection in P301S transgenic mouse neonates (P19 mouse model of tauopathy) to determine if overexpression of LEMD2 or CHMP7 can prevent tauopathy-related phenotypes. Sequences for the AAV constructs are set forth in SEQ ID NOS: 41-43.

Multiple AAV titers (5E10, 2.5E10, and 1.25E10) are tested. cDNA expression is followed by qRT-PCR. P301S heterozygote neonates are injected once the optimal AAV titer is determined, and a longitudinal study is run over the course of 6-9 months.

AAVs encoding mouse LEMD2 or CHMP7 or a mCherry control are also injected into 3-month old P301S animals at different AAV titers (1E11 or 5E10 VG/animal). cDNA expression is assessed by qRT-PCR. Once the AAV titer is optimized, P301S heterozygous 3-month old mice are injected, and a longitudinal study is run over the course of 6-9 months.

PS19 mouse model. The PS19 (Tau P301S (Line PS19); PS19Tg; B6;C3-Tg(Prnp-MAPT*P301S)PS19Vle/J) mouse line is a tauopathy model. The genetic background of this strain is C57BL/6 x C3H. PS19 transgenic mice express mutant human microtubule-associated protein tau, MAPT, driven by the mouse prion protein (Prnp) promoter. The transgene encodes the disease-associated P301S mutation and includes four microtubule-binding domains and one N-terminal insert (4R/1N). The transgene inserted at Chr3:140354280-140603283 (Build GRCm38/mm10), causing a 249 Kb deletion that does not affect any known genes. See Goodwin et al. (2019) Genome Res. 29(3):494-505, herein incorporated by reference in its entirety for all purposes. Expression of the mutant human tau is fivefold higher than that of the endogenous mouse protein. See Yoshiyama et al. (2007) Neuron 53(3):337-351, herein incorporated by reference in its entirety for all purposes. PS19 mice develop neuronal loss and brain atrophy by eight months of age. They also develop widespread tau aggregates, known as neurofibrillary tangle-like inclusions, in the neocortex, amygdala, hippocampus, brain stem, and spinal cord. See Yoshiyama et al. (2007). Prior to the appearance of overt tau pathology by histological methods, the brains of these mice were shown to display tau seeding activity. That is, tau aggregates present in brain homogenate can elicit further tau aggregation, presumably via a prion-like mechanism. See Holmes (2014) Proc. Natl. Acad. Sci. U.S.A. 111(41):E4376-E4385, herein incorporated by reference in its entirety for all purposes.

Cloning of cDNAs into lentiviral expression plasmids. The cDNA and protein sequences were obtained from Ensembl (ensembl.org) and confirmed in UniProt. The expression plasmids for the overexpression of cDNA sequences were generated by synthesizing and subcloning nucleic acid fragments into the pLVX-Efla IRES-Hyg expression plasmid vector. The cDNA fragments were inserted downstream of the Efla promoter by using Spe-I and Not-I restriction sites. The nucleic acid sequences were obtained by reverse translation of the protein sequences and codon optimized for expression in Homo sapiens and Mus Musculus, respectively. The protein sequences were obtained from UniProt with the following accession numbers: for Homo sapiens, BANF1 (075531), ANKLE2 (Q86XL3), LEMD2 (Q8NC56), LEMD2-isoform 2 (Q8NC56-2), LEMD3 (Q9Y2U8), and CHMP7 (Q8WUX9); for Mus Musculus, Lemd2 (Q6DVA0), Lemd3 (Q9WU40), and Chmp7-Isoform 1 (Q8R1T1). As a control cDNA, the coding sequence of the reporter gene luciferase was also cloned in the expression vector. Plasmids were sequence confirmed by Sanger sequencing, before lentiviral packaging.

Cloning of cDNAs into AAV expression plasmids. The cDNA and protein sequences were obtained from Ensembl and confirmed in UniProt. The nucleic acid sequences were codon optimized and synthesized into a Lentiviral backbone. PCR was used next to amplify the mouse Lemd2 and mouse Chmp7 cDNAs and add restriction sites for sub-cloning into our AAV backbone using a human synapsin 1 promoter. Specifically, 44 mL Invitrogen AccuPrime Pfx DNA Polymerase (Cat. No. 12344024) was combined with 0.2 mL each of the appropriate forward and reverse oligos at 100 mM concentration and 1 mL template. The PCR reaction was carried out for 29 cycles on Eppendorf’s MasterCycler ProS thermal cycler using the following parameters: 96° 2 min; (96° 20 sec + 50° 30 sec + 72° 45 sec) 29 times; 4° C.

The PCR products were cleaned up using QiaQuick PCR Purification Kit (Cat. No 28106) and then digested with New England BioLabs enzymes EcoRI-HF (Cat. No. R3101L) and NotI-HF (Cat. No. R3189L) in CutSmart buffer (Cat. No. B7204S) following manufacturer’s protocol. The digest products were visualized on an Invitrogen 1.2% eGel (Cat. No. 5018-01) before being cleaned up using QIAquick Spin Columns (Cat. No. 28115). The ssAAV backbone was digested as above and then run on a 1.2% agarose gel (Cat. No. 2120-100GM) for four hours prior to extracting the ssAAV backbone. The DNA fragment was extracted from the agar using QIAquick Spin Columns (Cat. No. 28115). The cDNA fragments were each ligated to the backbone using New England Biolabs T4 DNA Ligase (Cat. No. M0202L 400U/mL) following manufacturer’s protocol. The ligation was transformed into MAX Efficiency Stbl2 Competent Cells (Cat. No. 10268019) following manufacturer’s protocol and then allowed to recover for one hour at 30° C. in Invitrogen S.O.C recovery medium (Cat. No. 15544-034). One third of the recovery was plated on Teknova LB Agar Plates with Carbenicillin-100 (Cat. No. L1010) and grown overnight at 30° C. The resulting colonies were inoculated into five mL of Sigma-Aldrich Terrific Broth (Cat. No. T5574-500 mL) and grown overnight at 30° C. Plasmid DNA was harvested using QiaPrep Spin Miniprep Kit (Cat. No. 27106). Extracted DNA was sequence confirmed by Sanger Sequencing, before AAV packaging using a PhP.eB capsid to cross the blood brain barrier.

### Example 3. Overexpression of LEMD2, LEMD3, or CHMP7 in PS19 Mice

To further study Lemd2, Lemd3, and Chmp7 as genetic modifiers of tau aggregation, and especially as a therapeutical modality for diseases of tauopathy, we designed an experiment to test whether the overexpression of these mouse genes via introduction of cDNAs could delay the onset of tauopathy-related disease phenotypes in mouse models of tauopathy.

We took advantage of the AAV capsid PhP.eB, which enables the delivery of cargos, such as modifier cDNAs, across the blood-brain barrier and into the CNS. We tested two different routes of AAV injection, intraperitoneal (IP) and intracerebroventricular (ICV), in a transgenic mouse model of tauopathy (the PS19 (Tau P301S (Line PS19); PS19Tg; B6;C3-Tg(Prnp-MAPT*P301S)PS19Vle/J) mouse line described above).

The cDNA and protein sequences were obtained from Ensembl and confirmed in UniProt. The plasmids for the expression of cDNA sequences were generated by synthesizing and subcloning nucleic acids into the expression vectors for the mouse and control cDNAs. The nucleic acid sequences were obtained by reverse translation of the protein sequences (Reverse translator tool, MacVector 18.1.5) and codon optimized for expression in murine cells (mouse protein accession numbers: Lemd2 (Q6DVA0), Lemd3 (Q9WU40), and Chmp7 (Q8R1T1)). The coding sequence of the reporter gene mCherry or GFP was cloned in the same expression vectors, to serve as a non-specific control cDNA. Plasmid sequences were confirmed by Sanger Sequencing, before AAV packaging using a plasmid encoding the CNS-specific AAV_PhP.eB capsid. The sequences for the AAV-mLemd2, AAV-mLemd3, and AAV-mChmp7 vectors are set forth in SEQ ID NOS: 249-251, respectively.

We first optimized the titer needed for a robust cDNA expression. We injected mouse neonates (at p0 or p1) by ICV, with different doses of AAV_cDNAs (1.25E10 VG/mouse, 2.5E10 VG/mouse, 5E10 VG/mouse), and sacrificed animals after 4 months. We injected 2-month-old mice by IP, with different doses of AAV_cDNAs (5E10 VG/mouse, 7.5E10 VG/mouse, 1E11 VG/mouse), and sacrificed animals after 4 months. We collected tissues from liver, spinal cord and brain, from which we isolated RNA to conduct a qRT-PCR expression analysis. We showed that cDNA-driven expression was robust and reproducible among animals after ICV injection. The human synapsin-1 promoter allowed for more CNS-specificity in cDNA expression.

Three different therapeutical modalities such as preventing, delaying, and/or curing diseases of tauopathy are assessed. Lemd2, Lemd3, and Chmp7 cDNAs are introduced at birth, before the onset of the disease, at 3 months of age, or after the onset of the disease, at 6 months of age.

For the second round of experiments, PS19 neonates are injected by ICV with 2.5E10 VG/mouse for all of the cDNAs. PS19 heterozygous animals are also injected by IP with 1.5E11 VG/mouse.

A longitudinal study is conducted for the serum neurofilament light chain (sNfL), a biomarker of neuronal damage that is a strong indicator of neurodegenerative processes. To assess sNfL, animals are bled (by the submandibular method) at 4 and 6 months, which are considered as baseline with this mouse model; and thereafter every 5 weeks, in order to observe the levels of sNfL over time.

We claim:

1. A method of inhibiting tau aggregation in a cell, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell.

2. A method of inhibiting or reducing tau phosphorylation in a cell, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell.

3. (canceled)

4. (canceled)

5. A method of inhibiting or reducing accumulation of insoluble tau in a cell, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the cell.

6. The method of claim 1, comprising administering the LEMD2 or the nucleic acid encoding the LEMD2 to the cell.

7. The method of claim 6, wherein the LEMD2 is a human LEMD2, optionally wherein the LEMD2 comprises SEQ ID NO: 1 or 5, and optionally wherein the nucleic acid encoding the LEMD2 comprises SEQ ID NO: 2, 3, 6, or 7.

8. The method of claim 6, wherein the LEMD2 is a mouse LEMD2, optionally wherein the LEMD2 comprises SEQ ID NO: 10, and optionally wherein the nucleic acid encoding the LEMD2 comprises SEQ ID NO: 11, 12, 13, or 255.

9. The method of claim 1, comprising administering the CHMP7 or the nucleic acid encoding the CHMP7 to the cell.

10. The method of claim 9, wherein the CHMP7 is a human CHMP7, optionally wherein the CHMP7 comprises SEQ ID NO: 15, and optionally wherein the nucleic acid encoding the CHMP7 comprises SEQ ID NO: 16 or 17.

11. The method of claim 9, wherein the CHMP7 is a mouse CHMP7, optionally wherein the CHMP7 comprises SEQ ID NO: 19, and optionally wherein the nucleic acid encoding the CHMP7 comprises SEQ ID NO: 20 or 21.

12. The method of claim 1, comprising administering the LEMD3 or the nucleic acid encoding the LEMD3 to the cell.

13. The method of claim 12, wherein the LEMD3 is a human LEMD3, optionally wherein the LEMD3 comprises SEQ ID NO: 23, and optionally wherein the nucleic acid encoding the LEMD3 comprises SEQ ID NO: 24 or 25.

14. The method of claim 12, wherein the LEMD3 is a mouse LEMD3, optionally wherein the LEMD3 comprises SEQ ID NO: 27 or 29, and optionally wherein the nucleic acid encoding the LEMD3 comprises SEQ ID NO: 29 or 30.

15. The method of claim 1, wherein the nucleic acid encoding the LEMD2, the CHMP7, or the LEMD3 is administered to the cell, optionally wherein the nucleic acid is codon-optimized for expression in human cells or mouse cells.

16. The method of claim 15, wherein the nucleic acid comprises a complementary DNA encoding the LEMD2, the CHMP7, or the LEMD3.

17. The method of claim 15, wherein the nucleic acid comprises a messenger RNA encoding the LEMD2, the CHMP7, or the LEMD3.

18. The method of claim 15, wherein the method comprises administering an expression construct comprising the nucleic acid encoding the LEMD2, the CHMP7, or the LEMD3 operably linked to a promoter.

19. The method of claim 18, wherein the promoter is a heterologous promoter.

20. The method of claim 18, wherein the promoter is a constitutive promoter, a tissue-specific promoter, or an inducible promoter.

21. The method of claim 20, wherein the promoter is a neuron-specific promoter.

22. The method of claim 21, wherein the promoter is a synapsin-1 promoter.

23. The method of claim 22, wherein the promoter is a human synapsin-1 promoter.

24. The method of claim 15, wherein the nucleic acid is in a vector.

25. The method of claim 24, wherein the vector is a viral vector.

26. The method of claim 25, wherein the viral vector is a lentivirus vector or an adeno-associated virus (AAV) vector.

27. The method of claim 26, wherein the vector is the AAV vector, optionally wherein the AAV vector is an AAV-PHP.eB vector.

28. The method of claim 1, wherein the cell is a mammalian cell.

29. The method of claim 28, wherein the mammalian cell is a human cell, a rodent cell, a mouse cell, or a rat cell.

30. The method of claim 29, wherein the cell is the human cell.

31. The method of claim 1, wherein the cell is a neuron.

32. The method of claim 1, wherein the cell is in vivo in a subject.

33. The method of claim 32, wherein the cell is a neuron in the brain of the subject.

34. The method of claim 32, wherein the LEMD2, the CHMP7, or the LEMD3 or the nucleic acid encoding the LEMD2, the CHMP7, or the LEMD3 is administered to the subject via intracerebroventricular injection, intracranial injection, or intrathecal injection.

35. The method of claim 1, further comprising assessing one or more signs or symptoms of tauopathy or tau aggregation in the cell.

36. The method of claim 35, further comprising assessing phospho-tau levels in the cell.

37. The method of claim 1, wherein the method reduces the amount of new tau aggregate formation in the cell.

38. The method of claim 1, wherein the method reduces the amount of preexisting tau aggregate formation in the cell.

39. A method of treating a tauopathy in a subject, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the subject, wherein the LEMD2, the CHMP7, or the LEMD3 inhibits tau aggregation in a cell in the subject.

40. A method of preventing a tauopathy in a subject, comprising administering a LEM domain-containing protein 2 (LEMD2), a charged multivesicular body protein 7 (CHMP7), or an inner nuclear membrane protein Man 1 (LEMD3) or a nucleic acid encoding the LEMD2, the CHMP7, or the LEMD3 to the subject, wherein the LEMD2, the CHMP7, or the LEMD3 inhibits tau aggregation in a cell in the subject.

41-73. (canceled)

74. An expression construct comprising a nucleic acid encoding a LEM domain-containing protein 2 (LEMD2), a charged multivesicular body protein 7 (CHMP7), or an inner nuclear membrane protein Man 1 (LEMD3) operably linked to a heterologous promoter.

75-93. (canceled)
